(19) United States
(12) Patent Application Publication
(10) Pub. No.: US 2011/0263647 A1
(43) Pub. Date: Oct. 27, 2011

(54) FLUOROISOQUINOLINE SUBSTITUTED THIAZOLE COMPOUNDS AND METHODS OF USE

(75) Inventors: Qingping Zeng, Newbury Park, CA (US); Chester Chenguang Yuan, Newbury Park, CA (US); Guomin Yao, Camarillo, CA (US); Xianghong Wang, Dublin, CA (US); Seifu Tadesse, Simi Valley, CA (US); David J. ST. Jean, JR., Camarillo, CA (US); Andreas Reichelt, Moorpark, CA (US); Qingyian Liu, Camarillo, CA (US); Fang-Tsao Hong, Thousand Oaks, CA (US); Nianhe Han, Thousand Oaks, CA (US); Christopher H. Fotsch, Thousand Oaks, CA (US); Carl D. Davis, Thousand Oaks, CA (US); Matthew P. Bourbeau, Woodland Hills, CA (US); Kate S. Ashton, Westlake Village, CA (US); John G. Allen, Newbury Park, CA (US)

(73) Assignee: AMGEN INC., Thousand Oaks, CA (US)

(21) Appl. No.: 13/130,255

(22) PCT Filed: Jan. 13, 2010
(86) PCT No.: PCT/US10/20938

§ 371 Date: May 19, 2011

Related U.S. Application Data
(60) Provisional application No. 61/206,346, filed on Jan. 15, 2009.

(51) Int. Cl. A61K 31/4709; C07D 417/14; A61P 35/00; C07D 417/04

(52) U.S. Cl. 514/314; 546/167

(57) ABSTRACT

The invention relates to thiazole compounds of Formula I and compositions thereof useful for treating diseases mediated by protein kinase B (PKB) where the variables have the definitions provided herein.

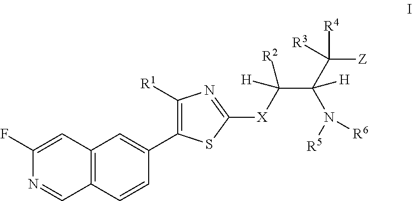

The invention also relates to the therapeutic use of such thiazole compounds and compositions thereof in treating disease states associated with abnormal cell growth, cancer, inflammation, and metabolic disorders.

### CROSS REFERENCES TO RELATED APPLICATIONS

This application claims the benefit of, and priority to, U.S. Provisional Application No. 61/206,346, filed on Jan. 15, 2009, which is hereby incorporated by reference in its entirety and for all purposes as if fully set forth herein.

### FIELD OF THE INVENTION

The invention relates to fluoroisoquinoline substituted thiazole compounds useful for treating diseases mediated by protein kinase B (PKB). The invention also relates to the therapeutic use of such thiazole compounds and compositions thereof in treating disease states associated with abnormal cell growth, cancer, inflammation, and metabolic disorders.

### BACKGROUND OF THE INVENTION

Protein kinases represent a large family of proteins which play a central role in the regulation of a wide variety of cellular processes, maintaining control over cellular function. A partial list of such kinases includes abl, bcr-abl, Blk, Brk, Btk, c-kit, c-met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, flt-1, Fps, Frk, Fyn, GSK3α, GSK3β, Hck, IGF-1R, INS-R, Jak, KDR, Lck, Lyn, MEK, MK2, MSK1, p38, PDGFR, PIK, PKB, PKA, PIM1, PIM2, PRAK, PRK2, PKC, PYK2, P70S6, ROCK2, ros, tie, tie2, TRK, Yes, and Zap70. Inhibition of such kinases has become an important therapeutic approach.

AKT (also known as protein kinase B (PKB) or RAC-PK), including three isoforms AKT1/PKBα/RAC-PKα, AKT2/PKBα/RAC-PKfβ, AKT3/PKBγ/RAC-PKγ, has been identified as a serine/threonine protein kinase. Testa et al., Proc. Natl. Acad. Sci., 2001, 98, 10983-10985; Brazil et al., Trends Biochem Sci., 2001, 11, 657-64; Lawlor et al., J. Cell Sci., 2001, 114, 2903-2910; Cheng, Proc. Natl. Acad. Sci. USA, 1992, 89, 9267-9271; Brodbeck, et al., J. Biol. Chem. 1999, 274, 9133-9136. PKB mediates many effects of IGF-1 and other growth factors on tumor growth and inhibition of apoptosis. Nicholson, et al., Cell. Signal., 2002, 14, 381-395. PKB plays an important role in cell proliferation, apoptosis and response to insulin. For these reasons, modulation of PKBs is of interest in the treatment of tumorigenesis, abnormal cell proliferation, and diabetes.

The molecular structure of the PKBs comprises a regulatory site near the carboxy terminus of the polypeptide, a catalytic domain with an activation loop having a threonine, and an amino-terminal pleckstrin homology domain. The pleckstrin homology domain permits anchorage of the enzyme to the cell membrane through interaction with phospholipids, which triggers the activation of the PKBs. The role of the pleckstrin homology domain requires phosphorylation of phosphatidylinositol at the D-3 position via phosphatidylinositol 3-kinase PI3K, an SH2 domain protein that associates with activated receptor tyrosine kinases, particularly IGF-1R. In particular, phosphoinositol-3-kinase, when activated by receptor tyrosine kinase, catalyzes the synthesis of phosphoinositol-3,4-diphosphate and phosphatidylinositol 3,4,5-triphosphate. The pleckstrin homology domain binds 3-phosphoinositides, which are synthesized by PI3K upon stimulation by growth factors such as platelet derived growth factor (PDGF), nerve growth factor (NGF) and insulin-like growth factor (IGF-1). Kulik et al., Mol. Cell. Biol., 1997, 17, 1595-1606; Hemmings, Science, 1997, 275, 628-630; Datta, et al. Genes Dev., 1999, 13, 2905-2927. Lipid binding to the pleckstrin homology domain promotes translocation of PKB to the plasma membrane. Further activation of PKB occurs by phosphorylation by another protein kinase, PDK1 at Thr308, Thr309, and Thr305 for the PKB isoforms α, β and γ, respectively. A third step of activation is catalyzed by a kinase that phosphorylates Ser473, Ser474 or Ser472 in the C-terminal tails of PKBα, β, and γ respectively. The Ser473 kinase activity has been identified to be associated with plasma membrane and is not due to PKB and PDK1 kinase activity. Hill et al., Current Biology, 2002, 12, 1251-1255; Hresko et al., J. Biol. Chem., 2003, 278, 21615-21622. The process produces the fully activated form of PKB.

Activation of PKB can also occur by inhibiting the D-3 phosphoinositide specific phosphatase, PTEN, which is a membrane-associated FYVE finger phosphatase commonly inactivated in many cancers due to genetic alteration, including prostate cancer. Besson, et al., Eur. J. Biochem., 1999, 263, 605-611; Li, et al., Cancer Res., 1997, 57, 2124-2129.

The catalytic domain of PKB is responsible for the phosphorylation of serine or threonine in the target protein.

Once activated, PKB mediates several cellular functions including proliferation, cell growth, and promotion of survival. Intracoronary, adenovirus-mediated akt gene transfer in heart limits infarct size following ischemia-reperfusion injury in vivo. Miao et al., J. Mol. Cell. Cardiol., 2000, 32, 2397-2402. The antiapoptotic function of PKB is reported to be mediated by its ability to phosphorylate apoptosis regulatory molecules including BAD, caspase 9, IKK-, and the forkhead transcriptional factor FKHRL1. Datta et al., at 2905. PKB signaling is also implicated in the physiological regulation of organ size (Verdu, et al., Nat. Cell Biol., 1999, 1, 500-506), glucose homeostasis (Czech, et al., J. Biol. Chem., 1999, 274, 1865-1868), vasomotor tone (Luo, et al. J. Clin. Invest. 1999, 106, 493-499), and angiogenesis (Kureishi, et al., Nat. Med., 2000, 6, 1004-1010).

Manifestations of altered PKB regulation appear in both injury and disease, the most important role being in cancer. PKB kinase activity is constitutively activated in tumors with PTEN mutation, PI 3-kinase mutation and overexpression, and receptor tyrosine kinase overexpression. PKB is also a mediator of normal cell functions in response to growth factor signaling. Expression of the PKB gene was found to be amplified in 15% of human ovarian carcinoma cases. Cheng, et al., Proc. Natl. Acad. Sci. U.S.A., 1992, 89, 9267-9271. PKB has also been found to be over expressed in 12% of pancreatic cancers. Cheng, et al., Proc. Natl. Acad. Sci. U.S.A., 1996, 93, 3636-3641. In particular, PKBfβ is over-expressed in 12% of ovarian carcinomas and in 50% of undifferentiated tumors, suggesting that PKB may be associated with tumor aggressiveness. Bellacosa, et al., Int. J. Cancer, 1995, 64, 280-285. PKB is also a mediator of normal cell functions. Khwaja, Nature, 1999, 401, 33-34; Yuan, et al., Oncogene, 2000, 19, 2324-2330; Namikawa, et al., J Neurosci., 2000, 20, 2875-2886.

Elucidation of the role of PKB in the increase of growth and inhibition of apoptosis is complicated by the many protein substrates of PKB, including BAD, Forkhead (FOXO family), GSK3, Tuberin (TSC2), p27 Kip1, p21Cip1/WAF1, Raf, Caspase-9, and Mdm2 Lin, et al., Proc. Natl. Acad. Sci. U.S.A., 2001, 98, 7200-7205; Blume-Jensen, et al., Nature 2001, 411, 355-365; Vivanco, et al., Nat. Rev. Cancer, 2002, 2, 489-501.

The various PKBs vary in their abundance in different mammalian cell types. For example, PKBfβ is especially abundant in highly insulin-responsive tissues, including brown fat; PKBα, is widely expressed in most of the tissues; and PKBγ is more abundant in brain and testes.

Modulation of PKB by small molecules can be achieved by identifying compounds that bind to and activate or inhibit one or more PKBs. Cao et al. in United States Publication No. 2004/0122016, published Jun. 24, 2004, disclose certain thiophene derivatives and thiophene analogs as inhibitors of protein kinases. In particular, the disclosure addresses compositions effective as inhibitors of Rho-associated coiled-coil forming protein serine/threonine kinase (ROCK), extracellular signal regulated kinase (ERK), glycogen synthase kinase (GSK), and members of the AGC sub-family of protein kinases. Id. at 4. The AGC sub-family of kinases includes protein kinase A (PKA), PDK, p70^{S6K}-1, p70^{S6K}-2 and PKB. Id.

Triciribine has been reported to inhibit cell growth in PKBfβ overexpressing cells, transformed cells, and was effective at a concentration of 50 nM. Yang et al., Cancer Res., 2004, 64, 4394-4399.

In other work, U.S. Pat. No. 5,232,921, issued Aug. 3, 1993, discloses thiazole derivatives that are active on the cholinergic system. The patent does not address modulation of PKB.

U.S. Patent Publication No. US 2005/0004134, published Jan. 6, 2005, discloses certain thiazole derivatives, a method of obtaining them, and pharmaceutical compositions containing them. The derivatives are described as adenosine antagonists useful in the prevention and/or treatment of cardiac and circulatory disorders, degenerative disorders of the central nervous system, respiratory disorders, and many diseases for which diuretic treatment is suitable.

Derivatives of thiazole were synthesized and used in treating conditions alleviated by antagonism of a 5-HT2b receptor in International Publication No. WO 03/068227. Thiazolyl substituted aminopyrimidines were also made and tested as fungicides in U.S. Patent Publication No. US 2005/0038059, published February, 2005. Derivatives of thiazole were also synthesized by Sanner et al. and indicated to have activity inhibiting cdk5, cdk2, and GSK-3. U.S. Patent Publication No. US 2003/0078252, published Apr. 24, 2003.

Thiadiazole compounds useful for treating diseases mediated by PKB are disclosed in WO 2006/044860, published on Apr. 27, 2006, and in U.S. Patent Publication No. US 2006/0154961, published on Jul. 13, 2006, which is hereby incorporated by reference in its entirety and for all purposes as if fully set forth herein. Thiadiazole compounds substituted with fluoroisoquinoline groups and useful for treating diseases mediated by PKB are disclosed in U.S. patent application Ser. No. 12/218,523, filed on Jul. 15, 2008, which is hereby incorporated by reference in its entirety and for all purposes as if fully set forth herein. Thiazole compounds useful for treating diseases mediated by PKB are disclosed in U.S. Patent Publication No. US 2007/0173506, published on Jul. 26, 2007, which is hereby incorporated by reference in its entirety and for all purposes as if fully set forth herein. Although the compounds disclosed in the references noted above have excellent PKB activity, a need remains for compounds with improved pharmacokinetic, pharmacodynamic, and other properties that improve performance as therapeutics for treating disease states modulated by PKB.

A continued need exists for new compounds that can be used to modulate PKB and can be used to treat various disease conditions associated with PKB. Surprisingly and unexpectedly, the fluoroisoquinoline substituent in the compounds of the present invention provide significant improvements in properties making them excellent therapeutic candidates.

### SUMMARY OF THE INVENTION

This invention encompasses novel compounds useful for treating diseases or conditions mediated by PKB. The invention also encompasses the therapeutic use of such compounds and compositions thereof in the treatment of disease states associated with abnormal cell growth, such as cancer, or metabolic disease states, such as diabetes, or inflammation. The invention further provides pharmaceutical compositions that include the compounds of the invention and the use of the compounds in the preparation of medicaments for treating various conditions and disease states.

In one aspect the invention comprises a compound of Formula I

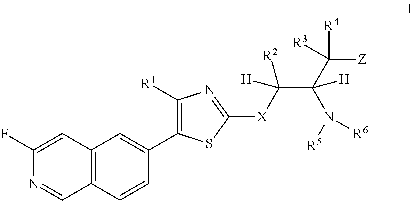

wherein:
X is selected from —N(R^{7a})— or —C(R^{7b}R^{7c})—;
R¹ is —H, halo, —OR⁸, C₁-C₆ alkyl, —(C₁-C₆ alkyl)-O—R⁸, —(C₁-C₆ haloalkyl)-O—R⁸, —(C₂-C₆ alkenyl)-O—R⁸, —(C₁-C₆ alkyl)N(R^{7d})₂, —(C₁-C₆ alkyl)aryl, —C(O)R⁸, —C(O)O—R⁸, —C(O)N(R^{7d})₂, —CHR¹¹—N(H)—R⁸, —CHR¹¹—O—R⁸, C₂-C₆ alkynyl, (C₂-C₆ alkynyl)-O—R⁸, —C≡N, —(C₂-C₆ alkynyl)(C₃-C₈ cycloalkyl), —(C₂-C₆ alkynyl)(C₅-C₈ cycloalkenyl), —(C₂-C₆ alkynyl)-N(R^{7d})S(O)₂—R⁸, aryl, heteroaryl, cycloalkyl, or heterocyclyl;
R² is —H, —OR⁸, —O—(C₁-C₆ alkyl)-O—R⁸, C₁-C₆ alkyl, C₁-C₆ alkenyl, —(C₁-C₆ alkyl)-O—R⁸, or —(C₁-C₆ alkyl)-O—C(O)—R⁸;
R³ is —H, or C₁-C₆ alkyl;
R⁴ is —H, —OR⁸, —O—(C₁-C₆ alkyl)-O—R⁸, C₁-C₆ alkyl, C₁-C₆ alkenyl, —(C₁-C₆ alkyl)-O—R⁸, —(C₁-C₆ alkyl)-O—C(O)—R⁸, —(C₁-C₆ alkyl)-S(O)—R⁸, or —(C₁-C₆ alkyl)-S(O)₂—R⁸;
R⁵ is —H, C₁-C₈ alkyl, —C(O)(CR⁹R¹⁰)ₜ)N(R^{7d})₂, —C(O)(CR⁹R¹⁰)ₜ(CR^{12a}R^{12b}R^{12c}), —C(O)₂(CR⁹R¹⁰)ₜ(CR^{12a}R^{12b}R^{12c}), (CR⁹R¹⁰)ₜ(aryl), —(CR⁹R¹⁰)ₜ(heteroaryl), —(CR⁹R¹⁰)ₜ(cycloalkyl), or —(CR⁹R¹⁰)ₜ(heterocyclyl);
R⁶ is selected from —H, C₁-C₈ alkyl, —(C₁-C₆ alkyl)aryl, or —C(O)(C₁-C₆ alkyl);
R^{7a} is absent if X is —C(R^{7b}R^{7c})— or is selected from —H, C₁-C₈ alkyl, —(C₁-C₆ alkyl)aryl, —C(O)O—(C₁-C₆ alkyl), or —C(O)(C₁-C₆ alkyl);

R^{7b} and R^{7c} are absent if X is —N(R^{7a})— or are independently selected from H and (C₁-C₄)alkyl;

R^{7d} may be absent or, if present, is in each instance selected from —H, C₁-C₈ alkyl, —(C₁-C₆ alkyl)aryl, C₃-C₇ cycloalkyl, or —C(O)(C₁-C₆ alkyl);
R⁸ may be absent or, if present, is selected from —H, C₁-C₆ alkyl, C₁-C₆ haloalkyl, —(C₁-C₆ alkyl)aryl, aryl, heteroaryl, C₁-C₆ hydroxyalkyl, or —(C₁-C₆ alkyl)-O—(C₁-C₆ alkyl), cycloalkyl, or heterocyclyl;
R⁹, R¹⁰ and R¹¹ may be absent or, if present, are independently selected from —H, C₁-C₆ alkyl, or aryl;
R^{12a}, R^{12b}, and R^{12c}, may be absent or, if present, are in each instance independently selected from —H, or C₁-C₆ alkyl;
each t is independently selected from 0, 1, 2, or 3; and
Z is selected from aryl, heteroaryl, C₃-C₇ heterocyclyl comprising 1 or 2 heteroatoms selected from O, S, or N, or a C₃-C₇ cycloalkyl;
wherein each of the above alkyl, aryl, heteroaryl, cycloalkyl, and heterocyclyl moieties are optionally and independently substituted by 1-3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—(C₁-C₆ alkyl);
C₁-C₆ alkyl, C₁-C₆ haloalkyl, C₁-C₆ hydroxyalkyl, C₁-C₆ alkoxy, C₁-C₆ haloalkoxy, C₁-C₆ hydroxyalkoxy, C₁-C₆ alkylamino, C₂-C₆ alkenyl, or C₂-C₆ alkynyl, wherein each of which may be interrupted by one or more hetero atoms,
cyano,
halo,
hydroxyl,
nitro,
oxo,
—NH(CO)—O—(C₁-C₆ alkyl)aryl, —NH(CO)—O—(C₁-C₆ alkyl), —N(C₁-C₆ alkyl)(CO)—O—(C₁-C₆ alkyl)aryl, —N(C₁-C₆ alkyl)(CO)—O—(C₁-C₆ alkyl), —C(O)OH, —C(O)O(C₁-C₆ alkyl), —C(O)NH₂, —C(O)N(H)—(C₁-C₆ alkyl), —C(O)N(C₁-C₆ alkyl)₂, —NH(C₁-C₆ alkyl), —N(C₁-C₆ alkyl)₂, —(C₂-C₄ alkenyl)heterocyclyl, or —(C₂-C₄ alkenyl)cycloalkyl, or
—O-aryl;
   or a pharmaceutically acceptable salt, stereoisomer, or mixture thereof.

In some embodiments, the invention comprises a compound of Formula I, wherein X is —N(R^{7a})—. In some such embodiments, R^{7a} is H

In some embodiments, the invention comprises a compound of Formula I, wherein X is —C(R^{7b}R⁷⁶)—. In some such embodiments, R^{7a} and R^{7c} are both H.

In some embodiments, the invention comprises a compound of Formula I, wherein R¹ is selected from —H, C₁-C₆ alkyl, —(C₁-C₆ alkyl)-O—R⁸, —C(O)O—R⁸, —C(O)N(R^{7d})₂, —CHR¹¹—O—R⁸, or C₂-C₆ alkynyl. In some such embodiments, R¹ is —H. In other such embodiments, R¹ is selected from —CH₂OCH₃, —CH₂OH, —C(O)₂Me, —C(O)N(H)(C₁-C₄ alkyl), —C(O)N(H)(C₃-C₇ cycloalkyl), or —C≡C—CH₃.

In some embodiments, the invention comprises a compound of Formula I, wherein R⁵ and R⁶ are each H.

In some embodiments, the invention comprises a compound of Formula I, wherein R² is H.

In some embodiments, the invention comprises a compound of Formula I, wherein R³ is H.

In some embodiments, the invention comprises a compound of Formula I, wherein R⁴ is —H.

In some embodiments, the invention comprises a compound of Formula I, wherein R⁴ is −OR⁸, —O—(C₁-C₆ alkyl)-O—R⁸, C₁-C₆ alkyl, —(C₁-C₆ alkyl)-O—R⁸, or —(C₁-C₆ alkyl)-S(O)₂—R⁸.

In one embodiment, the invention comprises a compound of Formula I, wherein R⁴ is selected from —CH₃, —CH₂OCH₃, —CH₂OH, —CH₂S(O)₂CH₃, —OH, or —OCH₂OCH₃.

In one embodiment, the invention comprises a compound of Formula I, wherein Z is selected from optionally substituted phenyl, optionally substituted indolyl, optionally substituted naphthyl, optionally substituted pyridinyl, optionally substituted imidazolyl, optionally substituted pyrazolyl, optionally substituted pyrimidinyl, optionally substituted pyridinonyl, optionally substituted thiophenyl, or optionally substituted piperidinyl. In some such embodiments, Z is selected from optionally substituted phenyl and optionally substituted pyridinyl. In some embodiments, Z is selected from phenyl, indolyl, naphthyl, pyridinyl, imidazolyl, pyrazolyl, pyrimidinyl, pyridinonyl, thiophenyl, or piperidinyl, each of which is optionally substituted with 1-3 substituents selected from —Cl, —F, —CF₃, —CF₂CH₃, —CH₃, —CHF₂, or —C(O)O(C₁-C₆ alkyl).

In some embodiments, the invention comprises a compound of Formula I, wherein Z is selected from one of the following groups, wherein the wavy line indicates the point of attachment to the rest of the molecule

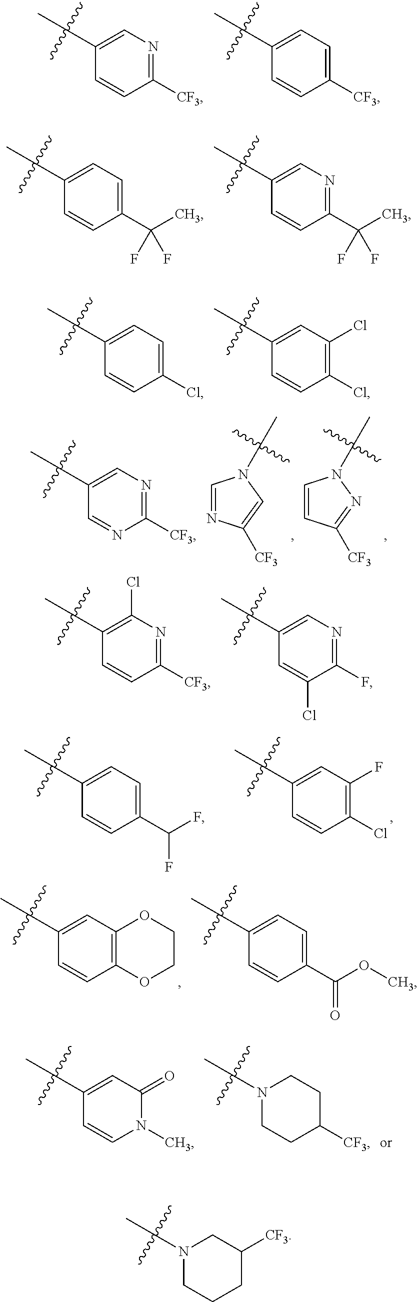

In some embodiments, the compound of Formula I has the Formula IA

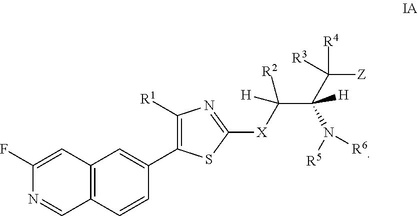

In some embodiments, the compound of Formula I has the Formula IB

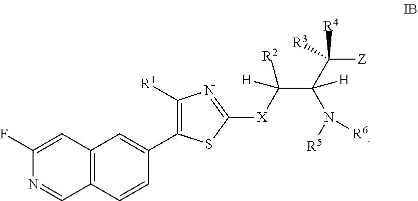

In some embodiments, the compound of Formula I has the Formula IC

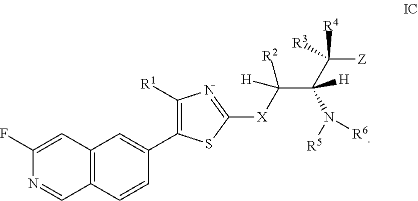

In some embodiments, the compound of Formula I has the Formula ID

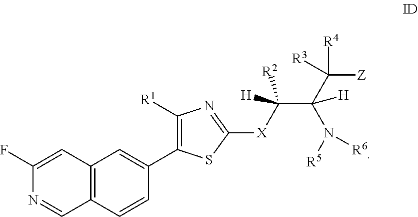

In some embodiments, the compound of Formula I has the Formula IE

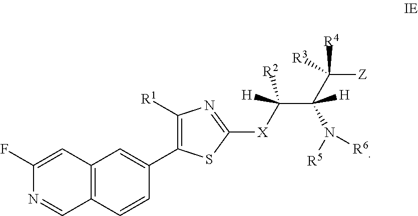

In another aspect, the invention comprises a pharmaceutically acceptable salt of a compound of Formula I. In one embodiment, the pharmaceutically acceptable salt of Formula I is selected from ammonium trifluoroacetate and ammonium chloride.

In another aspect, the invention comprises a pharmaceutical composition comprising a pharmaceutically-acceptable carrier and a compound of Formula I, a compound of any of the embodiments described herein, and/or a salt of any of the compounds of any of the embodiments. In some embodiments, the invention also provides the use of a compound of any of the embodiments in the manufacture of a medicament for carrying out any of the methods of any of the embodiments of the invention. Such compositions and medicaments may further include one or more additional therapeutic agent. Therefore, in some embodiments, the composition or medicament includes at least one additional therapeutic agent.

In another aspect, the invention comprises a method for treating a kinase-mediated disorder in a mammal comprising administering to the mammal a therapeutically effective amount of a compound of Formula I or a pharmaceutical composition of the invention. In some embodiments, the invention provides the use of a compound of Formula I or a pharmaceutical composition of the invention for treating a kinase-mediated disorder in a mammal. The disorder can be one that is mediated by kinases including IGF-1R, Insulin Receptor, KDR, Tie2, EGFR, PKA, PKB, PKC, FKHR, TSC1/2, SGK, LCK, BTK, Erk, MSK, MK2, MSK, p38, P70S6K, PIM1, PIM2, ROCK2, GSK3, or a CDK complex. In some embodiments, the disorder is mediated by PKB, and in some embodiments is mediated by PKBα. In some embodiments, the method comprises selective inhibition of PKB. In some such embodiments, the method comprises selective inhibition of PKBα.

In another embodiment, the invention encompasses Formula I that have selective kinase activity—i.e., they possess significant activity against one specific kinase while possessing less or minimal activity against a different kinase. In some embodiments, the compounds have selective PKB inhibition activity. In some such embodiments, the compounds have selective PKBα inhibition activity. In other embodiments, the invention provides the use of a compound of Formula I or a pharmaceutical composition of the invention for selectively inhibiting a kinase activity. In some embodiments, PKB is selectively inhibited. In some such embodiments, PKBα is selectively inhibited.

In one embodiment, the invention provides a method of treating a proliferation-related disorder in a mammal in need thereof. Such methods include administering to the mammal a therapeutically effective amount of a compound of any of the embodiments described herein or a pharmaceutical composition comprising the compound. Another embodiment of the invention comprises treating abnormal cell growth by administering a therapeutically effective amount of a compound of the invention or a pharmaceutical composition of the invention to a subject in need thereof. In some embodiments, the invention provides the use of a compound of Formula I or a pharmaceutical composition of the invention for treating abnormal cell growth. The abnormal cell growth can be a benign growth or a malignant growth. In particular, the abnormal cell growth can be a carcinoma, sarcoma, lymphoma, or leukemia. In one embodiment of this method, the abnormal cell growth is a cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. The method of the invention also comprises treating a patient having cancer wherein the cancer is selected from the group consisting of small cell lung carcinoma, non-small cell lung carcinoma, esophageal cancer, kidney cancer, pancreatic cancer, melanoma, bladder cancer, breast cancer, colon cancer, liver cancer, lung cancer, sarcoma, stomach cancer, cholangiocarcinoma, mesothelioma, or prostate cancer. In another embodiment of said method, said abnormal cell growth is a benign proliferative disease, including, but not limited to, psoriasis, benign prostatic hypertrophy or restenosis.

In another embodiment, the invention comprises a method of administering a therapeutically effective amount of a Formula I compound to a mammal for treating disease states or conditions selected from diabetes, inflammation, and metabolic disorders. In other embodiments, the invention provides the use of a compound of Formula I or a pharmaceutical composition of the invention for treating a disease state or a condition selected from diabetes, inflammation, and metabolic disorders.

In another embodiment, the invention encompasses a method for treating or preventing cancer in a patient in need thereof, comprising administering to the patient a therapeutically or prophylactically effective amount of a compound according to Formula I and a pharmaceutically acceptable excipient, carrier, or vehicle. In other embodiments, the invention provides the use of a compound of Formula I or a pharmaceutical composition of the invention for treating or preventing cancer in a patient such as in a human cancer patient. In some embodiments, the cancer is a tumor.

In another aspect, the invention encompasses a method for treating or preventing cancer in a patient in need thereof, comprising administering to the patient a therapeutically or prophylactically effective amount of a Formula I compound and at least one additional therapeutic agent.

Further objects, features, and advantages of the invention will be apparent from the following detailed description.

### BRIEF DESCRIPTION OF THE DRAWINGS

FIG. 1 is a graph showing that fluorine substitution in the isoquinoline of the isoquinoline thiazole compounds of the present invention dramatically decreases inhibition of CYP2D6.

FIG. 2 is a graph showing that fluorine substitution in the isoquinoline of the isoquinoline thiazole compounds of the present invention dramatically decreases inhibition of CYP3A4.

FIGS. 3A and 3B are graphs showing that Clearance (CL) is lowered when fluorine is a substituent on the isoquinoline thiazole compounds of the present invention.

FIGS. 4A and 4B are graphs showing that Volume of Distribution (Vss) is not markedly impacted by the presence of fluorine on the isoquinoline thiazole compounds of the present invention.

FIGS. 5A and 5B are graphs showing that IV AUClast is increased when fluorine is a substituent on the isoquinoline thiazole compounds of the present invention.

FIGS. 6A and 6B are graphs showing that oral Cmax is increased when fluorine is a substituent on the isoquinoline thiazole compounds of the present invention.

FIGS. 7A and 7B are graphs showing that Oral AUClast is increased when fluorine is a substituent on the isoquinoline thiazole compounds of the present invention.

### DETAILED DESCRIPTION OF THE INVENTION

### 1.1 Definitions

Where the following terms are used in this specification, they are used as defined below:

The terms “comprising” and “including” are used herein in their open, non-limiting sense.

As used herein, unless otherwise specified, the term “alkyl” means a saturated straight chain or branched non-cyclic hydrocarbon having from 1 to 20 carbon atoms, preferably 1-10 carbon atoms and most preferably 1-4 carbon atoms. Representative saturated straight chain alkyls include, but are not limited to, -methyl, -ethyl, -n-propyl, -n-butyl, -n-pentyl, -n-hexyl, -n-heptyl, -n-octyl, -n-nonyl and -n-decyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted. An alkyl group may be designated as having a certain number of carbon atoms. For example, an alkyl group having from 1 to 8 carbon atoms may be designated as a C₁-C₈ alkyl group whereas an alkyl group having from 1 to 6 carbon atoms may be designated as a C₁-C₆ alkyl group. When such terms are used in conjunction with others such as in the term “—(C₁-C₆ alkyl)aryl”, the “—” symbol indicates the point of attachment to the rest of the molecule, and the term indicates that one of the hydrogens of the alkyl group is replaced by a bond to an aryl group. For example, a —(C₁-C₂ alkyl)aryl includes such groups as —CH₂Ph, —CH₂CH₂Ph, and —CH(Ph)CH₃.

When so designated, an alkyl group can be interrupted by one or more heteroatoms such as N, O, S, or Si atoms. Insertion of a heteroatom in the alkyl group forms a heteroalkyl group. In some embodiments, the heteroatom is a N, O, or S atom. The term “heteroalkyl,” by itself or in combination with another term, means, unless otherwise stated, a stable straight or branched chain radical, or combination thereof, that includes carbon atoms and from one to three heteroatoms selected from the group consisting of O, N, and S. The nitrogen and sulfur atoms may optionally be oxidized, and the nitrogen heteroatom may optionally be quaternized. The heteroatom(s) O, N, and S may be placed at any position in the heteroalkyl group. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, and —CH₂—CH₂—S(O)₂—CH₃. Up to two heteroatoms may be consecutive or adjacent to one another, such as, for example, in —CH₂—NH—OCH₃. When a prefix such as (C₂-C₈) is used to refer to a heteroalkyl group, the number of carbons (2 to 8, in this example) is meant to include the heteroatoms as well. For example, a C₂-heteroalkyl group is meant to include, for example, —CH₂OH (one carbon atom and one heteroatom replacing a carbon atom) and —CH₂SH.

To further illustrate the definition of a heteroalkyl group, where the heteroatom is oxygen, a heteroalkyl group is an oxyalkyl group. For instance, (C₂-C₅)oxyalkyl is meant to include, for example —CH₂—O—CH₃ (a C₃-oxyalkyl group with two carbon atoms and one oxygen replacing a carbon atom), —CH₂CH₂CH₂CH₂OH, and the like.

As used herein, unless otherwise specified, the term “alkenyl” means an unsaturated straight chain or branched non-cyclic hydrocarbon having from 2 to 20 carbon atoms and at least one carbon-carbon double bond. Preferably, an alkenyl has 2 to 10 carbon atoms and most preferably has 2 to 4 carbon atoms. Exemplary straight chain alkenyls include, but are not limited to, -but-3-ene, -hex-4-ene, and -oct-1-ene. Exemplary branched chain alkenyls include, but are not limited to, -2-methyl-but-2-ene, -1-methyl-hex-4-ene, and -4-ethyl-oct-1-ene. An alkenyl group can be substituted or unsubstituted. An alkenyl group may be designated as having a certain number of carbon atoms. For example, an alkenyl group having from 2 to 8 carbon atoms may be designated as a C₂-C₈ alkenyl group whereas an alkenyl group having from 2 to 6 carbon atoms may be designated as a C₂-C₆ alkenyl group.

As used herein, and unless otherwise specified, the term “alkynyl” means an alkyl group in which one or more carbon-carbon single bonds is replaced with an equivalent number of carbon-carbon triple bonds. An alkynyl group must comprise at least two carbon atoms, and can be substituted or unsubstituted. An alkynyl group may be designated as having a certain number of carbon atoms. For example, an alkynyl group having from 2 to 8 carbon atoms may be designated as a C₂-C₈ alkynyl group whereas an alkynyl group having from 2 to 6 carbon atoms may be designated as a C₂-C₆ alkynyl group.

As used herein, the term “halo” means a halogen atom such as a fluorine, chlorine, bromine, or iodine atom (—F, —Cl, —Br, or —I).

As used herein, unless otherwise specified, the term “haloalkyl” means an alkyl group in which one or more hydrogens has been replaced by a halogen atom. A halogen atom is a fluorine, chlorine, bromine, or iodine atom. The number of halogen atoms in a haloalkyl group may range from one to (2 m′+1), where m′ is the total number of carbon atoms in the alkyl group. For example, the term “halo(C₁-C₄)alkyl” is meant to include trifluoromethyl, 2,2,2-trifluoroethyl, 4-chlorobutyl, 3-bromopropyl, and the like. Thus, the term “haloalkyl” includes monohaloalkyl (alkyl substituted with one halogen atom) and polyhaloalkyl (alkyl substituted with halogen atoms in a number ranging from two to (2 m′+1) halogen atoms). The term “perhaloalkyl” means, unless otherwise stated, an alkyl substituted with (2 m′+1) halogen atoms, where m′ is the total number of carbon atoms in the alkyl group. For example, the term “perhalo(C₁-C₄)alkyl”, is meant to include trifluoromethyl, pentachloroethyl, 1,1,1-trifluoro-2-bromo-2-chloroethyl, and the like.

As used herein, the term “cyano” means a —C≡N group.

As used herein, the term “nitro” means a —NO₂ group.

As used herein, the term “oxo” means a ═O group.

As used herein, the terms “hydroxy” and “hydroxyl” mean an —OH group.

As used herein, unless otherwise specified, the term “hydroxyalkyl” means an alkyl group in which one or more hydrogens has been replaced with a hydroxyl group.

As used herein, unless otherwise specified, the term “hydroxyalkenyl” means an alkenyl group in which one or more hydrogens has been replaced with a hydroxyl group.

As used herein, unless otherwise specified, the term “hydroxyalkynyl” means an alkynyl group in which one or more hydrogens has been replaced with a hydroxyl group.

The term “alkoxy” means a structure of the formula —O-alkyl where alkyl has the meaning set forth above.

The term “haloalkoxy” means an alkoxy group in which one or more hydrogen is replaced by a halogen atom.

The term “hydroxyalkoxy” means an alkoxy group in which one or more hydrogen is replaced by a hydroxy group.

The term “alkylsulfonyl” means a structure of the formula —S(O)₂-alkyl.

The term “amino” means an —NH₂ group.

The terms “alkylamino” and “dialkylamino” mean a structure of the formula —NH-alkyl and —N(alkyl)alkyl, respectively, wherein the alkyl is as defined above. The alkyl groups in dialkylamino groups may be the same or different.

The term “alkanoyl”, alone or in combination with another term, means a radical of the type “R—C(O)—” wherein “R” is an alkyl radical as defined above and “—C(O)—” is a carbonyl radical. Examples of such alkanoyl radicals include, but are not limited to, acetyl, trifluoroacetyl, hydroxyacetyl, propionyl, butyryl, valeryl, 4-methylvaleryl, and the like. The terms “alkanoylamino,” and “alkanoyloxy” mean —NH-alkanoyl and —O-alkanoyl, respectively.

The term “alkoxy carbonyl amino” means a structure of the formula —NHC(O)O-alkyl.

The term “alkylsulfonyl amino” means a structure of the general formula —NHS(O)₂-alkyl.

As used herein, the terms “carbocyclic ring system” and “carbocyclic” mean a ring system in which all the ring members are carbon atoms. Carbocyclic ring systems typically include from 3 to 14 ring atoms. Carbocyclic ring systems may be aromatic or may be non-aromatic. Carbocyclic ring systems include cycloalkyl rings and may also include fused ring systems. Examples of fused ring carbocyclic ring systems include, but are not limited to, decalin, norbornane, tetrahydronaphthalene, naphthalene, indene, and adamantane. The ring atoms in a carbocyclic ring system may be substituted or unsubstituted.

As used herein, the terms “heterocyclic ring system”, “heterocyclic” and “heterocyclyl” means a carbocyclic ring system in which at least one ring atom is a heteroatom such as a N, O, S, or Si. In some embodiments, the heterocyclic ring system includes from 1 to 4 heteroatoms. In some embodiments, the heteroatom is selected from N, O, or S. Heterocyclic ring systems may include one ring or may include fused ring systems. By way of nonlimiting example, heterocyclic ring systems may include two six membered rings that are fused to one another or may include one five membered ring and one six membered ring that are fused to one another. Heterocyclic ring systems may be aromatic or may be non-aromatic and may be unsaturated, partially unsaturated, or saturated. The ring atoms in a heterocyclic ring system may be substituted or unsubstituted.

As used herein, unless otherwise specified the term “aryl” means a carbocyclic ring or ring system containing from 6 to 14 ring atoms wherein at least one ring is aromatic. The ring atoms of a carbocyclic aryl group are all carbon atoms. Aryl groups include mono-, bi-, and tricyclic groups as well as benzo-fused carbocyclic moieties such as, but not limited to, 5,6,7,8-tetrahydronaphthyl and the like. In some embodiments, the aryl group is a monocyclic ring or is a bicyclic ring. Representative aryl groups include, but are not limited to, phenyl, tolyl, anthracenyl, fluorenyl, indenyl, azulenyl, phenanthrenyl and naphthyl. An aryl group can be unsubstituted or substituted.

The term “heteroaryl” means an aryl group in which one or more, but not all, of the ring carbon atoms in any ring, whether aromatic or not, is replaced by a hetero atom. For example pyridine is a heteroaryl group as is a compound in which benzene is fused to a nonaromatic ring that includes at least one heteroatom. Exemplary heteroatoms are N, O, and S. In some embodiments, the heteroatoms are N, O, or S. A heteroaryl group can be unsubstituted or substituted. Non-limiting examples of aryl and heteroaryl groups include phenyl, 1-naphthyl, 2-naphthyl, 4-biphenyl, 1-pyrrolyl, 2-pyrrolyl, 3-pyrrolyl, 1-pyrazolyl, 3-pyrazolyl, 5-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furanyl, 3-furanyl, dibenzofuryl, 2-thienyl (2-thiophenyl), 3-thienyl (3-thiophenyl), 2-pyridinyl, 3-pyridinyl, 4-pyridinyl, 2-pyrimidinyl, 4-pyrimidinyl, 5-pyrimidinyl, 3-pyridazinyl, 4-pyridazinyl, 5-benzothiazolyl, 2-benzoxazolyl, 5-benzoxazolyl, benzo[c][1,2,5]oxadiazolyl, purinyl, 2-benzimidazolyl, 5-indolyl, 1H-indazolyl, carbazolyl, la-carbolinyl, β-carbolinyl, γ-carbolinyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl, and 8-quinolyl. Non-limiting examples of other heteroaryl groups include pyridinyl, indazolyl, isoquinolinyl, thiazolopyridinyl, benzothiazolonyl, dihydroquinolinonyl, benzoisoxazolyl, benzooxazolonyl, indolinonyl, benzoimidazolonyl, phthalazinyl, naphthyridinyl, thienopyridinyl, benzodioxolyl, isoindolinonyl, quinazolinyl, or cinnolinyl. The nonaromatic rings in aryl and heteroaryl groups that include nonaromatic rings may be substituted with various groups as described herein including the oxo (═O) group for example in groups such as, but not limited to, the benzo[d]thiazol-2(3H)-onyl group.

The term “cycloalkyl” means an unsaturated or saturated hydrocarbon that forms at least one ring, having from 3 to 20 ring carbon atoms, and in some embodiments, from 3 to 10 ring, from 3 to 8, or from 3 to 6 carbon atoms. The rings in a cycloalkyl group are not aromatic. A cycloalkyl group can be unsubstituted or substituted.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “PKB” refers to protein kinase B, also known as AKT.

The term “treating” refers to:

(i) preventing a disease, disorder, or condition from occurring in a mammal that may be predisposed to the disease, disorder and/or condition, but may not yet have been diagnosed as having it;

(ii) inhibiting the disease, disorder, or condition, i.e., arresting its development; and

(iii) relieving the disease, disorder, or condition, i.e., causing regression of the disease, disorder, and/or condition, or one or more of its symptoms.

The term “preventing” refers to the ability of a compound or composition of the invention to prevent a disease identified herein in mammals diagnosed as having the disease or who are at risk of developing such disease. The term also encompasses preventing further progression of the disease in mammals that are already suffering from or have symptoms of the disease.

The term “mammal” refers to non-human animals or humans.

As used herein, the term “patient” or “subject” means an animal (e.g., cow, horse, sheep, pig, chicken, turkey, quail, cat, dog, mouse, rat, rabbit, guinea pig, etc.) or a mammal, including chimeric and transgenic animals and mammals. In the treatment or prevention of a cancer, the term “patient” or “subject” preferably means a monkey or a human, most preferably a human. In a specific embodiment, the patient or subject is afflicted by a cancer.

As used herein, a “therapeutically effective amount” refers to an amount of a compound of the invention, or prodrug thereof, sufficient to provide a benefit in the treatment or prevention of a condition or disease such as cancer, to delay or minimize symptoms associated with the condition or disease, or to cure or ameliorate the disease or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

As used herein, a “prophylactically effective amount” refers to an amount of a compound of the invention or other active ingredient sufficient to result in the prevention of a condition or disease such as cancer, or recurrence or metastasis of cancer. A prophylactically effective amount may refer to an amount sufficient to prevent initial disease or the recurrence or spread of the disease. The term preferably encompasses a non-toxic amount that improves overall prophylaxis or enhances the prophylactic efficacy of or synergies with another prophylactic or therapeutic agent.

As used herein, “in combination” refers to the use of more than one prophylactic and/or therapeutic agents simultaneously or sequentially. The agents may be selected and administered in such a manner that their respective effects are additive or synergistic.

As used herein, the term “pharmaceutically acceptable salts” refers to salts prepared from pharmaceutically acceptable non-toxic acids or bases including inorganic and organic acids and bases. If the Formula I is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the Formula I compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The neutral forms of the compounds may be regenerated from the salt by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. The term “prodrug” is intended to mean any chemical entity that, after administration, is converted to a different therapeutically effective chemical entity. Prodrugs of the compounds described herein are those compounds that readily undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. Additional examples include peptidyl derivatives of a compound.

As used herein, “solvate” refers to a compound of the present invention or a salt thereof, that further includes a stoichiometric or non-stoichiometric amount of solvent bound by non-covalent intermolecular forces. Where the solvent is water, the solvate is a hydrate.

The compounds of this invention may contain one or more asymmetric centers and thus occur as racemates and racemic mixtures, scalemic mixtures, single enantiomers, individual diastereomers, and diastereomeric mixtures. All such isomeric forms of these compounds are expressly included in the present invention.

As used herein and unless otherwise indicated, the term “optically pure” or “stereomerically pure” means a composition that comprises one stereoisomer of a compound and is substantially free of other stereoisomers of that compound. For example, a stereomerically pure compound having one chiral center will be substantially free of the opposite enantiomer of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al. (1997) Tetrahedron 33:2725; Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

The compounds of the invention may exhibit the phenomenon of tautomerism. While the structural formulas set forth herein cannot expressly depict all possible tautomeric forms, it is to be understood that these structures are intended to represent all tautomeric forms of the depicted compound and are not to be limited merely to the specific compound form depicted by the formula drawings.

Certain compounds of the invention may exist in multiple crystalline or amorphous forms. In general, all physical forms are equivalent for the uses contemplated by the invention and are intended to be within the scope of the invention.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention.

### 1.2 Compounds

The compounds described herein are useful for treating diseases or conditions mediated by various kinases such as PKB. The invention encompasses the therapeutic use of such compounds and compositions thereof in the treatment of disease states associated with abnormal cell growth, such as cancer, or metabolic disease states, such as diabetes, or inflammation. The invention further provides pharmaceutical compositions that include the compounds of the invention and the use of the compounds in the preparation of medicaments or pharmaceutical formulations or compositions for treating various conditions and disease states.

In one aspect the invention comprises a compound of Formula I

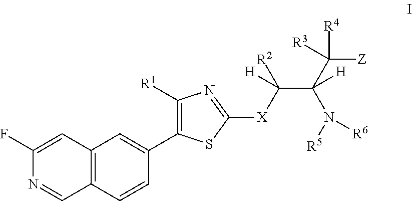

wherein:
X is selected from —N(R^{7a})— or —C(R^{7b}R^{7e})—;
R¹ is —H, halo, —OR⁸, C₁-C₆ alkyl, —(C₁-C₆ alkyl)-O—R⁸, —(C₁-C₆ haloalkyl)-O—R⁸, —(C₂-C₆ alkenyl)-O—R⁸, —(C₁-C₆ alkyl)N(R^{7d})₂, —(C₁-C₆ alkyl)aryl, —C(O)R⁸, —C(O)O—R⁸, —C(O)N(R^{7d})₂, —CHR¹¹—N(H)—R⁸, —CHR¹¹—O—R⁸, C₂-C₆ alkynyl, (C₂-C₆ alkynyl)-O—R⁸, —C≡N, —(C₂-C₆ alkynyl)(C₃-C₈ cycloalkyl), —(C₂-C₆ alkynyl)(C₅-C₈ cycloalkenyl), —(C₂-C₆ alkynyl)-N(R^{7d})S(O)₂—R⁸, aryl, heteroaryl, cycloalkyl, or heterocyclyl;
R² is —H, —OR⁸, —O—(C₁-C₆ alkyl)-O—R⁸, C₁-C₆ alkyl, C₁-C₆ alkenyl, —(C₁-C₆ alkyl)-O—R⁸, or —(C₁-C₆ alkyl)-O—C(O)—R⁸;
R³ is —H, or C₁-C₆ alkyl;
R⁴ is —H, —OR⁸, —O—(C₁-C₆ alkyl)-O—R⁸, C₁-C₆ alkyl, C₁-C₆ alkenyl, —(C₁-C₆ alkyl)-O—R⁸, —(C₁-C₆ alkyl)-O—C(O)—R⁸, —(C₁-C₆ alkyl)-S(O)—R⁸, or —(C₁-C₆ alkyl)-S(O)₂—R⁸;
R⁵ is —H, C₁-C₈ alkyl, —C(O)(CR⁹R¹⁰)ₜ)N(R^{7d})₂, —C(O)(CR⁹R¹⁰)ₜ(CR^{12a}R^{12b}R^{12c}), —C(O)₂(CR⁹R¹⁰)(CR^{12a}R^{12b}R^{12c}), (CR⁹R¹⁰)ₜ(aryl), —(CR⁹R¹⁰)ₜ(heteroaryl), —(CR⁹R¹⁰)ₜ(cycloalkyl), or —(CR⁹R¹⁰)ₜ(heterocyclyl);
R⁶ is selected from —H, C₁-C₈ alkyl, —(C₁-C₆ alkyl)aryl, or —C(O)(C₁-C₆ alkyl);
R^{7a} is absent if X is —C(R^{7b}R^{7c})— or is selected from —H, C₁-C₈ alkyl, —(C₁-C₆ alkyl)aryl, —C(O)O—(C₁-C₆ alkyl), or —C(O)(C₁-C₆ alkyl);
R^{7b} and R^{7c} are absent if X is —N(R^{7a})— or are independently selected from H and (C₁-C₄)alkyl;
R^{7d} may be absent or, if present, is in each instance selected from —H, C₁-C₈ alkyl, —(C₁-C₆ alkyl)aryl, C₃-C₇ cycloalkyl, or —C(O)(C₁-C₆ alkyl);
R⁸ may be absent or, if present, is selected from —H, C₁-C₆ alkyl, C₁-C₆ haloalkyl, —(C₁-C₆ alkyl)aryl, aryl, heteroaryl, C₁-C₆ hydroxyalkyl, or —(C₁-C₆ alkyl)-O—(C₁-C₆ alkyl), cycloalkyl, or heterocyclyl;
R⁹, R¹⁰ and R¹¹ may be absent or, if present, are independently selected from —H, C₁-C₆ alkyl, or aryl;
R^{12a}, R^{12b}, and R^{12c}, may be absent or, if present, are in each instance independently selected from —H, or C₁-C₆ alkyl;
each t is independently selected from 0, 1, 2, or 3; and
Z is selected from aryl, heteroaryl, C₃-C₇ heterocyclyl comprising 1 or 2 heteroatoms selected from O, S, or N, or a C₃-C₇ cycloalkyl;
wherein each of the above alkyl, aryl, heteroaryl, cycloalkyl, and heterocyclyl moieties are optionally and independently substituted by 1-3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—(C₁-C₆ alkyl);
C₁-C₆ alkyl, C₁-C₆ haloalkyl, C₁-C₆ hydroxyalkyl, C₁-C₆ alkoxy, C₁-C₆ haloalkoxy, C₁-C₆ hydroxyalkoxy, C₁-C₆ alkylamino, C₂-C₆ alkenyl, or C₂-C₆ alkynyl, wherein each of which may be interrupted by one or more hetero atoms,
cyano,
halo,
hydroxyl,
nitro,
oxo,
—NH(CO)—O—(C₁-C₆ alkyl)aryl, —NH(CO)—O—(C₁-C₆ alkyl), —N(C₁-C₆ alkyl)(CO)—O—(C₁-C₆ alkyl)aryl, —N(C₁-C₆ alkyl)(CO)—O—(C₁-C₆ alkyl), —C(O)OH, —C(O)O(C₁-C₆ alkyl), —C(O)NH₂, —C(O)N(H)—(C₁-C₆ alkyl), —C(O)N(C₁-C₆ alkyl)₂, —NH(C₁-C₆ alkyl), —N(C₁-C₆ alkyl)₂, —(C₂-C₄ alkenyl)heterocyclyl, or —(C₂-C₄ alkenyl)cycloalkyl, or
—O-aryl;
   or a pharmaceutically acceptable salt, stereoisomer, or mixture thereof.

In some embodiments, the invention comprises a compound of Formula I, wherein X is —N(R^{7a})—. In some such embodiments, R^{7a} is H

In some embodiments, the invention comprises a compound of Formula I, wherein X is —C(R^{7b}R^{7c})—. In some such embodiments, R^{7b} and R^{7c} are both H.

In some embodiments, the invention comprises a compound of Formula I, wherein R¹ is selected from —H, C₁-C₆ alkyl, —(C₁-C₆ alkyl)-O—R⁸, —C(O)O—R⁸, —C(O)N(R^{7d})₂, —CHR¹¹—O—R⁸, or C₂-C₆ alkynyl. In some such embodiments, R¹ is —H. In other such embodiments, R¹ is selected from —CH₂OCH₃, —CH₂OH, —C(O)₂Me, —C(O)N(H)(C₁-C₄ alkyl), —C(O)N(H)(C₃-C₇ cycloalkyl), or —C≡C—CH₃.

In some embodiments, the invention comprises a compound of Formula I, wherein R⁵ and R⁶ are each H.

In some embodiments, the invention comprises a compound of Formula I, wherein R² is H.

In some embodiments, the invention comprises a compound of Formula I, wherein R³ is H.

In some embodiments, the invention comprises a compound of Formula I, wherein R⁴ is —H.

In some embodiments, the invention comprises a compound of Formula I, wherein R⁴ is —OR⁸, —O—(C₁-C₆ alkyl)-O—R⁸, C₁-C₆ alkyl, —(C₁-C₆ alkyl)-O—R⁸, or —(C₁-C₆ alkyl)-S(O)₂—R⁸.

In some embodiments, the invention comprises a compound of Formula I, wherein R⁴ is selected from —CH₃, —CH₂OCH₃, —CH₂OH, —CH₂S(O)₂CH₃, —OH, or —OCH₂OCH₃.

In some embodiments, the invention comprises a compound of Formula I, wherein Z is selected from optionally substituted phenyl, optionally substituted indolyl, optionally substituted naphthyl, optionally substituted pyridinyl, optionally substituted imidazolyl, optionally substituted pyrazolyl, optionally substituted pyrimidinyl, optionally substituted pyridinonyl, optionally substituted thiophenyl, or optionally substituted piperidinyl. In some such embodiments, Z is selected from optionally substituted phenyl and optionally substituted pyridinyl. In some embodiments, Z is selected from phenyl, indolyl, naphthyl, pyridinyl, imidazolyl, pyrazolyl, pyrimidinyl, pyridinonyl, thiophenyl, or piperidinyl, each of which is optionally substituted with 1-3 substituents selected from —Cl, —F, —CF₃, —CF₂CH₃, —CH₃, —CHF₂, or —C(O)O(C₁-C₆ alkyl).

In some embodiments of the compound of Formula I, Z is selected from phenyl, indolyl, naphthyl, pyridinyl, thiophenyl, 4-chlorophenyl, 4-trifluoromethylphenyl, 3-chlorophenyl, 3-trifluoromethylphenyl, 4-methoxyphenyl, 3-fluoro-4-trifluoromethylphenyl, 4-chloro-3-fluorophenyl, 4-(3-chloropropoxy)phenyl, 4-(3-hydroxypropoxy)phenyl, 3,4-dichlorophenyl, 4-fluorophenyl, 2,4-dichlorophenyl, 4-methylphenyl, 3,4-difluorophenyl, 3-fluoro-4-methoxyphenyl, 3,5-difluorophenyl, 6-trifluoromethylpyridin-3-yl, 5-methoxy-6-trifluoromethylpyridin-3-yl, 2-fluoro-4-trifluoromethylphenyl, 4-trifluoromethoxyphenyl, 2,3-difluoro-4-trifluoromethylphenyl, 4-hydroxyphenyl, 3-methoxy-4-trifluoromethylphenyl, 3-hydroxy-4-trifluoromethylphenyl, 5-chlorothiophen-2-yl, 3-fluoro-4-hydroxyphenyl, or a phenyl substituted in the 4 position with —NH—C(O)—O—CH₂-phenyl.

In some embodiments, the invention comprises a compound of Formula I, wherein Z is selected from one of the following groups, wherein the wavy line indicates the point of attachment to the rest of the molecule

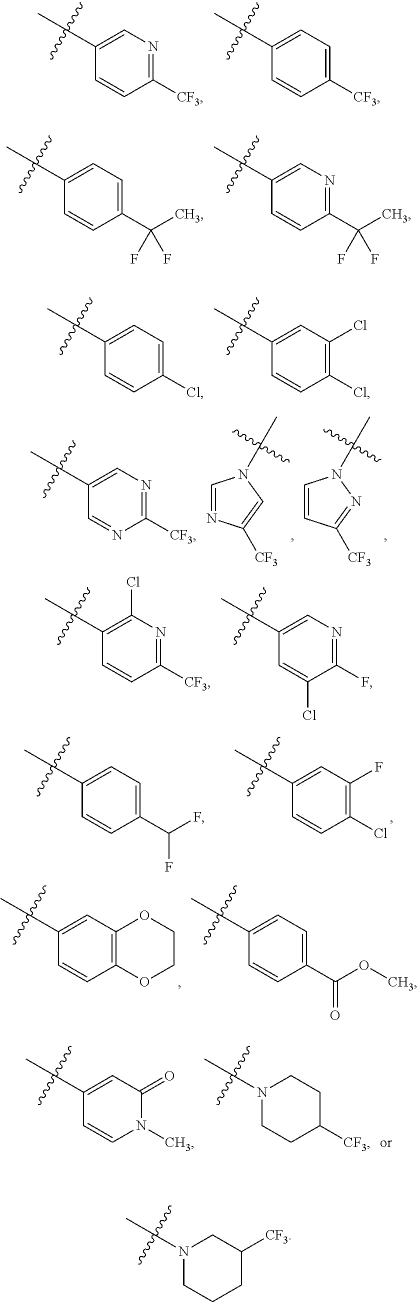

In some embodiments, the invention comprises a compound of Formula I, wherein Z is selected from one of the following groups, wherein the wavy line indicates the point of attachment to the rest of the molecule

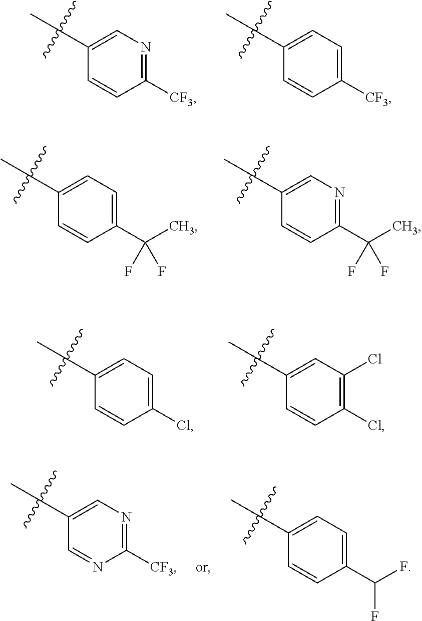

In some embodiments, the invention comprises a compound of Formula I, wherein Z is the following group, wherein the wavy line indicates the point of attachment to the rest of the molecule

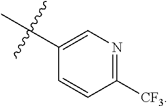

In some embodiments, the invention comprises a compound of Formula I, wherein Z is the following group, wherein the wavy line indicates the point of attachment to the rest of the molecule

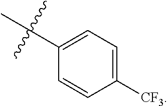

In some embodiments, the invention comprises a compound of Formula I, wherein Z is the following group, wherein the wavy line indicates the point of attachment to the rest of the molecule

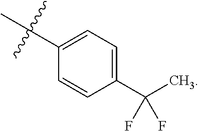

In some embodiments, the invention comprises a compound of Formula I, wherein Z is the following group, wherein the wavy line indicates the point of attachment to the rest of the molecule

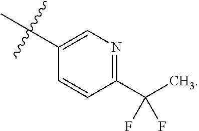

In some embodiments, the compound of Formula I has the Formula IA

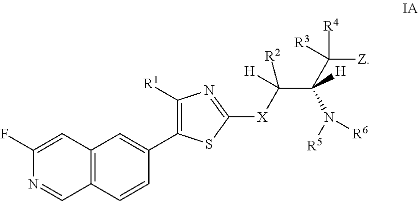

In some embodiments, the compound of Formula I has the Formula IB

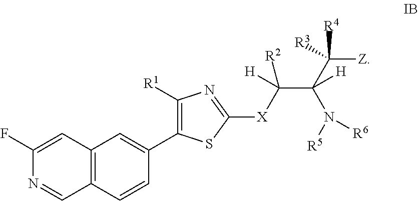

In some embodiments, the compound of Formula I has the Formula IC

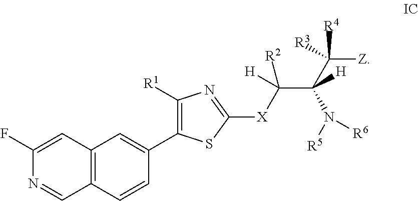

In some embodiments, the compound of Formula I has the Formula ID

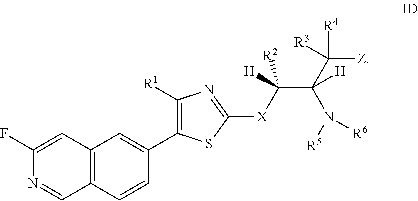

In some embodiments, the compound of Formula I has the Formula IE

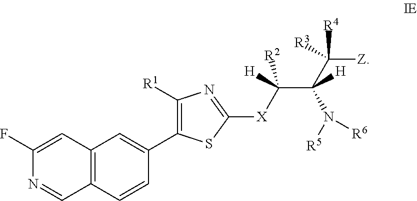

In some embodiments, R², R³, R⁵, and R⁶ are all H. In some such embodiments X is NH whereas in other such embodiments X is CH₂. In some such embodiments, Z is aryl or heteroaryl. In some such embodiments where Z is heteroaryl, Z is a 5 or 6 membered heteroaryl ring comprising one or two N atom ring members. In other such embodiments, R⁴ is H. In still other such embodiments, R¹ is H.

In another aspect, the invention comprises a pharmaceutically acceptable salt of a compound of Formula I. In one embodiment, the pharmaceutically acceptable salt of Formula I is selected from ammonium trifluoroacetate and ammonium chloride.

In another aspect, the invention comprises a pharmaceutical composition comprising a pharmaceutically-acceptable carrier and a compound of Formula I, a compound of any of the embodiments described herein, and/or a salt of any of the compounds of any of the embodiments. In some embodiments, the invention also provides the use of a compound of any of the embodiments in the manufacture of a medicament for carrying out any of the methods of any of the embodiments of the invention. Such compositions and medicaments may further include one or more additional therapeutic agent. Therefore, in some embodiments, the composition or medicament includes at least one additional therapeutic agent.

In one embodiment, the invention comprises one or more compound selected from any one or all of the Example compounds described herein or a pharmaceutically acceptable salt, or stereoisomer thereof. Each of the different groups of the Example compounds that correspond to any of the variables in the compounds of Formula I is preferred.

### 1.3 Pharmaceutical Compositions and Dosage Forms

Compounds of Formula I or any of the embodiments thereof, or a pharmaceutically acceptable salt, hydrate, or stereoisomer thereof may be used to prepare pharmaceutical compositions and single unit dosage forms. Therefore, in some embodiments, the invention provides a pharmaceutical composition that includes a compound of Formula I, or a pharmaceutically acceptable salt, or stereoisomer thereof. Pharmaceutical compositions and individual dosage forms of the invention may be suitable for oral, mucosal (including sublingual, buccal, rectal, nasal, or vaginal), parenteral (including subcutaneous, intramuscular, bolus injection, intra-arterial, or intravenous), transdermal, or topical administration. Pharmaceutical compositions and dosage forms of the invention typically also comprise one or more pharmaceutically acceptable carrier, excipient, or diluent. Sterile dosage forms are also contemplated.

The term “composition” as used herein is intended to encompass a product comprising the specified ingredients (and in the specified amounts, if indicated), as well as any product which results, directly or indirectly, from combination of the specified ingredients. The term “pharmaceutically acceptable” carrier, excipient, or diluent means that the carrier, excipient, or diluent is compatible with the other ingredients of the formulation and is not deleterious to the recipient thereof. Composition formulation may improve one or more pharmacokinetic properties (e.g., oral bioavailability, membrane permeability) of a compound of the invention (herein referred to as the active ingredient).

The pharmaceutical compositions of the invention may conveniently be presented in unit dosage form and may be prepared by any of the methods well known in the art. All methods include the step of bringing the active ingredient such as a compound of any of the embodiments into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect in the subject.

In some embodiments, pharmaceutical compositions include a Formula I compound of the invention, or a pharmaceutically acceptable salt, hydrate or stereoisomer thereof, and at least one additional therapeutic agent. Examples of additional therapeutic agents include, but are not limited to, those listed above. Such compositions may include one or more pharmaceutically acceptable carrier, excipient, or diluent.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 20th ed., Mack Publishing, Easton Pa. 2000. Examples of dosage forms include, but are not limited to, tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms particularly suitable for parenteral administration to a patient; and sterile solids (e.g., crystalline or amorphous solids) that can be reconstituted to provide liquid dosage forms suitable for parenteral administration to a patient.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,160,452, and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The pharmaceutical compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

For topical use, creams, ointments, jellies, solutions, or suspensions, etc., containing the compounds of the invention are employed. As used herein, topical application is also meant to include the use of mouthwashes and gargles.

Like the amounts and types of excipients, the amounts and specific types of active ingredients in a dosage form may differ depending on factors such as, but not limited to, the route by which it is to be administered to patients. However, typical dosage forms of the invention comprise a Formula I compound of the invention, or a pharmaceutically acceptable salt, hydrate, or stereoisomer thereof in an amount of from 0.1 mg to 1500 mg per unit to provide doses of about 0.01 to 200 mg/kg per day.

The invention further provides the use of a compound of Formula I or any of the embodiments thereof, or a pharmaceutically acceptable salt, hydrate, or stereoisomer thereof, in the preparation of a pharmaceutical composition or medicament. In some embodiments, the composition or medicament may be used to treat a disease mediated by a kinase such as PKB. In some embodiments, the disease is mediated by PKBα. In some embodiments, the disease is cancer and in some such embodiments, the cancer is a solid tumor.

### 1.4 Methods of Treatment and Prevention of Disease States

The compounds of the invention may be used to treat or prevent various kinase-related disorders. Thus, the present invention provides methods for treating or preventing such disorders. In some embodiments, the invention provides a method for treating a kinase-mediated disorder in a subject that includes administering a therapeutically effective amount of a compound of any of the embodiments of the invention or a pharmaceutical composition to the subject. In some embodiments, the subject is a mammal, and in some such embodiments is a human. In some embodiments the disorder is mediated by IGF-1R, Insulin Receptor, KDR, Tie2, EGFR, PKA, PKB, PKC, FKHR, TSC1/2, SGK, LCK, BTK, Erk, MSK, MK2, MSK, p38, P70S6K, PIM1, PIM2, ROCK2, GSK3, or a CDK complex. In some such embodiments, the disorder is mediated by PKB. In some such embodiments, the administration of the compound or pharmaceutical composition produces selective inhibition of PKB, and in some cases PKBα, in the subject after administration. In some embodiments, the disorder is cancer. The present invention thus provides methods for treating or preventing PKB-mediated disease states, such as cancer. In some embodiments, the cancer is a tumor such as a solid tumor.

The compounds of the invention may also be used to treat proliferation-related disorders. Thus, the invention further provides methods for treating such proliferation-related disorders in a subject. Such methods include administering to a subject in need thereof a therapeutically effective amount of the compound or pharmaceutical composition of any of the embodiments. In some embodiments, the subject is a mammal. In some such embodiments, the mammal is a human. In some embodiments, the proliferation-related disorder is abnormal cell growth. In other embodiments, the disorder is inflammation or an inflammation-related disorder. In still other embodiments, the disorder is a metabolic disease such as diabetes. In still other embodiments, the disorder is cancer. In some such embodiments, the cancer is a solid tumor.

The magnitude of a prophylactic or therapeutic dose of a Formula I compound of the invention or a pharmaceutically acceptable salt, solvate, hydrate, or stereoisomer thereof in the acute or chronic treatment or prevention of a cancer or other disease or condition will vary with the nature and aggressiveness of the condition, and the route by which the active ingredient is administered. The dose, and in some cases the dose frequency, will also vary according to the condition to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. In general, the dose per day is in the range of from about 0.001 to 100 mg/kg, preferably about 1 to 25 mg/kg, more preferably about 1 to about 5 mg/kg. For treatment of humans having a cancer, about 0.1 mg to about 15 g per day is administered in about one to four divisions a day, preferably 10 mg to 12 g per day, more preferably from 40 mg to 500 mg per day. In one embodiment the compounds of the invention are administered from 40 mg to 500 mg per day in about one to four divisions a day. Additionally, the recommended daily dose can be administered in cycles as single agents or in combination with other therapeutic agents. In one embodiment, the daily dose is administered in a single dose or in equally divided doses. In a related embodiment, the recommended daily dose can be administered one time per week, two times per week, three times per week, four times per week or five times per week.

The compounds of the invention can be administered to provide systemic distribution of the compound within the patient. Therefore, in some embodiments, the compounds of the invention are administered to produce a systemic effect in the body.

The compounds of the invention may also be administered directly to a site affected by a condition, as, for example, an in the treatment of an accessible area of skin or an esophageal cancer.

As indicated above, the compounds of the invention may be administered via oral, mucosal (including sublingual, buccal, rectal, nasal, or vaginal), parenteral (including subcutaneous, intramuscular, bolus injection, intra-arterial, or intravenous), transdermal, or topical administration. In some embodiments, the compounds of the invention are administered via mucosal (including sublingual, buccal, rectal, nasal, or vaginal), parenteral (including subcutaneous, intramuscular, bolus injection, intra-arterial, or intravenous), transdermal, or topical administration. In other embodiments, the compounds of the invention are administered via oral administration. In still other embodiments, the compounds of the invention are not administered via oral administration.

Different therapeutically effective amounts may be applicable for different conditions, as will be readily known by those of ordinary skill in the art. Similarly, amounts sufficient to treat or prevent such conditions, but insufficient to cause, or sufficient to reduce, adverse effects associated with conventional therapies are also encompassed by the above described dosage amounts and dose frequency schedules.

Some methods of the invention comprise the administration of a compound of the invention and an additional therapeutic agent (i.e., a therapeutic agent other than a compound of the invention). Thus, the compounds of the invention can be used in combination with at least one other therapeutic agent. Examples of additional therapeutic agents include, but are not limited to, antibiotics, anti-emetic agents, antidepressants, antifungal agents, anti-inflammatory agents, antineoplastic agents, antiviral agents, cytotoxic agents, and other anticancer agents, immunomodulatory agents, alpha-interferons, β-interferons, alkylating agents, hormones, and cytokines. In one embodiment, the invention encompasses administration of an additional therapeutic agent that demonstrates anti-cancer activity. In another embodiment, an additional therapeutic agent that demonstrates cytotoxic activity is administered to a subject such as a cancer patient.

The compounds of the invention and the other therapeutics agent can act additively or, preferably, synergistically. In some embodiments, a composition comprising a compound of the invention is administered concurrently with the administration of another therapeutic agent, which can be part of the same composition or can be in a different composition from the one that comprises the compound of the invention. In other embodiments, a compound of the invention is administered prior to, or subsequent to, administration of another therapeutic agent. In still other embodiments, a compound of the invention is administered to a patient who has not previously undergone or is not currently undergoing treatment with another therapeutic agent. A compound of the invention may be administered to a subject that has had, is currently undergoing, or is scheduled to receive radiation therapy. In some such embodiments, the subject is a cancer patient.

When administered as a combination, the therapeutic agents can be formulated as separate compositions that are administered at the same time or sequentially at different times, or the therapeutic agents can be given as a single composition. The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent. Specifically, the administration of compounds of the present invention may be in conjunction with additional therapies known to those skilled in the art in the prevention or treatment of neoplasia, such as with radiation therapy or with cytostatic or cytotoxic agents.

If formulated as a fixed dose, such combination products employ the compounds of this invention within the accepted dosage ranges. Compounds of Formula I may also be administered sequentially with known anticancer or cytotoxic agents when a combination formulation is inappropriate. The invention is not limited in the sequence of administration as compounds of the invention may be administered either prior to, simultaneous with, or after administration of a known anticancer or cytotoxic agent.

There are large numbers of antineoplastic agents available in commercial use, in clinical evaluation and in pre-clinical development, which may be selected for treatment of neoplasia by combination drug chemotherapy. Such antineoplastic agents

fall into several major categories, namely, antibiotic-type agents, alkylating agents, antimetabolite agents, hormonal agents, immunological agents, interferon-type agents and a category of miscellaneous agents.

A first family of antineoplastic agents which may be used in combination with compounds of the present invention consists of antimetabolite-type/thymidilate synthase inhibitor antineoplastic agents. Suitable antimetabolite antineoplastic agents may be selected from, but are not limited to, the group consisting of 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT, and uricytin.

A second family of antineoplastic agents which may be used in combination with compounds of the present invention consists of alkylating-type antineoplastic agents. Suitable alkylating-type antineoplastic agents may be selected from, but are not limited to, the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin, and trimelamol.

A third family of antineoplastic agents which may be used in combination with compounds of the present invention consists of antibiotic-type antineoplastic agents. Suitable antibiotic-type antineoplastic agents may be selected from, but are not limited to, the group consisting of Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, and zorubicin.

A fourth family of antineoplastic agents which may be used in combination with compounds of the present invention consists of a miscellaneous family of antineoplastic agents, including tubulin interacting agents, topoisomerase II inhibitors, topoisomerase I inhibitors and hormonal agents, selected from, but not limited to, the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, with anolides, and Yamanouchi YM-534.

Alternatively, the present compounds may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bc1-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

The compounds of the invention may further be used with VEGFR inhibitors. Other compounds described in the following patents and patent applications can be used in combination therapy: U.S. Pat. No. 6,258,812, US 2003/0105091, WO 01/37820, U.S. Pat. No. 6,235,764, WO 01/32651, U.S. Pat. No. 6,630,500, U.S. Pat. No. 6,515,004, U.S. Pat. No. 6,713,485, U.S. Pat. No. 5,521,184, U.S. Pat. No. 5,770,599, U.S. Pat. No. 5,747,498, WO 02/68406, WO 02/66470, WO 02/55501, WO 04/05279, WO 04/07481, WO 04/07458, WO 04/09784, WO 02/59110, WO 99/45009, WO 00/59509, WO 99/61422, U.S. Pat. No. 5,990,141, WO 00/12089, and WO 00/02871.

In some embodiments, the combination comprises a composition of the present invention in combination with at least one anti-angiogenic agent. Agents are inclusive of, but not limited to, in vitro synthetically prepared chemical compositions, antibodies, antigen binding regions, radionuclides, and combinations and conjugates thereof. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-tumor agents include HERCEPTIN™ (trastuzumab), which may be used to treat breast cancer and other forms of cancer, and RITUXAN™ (rituximab), ZEVALIN™ (ibritumomab tiuxetan), and LYMPHOCIDE™ (epratuzumab), which may be used to treat non-Hodgkin's lymphoma and other forms of cancer, GLEEVAC™ which may be used to treat chronic myeloid leukemia and gastrointestinal stromal tumors, and BEXXAR™ (iodine 131 tositumomab) which may be used for treatment of non-Hodgkins's lymphoma.

Exemplary anti-angiogenic agents include ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTINT™ or VEGF-TRAPT™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

Other anti-angiogenic agents include Campath, IL-8, B-FGF, Tek antagonists (Ceretti et al., U.S. Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., U.S. Publication No. 2002/0042368), specifically binding anti-eph receptor and/or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide.(Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); cancer therapy, (University of South Carolina, USA); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (Pro1X, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-1 alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sirna, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

Alternatively, the present compounds may also be used in co-therapies with other anti-neoplastic agents, such as VEGF antagonists, other kinase inhibitors including p38 inhibitors, KDR inhibitors, EGF inhibitors and CDK inhibitors, TNF inhibitors, matrix metalloproteinases (MMP) inhibitors, COX-2 inhibitors including celecoxib, NSAID's, or αᵥβ₃ inhibitors.

### 2. Working Examples

The compounds of Formula I were prepared according to the following synthetic schemes and individual examples detailed herein. The compounds were named using Chemdraw Ultra, v.8.07. These schemes and examples are provided for the purpose of illustration only and are not intended to limit the scope of the invention.

Unless otherwise noted, all materials were obtained from commercial suppliers and were used without further purification. Anhydrous solvents such as DMF, THF, DCM, and toluene were generally obtained from the Aldrich Chemical Company. All reactions involving air- or moisture-sensitive compounds were performed under a nitrogen atmosphere unless otherwise noted. Flash chromatography was performed using Aldrich Chemical Company silica gel (200-400 mesh, 60A) or Biotage pre-packed column. Thin-layer chromatography (TLC) was performed with Analtech gel TLC plates (250 mμ.). Preparative TLC was performed with Analtech silica gel plates (1000-2000.mu.). Preparative HPLC was conducted on a Varian, Shimadzu, Beckman, or Waters HPLC system with 0.1% TFA/H₂O and 0.1% TFA/CH₃CN as mobile phase. The flow rate was at 20 mL/minute and the gradient method was used. ¹H NMR spectra were obtained with super conducting FT NMR spectrometers operating at 400 MHz or a Varian 300 MHz instrument. Chemical shifts are expressed in ppm downfield from the tetramethylsilane internal standard. All compounds showed NMR spectra consistent with their assigned structures. Mass spectra (MS) were obtained using a Perkin Elmer-SCIEX API 165 electrospray mass spectrometer (positive and/or negative) or an HP 1100 MSD LC-MS with electrospray ionization and quadrupole detection. All parts are by weight and temperatures are in degrees centigrade unless otherwise indicated.

The following abbreviations are used: AcOH (acetic acid), ATP (adenosine triphosphate), Boc (tert-butyloxycarbonyl), Boc₂O (Boc anhydride), Br₂ (bromine), t-BuOH (tert-butanol), CH₃CN or ACN (acetonitrile), MeI (iodomethane or methyl iodide), CCl₄ (carbon tetrachloride), CHCl₃ (chloroform), CDCl₃ (deuterated chloroform), CD₃OD (d₄-methanol), CO₂ (carbon dioxide), Cs₂CO₃ (cesium carbonate), CuI (copper iodide), DCM (dichloromethane), DIBAL-H (diisobutylaluminum hydride), dppf (1,1-diphenylphosphinoferrocene), DMAP (4-(dimethylamino)pyridine), DMF (dimethylformamide), DMSO (dimethylsulfoxide), EDC 1-(3-dimethylaminopropyl)-3 (ethylcarbodiimide hydrochloride), EtOAc (ethyl acetate), EtOH (ethanol), Et₂O (diethyl ether), Fe (iron), g (gram), h (hour), H₂ (hydrogen), H₂O (water), HCl (hydrochloric acid), H₂SO₄ (sulfuric acid), K₂CO₃ (potassium carbonate), KOAc (potassium acetate), KOH (potassium hydroxide), LAH (lithium aluminum hydride), LCMS (liquid chromatography mass spectrometry), LiCl (lithium chloride), MeOH (methanol), MgSO₄ (magnesium sulfate), mg (milligram), min (minute), mL (milliliter), NBS (N-bromosuccinimide), Na₂SO₄ (sodium sulfate), NaHCO₃ (sodium bicarbonate), Na₂CO₃ (sodium carbonate), NaCl (sodium chloride), NaH (sodium hydride), NaHMDS (sodium hexamethylsilazane), NaOH (sodium hydroxide), NaBH₄ (sodium borohydride), NH₄Cl (ammonium chloride), Pd/C (palladium on carbon), PdCl₂(PPh₃)₂ (palladium chloride bis(triphenylphosphine)), Pd₂(dba)₃ (palladium dibenzylideneacetone), PdCl₂(dppf) (1,1-bis(diphenylphosphino)ferrocene, palladium chloride), Pd(PPh₃)₄ (palladium tetrakis triphenylphosphine), Pd(OH)₂ (palladium hydroxide), Pd(OAc)₂ (palladium acetate), PPh₃ (triphenylphosphine), RT (room temperature), SiO₂ (silica), SOCl₂ (thionyl chloride), TEA (triethylamine), TFA (trifluoroacetic acid), THF (tetrahydrofuran), and Zn (zinc).

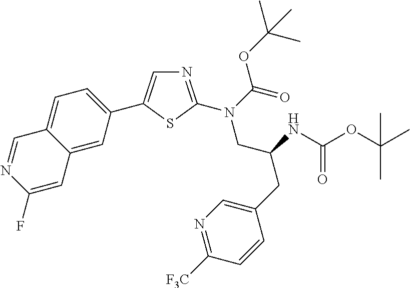

### Example 1

### tert-Butyl ((2S)-2-((tert-butoxycarbonyl)amino)-3-(6-(trifluoromethyl)-3-pyridinyl)propyl)(5-(3-fluoro-6-isoquinolinyl)-1,3-thiazol-2-yl)carbamate

This compound was synthesized as shown in Scheme 1 using tert-butyl (5-bromo-1,3-thiazol-2-yl)((2S)-2-((tert-butoxycarbonyl)amino)-3-(6-(trifluoromethyl)-3-pyridinyl)propyl)carbamate. The synthesis of tert-butyl (5-bromo-1,3-thiazol-2-yl)((2S)-2-((tert-butoxycarbonyl)amino)-3-(6-(trifluoromethyl)-3-pyridinyl)propyl)carbamate is shown in Scheme 2.

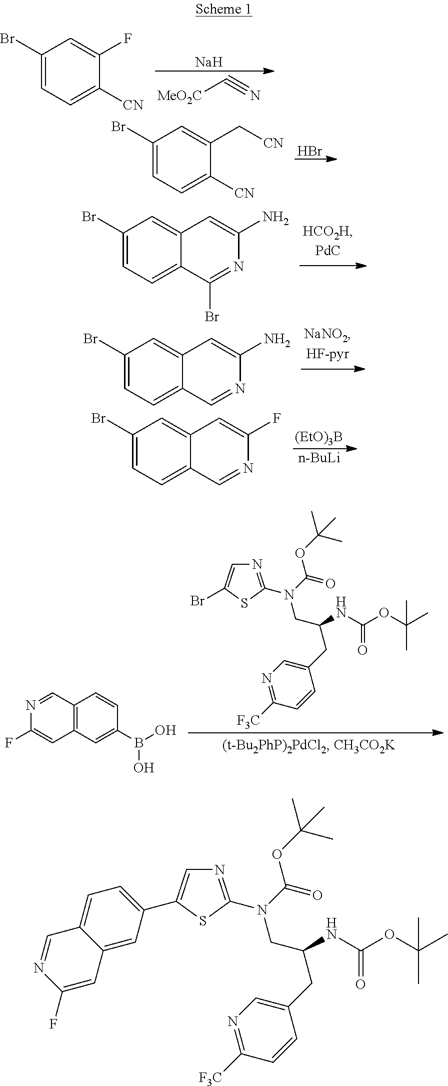

4-Bromo-2-(cyanomethyl)benzonitrile: NaH (47.2 g, 1.18 mol, Aldrich) was suspended in DMSO (320 mL) and cooled to 0° C. in an ice-water bath. The mixture became viscous as the DMSO froze. Methyl cyanoacetate (104 mL, 1.18 mol, Aldrich) was added slowly causing a slight temperature increase and thus a more easily stirred solution. The internal temperature stayed below 30° C. The mixture was stirred for 30 minutes at room temperature before 4-bromo-2-fluorobenzonitrile (118.0 g, 590 mmol, 3B Scientific Corporation Product List 3B3-007315) was added via cannula as a solution in DMSO (500 mL). The mixture was heated with a heating mantle to an internal temperature of 90° C. Upon reaching 90° C., the reaction was shown to be complete by LCMS. The mixture was allowed to stand at room temperature for 16 hours. Water (1.2 L) was added, and the temperature was then brought up slowly to an internal temperature of 104° C. Water (2.3 L) was added and the mixture was heated at reflux for 20 hours. The mixture was cooled to 5° C. HCl (700 mL, 0.2 N) was then added quickly, and the resulting mixture was stirred at 5° C. for about 30 minutes. The resulting precipitate was filtered, washed with water, and dried to afford 4-bromo-2-(cyanomethyl)benzonitrile (102 g, 78%).

1,6-Dibromoisoquinolin-3-amine: 4-Bromo-2-(cyanomethyl)benzonitrile (75.0 g, 339 mmol) was added to 2,2-dichloroacetic acid (150 mL, 339 mmol, Aldrich). The resulting solution was cooled to 0° C. in an ice-water bath. HBr (Aldrich) was bubbled through the cold solution until a yellow precipitate crashed out of solution, resulting in a yellow slurry. The HBr was bubbled through the slurry for an additional 5 minutes. The solution was allowed to warm to room temperature for about an hour. LCMS indicated complete conversion. The slurry was then cooled to 0° C. in an ice-water bath and diethyl ether (200 mL) was added rapidly. The mixture was stirred for 20 minutes at about 5° C. and then was filtered, washed with ether, and dried to provide 1,6-dibromoisoquinolin-3-amine as a yellow solid (42 g, 41%): LCMS (API-ES) m/z: 302.9 [M+H]⁺.

7-Bromoquinazolin-2-amine: A mixture of 4,7-dibromoquinazolin-2-amine (1.00 g, 3.30 mmol), ammonium formate (0.445 g, 7.06 mmol, Aldrich) and tetrakis(triphenylphosphine)palladium (0) (0.300 g, 0.260 mmol, Aldrich) in DMF (10 mL) was heated at 50° C. in a screw-cap sealed flask for 21.5 hours. The reaction was cooled to room temperature and diluted with MeOH. Charcoal was added to the mixture and the resulting mixture was stirred for 5 minutes. The mixture was filtered through Celite® brand filter aid and the filter cake was washed with MeOH. The filtrate was concentrated in vacuo. The residue was boiled in iPrOH for 1 hour and then the mixture was cooled overnight. The solid was filtered, washed with iPrOH and was then dried to provide 7-bromoquinazolin-2-amine as a yellow amorphous solid (347 mg, 47%). LCMS (API-ES) m/z: 222.9, 224.9 [M+H]

6-Bromo-3-fluoroisoquinoline: To a mixture of 6-bromoisoquinolin-3-amine (0.710 g, 3.18 mmol) in pyridine hydrofluoride (10.0 mL, 3.18 mmol, Aldrich) at −78° C. was carefully added sodium nitrite (0.264 g, 3.82 mmol, Aldrich). The reaction mixture was stirred at −78° C. for 5 minutes. The reaction mixture was then warmed to room temperature and stirred for 40 minutes. The mixture was then poured into an ice bath and the pH was adjusted to >9 with Na₂CO₃. The mixture was filtered to recover a yellow-purple solid. The solid was dissolved in EtOAc—water with stirring. The resulting mixture was then extracted with EtOAc (3×200 mL). The EtOAc layers were combined, washed with brine, dried over Na₂SO₄, filtered and concentrated. The residue was taken up in DCM-MeOH and adsorbed onto silica gel. Purification by chromatography on silica gel (eluting with EtOAc 0-7% in hexanes) provided 6-bromo-3-fluoroisoquinoline (500 mg, 70%). LCMS (API-ES) m/z: 226.0, 228.0 [M+H]¹.

3-Fluoroisoquinolin-6-ylboronic acid: To a solution of 6-bromo-3-fluoroisoquinoline (3.66 g, 16.2 mmol) and triethyl borate (5.5 mL, 32.4 mmol, Aldrich) in THF (40 mL) at −78° C., was added n-butyllithium (1.6 M solution in hexanes, 20.2 mL, Aldrich) dropwise over 45 minutes. The reaction mixture was stirred at −78° C. for another 3 hours after the addition. The resulting mixture was quenched with 5 N hydrochloric acid solution (120 mL), diluted with water (100 mL), and then extracted with EtOAc (3×200 mL). The combined organic phases were dried over Na₂SO₄, filtered and concentrated to give a solid residue. The solid residue was triturated with DCM (200 mL) and filtered. The off-white solid was collected and dried on vacuum overnight to give the title compound (1.8 g, 58%): LCMS (API-ES) m/z: 192 (M⁺+H); ¹H NMR (300 MHz, DMSO-d₆) δ ppm 7.60 (s, 1H) 7.90-7.98 (m, 1H) 8.12 (d, J=8.33 Hz, 1H) 8.39 (s, 1H) 8.44 (s, 2H) 9.10 (s, 1H).

(2S)—N-(5-(3-Fluoro-6-isoquinolinyl)-1,3-thiazol-2-yl)-3-(6-(trifluoromethyl)-3-pyridinyl)-1,2-propanediamine: A mixture of potassium acetate (4.49 g, 45.7 mmol), tert-butyl (5-bromo-1,3-thiazol-2-yl)((2S)-2-((tert-butoxycarbonyl)amino)-3-(6-(trifluoromethyl)-3-pyridinyl)propyl)carbamate (3.80 g, 6.54 mmol, prepared as shown in Scheme 2), and 3-fluoroisoquinolin-6-ylboronic acid (1.62 g, 8.50 mmol) in acetonitrile (70 mL) and water (21 mL) was degassed with nitrogen for 30 seconds. To this mixture was then added (t-Bu₂PhP)₂PdCl₂ (365 mg, 0.59 mmol, prepared according to Org. Lett., 2006, 8(9), 1787). The resulting mixture was stirred for 4 hours at 85° C. The solvents were removed under reduced pressure, and the residue was diluted with water (100 mL), extracted with EtOAc (2×200 mL), and dried over Na₂SO₄. After filtration and concentration, the title compound was obtained by silica gel flash column chromatography as an off-white solid (3.29 g, 77%): LCMS (API-ES) m/z: 648 (M⁺+H); ¹H NMR (300 MHz, CDCl₃) δ ppm 1.29 (s, 9H) 1.53 (s, 9H) 2.88-3.13 (m, 2 H) 4.12 (d, J=7.16 Hz, 1H) 4.29 (d, J=13.30 Hz, 2H) 5.22-5.39 (m, 1H) 7.19 (s, 1H) 7.66 (d, J=8.04 Hz, 1H) 7.73 (dd, J=8.62, 1.46 Hz, 1H) 7.77 (s, 1H) 7.86 (s, 2H) 7.98 (d, J=8.62 Hz, 1H) 8.63 (s, 1H) 8.90 (s, 1H).

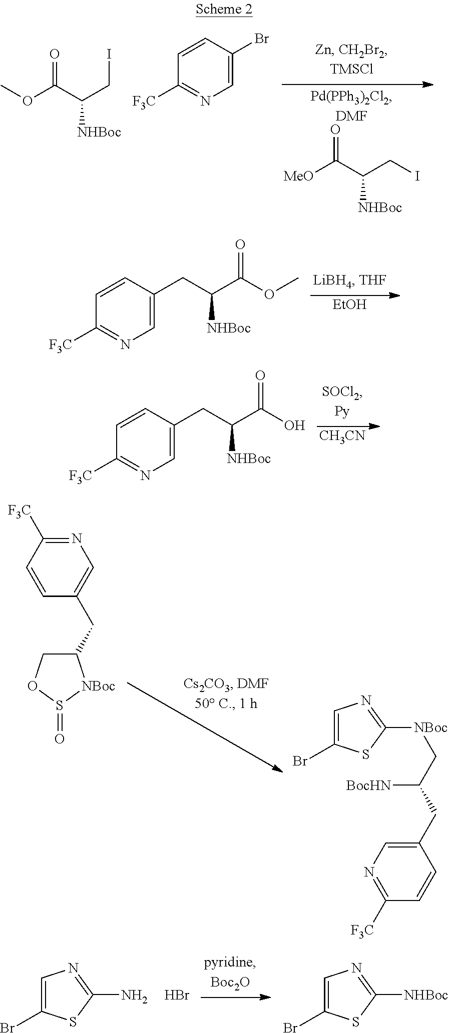

(S)-Methyl 2-(tert-butoxycarbonylamino)-3-(6-(trifluoromethyl)pyridin-3-yl)propanoate: To a 250 mL round-bottomed flask was added zinc, nanosize activated powder (0.75 mL, 82 mmol, Aldrich), and DMF (14 mL, 177 mmol). The mixture was stirred and treated dropwise with 1,2-dibromoethane (0.35 mL, 4.1 mmol, Aldrich). The resulting mixture was then stirred at 90° C. for 30 minutes. After cooling, chlorotrimethylsilane (0.10 mL, 0.82 mmol, Aldrich) was added, and the mixture was stirred at room temperature for 30 minutes. To this stirred mixture, Boc-3-iodo-L-alanine methyl ester (4.5 g, 14 mmol, Aldrich) in 10 mL DMF was added dropwise via an addition funnel. After addition, the combined mixture was stirred at room temperature for 4 hours. To this mixture was then added dichlorobis(triphenylphosphine)palladium(0) (0.48 g, 0.68 mmol, Aldrich) and a 10 mL DMF solution of 5-bromo-2-(trifluoromethyl)pyridine (4.0 g, 18 mmol, Aldrich). The resulting mixture was stirred at 25° C. overnight. The reaction mixture was filtered through Celite® brand filter aid, diluted with NH₄Cl and water (70 mL each), and diluted with EtOAc (200 mL). The aqueous layer was extracted with EtOAc (2×100 mL), and the combined organic layers were then washed with saturated sodium chloride (1×50 mL), and water (1×5 mL), and then dried over Na₂SO₄, filtered, and concentrated in vacuo. The remaining residue was adsorbed onto a plug of silica gel and chromatographed through a Redi-Sep® pre-packed silica gel column (40 g), eluting with a gradient (5-50% EtOAc in hexane) to provide (S)-methyl 2-(tert-butoxycarbonylamino)-3-(6-(trifluoromethyl)pyridin-3-yl)propanoate (4.39 g, 93%): LCMS (API-ES) m/z (%): 349.3 (100%, M⁺+H).

(S)-tert-Butyl 1-hydroxy-3-(6-(trifluoromethyl)pyridin-3-yl)propan-2-ylcarbamate: A 2 L, round bottom flask equipped with a large magnetic stirbar was charged with (S)-methyl 2-(tert-butoxycarbonylamino)-3-(6-(trifluoromethyl)pyridin-3-yl)propanoate (48.3 g, 139 mmol), which was dissolved in a mixture of anhydrous THF (800 mL) and EtOH (240 mL). The mixture was cooled in an ice bath and then lithium borohydride (6.04 g, 277 mmol, Aldrich) was added in three portions of 1, 2, and 3 g. The resulting mixture was allowed to warm to room temperature. After 15 hours, the reaction was quenched by slow addition of water (250 mL) and 5% citric acid (250 mL), resulting in additional gas evolution. The brown-black mixture was then carefully concentrated by rotary evaporation until a black oil separated from the colorless aqueous layer. The mixture was then extracted with EtOAc (800 mL), and the pH 10 aqueous layer was separated and extracted with EtOAc (200 mL). The black organic phase was washed with water (300 mL, to pH 9) and saturated brine (300 mL, to pH 7-8), and then was dried over anhydrous Na₂SO₄. The mixture was filtered through a pad of Celite® brand filter aid (to remove black particulates), and concentrated to a brown black oil. The oil was dried under vacuum to yield (S)-tert-butyl 1-hydroxy-3-(6-(trifluoromethyl)pyridin-3-yl)propan-2-ylcarbamate as a gummy foam that was carried forward without further purification (42.3 g, 95%): LCMS (API-ES) m/z: 321.

(S)-3-(tert-Butyloxycarbonyl)-4-(6-(trifluoromethyl)pyridin)[1,2,3]-oxathiazolidine-2-oxide: To a 150 mL round-bottomed flask was added SOCl₂ (1.1 mL, 15 mmol, Aldrich), and acetonitrile (300 mL). The solution was stirred at −78° C. and treated dropwise via addition funnel with (S)-tert-butyl 1-hydroxy-3-(6-(trifluoromethyl)pyridin-3-yl)propan-2-ylcarbamate (1.9 g, 5.9 mmol) in 10 mL CH₃CN. The mixture was stirred at −78° C. for 30 minutes and treated in one portion with anhydrous pyridine (2.4 mL, 30 mmol). The suspension was warmed to room temperature and stirred overnight. The solution was concentrated under reduced pressure. The reaction mixture was diluted with water and EtOAc 1:1 (200 mL) and extracted with EtOAc (3×50 mL). The organic layers were combined and washed with saturated sodium chloride (1×50 mL) and water (1×50 mL), and then were dried over Na₂SO₄, filtered, and concentrated in vacuo. The product thus obtained was adsorbed onto a plug of silica gel and chromatographed through a Redi-Sep® pre-packed silica gel column (40 g), eluting with gradient (5-50% EtOAc in hexane) to provide (S)-3-(tert-butyloxycarbonyl)-4-((6-(trifluoromethyl)pyridin)[1,2,3]-oxathiazolidine-2-oxide (1.5 g, 69%): LCMS (API-ES) m/z (%): 367.3 (100%, M⁺+H).

tert-Butyl 5-bromothiazol-2-ylcarbamate: A suspension of 5-bromothiazol-2-amine hydrobromide (325 g, 1250 mmol) in acetonitrile (3.0 L) was stirred at room temperature (22° C.) and treated with pyridine (506 mL, 6251 mmol) followed by di-tert-butyl dicarbonate (435 mL, 1875 mmol, Aldrich). The reaction mixture was stirred at room temperature for 22 hours. The solvent was reduced in vacuo and the mixture was partitioned between EtOAc and 1 N HCl. The aqueous layer was extracted again with EtOAc and the combined organic phases were washed with 1 N HCl, saturated NaHCO₃, and saturated NaCl. The organic layer was then dried over Na₂SO₄ and filtered. The reaction mixture was concentrated, loaded onto silica, and purified by flash chromatography (0-25%, EtOAc in hexanes). The fractions were concentrated in vacuo to provide tert-butyl 5-bromothiazol-2-ylcarbamate (160 g, 46%) as a white crystalline solid.

tert-Butyl (5-bromo-1,3-thiazol-2-yl)((2S)-2-((tert-butoxycarbonyl)amino)-3-(6-(trifluoromethyl)-3-pyridinyl)propyl)carbamate: To a 100 mL round-bottomed flask was added tert-butyl 5-bromothiazol-2-ylcarbamate (1.5 g, 5.4 mmol), Cs₂CO₃ (3.5 g, 11 mmol), and DMF (0.41 mL, 5.4 mmol). The mixture was stirred at 50° C. and treated dropwise via syringe with (S)-3-(tert-butyloxycarbonyl)-4-((6-(trifluoromethyl)pyridin)[1,2,3]-oxathiazolidine-2-oxide (2.4 g, 6.4 mmol) in DMF (1 mL). The mixture was then stirred at 50° C. for 1 hour. The mixture was diluted with ether and washed with brine, dried over Na₂SO₄, filtered, and concentrated in vacuo. The product thus obtained was adsorbed onto a plug of silica gel and chromatographed through a Redi-Sep® pre-packed silica gel column (40 g), eluting with gradient (5-50% EtOAc in hexane), to provide tert-butyl (5-bromo-1,3-thiazol-2-yl)((2S)-2-((tert-butoxycarbonyl)amino)-3-(6-(trifluoromethyl)-3-pyridinyl)propyl)carbamate (1.35 g, 43%): LCMS (API-ES) m/z (%): 582.2 (100%, M⁺+H).

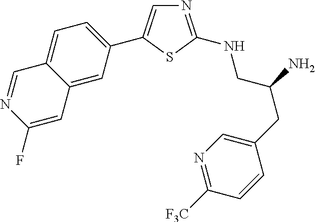

### Example 2

### (2S)—N-(5-(3-Fluoro-6-isoquinolinyl)-1,3-thiazol-2-yl)-3-(6-(trifluoromethyl)-3-pyridinyl)-1,2-propanediamine

To a solution of tert-butyl (5-bromo-1,3-thiazol-2-yl)((2S)-2-((tert-butoxycarbonyl)amino)-3-(6-(trifluoromethyl)-3-pyridinyl)propyl)carbamate (3.20 g, 4.94 mmol, prepared as shown for Example 1) in DCM (22 mL) was added TFA (22 mL, Aldrich). The mixture was stiffed for 30 minutes at room temperature. After concentration, the residue was dried under vacuum for 30 minutes. The residue was taken into EtOAc (300 mL) and treated with saturated NaHCO₃ (300 mL) cautiously. The organic layer was separated and dried over Na₂SO₄. After filtration and concentration, the title compound was obtained through silica gel flash column chromatography (200 g silica gel, eluted with DCM:2N NH₃ in MeOH, 19:1) as a light yellow solid (1.80 g, 81%): LCMS (API-ES) m/z: 448 (M⁺+H); ¹H NMR (400 MHz, DMSO-d₆) δ ppm 2.58-2.70 (m, 1H) 2.86-2.96 (m, 1H) 3.11-3.25 (m, 2H) 3.30-3.38 (m, 1H) 7.51 (s, 1H) 7.77 (s, 1H) 7.82 (s, 1H) 7.84 (s, 1H) 7.86-7.92 (m, 1 H) 7.94-8.00 (m, 1H) 8.06-8.11 (m, 1H) 8.14-8.28 (m, 1H) 8.61-8.69 (m, 1H) 8.98 (s, 1H).

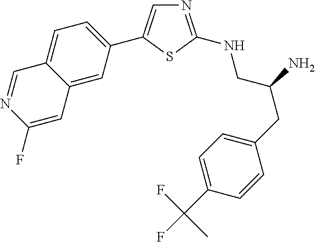

### Example 3

### N—((S)-2-Amino-3-(4-(1,1-difluoroethyl)phenyl)propyl)-5-(3-fluoroisoquinolin-6-yl)thiazol-2-amine

This compound was synthesized as shown in Scheme 3.

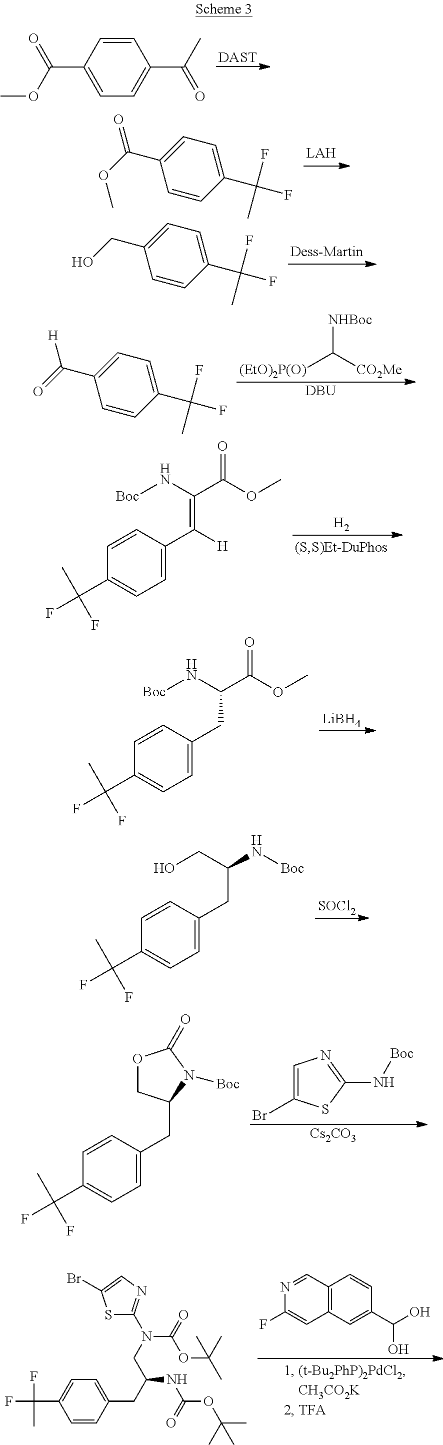

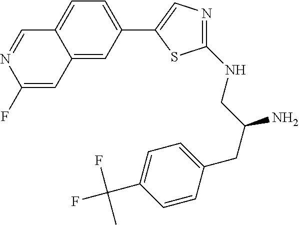

Methyl 4-(1,1-difluoroethyl)benzoate: To a 200 mL high pressure reaction tube with a solution of 4-acetylbenzoic acid methyl ester (10.0 g, 56 mmol, Aldrich) and (diethylamino)trifluorosulfur (22 mL, 168 mmol, Aldrich) in chloroform (30 mL) was added EtOH (0.3 mL, 6 mmol). The resulting mixture was sealed and stiffed overnight at 80° C. After cooling to room temperature, the reaction mixture was poured into a saturated solution of NaHCO₃ (300 mL), and then extracted with DCM (2×300 mL), and dried over MgSO₄. After filtration and concentration, the title compound was obtained by silica gel flash column chromatography (eluted with hexane:EtOAc, 4:1) as an oil (10.0 g, 89%): LCMS (API-ES) m/z: 201 (M⁺+H). ¹H NMR (300 MHz, CDCl₃) δ ppm 1.93 (t, J=18.20 Hz, 3H) 3.90-3.99 (m, 3H) 7.58 (d, J=8.62 Hz, 2H) 8.09 (d, J=8.62 Hz, 2H).

(4-(1,1-Difluoroethyl)phenyl)methanol: A mixture of LAH (2.92 g, 77 mmol, Aldrich) in THF (50 mL) was stirred for 3 hours at 50° C. The resulting mixture was cooled to room temperature with a cold water bath, and to the mixture was added a solution of ethyl 4-(1,1-difluoroethyl)benzoate (10.0 g, 46.7 mmol) in THF (20 mL). The resulting reaction mixture was stirred at 0° C. for 1 hour and at room temperature for another 1 hour. The reaction mixture was quenched with saturated NaH₂PO₄ solution, extracted with EtOAc, and the organic phase was dried over Na₂SO₄. After filtration and concentration, the title compound was obtained by silica gel flash column chromatography (eluted with hexane:EtOAc, 4:1) as an oil (7.02 g, 87%): LCMS (API-ES) m/z: 155 (M⁺−H₂O); ¹H NMR (300 MHz, CDCl₃) δ ppm 1.92 (t, J=18.12 Hz, 3H) 4.73 (d, J=4.82 Hz, 2H) 7.42 (d, J=8.48 Hz, 2H) 7.52 (d, J=8.33 Hz, 2H).

4-(1,1-Difluoroethyl)benzaldehyde: To a mixture of (4-(1,1-difluoroethyl)phenyl)methanol (9.00 g, 52.3 mmol) and sodium bicarbonate (43.9 g, 0.52 mol) in 100 mL of DCM was slowly added Dess-Martin Periodinane (25.3 g, 59.6 mmol, Aldrich). The mixture was stirred for 30 minutes and was then quenched with 200 mL of aqueous Na₂S₂O₃ and 200 mL of aqueous NaHCO₃, and diluted with 100 mL of water. The mixture was stirred for 20 minutes and partitioned. The aqueous portion was extracted with 2×300 mL of DCM and the combined organic layers were dried over MgSO₄. After filtration and concentration, the title compound was obtained by silica gel flash column chromatography (eluted with hexane:EtOAc, 4:1) as an oil (4.71 g, 52%). LCMS (API-ES) m/z: 171 (M⁺+H). ¹H NMR (300 MHz, CDCl₃) δ ppm 1.94 (t, J=18.12 Hz, 3H) 7.68 (d, J=8.18 Hz, 2H) 7.95 (d, J=8.62 Hz, 2H) 10.07 (s, 1H).

Methyl (2Z)-3-(4-(1,1-difluoroethyl)phenyl)-2-((((1,1-dimethylethyl)oxy)carbonyl)amino)-2-propenoate: To a solution of N-tert-butyloxycarbonyl-α-phosphonoglycine trimethyl ester (11.0 g, 37.0 mmol, Aldrich) in DCM (30 mL) was added 1,8-diazabicyclo[5.4.0]undec-7-ene (5.5 mL, 37 mmol, Aldrich) at 0° C. The resulting mixture was then stirred for 30 minutes. To this clear solution was slowly added a solution of 4-(1,1-difluoroethyl)benzaldehyde (5.20 g, 31 mmol) in DCM (30 mL) at 0° C. The mixture was stirred for 1.5 hours at 0-10° C. The resulting mixture was then quenched with saturated NaH₂PO₄ solution, extracted with DCM, and dried over Na₂SO₄. After filtration and concentration, the title compound was obtained by silica gel flash column chromatography (eluted with hexane:EtOAc, 4:1) as an oil. LCMS (API-ES) m/z: 364 (M⁺+Na). ¹H NMR (300 MHz, CDCl₃) δ ppm 1.39 (s, 9H) 1.91 (t, J=18.12 Hz, 2H) 3.87 (s, 3H) 6.14-6.36 (m, 1H) 7.28 (s, 1H) 7.42-7.63 (m, 4H).

(S)-Methyl 2-(tert-butoxycarbonylamino)-3-(4-(1,1-difluoroethyl)phenyl)propanoate: (Z)-Methyl 2-(tert-butoxycarbonylamino)-3-(4-(1,1-difluoroethyl)phenyl)acrylate (2.27 g, 6.65 mmol) was taken up in 50 mL of EtOH in a pressurizable tube and purged with nitrogen for 5 minutes. (+)-1,2-Bis((2S,5S)-2,5-diethylphospholano)benzene (cyclooctadiene)rhodium (I) trifluoromethanesulfonate (48 mg, 0.0665 mmol, Strem Chemicals, Inc.) was added. The tube was purged three times with hydrogen and pressurized to 30 psi. After 16 hours, the solvent was removed under reduced pressure and the residue was purified by flash chromatography on silica gel (10 to 30% EtOAc/hexanes) affording the title compound (1.83 g, 80.1%) as a white solid. LCMS (API-ES) m/z: 244 (M⁺+H-Boc); ¹H NMR (300 MHz, CDCl₃) δ ppm 1.41 (s, 9 H) 1.79-2.12 (m, 3H) 2.90-3.27 (m, 2H) 3.73 (s, 3H) 4.51-4.71 (m, 1H) 4.89-5.12 (m, 1H) 7.18 (d, J=7.89 Hz, 2H) 7.43 (d, J=8.18 Hz, 2H).

(S)-tert-Butyl 3-(4-(1,1-difluoroethyl)phenyl)-1-hydroxypropan-2-ylcarbamate: To a solution of (S)-methyl 2-(tert-butoxycarbonylamino)-3-(4-(1,1-difluoroethyl)phenyl)propanoate (1.80 g, 5.24 mmol) and EtOH (9.18 mL) in THF (20 mL) at 0° C. was slowly added LiBH₄ (228 mg, 10.48 mmol, Aldrich). The resulting mixture was stirred at 0° C. for 1 hour and was then stirred at room temperature for 3 hours. The reaction mixture was quenched with saturated NaH₂PO₄ solution, extracted with EtOAc, and the organic layer was dried over Na₂SO₄. After filtration and concentration, the title compound was obtained by silica gel flash column chromatography (eluted with hexane:EtOAc, 4:1) as a solid. LCMS (API-ES) m/z: 338 (M⁺+Na).

1,1-Dimethylethyl (4S)-4-((4-(1,1-difluoroethyl)phenyl)methyl)-1,2,3-oxathiazolidine-3-carboxylate 2-oxide: To a solution of (S)-tert-butyl 3-(4-(1,1-difluoroethyl)phenyl)-1-hydroxypropan-2-ylcarbamate (1.26 g, 4.0 mmol) in ACN (3 mL) and DCM (3 mL) was slowly added SOCl₂ (0.73 mL, 10 mmol, Aldrich) through a syringe. After addition, the reaction mixture was stirred at −60° C. for 10 minutes and pyridine (1.62 mL, 20 mmol) was added through a syringe while reaction mixture was maintained at −60° C. Upon completion of addition, the reaction mixture was warmed to room temperature, and the resulting mixture was stirred overnight. The mixture was quenched with saturated NaH₂PO₄, extracted with DCM (2×100 mL), and dried over MgSO₄. After filtration and concentration, the title compound was obtained by silica gel flash column chromatography (eluted with hexane:EtOAc, 4:1) as a solid. LCMS (API-ES) m/z: 384 (M⁺+Na); ¹H NMR (400 MHz, CDCl₃) δ ppm 1.54 (d, J=11.35 Hz, 9H) 1.91 (td, J=18.14, 1.86 Hz, 3H) 2.62-2.94 (m, 1H) 3.15-3.66 (m, 1H) 4.20-4.34 (m, 1H) 4.37-4.57 (m, 1H) 4.73-4.89 (m, 1H) 7.25-7.31 (m, 2H) 7.47 (dd, J=8.02, 4.50 Hz, 2H).

1,1-Dimethylethyl 5-bromo-1,3-thiazol-2-yl((2S)-3-(4-(1,1-difluoroethyl)phenyl)-2-((((1,1-dimethylethyl)oxy)carbonyl)amino)propyl)carbamate: To a mixture of cesium carbonate (1.00 g, 3.0 mmol) and tert-butyl 5-bromothiazol-2-ylcarbamate (643 mg, 2.3 mmol, prepared as shown for Example 1) in DMF (3 mL) at 60° C. was added a solution of 1,1-dimethylethyl (4S)-4-((4-(1,1-difluoroethyl)phenyl)methyl)-1,2,3-oxathiazolidine-3-carboxylate 2-oxide (833 mg, 2.30 mmol) in DMF (3 mL) dropwise through a syringe. Upon completion of addition, the reaction mixture was stirred at 60° C. overnight. The resulting mixture was cooled to room temperature, diluted with water, extracted with EtOAc (3×20 mL), and the combined organic extracts were dried over Na₂SO₄. After filtration and concentration, the title compound was obtained by silica gel flash column chromatography (eluted with hexane:EtOAc, 4:1) as a white solid. LCMS (API-ES) m/z: 476 (M⁺-Boc); ¹H NMR (300 MHz, CDCl₃) δ ppm 1.28-1.37 (s, 9H) 1.43 (s, 9H) 1.90 (t, J=18.12 Hz, 3H) 2.71-2.87 (m, 1H) 2.95-3.10 (m, 1H) 3.83-4.03 (m, 1H) 4.10-4.23 (m, 1H) 4.24-4.31 (m, 2H) 7.27-7.34 (m, 3H) 7.44 (d, J=8.18 Hz, 2H).

N—((S)-2-Amino-3-(4-(1,1-difluoroethyl)phenyl)propyl)-5-(3-fluoroisoquinolin-6-yl)thiazol-2-amine: The title compound was prepared in a manner similar to Example 1 using 1,1-dimethylethyl 5-bromo-1,3-thiazol-2-yl((2S)-3-(6-(1,1-difluoroethyl)-3-pyridinyl)-2-(((1,1-dimethylethyl)oxy)carbonyl)-amino)propyl)carbamate instead of 1,1-dimethylethyl 5-bromo-1,3-thiazol-2-yl((2S)-2-((((1,1-dimethylethyl)oxy)carbonyl)amino)-3-(6-(trifluoromethyl)-3-pyridinyl)propyl)carbamate as the substrate. LCMS (API-ES) m/z: 443 (M⁺+H); ¹H NMR (300 MHz, CDCl₃) δ ppm 1.57 (br. s., 2H) 1.93 (t, J=18.20 Hz, 3H) 2.66 (dd, J=13.45, 8.33 Hz, 1H) 2.94 (dd, J=13.52, 5.04 Hz, 1H) 3.14-3.29 (m, 1H) 3.36 (dd, J=7.82, 4.17 Hz, 1H) 3.52 (dd, J=12.57, 3.80 Hz, 1H) 7.16 (s, 1H) 7.26 (s, 2H) 7.29 (s, 1H) 7.48 (d, J=8.18 Hz, 2H) 7.56-7.69 (m, 3H) 7.90 (d, J=8.62 Hz, 1H) 8.85 (s, 1H).

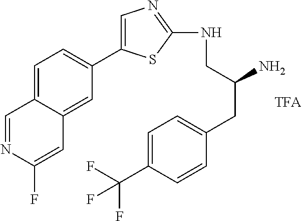

### Example 4

### N—((S)-2-Amino-3-(4-(trifluoromethyl)phenyl)propyl)-5-(3-fluoroisoquinolin-6-yl)thiazol-2-amine trifluoroacetate

This compound was prepared as shown in Scheme 4.

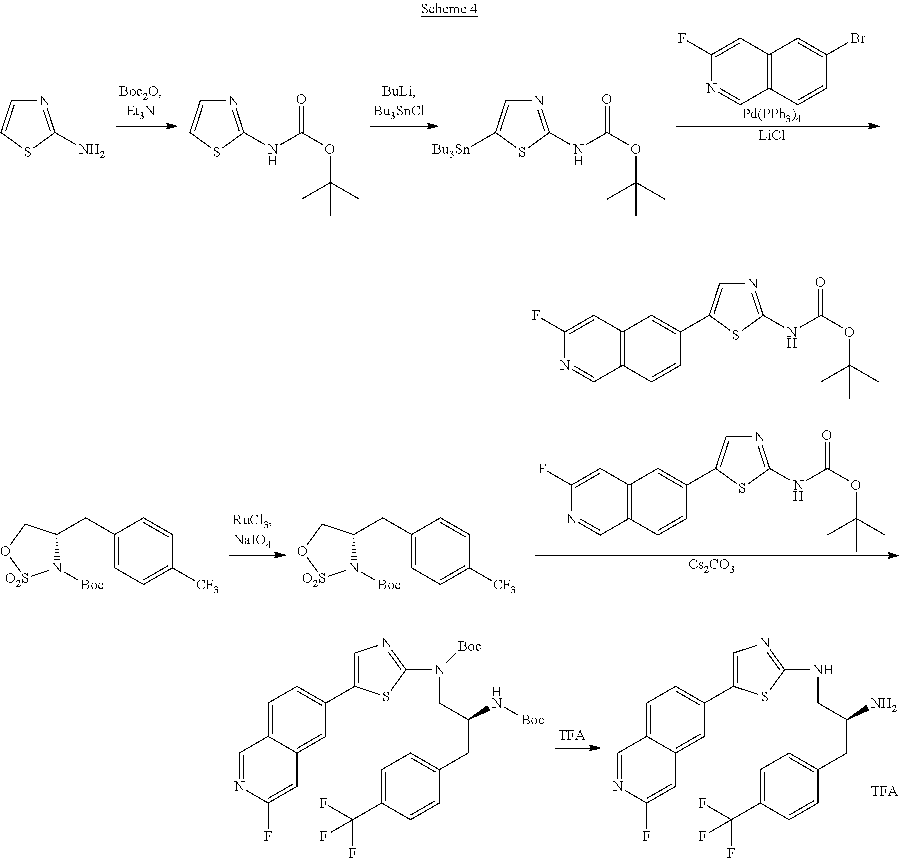

tert-Butyl thiazol-2-ylcarbamate: 2-Aminothiazole (10.0 g, 100 mmol, Aldrich) was dissolved in THF (50 mL) and di-tert-butyl dicarbonate was added (24 g, 110 mmol, Aldrich) followed by TEA (17 mL, 120 mmol). The resulting mixture was then stirred at ambient temperature for 16 hours. The solids were filtered and washed with ether to afford tert-butyl thiazol-2-ylcarbamate (13.5 g, 68%). LCMS (API-ES) m/z: 201 (M+H⁺).

tert-Butyl 5-(tributylstannyl)thiazol-2-ylcarbamate: To a 500 mL round-bottomed flask was added tert-butyl thiazol-2-ylcarbamate (2.9 g, 14 mmol) and THF (200 mL). The solution was stirred at −78° C. and treated dropwise via addition funnel with n-butyllithium (2.5 M in hexanes (12 mL, 30 mmol, Aldrich)). The suspension was stirred at −78° C. for 30 minutes and was then treated dropwise via addition funnel with tributyltin chloride (4.3 mL, 16 mmol, Aldrich). The resulting pale yellow solution was stirred at −78° C. for 30 minutes, allowed to warm to room temperature, and then stirred for an additional 2.5 hours. The reaction was quenched with NH₄Cl (100 mL). The layers were separated and the aqueous layer was extracted with ether (3×75 mL). The combined organic phases were washed with brine (100 mL), dried over MgSO₄, filtered, and concentrated in vacuo to give a viscous oil mixed with a white solid. The product thus obtained was adsorbed onto a plug of silica gel and chromatographed through a Redi-Sep® pre-packed silica gel column (40 g), eluting with a gradient of 10 to 20% EtOAc in hexane, to provide tert-butyl 5-(tributylstannyl)thiazol-2-ylcarbamate (4.6 g, 65%). LCMS (API-ES) m/z: 491 (M+H⁺).

1,1-Dimethylethyl 5-(3-fluoro-6-isoquinolinyl)-1,3-thiazol-2-ylcarbamate: A microwave reaction vessel was charged with 6-bromo-3-fluoroisoquinoline (0.212 g, 0.938 mmol, prepared as shown in Example 1), Pd(PPh₃)₄ (0.0542 g, 0.0469 mmol, Aldrich), lithium chloride (0.398 g, 9.38 mmol, Aldrich) and 1,1-dimethylethyl 5-(tributylstannnyl)-1,3-thiazol-2-ylcarbamate (0.734 g, 1.50 mmol) in DMF (0.5 mL) and purged with nitrogen for 10 minutes. The reaction vessel was then sealed and stirred at 80° C. overnight. The solvents were removed at 80° C. under reduced pressure, and the residue was diluted with water, extracted with EtOAc (3×100 mL), and the combined extracts were washed with saturated NaCl, and dried over Na₂SO₄. After filtration and concentration, the title compound was obtained by silica gel flash column chromatography (eluting with 0-25% EtOAc-hexane) as a light yellow solid (154 mg, 45%). LCMS (API-ES) m/z: 346 (M⁺+H); ¹H NMR (400 MHz, DMSO-d₆) δ ppm 1.52 (s, 9H) 7.56 (s, 1H) 7.98 (dd, J=8.71, 1.66 Hz, 1H) 8.08 (s, 1H) 8.12 (s, 1H) 8.19 (d, J=8.80 Hz, 1H) 9.05 (s, 1H) 11.74 (br. s., 1H).

tert-Butyl (4S)-4-(4-(trifluoromethyl)benzyl)-1,2,3-oxathiazolidine-3-carboxylate 2,2-dioxide: To a 1 L round-bottomed flask was added tert-butyl (4S)-4-(4-(trifluoromethyl)benzyl)-1,2,3-oxathiazolidine-3-carboxylate 2-oxide (15.2 g, 41.6 mmol, prepared according to Scheme 2, but using (S)-methyl 2-(tert-butoxycarbonylamino)-3-(4-(trifluoromethyl)phenyl)propanoate (Aldrich) instead of (S)-methyl 2-(tert-butoxycarbonylamino)-3-(6-(trifluoromethyl)pyridin-3-yl)propanoate) and EtOAc/CH₃CN/water and the mixture was stirred for 10 minutes. Sodium periodate (35.6 g, 166 mmol, Aldrich) and ruthenium(III) chloride (0.0259 g, 0.125 mmol, Aldrich) were added. After 18 hours, ether (500 mL) and water (400 mL) were added, the layers were separated, and the ether layer was washed with water (300 mL). The aqueous layer was washed with ether (4×500 mL) and the combined ether extracts were dried with MgSO₄, filtered, and evaporated to provide tert-butyl (4S)-4-(4-(trifluoromethyl)benzyl)-1,2,3-oxathiazolidine-3-carboxylate 2,2-dioxide as a white solid (15.3 g, 97%).

tert-Butyl ((2S)-2-((tert-butoxycarbonyl)amino)-3-(4-(trifluoromethyl)phenyl)propyl)(5-(3-fluoro-6-isoquinolinyl)-1,3-thiazol-2-yl)carbamate: A flame-dried flask (25 mL) was charged with tert-butyl 5-(3-fluoroisoquinolin-6-yl)thiazol-2-ylcarbamate (40 mg, 0.12 mmol), 1,1-dimethylethyl (4S)-4-((4-(trifluoromethyl)phenyl)methyl)-1,2,3-oxathiazolidine-3-carboxylate 2,2-dioxide (66 mg, 0.17 mmol) and cesium carbonate (75 mg, 0.23 mmol) in DMF (0.50 mL). The reaction mixture was stirred at 50° C. for 30 minutes. The solvents were removed at 80° C. under reduced pressure, and the residue was diluted with water, extracted with EtOAc (3×100 mL), washed with saturated NaCl, and dried over Na₂SO₄. After filtration and concentration, tert-butyl ((2S)-2-((tert-butoxycarbonyl)amino)-3-(4-(trifluoromethyl)phenyl)propyl)(5-(3-fluoro-6-isoquinolinyl)-1,3-thiazol-2-yl)carbamate was obtained by silica gel flash column chromatography (eluting with 0-25% EtOAc-hexane) as a light yellow solid (154 mg, 45%).

N—((S)-2-Amino-3-(4-(trifluoromethyl)phenyl)propyl)-5-(3-fluoroisoquinolin-6-yl)thiazol-2-amine trifluoroacetate: To a solution of tert-butyl ((2S)-2-((tert-butoxycarbonyl)amino)-3-(4-(trifluoromethyl)phenyl)propyl)(5-(3-fluoro-6-isoquinolinyl)-1,3-thiazol-2-yl)carbamate (60 mg, 0.09 mmol) in DCM (2 mL) was added TFA (2 mL, Aldrich). The mixture was stirred for 30 minutes at room temperature. After concentration, the residue was dried under vacuum overnight to give the title compound as a light yellow solid (63 mg, 99%). LCMS (API-ES) m/z: 447 (M⁺+H). ¹H NMR (400 MHz, DMSO-d₆) δ ppm 2.87 (d, J=6.65 Hz, 1H) 3.28-3.42 (m, 4H) 7.35 (s, 1H) 7.40 (d, J=8.02 Hz, 2H) 7.58 (d, J=8.22 Hz, 2H) 7.65 (s, 1H) 7.69 (s, 1H) 7.74 (dd, J=8.80, 1.57 Hz, 1H) 7.86 (br. s., 3H) 7.96 (d, J=8.80 Hz, 1H) 7.97 (d, 1H) 8.12 (t, J=5.28 Hz, 1H) 8.84 (s, 1H).

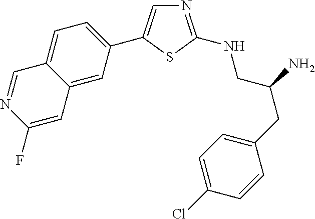

### Example 5

### N—((S)-2-Amino-3-(4-chlorophenyl)propyl)-5-(3-fluoroisoquinolin-6-yl)thiazol-2-amine

This compound was synthesized in a manner similar to Example 4 using (S)-methyl 2-(tert-butoxycarbonylamino)-3-(4-chlorophenyl)propanoate instead of (S)-methyl 2-(tert-butoxycarbonylamino)-3-(6-(trifluoromethyl)pyridin-3-yl)propanoate) as the intermediate. The title compound was obtained through silica gel flash column chromatography (eluted with DCM:2N NH₃ in MeOH, 19:1) as a light yellow solid (14 mg, 87%). LCMS (API-ES) m/z: 413 (M⁺+H); ¹H NMR (400 MHz, CDCl₃) δ ppm 1.62 (br. s, 3H) 2.57-2.66 (m, 1H) 2.88 (dd, J=13.50, 5.28 Hz, 1H) 3.16-3.25 (m, 1H) 3.29-3.36 (m, 1H) 3.51 (dd, J=12.62, 3.81 Hz, 1H) 7.13-7.18 (m, 3H) 7.31 (d, J=8.41 Hz, 2H) 7.58 (s, 1H) 7.63 (s, 1H) 7.65 (dd, J=8.71, 1.66 Hz, 1H) 7.91 (d, J=8.61 Hz, 1H) 8.85 (s, 1H).

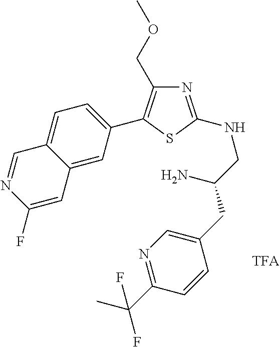

### Example 6

### N—((S)-2-Amino-3-(6-(1,1-difluoroethyl)pyridin-3-yl)propyl)-5-(3-fluoroisoquinolin-6-yl-4-(methoxymethyl)thiazol-2-amine trifluoroacetate

This compound was synthesized as shown in Scheme 5.

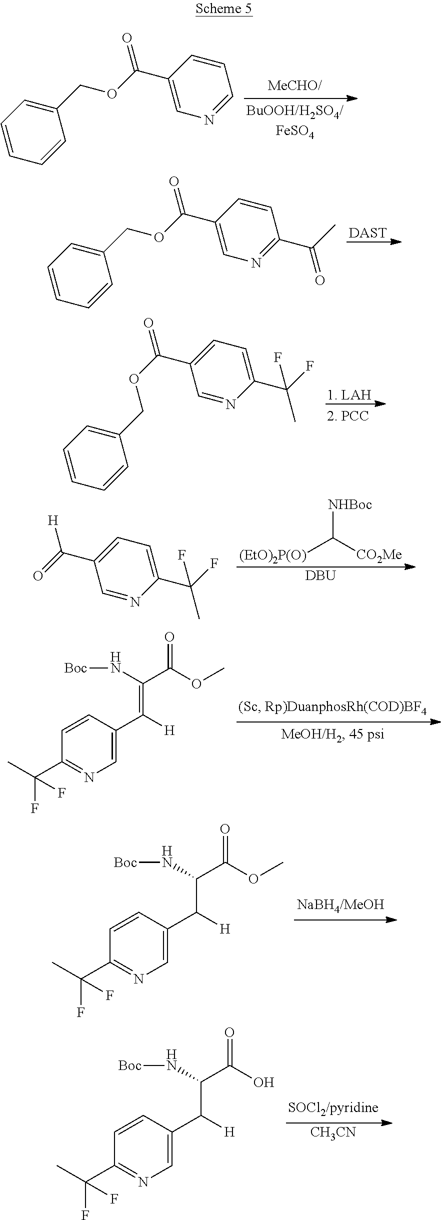

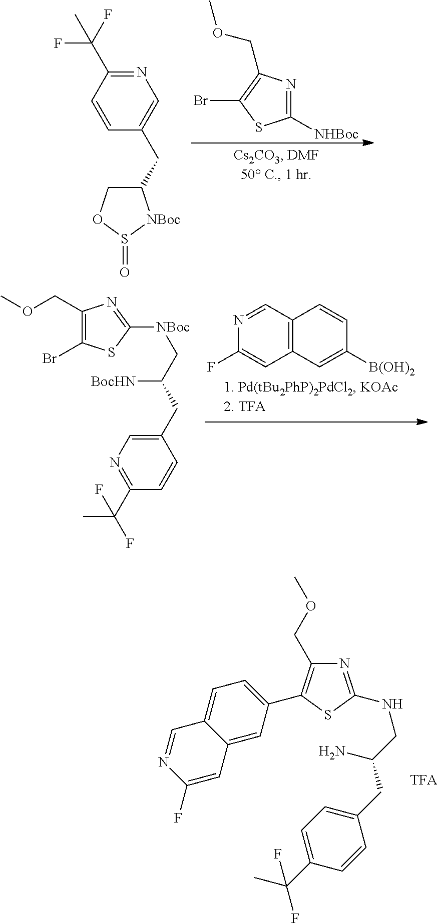

Benzyl 6-acetylnicotinate: To a solution of benzyl nicotinic acid, benzyl ester (21 mL, 117 mmol, Fluka), sulfuric acid 95-97% (10 mL, 117 mmol), and acetaldehyde (13 g, 291 mmol, Aldrich) in degassed water (50 mL) at 5-10° C. under argon, were simultaneously added dropwise a solution of ferrous sulfate, heptahydrate (43 mL, 291 mmol, Aldrich) in degassed water (200 mL) and t-BuOOH in water (40 mL, 70%, Fluka). The mixture was stirred for 15 minutes and then extracted with CHCl₃ (2×350 mL). The combined organic layers were then washed with brine (100 mL). After drying, the solvent was removed under vacuum. The mixture was purified by silica gel chromatography, eluting with 0 to 10% EtOAc/hexane, giving benzyl 6-acetylnicotinate (5.1 g, 17%): LCMS m/z: 256 (M+1).

Benzyl 6-(1,1-difluoroethyl)nicotinate. To a 200 mL high pressure reaction tube containing a solution of benzyl 6-acetylnicotinate (5.1 g, 20 mmol) and (diethylamino)trifluorosulfur (8 mL, 60 mmol, Aldrich) in chloroform (10 mL) was added EtOH (0.1 mL, 2 mmol). The resulting mixture was sealed and stirred at 60° C. overnight. After cooling to room temperature, the reaction mixture was poured into a saturated solution of NaHCO₃ (200 mL) at 0° C. and extracted with DCM (2×250 mL). The combined organic layers were dried over MgSO₄. After filtration and concentration, the residue was purified by silica gel chromatography, eluting with 0 to 10% EtOAc/hexane to give benzyl 6-(1,1-difluoroethyl)nicotinate as a oil (4.5 g, 81%). MS m/z: 278 (M+1).

(6-(1,1-Difluoroethyl)pyridin-3-yl)methanol: To a 250 mL of round bottom flask was added benzyl 6-(1,1-difluoroethyl)nicotinate (4.46 g, 16 mmol) and 50 mL THF. LAH (1.0 M solution in THF (24 mL, 24 mmol, Aldrich)) was added dropwise to the reaction mixture at 0° C. After 1 hour, LC-MS showed that the reaction was complete. The reaction was slowly quenched with 5 mL MeOH and then 30 mL of 10 M NaOH solution was added. The reaction mixture was filtered through Celite® brand filter aid and extracted with 150 mL of EtOAc twice. The organic layers were combined and concentrated. The residue thus obtained was purified by silica gel chromatography, eluting with 0-35% EtOAc/hexane to give (6-(1,1-difluoroethyl)pyridin-3-yl)methanol (2.5 g, 90%). MS m/z: 174 (M+1).

6-(1,1-Difluoroethyl)nicotinaldehyde: To a 25 mL round bottom flask was added (6-(1,1-difluoroethyl)pyridin-3-yl)methanol (2.4 g, 14 mmol), pyridinium chlorochromate (6.0 g, 28 mmol, Aldrich), 3 g of silica gel and 5 mL of DCM. After 3 hours, the reaction mixture was filtered through silica, rinsed with 75% EtOAc/hexane and then concentrated to give 6-(1,1-difluoroethyl)nicotinaldehyde (2.3 g, 97%). MS m/z: 172 (M+1).

(Z)-Methyl 2-(tert-butoxycarbonylamino)-3-(6-(1,1-difluoroethyl)pyridin-3-yl)acrylate: To a solution of methyl 2-(tert-butoxycarbonylamino)-2-(diethoxyphosphinooxy)acetate (4.8 g, 16 mmol, Fluka) in DCM (30 mL) was added 1,8-diazabicyclo[5.4.0]undec-7-ene (2.4 mL, 16 mmol, Aldrich) at 0° C. The resulting mixture was then stirred for 30 minutes. To the clear mixture was then slowly added a solution of 6-(1,1-difluoroethyl)nicotinaldehyde (2.3 g, 13 mmol) in DCM (30 mL) at 0° C. The mixture was stirred for 1.5 hours at 0-10° C. The resulting mixture was quenched with 1 N HCl solution, extracted with DCM, and dried over Na₂SO₄. After filtration and concentration, the desired product was obtained through silica gel chromatography, eluting with hexane:EtOAc, 4:1 (4.1 g, 89%). MS m/z: 343 (M+1).

Methyl 2-(tert-butoxycarbonylamino)-3-(6-(1,1-difluoroethyl)pyridin-3-yl)propanoate: A 250 mL tube was charged with (Z)-methyl 2-(tert-butoxycarbonylamino)-3-(6-(1,1-difluoroethyl)pyridin-3-yl)acrylate (3.05 g, 8.9 mmol), 30 mL of MeOH, and (Sc,Rp)DuanphosRh(COD)BF₄ (0.18 g, 0.27 mmol, Chiral The mixture was degassed for 3 minutes and then placed under 45 psi of hydrogen. After 3 hours, LCMS showed that the reaction was complete. The mixture was concentrated and passed through a small pad of silica, rinsing with 2:1 hexane/EtOAc. The filtrate was concentrated to give (S)-methyl 2-(tert-butoxycarbonylamino)-3-(6-(1,1-difluoroethyl)pyridin-3-yl)propanoate (2.5 g, 81%). MS m/z: 345 (M+1).

(S)-tert-Butyl 3-(6-(1,1-difluoroethyl)pyridin-3-yl)-1-hydroxypropan-2-ylcarbamate. To a solution of (S)-methyl 2-(tert-butoxycarbonylamino)-3-(6-(1,1-difluoroethyl)pyridin-3-yl)propanoate (1.2 g, 3.5 mmol) in EtOH (10 mL) was added NaBH₄ (0.13 g, 3.5 mmol, Fluka). After addition, the reaction mixture was stirred at room temperature for 1 hour. The mixture was then concentrated and then 50 mL of EtOAc and 20 mL saturated NaHCO₃ were added. The organic layer was concentrated and purified by silica gel chromatography, eluting with 0-35% EtOAc/hexane to give, (S)-tert-butyl 3-(6-(1,1-difluoroethyl)pyridin-3-yl)-1-hydroxypropan-2-ylcarbamate (1.0 g, 91%). MS m/z: 317 (M+1).

tert-Butyl (4S)-4-((6-(1,1-difluoroethyl)-3-pyridinyl)methyl)-1,2,3-oxathiazolidine-3-carboxylate 2-oxide. SOCl₂ (0.45 mL, 6.2 mmol, Aldrich) was taken up in 10 mL of CH₃CN and chilled to −55° C. (S)-tert-Butyl 3-(6-(1,1-difluoroethyl)pyridin-3-yl)-1-hydroxypropan-2-ylcarbamate (0.78 g, 2.5 mmol) was then added slowly in 10 mL of ACN. After 15 minutes, pyridine (1.00 mL, 12 mmol) was added, and the mixture was warmed to room temperature. The mixture was next concentrated under reduced pressure. The residue was taken up in 50 mL of EtOAc and 50 mL of water. The mixture was partitioned, and the aqueous portion was extracted with 50 mL of EtOAc. The combined organic extracts were washed with 50 mL of brine and dried over MgSO₄. Filtration and concentration under reduced pressure, followed by flash chromatography on silica gel (0% to 25% EtOAc/hexanes) afforded tert-butyl (4S)-4-((6-(1,1-difluoroethyl)-3-pyridinyl)methyl)-1,2,3-oxathiazolidine-3-carboxylate 2-oxide (0.80 g, 90%). MS m/z: 363 (M+1).

tert-Butyl (5-bromo-4-(methoxymethyl)-1,3-thiazol-2-yl)((2S)-2-((tert-butoxycarbonyl)amino)-3-(6-(1,1-difluoroethyl)-3-pyridinyl)propyl)carbamate. tert-Butyl 5-bromo-4-(methoxymethyl)thiazol-2-ylcarbamate (322 mg, 1.0 mmol) was taken up in 5 mL of DMF and heated to 50° C. Cs₂CO₃ (0.74 g, 2.26 mmol) was added, followed by slow addition of tert-butyl (4S)-4-((6-(1,1-difluoroethyl)-3-pyridinyl)methyl)-1,2,3-oxathiazolidine-3-carboxylate 2-oxide (0.41 g, 1.13 mmol) in 5 mL of DMF. After 16 hours, the solvent was removed under reduced pressure. The residue was taken up in 20 mL of EtOAc and 20 mL of 2 M aqueous HCl was then slowly added. The mixture was stirred for 20 minutes. The mixture was partitioned, and the aqueous portion was extracted twice with 20 mL of EtOAc. The combined organic extracts were washed with 20 mL of brine and dried over MgSO₄. Filtration and concentration under reduced pressure, followed by flash chromatography on silica gel (0% to 25% EtOAc/hexanes) afforded the title compound (0.52 g, 90%). MS m/z: 621 (M+1).

tert-Butyl ((2S)-2-((tert-butoxycarbonyl)amino)-3-(6-(1,1-difluoroethyl)-3-pyridinyl)propyl)(5-(3-fluoro-6-isoquinolinyl)-4-(methoxymethyl)-1,3-thiazol-2-yl)carbamate. To a 25 mL round-bottom flask was added potassium acetate (0.17 g, 1.68 mmol), PdCl₂(t-butylPPh₃)₂ (13.4 mg, 21.6 μmol, Johnson Matthey catalog number Pd-122), 3-fluoroisoquinolin-7-ylboronic acid (68.7 mg, 0.36 mmol), tert-butyl (5-bromo-4-(methoxymethyl)-1,3-thiazol-2-yl)((2S)-2-((tert-butoxycarbonyl)amino)-3-(6-(1,1-difluoroethyl)-3-pyridinyl)propyl)carbamate (0.15 g, 0.24 mmol), 2.4 mL of CH₃CN, and 0.8 mL of water. The reaction mixture was heated at 85° C. for 3 hours and was then concentrated and purified by silica gel chromatography, eluting with 0-50% EtOAc/hexane to give tert-butyl ((2S)-2-((tert-butoxycarbonyl)amino)-3-(6-(1,1-difluoroethyl)-3-pyridinyl)propyl)(5-(3-fluoro-6-isoquinolinyl)-4-(methoxymethyl)-1,3-thiazol-2-yl)carbamate. MS m/z: 688 (M+1).

N—((S)-2-Amino-3-(6-(1,1-difluoroethyl)pyridin-3-yl)propyl)-5-(3-fluoroisoquinolin-6-yl)-4-(methoxymethyl)thiazol-2-amine trifluoroacetate. A mixture of 70% TFA in DCM (3 mL) was added to tert-butyl ((2S)-2-((tert-butoxycarbonyl)amino)-3-(6-(1,1-difluoroethyl)-3-pyridinyl)propyl)(5-(3-fluoro-6-isoquinolinyl)-4-(methoxymethyl)-1,3-thiazol-2-yl)carbamate. After 30 minutes, the reaction mixture was concentrated and purified by preparative HPLC (10-100% CH₃CN/H₂O, 0.1% TFA) to give the title compound (70 mg, 60% over two steps). MS m/z: 488 (M+1); ¹H NMR (400 MHz, CD₃OD): ppm δ 1.95-2.04 (m, 3H) 3.08-3.18 (m, 2H) 3.45 (s, 3H) 3.57-3.61 (m, 1H) 3.71-3.76 (m, 1H) 3.90 (dd, J=7.53, 4.21 Hz, 1H) 4.44 (s, 2H) 7.44 (s, 1H) 7.66 (dd, J=8.80, 1.76 Hz, 1H) 7.74 (d, J=8.22 Hz, 1H) 7.93-7.98 (m, 2H) 8.16 (d, J=8.80 Hz, 1H) 8.62 (s, 1H) 9.01 (s, 1H).

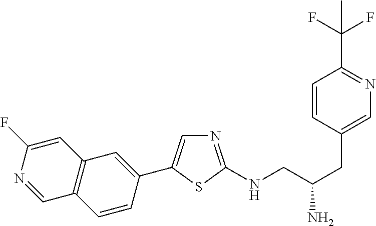

### Example 7

### N—((S)-2-Amino-3-(6-(1,1-difluoroethyl)pyridin-3-yl)propyl)-5-(3-fluoroisoquinolin-6-yl)thiazol-2-amine

The title compound was synthesized in a manner similar to that described for Example 6, but using tert-butyl 5-bromothiazol-2-ylcarbamate prepared according to Scheme 2 for Example 1, instead of tert-butyl 5-bromo-4-(methoxymethyl)thiazol-2-ylcarbamate. MS m/z: 444 (M+1). ¹H NMR (400 MHz, MeOH) δ ppm 1.98 (t, J=18.75 Hz, 3H), 3.10 (d, J=6.85 Hz, 1H), 3.13-3.20 (m, 1H), 3.57-3.63 (m, 1H), 3.72 (d, J=3.91 Hz, 1H), 3.89 (dd, J=7.04, 4.11 Hz, 1H), 7.38 (s, 1H), 7.72-7.75 (m, 2H), 7.80-7.85 (m, 2H), 7.95 (dd, J=8.02, 2.15 Hz, 1 H), 8.09 (d, J=8.61 Hz, 1H), 8.61 (d, J=1.37 Hz, 1H), 8.92 (s, 1H).

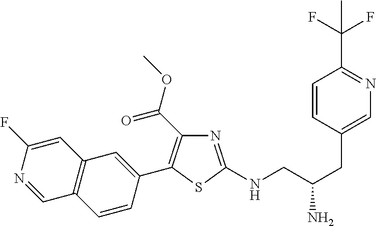

### Example 8

### Methyl 2-((S)-2-amino-3-(6-(1,1-difluoroethyl)pyridin-3-yl)propylamino)-5-(3-fluoroisoquinolin-6-yl)thiazole-4-carboxylate

The title compound was synthesized in a manner similar to that described for Example 6, but using methyl 5-bromo-2-(tert-butoxycarbonylamino)thiazole-4-carboxylate (Synchem catalog number C-21889) instead of tert-butyl 5-bromo-4-(methoxymethyl)thiazol-2-ylcarbamate. MS m/z: 502 (M+1). ¹H NMR (400 MHz, CD₃OD): δ ppm 1.95 (m, 3H) 3.06-3.18 (m, 2H) 3.60-3.64 (m, 1H) 3.71 (s, 3H) 3.73 (d, J=3.52 Hz, 1H) 3.90 (d, J=7.04 Hz, 1H) 7.47 (s, 1H) 7.66 (dd, J=8.61, 1.76 Hz, 1H) 7.74 (d, J=8.22 Hz, 1H) 7.97-7.99 (dd, J=1.96, 0.39 Hz, 1H) 8.02 (s, 1H) 8.15 (d, J=8.61 Hz, 1H), 8.63-8.62 (d, J=4.0 Hz, 1H) 9.05 (s, 1H).

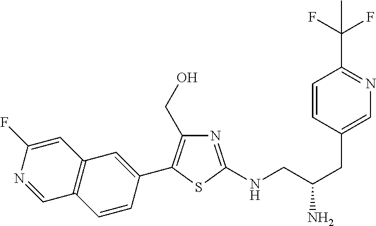

### Example 9

### (2-((S)-2-Amino-3-(6-(1,1-difluoroethyl)pyridin-3-yl)propylamino)-5-(3-fluoroisoquinolin-6-yl)thiazol-4-yl)methanol

To a 25 mL round-bottom flask was added methyl 2-((S)-2-amino-3-(6-(1,1-difluoroethyl)pyridin-3-yl)propylamino)-5-(3-fluoroisoquinolin-6-yl)thiazole-4-carboxylate (30 mg, 60 μmol, prepared according to Example 8), NaBH₄ (12 mg, 315 μmol) and 2 mL of MeOH. The reaction mixture was stirred for 2 hours. 10 mL of water was added, and the mixture was then extracted twice with 20 mL of EtOAc. The organic layers were combined, concentrated, and purified by preparative HPLC (10-100% CH₃CN/H₂O, 0.1% TFA) to give the title compound (15 mg, 53%). MS m/z: 474 (M+1); ¹H NMR (400 MHz, MeOH) δ ppm 1.99 (t, J=18.78 Hz, 3H), 3.06-3.20 (m, 2H), 3.56-3.62 (m, 1H), 3.73 (d, J=3.52 Hz, 1H), 3.91 (d, J=3.52 Hz, 1H), 4.61 (s, 2H), 7.44 (s, 1H), 7.68 (dd, J=8.61, 1.57 Hz, 1H), 7.74 (d, J=8.22 Hz, 1H), 7.96 (s, 1H), 7.98 (d, J=2.15 Hz, 1H), 8.16 (d, J=8.61 Hz, 1H), 8.62 (s, 1H), 9.00 (s, 1H).

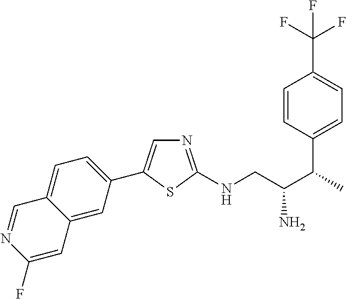

### Example 10

### N-((2S,3S)-2-Amino-3-(4-(trifluoromethyl)phenyl)butyl)-5-(3-fluoroisoquinolin-6-yl)thiazol-2-amine

The title compound was synthesized in a manner similar to that described for Example 7, but using (4S,5S)-5-methyl-4-(4-(trifluoromethyl)benzyl)-1,2,3-oxathiazolidine-3-carboxylate 2,2-dioxide instead of tert-butyl (4S)-4-((6-(1,1-difluoroethyl)-3-pyridinyl)methyl)-1,2,3-oxathiazolidine-3-carboxylate 2-oxide. MS m/z: 461 (M+1). ¹H NMR (400 MHz, CD₃OD): δ ppm 1.50 (d, J=7.04 Hz, 3H); 3.69-3.82 (m, 2H) 3.90-3.93 (m, 1H) 7.38 (s, 1H) 7.58 (d, J=8.02 Hz, 2H) 7.73 (t, J=4.11 Hz, 3H) 7.80-7.85 (m, 2H) 8.09 (d, J=8.61 Hz, 1H) 8.92 (s, 1H). (4S,5S)-5-Methyl-4-(4-(trifluoromethyl)benzyl)-1,2,3-oxathiazolidine-3-carboxylate 2,2-dioxide was synthesized according to Scheme 6.

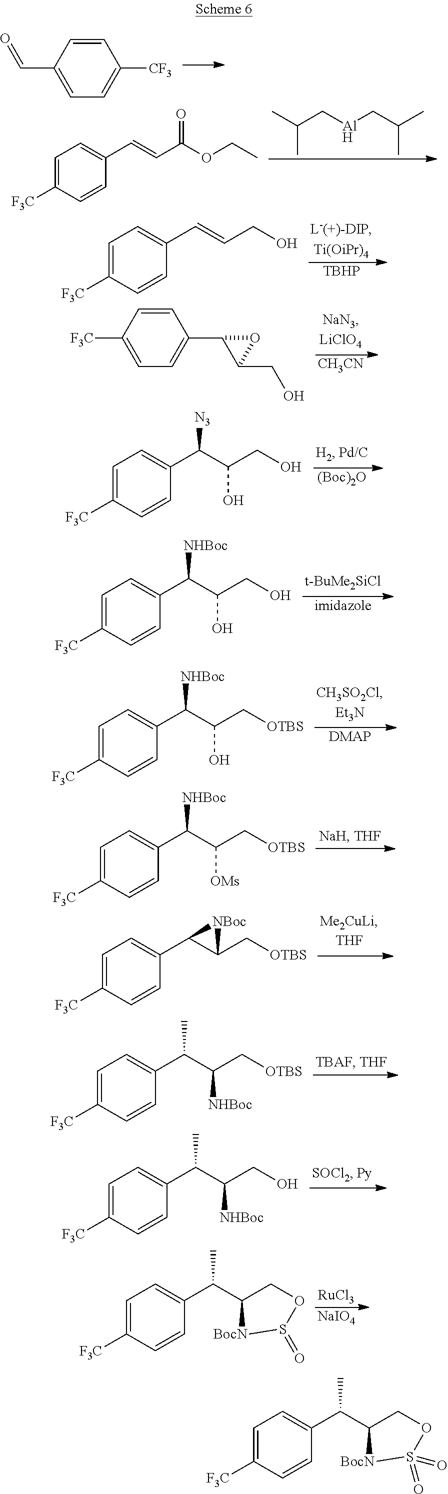

(E)-Ethyl 3-(4-(trifluoromethyl)phenyl)acrylate. To a solution of (carbethoxymethylene)triphenylphosphorane (55.3 g, 159 mmol, Aldrich) in 150 mL DCM, was added 4-(trifluoromethyl)benzaldehyde (25.0 g, 144 mmol, Aldrich) in 75 mL DCM. The reaction was exothermic. The mixture was heated at reflux for 90 minutes. After removing the solvent, hexane was added to the resulting residue. A precipitate appeared and was filtered through filter paper. The collected solid was purified by silica gel chromatography with 100% hexane as the eluant to afford a white solid ((E)-ethyl 3-(4-(trifluoromethyl)phenyl)acrylate (25.0 g, 71%). LCMS (API-ES) m/z (%): 245.1 (100%, M⁺+H); ¹H NMR (400 MHz, CD₃OD) δ ppm 1.35 (t, J=7.14 Hz, 3H) 4.28 (q, J=7.04 Hz, 2H) 6.68 (d, J=16.04 Hz, 1H) 7.70-7.77 (m, 3H) 7.80-7.84 (m, 2H).

(E)-3-(4-(Trifluoromethyl)phenyl)prop-2-en-1-ol. (E)-Ethyl 3-(4-(trifluoromethyl)phenyl)acrylate (25.0 g, 102 mmol) in 100 mL ether was cooled in an ice-water bath. To this solution was added di-iso-butylaluminum hydride (205 mL, 205 mmol, Aldrich) in hexane. After addition, the ice-water bath was removed. After 2 hours of stirring at room temperature, the reaction mixture was diluted with 200 mL diethyl ether, cooled to 0° C. and quenched with careful addition of 200 mL brine and 200 mL of 5.0 M HCl. The aqueous solution was extracted twice with diethyl ether (200 mL each time). The combined organic phases were washed with brine and dried over sodium sulfate. After filtration and solvent removal, the product was chromatographed eluting with 20% EtOAc in hexane. After removing the solvent, a white solid was obtained as the desired product (15.3 g, 74%). ¹H NMR (400 MHz, CD₃OD) δ ppm 4.28 (dd, J=5.28, 1.57 Hz, 2H) 6.55 (dt, J=15.94, 5.23 Hz, 1H) 6.67-6.75 (m, 1H) 7.58-7.67 (m, 4H).

((2S,3S)-3-(4-(Trifluoromethyl)phenyl)oxiran-2-yl)methanol. Into a 2 L flame-dried flask were introduced dry powdered 4 Å molecular sieves (9.0 g) and anhydrous DCM (1.0 L) under nitrogen. After cooling to −20° C., the following reagents were introduced sequentially via cannula under stirring: diisopropyl 1-tartrate (5 g, 21 mmol, Aldrich); titanium tetraisopropoxide (4 mL, 14 mmol, Aldrich); and a 5.5 M solution of t-butylhydroperoxide (101 mL, 0.55 mol, Aldrich). The mixture was stirred 1 hour at −20° C. and a solution of (E)-3-(4-(trifluoromethyl)phenyl)prop-2-en-1-ol (56.0 g, 0.28 mol) in 150 mL DCM was added over a 30 minute period. After 8 hours of stirring at the same temperature, the reaction was quenched by addition of 24 mL of a 10% aqueous solution of NaOH saturated with NaCl (100 mL of a 10% solution were prepared by adding 10 g of NaCl to a solution of 10 g of NaOH in 95 mL water). Diethyl ether (300 mL)was added dropwise while the cold bath was maintained at −20° C. After the ether addition, the cold bath was removed, and the mixture was allowed to warm to 10° C. Stirring was maintained for an additional 15 minutes at 10° C., and anhydrous MgSO₄ (24 g) and Celite® brand filter aid (3 g) were added. After a final 30 minutes of stirring, the mixture was allowed to settle, and the upper portion was filtered through a pad of Celite® brand filter aid. The Celite® brand filter aid was washed with 20 mL of ether. The solvents were evaporated, and tert-butyl hydroperoxide was removed by azeotropic evaporation with toluene (3×100 mL) under high vacuum. The product thus obtained was then chromatographed eluting with 30% EtOAc in hexane. After removing the solvent, a colorless oil was obtained as the desired product (54 g, 90%). ¹H NMR (400 MHz, CD₃OD) δ ppm 3.17 (ddd, J=4.74, 2.89, 2.15 Hz, 1H) 3.72 (dd, J=12.72, 4.70 Hz, 1H) 3.90 (dd, J=12.72, 2.93 Hz, 1H) 3.96 (d, J=1.96 Hz, 1H) 7.50 (d, J=8.02 Hz, 2H) 7.66 (d, J=8.22 Hz, 2H).

(2R,3R)-3-Azido-3-(4-(trifluoromethyl)phenyl)propane-1,2-diol. To a mixture of ((2S,3S)-3-(4-(trifluoromethyl)phenyl)oxiran-2-yl)methanol (15.0 g, 68.8 mmol) in 400 mL ACN was added lithium perchlorate (75.3 mL, 1.72 mol). The reaction mixture was a suspension. After stirring for 15 minutes, sodium azide (12.1 mL, 344 mmol, Aldrich) was added, and the mixture was heated at 65° C. for 24 hours under nitrogen. After the reaction mixture was cooled, the solvent was evaporated under reduced pressure. 500 mL distilled water was added, and the resulting mixture was extracted with diethyl ether (3×400 mL). The combined ether layers were directly dried over MgSO₄. After filtration and solvent removal, a colorless oil was obtained as the desired product (14.5 g, 81%). ¹H NMR (400 MHz, CD₃OD) δ ppm 3.50-3.56 (m, 1H) 3.58-3.63 (m, 1H) 3.84-3.90 (m, 1H) 4.78 (d, J=6.53 Hz, 1H) 7.62 (d, J=8.03 Hz, 2H) 7.67-7.73 (m, 2H).

tert-Butyl (1R,2R)-2,3-dihydroxy-1-(4-(trifluoromethyl)phenyl)-propylcarbamate. To (2R,3R)-3-azido-3-(4-(trifluoromethyl)phenyl)propane-1,2-diol (14.50 g, 56 mmol) in 120 mL EtOAc, were added di-tert-butyldicarbonate (17 g, 78 mmol, Aldrich) and 10% Pd/C (1.45 g, 14 mmol, Aldrich). The mixture was hydrogenated at atmospheric pressure until no starting material could be observed by TLC (about 36 hours). The reaction mixture was filtered through Celite® brand filter aid. The filtrate was washed twice with water and twice with brine solution and then dried over sodium sulfate. After filtration and solvent removal, 100 mL hexane was added into the residue and a precipitate appeared. The resulting precipitate was filtered and washed with cold hexane. The white solid was air-dried and was obtained as the desired product (11.0 g, 60%). ¹H NMR (400 MHz, CD₃OD) δ ppm 1.42 (s, 9H) 3.43-3.52 (m, 2H) 3.84 (q, J=5.41 Hz, 1H) 4.75 (d, J=5.67 Hz, 1H) 7.52-7.57 (m, 2H) 7.60-7.64 (m, 2H).

tert-Butyl (1R,2R)-3-(tert-butyldimethylsilyloxy)-2-hydroxy-1-(4-(trifluoromethyl)phenyl)propylcarbamate. tert-Butyl (1R,2R)-2,3-dihydroxy-1-(4-(trifluoromethyl)phenyl)-propylcarbamate (11 g, 32.8 mol) in 100 mL DMF was cooled in an ice-water bath. 1H-imidazole (8.2 mL, 72 mol) was added in one portion, and the mixture was stirred for 10 minutes under nitrogen. tert-Butyldimethylsilylchloride (5.43 g, 36.0 mmol, Aldrich) in 20 mL DMF was added via syringe. The reaction was monitored by TLC. After 16 hours, DMF was evaporated under high vacuum. Distilled water (150 mL) was added, and the resulting mixture was extracted into diethyl ether (2×200 mL). The ether layer was washed with saturated aqueous ammonia chloride and dried over sodium sulfate. After filtration and solvent removal, the product was obtained as a white solid (14.0 g, 95%). ¹H NMR (400 MHz, CD₃OD) δ ppm 0.08-0.12 (m, 6H) 0.97 (s, 9H) 1.43 (s, 9H) 3.50 (s, 1H) 3.63 (s, 1H) 3.85 (s, 1H) 4.81 (s, 1H) 7.53-7.58 (m, 2H) 7.60-7.66 (m, 2H).

(1R,2R)-1-(tert-butoxycarbonylamino)-3-(tert-butyldimethylsilyloxy)-1-(4-(trifluoromethyl)phenyl)propan-2-yl methanesulfonate. To a solution of tert-butyl (1R, 2R)-3-(tert-butyldimethylsilyloxy)-2-hydroxy-1-(4-(trifluoromethyl)phenyl)propylcarbamate (14.50 g, 32.3 mmol) in 50 mL DCM at −15° C. were added TEA (4.57 g, 45.2 mmol), N,N-dimethylpyridin-4-amine (0.197 g, 1.61 mmol), and methanesulfonyl chloride (3.26 mL, 41.9 mmol, Aldrich). The mixture was allowed to warm to room temperature. 200 mL distilled water was added, and the aqueous phase was extracted into DCM (2×200 mL). The combined organic layer was washed with cold 5% HCl, saturated sodium bicarbonate, and water. After removing the solvent, the product thus obtained was chromatographed eluting with 15% EtOAc in hexane. After removing the solvent, the product was obtained as a colorless oil (15 g, 88%). ¹H NMR (400 MHz, CD₃OD) δ ppm 0.11 (d, J=3.91 Hz, 6H) 0.93-0.98 (m, 9H) 1.44 (s, 9H) 2.84 (s, 3H) 3.80-3.87 (m, 2H) 4.84-4.86 (m, 1H) 5.13 (d, J=5.28 Hz, 1H) 7.58 (d, J=8.02 Hz, 2H) 7.69 (d, J=8.22 Hz, 2H).

(2R,3R)-tert-Butyl 2-((tert-butyldimethylsilyloxy)methyl)-3-(4-(trifluoromethyl)phenyl)aziridine-1-carboxylate. To a suspension of 4.0 g NaH (60% dispersion in mineral oil, Aldrich) in 50 mL THF at 0° C. was added a solution (1R,2R)-1-(tert-butoxycarbonylamino)-3-(tert-butyldimethylsilyloxy)-1-(4-(trifluoromethyl)phenyl)propan-2-yl methanesulfonate (13.5 g, 25.6 mmol) in 60 mL THF. The reaction progress was monitored by TLC (20% EtOAc in hexane). When no more starting material could be detected, 4 grams of MeOH was added to the mixture to remove the excess NaH. The solvent was removed at reduced pressure, and 200 mL of distilled water was added to the residue. The aqueous phase was extracted (3×150 mL) with EtOAc. The combined organic layers were washed with brine and dried over sodium sulfate. The organic layer was then filtered and the solvent was removed. The initially obtained product was then chromatographed eluting with 3% EtOAc in hexane. After removing the solvent, the product was obtained as a colorless oil (6.5 g, 58%). ¹H NMR (400 MHz, CD₃OD) δ ppm 0.14-0.18 (m, 6H) 0.95-0.98 (m, 9H) 1.46 (s, 9H) 2.78 (q, J=2.80 Hz, 1H) 3.64 (d, J=2.93 Hz, 1H) 4.11 (ddd, J=18.19, 11.93, 2.54 Hz, 2H) 7.48 (d, J=8.22 Hz, 2H) 7.66 (d, J=8.02 Hz, 2H).

tert-Butyl (2S,3S)-1-(tert-butyldimethylsilyloxy)-3-(4-(trifluoromethyl)phenyl)butan-2-ylcarbamate. To a stirred slurry of cuprous iodide (7.9 g, 42 mmol, Aldrich) in 150 mL ether at 0° C. was added methyllithium (1.6 M solution in diethyl ether (52 mL, 83 mmol), Aldrich). The mixture was stirred at this temperature for 20 minutes. A solution of (2R,3R)-tert-butyl 2-((tert-butyldimethylsilyloxy)methyl)-3-(4-(trifluoromethyl)phenyl)aziridine-1-carboxylate (6.0 g, 14 mmol) in 150 mL ether was added via cannula to the lithium dimethylcuprate solution. The mixture was stirred at 0° C. and monitored by TLC. When no starting material could be detected (ca.7 hours), 250 mL of an 8:1 mixture of saturated aqueous ammonia chloride and ammonia hydroxide (28-30% in water) was added to the reaction. The resulting reaction mixture was extracted with diethyl ether (2×300 mL). The combined organic layers were washed twice with brine and dried over sodium sulfate. The organic layer was filtered and the solvent was removed. The product thus obtained was then chromatographed eluting with 3% EtOAc in hexane. After removing the solvent, the desired product was obtained as a colorless oil (2.0 g, 32%). ¹H NMR (400 MHz, CD₃OD) δ ppm 0.09 (s, 6H) 0.90-0.96 (m, 9H) 1.25-1.33 (m, 12H) 3.00-3.11 (m, 1H) 3.68-3.80 (m, 3H) 7.43 (d, J=8.02 Hz, 2H) 7.56 (d, J=8.02 Hz, 2H).

tert-Butyl (2S,3S)-1-hydroxy-3-(4-(trifluoromethyl)phenyl)butan-2-ylcarbamate. To tert-butyl (2S,3S)-1-(tert-butyldimethylsilyloxy)-3-(4-(trifluoromethyl)phenyl)butan-2-ylcarbamate (2.0 g, 4.5 mmol) in 25 mL ether at 0° C. was added 1.0 M tetrabutylammonium fluoride in THF (8.9 mL, 8.9 mmol, Aldrich). After addition, the ice-bath was taken away. The reaction progress was monitored by TLC. After 60 minutes, the solvent was evaporated and 100 mL diethyl ether was added. The organic layer was washed with water and brine solution, and then dried over sodium sulfate. The organic layer was filtered and the solvent was removed. The product thus obtained was then chromatographed eluting with 30% EtOAc in hexane. After removing the solvent, the desired product was obtained as a white solid (1.25 g, 84%). ¹H NMR (400 MHz, CD₃OD) δ ppm 1.26-1.31 (m, 9H) 1.34 (d, J=7.04 Hz, 3H) 3.02-3.13 (m, 1H) 3.61-3.67 (m, 2H) 3.75 (dd, J=8.71, 4.60 Hz, 1H) 7.44 (d, J=8.02 Hz, 2H) 7.57 (d, J=8.22 Hz, 2H).

Mixture of (R)-tert-butyl 4-((S)-1-(4-(trifluoromethyl)phenyl)-1-S-ethyl)-1,2,3-oxathiazolidine-3-carboxylate, 2-oxide and (S)-tert-butyl 4-((S)-1-(4-(trifluoromethyl)phenyl)-1-S-ethyl)-1,2,3-oxathiazolidine-3-carboxylate, 2-oxide. To a solution of SOCl₂ (0.6 mL, 8 mmol, Aldrich) in 10 mL of CH₃CN at −60° C. was dropwise added tert-butyl (2 S,3S)-1-hydroxy-3-(4-(trifluoromethyl)phenyl)butan-2-ylcarbamate (1.1 g, 3 mmol) in 20 mL of CH₃CN via syringe. After 10 minutes, pyridine (1 mL, 16 mmol) was added dropwise while keeping the cold bath temperature at −60° C. The mixture was allowed to warm to room temperature and stirred 16 hours. During the warm up period, the reaction mixture was still a suspension. After 16 hours of stirring, the reaction became a clear brown solution. The solvent was then removed under reduced pressure. The residue was taken up in 100 mL of EtOAc. The mixture was transferred to a separatory funnel and washed twice with 100 mL of water and once with 100 mL of brine. The organic layer was dried over Na₂SO₄. Filtration and concentration under reduced pressure, followed by flash chromatography on silica gel (5% to 10% EtOAc/hexanes) afforded 1.0 g of the mixture of diastereomers as a yellow solid (900 mg, 70%): ¹H NMR (400 MHz, CDCl₃) δ ppm 1.40-1.44 (m, 9H) 1.51 (m, 3H) 3.62-3.70 (m, 1H) 4.37-4.46 (m, 2H) 4.79-4.89 (m, 1H) 7.38-7.43 (m, 2H) 7.59 (t, J=8.90 Hz, 2H).

(S)-tert-Butyl 4-((S)-1-(4-(trifluoromethyl)phenyl)ethyl)-1,2,3-oxathiazolidine-3-carboxylate, 2,2-dioxide. Sodium periodate (2.30 g, 9.5 mmol), ruthenium(III) chloride hydrate (2.67 mg, 0.012 mmol) and a mixture of (R)-tert-butyl 4-((S)-1-(4-(trifluoromethyl)phenyl)-1-S-ethyl)-1,2,3-oxathiazolidine-3-carboxylate, 2-oxide and (S)-tert-butyl 4-((S)-1-(4-(trifluoromethyl)phenyl)-1-S-ethyl)-1,2,3-oxathiazolidine-3-carboxylate, 2-oxide (900 mg, 2.37 mmol) were mixed together in a 500 mL round bottom flask. The ratio of the solvent by volume was as follows: ACN:water:EtOAc=30:10:5. 45 mL of ACN was used. The solids were suspended by 17 minutes sonication. The mixture was filtered through filter paper and washed with DCM. The solvent was evaporated. The resulting mixture was taken up in DCM and washed with water and brine solution. The organic layer was dried over sodium sulfate, filtered, and evaporated to provide (S)-tert-butyl 4-((S)-1-(4-(trifluoromethyl)phenyl)ethyl)-1,2,3-oxathiazolidine-3-carboxylate, 2,2-dioxide (840 mg, 88%): ¹H NMR (400 MHz, CDCl₃) δ ppm 1.44-1.49 (m, 12H) 3.51-3.59 (m, J=6.90, 6.90, 6.90, 6.90 Hz, 1H) 4.40-4.50 (m, 3H) 7.43 (d, J=8.22 Hz, 2H) 7.61 (d, J=8.22 Hz, 2H).

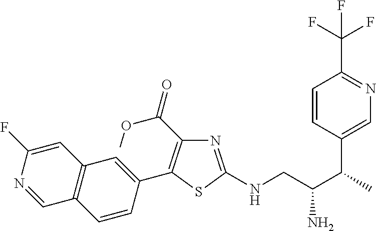

### Example 11

### Methyl 2-((2S,3S)-2-amino-3-(6-(trifluoromethyl)pyridin-3-yl)butylamino)-5-(3-fluoroisoquinolin-6-yl)thiazole-4-carboxylate

The title compound was synthesized in a manner similar to that described for Example 8, but using (4S)-4-((1S)-1-(6-(trifluoromethyl)-3-pyridinyl)ethyl)-1,2,3-oxathiazolidine-3-carboxylate 2,2-dioxide instead of tert-butyl (4S)-4-((6-(1,1-difluoroethyl)-3-pyridinyl)methyl)-1,2,3-oxathiazolidine-3-carboxylate 2-oxide. (4S)-4-((1S)-1-(6-(Trifluoromethyl)-3-pyridinyl)ethyl)-1,2,3-oxathiazolidine-3-carboxylate 2,2-dioxide was synthesized in a manner similar to that described for (S)-tert-butyl 4-(S)-1-(4-(trifluoromethyl)phenyl)ethyl)-1,2,3-oxathiazolidine-3-carboxylate, 2,2-dioxide (Scheme 6), but using 6-(trifluoromethyl)nicotinaldehyde (Aldrich) instead of 4-(trifluoromethyl)benzaldehyde as the starting material. MS m/z: 520 (M+1). ¹H NMR (400 MHz, CD₃OD): δ ppm 1.54 (d, J=7.04 Hz, 3H) 3.70 (s, 3H) 3.79-3.90 (m, 2H) 3.95-4.00 (m, 1H) 7.49 (d, J=0.39 Hz, 1H) 7.67 (dd, J=8.61, 1.76 Hz, 1H) 7.89 (d, J=8.22 Hz, 1H) 8.03 (m, 1H) 8.09-8.12 (m, 1H) 8.11 (s, 1H) 8.16 (d, J=8.61 Hz, 1H) 8.75 (m, 1H) 9.06 (s, 1H).

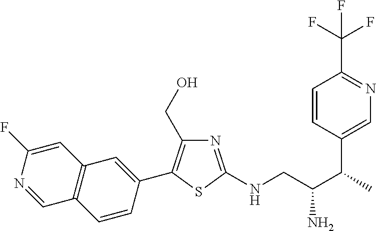

### Example 12

### (2-((2S,3S)-2-Amino-3-(6-(trifluoromethyl)pyridin-3-yl)butylamino)-5-(3-fluoroisoquinolin-6-yl)thiazol-4-yl)methanol

The title compound was synthesized in a manner similar to that described for Example 9 but using (4S)-4-((1S)-1-(6-(trifluoromethyl)-3-pyridinyl)ethyl)-1,2,3-oxathiazolidine-3-carboxylate 2,2-dioxide instead of tert-butyl (4S)-4-((6-(1,1-difluoroethyl)-3-pyridinyl)methyl)-1,2,3-oxathiazolidine-3-carboxylate 2-oxide. (4S)-4-((1S)-1-(6-(Trifluoromethyl)-3-pyridinyl)ethyl)-1,2,3-oxathiazolidine-3-carboxylate 2,2-dioxide was synthesized in a manner similar to that described for (S)-tert-butyl 4-((S)-1-(4-(trifluoromethyl)phenyl)ethyl)-1,2,3-oxathiazolidine-3-carboxylate, 2,2-dioxide (Scheme 6), but using 6-(trifluoromethyl)nicotinaldehyde (Aldrich) instead of 4-(trifluoromethyl)benzaldehyde as the starting material. MS m/z: 492 (M+1). ¹H NMR (400 MHz, CD₃OD): δ ppm 1.54 (d, J=7.04 Hz, 3H) 3.70 (s, 3H) 3.79-3.90 (m, 2H) 3.95-4.00 (m, 1H) 7.49 (s, 1H) 7.67 (dd, J=8.61, 1.76 Hz, 1H) 7.89 (d, J=8.22 Hz, 1H) 8.03 (m, 1H) 8.10 (dt, J=8.17, 1.20 Hz, 1H) 8.16 (d, J=8.41 Hz, 1H) 8.75 (m, 1H) 9.06 (s, 1H).

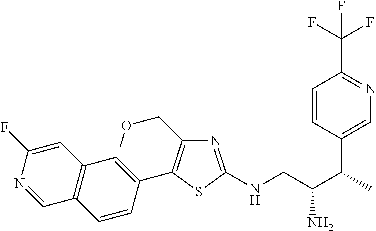

### Example 13

### N-((2S,3S)-2-Amino-3-(6-(trifluoromethyl)pyridin-3-yl)butyl)-5-(3-fluoroisoquinolin-6-yl)-4-(methoxymethyl)thiazol-2-amine

The title compound was synthesized in a manner similar to that described for Example 6, but using (4S)-4-((1S)-1-(6-(trifluoromethyl)-3-pyridinyl)ethyl)-1,2,3-oxathiazolidine-3-carboxylate 2,2-dioxide instead of tert-butyl (4S)-4-((6-(1,1-difluoroethyl)-3-pyridinyl)methyl)-1,2,3-oxathiazolidine-3-carboxylate 2-oxide. (4S)-4-((1S)-1-(6-(Trifluoromethyl)-3-pyridinyl)ethyl)-1,2,3-oxathiazolidine-3-carboxylate 2,2-dioxide was synthesized in a manner similar to that described for (S)-tert-butyl 4-((S)-1-(4-(trifluoromethyl)phenyl)ethyl)-1,2,3-oxathiazolidine-3-carboxylate, 2,2-dioxide (Scheme 6), but using 6-(trifluoromethyl)nicotinaldehyde (Aldrich) instead of 4-(trifluoromethyl)benzaldehyde as the starting material. MS m/z: 506 (M+1). ¹H NMR (400 MHz, CD₃OD): δ ppm 1.54 (d, J=7.04 Hz, 3H) 2.67 (s, 2H) 3.43 (s, 3H) 3.74-3.77 (m, 1H) 3.85-3.91 (m, 2H) 4.42 (s, 1H) 7.45 (s, 1H) 7.66 (dd, J=8.41, 1.56 Hz, 1H) 7.87 (d, J=8.22 Hz, 1H) 7.94-7.93 (m, 1H) 8.07-8.09 (m, 1H) 8.17 (d, J=8.80 Hz, 1H) 8.74 (d, J=1.76 Hz, 1H) 8.74 (s, 1H) 9.01 (s, 1H).

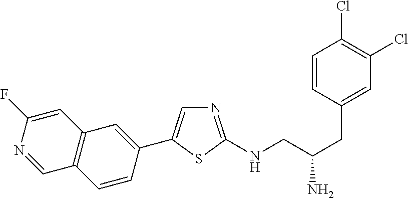

### Example 14

### N—((S)-2-amino-3-(3,4-dichlorophenyl)propyl)-5-(3-fluoroisoquinolin-6-yl)thiazol-2-amine

The title compound was synthesized in a manner similar to that described for Example 2, but using (4S)-4-(3,4-dichlorobenzyl)-1,2,3-oxathiazolidine-3-carboxylate 2,2-dioxide instead of tert-butyl (4S)-4-((6-(1,1-difluoroethyl)-3-pyridinyl)methyl)-1,2,3-oxathiazolidine-3-carboxylate 2-oxide. MS m/z: 447 (M+1). ¹H NMR (400 MHz, CD₃OD): δ ppm 3.00 (d, J=6.85 Hz, 1H) 3.04 (d, J=7.63 Hz, 1H) 3.55-3.58 (m, 1H) 3.69-3.73 (m, 1H) 3.81 (d, J=3.52 Hz, 1H) 7.29 (dd, J=8.22, 1.96 Hz, 1H) 7.38 (s, 1H) 7.54-7.57 (m, 2H) 7.73 (s, 1H) 7.81-7.85 (m, 2H) 8.09 (d, J=8.61 Hz, 1H) 8.92 (s, 1H). (4S)-4-(3,4-Dichlorobenzyl)-1,2,3-oxathiazolidine-3-carboxylate 2,2-dioxide was prepared as shown in Scheme 7.

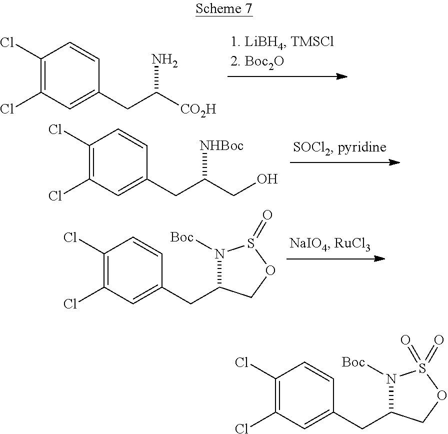

(S)-tert-Butyl 3-(3,4-dichlorophenyl)-1-hydroxypropan-2-ylcarbamate. In a 500 mL round bottom flask, LiBH₄ (2 g, 107 mmol, Fluka) was dissolved in THF (100 mL) at 0° C., and Me₃SiCl (27 mL, 214 mmol, Aldrich) was added in one portion. The reaction mixture was stirred for 20 minutes at 23° C., cooled to 0° C. and then (S)-2-amino-3-(3,4-dichlorophenyl)propanoic acid (5 g, 21 mmol, Chem-Impex) was added. The reaction mixture was stirred for 15 hours at 23° C., cooled to 0° C. and then 20 mL of MeOH was added dropwise via an addition funnel. After addition, 60 mL of 10 N NaOH was added to the mixture. The reaction mixture was separated and the organic layer was concentrated to give the amino alcohol. MS m/z: 220 (M+1). The amino alcohol was dissolved in THF (40 mL) and then di-tert-butyldicarbonate (6 mL, 26 mmol, Aldrich) in 30 mL of THF was added. After 30 minutes, the reaction mixture was washed with saturated NaHCO₃ solution and brine. The product was obtained by concentration and purification by silica gel chromatography, eluting with 0-40% EtOAc/hexane to give (S)-tert-butyl 3-(3,4-dichlorophenyl)-1-hydroxypropan-2-ylcarbamate (5.77 g, 84% over two steps). MS m/z: 320 (M+1).

tert-Butyl (4S)-4-(3,4-dichlorobenzyl)-1,2,3-oxathiazolidine-3-carboxylate 2-oxide. SOCl₂ (5.36 g, 45.0 mmol, Aldrich) was taken up in 14 mL of ACN and chilled to −55° C. (S)-tert-Butyl 3-(3,4-dichlorophenyl)-1-hydroxypropan-2-ylcarbamate (5.8 g, 18.0 mmol) was then added slowly in 80 mL of ACN. After 15 minutes, pyridine (7.13 g, 90.1 mmol) was added, and the mixture was warmed to room temperature. The mixture was concentrated under reduced pressure, and the residue was taken up in 100 mL of EtOAc and 100 mL of water. The mixture was partitioned, and the aqueous portion was extracted with 100 mL of EtOAc. The combined organic extracts were washed with 70 mL of brine and dried over MgSO₄. Filtration and concentration under reduced pressure, followed by flash chromatography on silica gel (0% to 20% EtOAc/hexanes) afforded tert-butyl (4S)-4-(3,4-dichlorobenzyl)-1,2,3-oxathiazolidine-3-carboxylate 2-oxide (4.93 g, 75%). MS m/z: 366 (M+1).

tert-Butyl (4S)-4-(3,4-dichlorobenzyl)-1,2,3-oxathiazolidine-3-carboxylate 2,2-dioxide. To a solution of tert-butyl (4S)-4-(3,4-dichlorobenzyl)-1,2,3-oxathiazolidine-3-carboxylate 2-oxide (4.9 g, 13 mmol) in ACN (120 mL) and EtOAc (20 mL) at 0° C. was added sodium periodate (1.28 g, 5.98 mmol, Aldrich) in 40 mL of water and ruthenium(iii) chloride (15 mg, 67 mmol, Aldrich). The mixture was allowed to warm to room temperature. After 6 hours, the ACN was removed by rotary evaporation. The aqueous mixture and precipitate were dissolved in EtOAc (200 mL) and washed with brine (2×100 mL). The brine was extracted with EtOAc (150 mL), and the combined organic layers were dried over sodium sulfate, filtered, and evaporated providing tert-butyl (4S)-4-(3,4-dichlorobenzyl)-1,2,3-oxathiazolidine-3-carboxylate 2,2-dioxide (4.8 g, 94%). MS m/z: 382 (M+1).

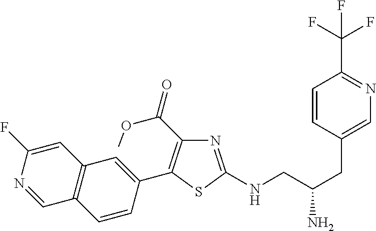

### Example 15

### Methyl 2-((S)-2-amino-3-(6-(trifluoromethyl)pyridin-3-yl)propylamino)-5-(3-fluoroisoquinolin-6-yl)thiazole-4-carboxylate

The title compound was synthesized in a manner similar to that described for Example 8, but using (4S)-4-(6-(trifluoromethyl)-3-pyridinyl)methyl)-1,2,3-oxathiazolidine-3-carboxylate 2-oxide (prepared as shown in Scheme 2) instead of tert-butyl (4S)-4-((6-(1,1-difluoroethyl)-3-pyridinyl)methyl)-1,2,3-oxathiazolidine-3-carboxylate 2-oxide. MS m/z: 506 (M+1). ¹H NMR (400 MHz, CD₃OD): δ ppm 3.11-3.17 (m, 1H) 3.19-3.25 (m, 1H) 3.61-3.64 (m, 1H) 3.72 (s, 3H) 3.74-3.78 (m, 1H) 3.92-3.99 (m, 1H) 7.47 (s, 1H) 7.66 (dd, J=8.61, 1.37 Hz, 1H) 7.86 (d, J=8.22 Hz, 1H) 8.02 (s, 1H) 8.09 (s, 1H) 8.15 (d, J=8.61 Hz, 1H) 8.73 (s, 1H) 9.05 (s, 1H).

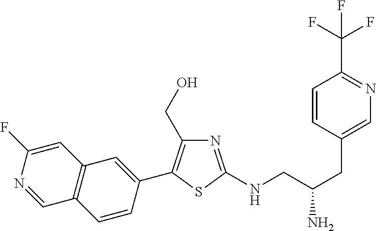

### Example 16

### (2-((S)-2-Amino-3-(6-(trifluoromethyl)pyridin-3-yl)propylamino)-5-(3-fluoroisoquinolin-6-yl)thiazol-4-yl)methanol

The title compound was synthesized in a manner similar to that described for Example 9 but using (4S)-4-((6-(trifluoromethyl)-3-pyridinyl)methyl)-1,2,3-oxathiazolidine-3-carboxylate 2-oxide (prepared as shown in Scheme 2) instead of tert-butyl (4S)-4-((6-(1,1-difluoroethyl)-3-pyridinyl)methyl)-1,2,3-oxathiazolidine-3-carboxylate 2-oxide. MS m/z: 478 (M+1). ¹H NMR (400 MHz, CD₃OD): δ ppm 3.10-3.23 (m, 2H) 3.57-3.62 (m, 1H) 3.74-3.78 (m, 1H) 3.91-3.96 (m, 1H) 4.61 (s, 2H) 7.44 (s, 1H) 7.68 (dd, J=8.51, 1.47 Hz, 1H) 7.86 (d, J=8.22 Hz, 1H) 7.96 (s, 1H) 8.06 (d, J=9.00 Hz, 1H) 8.16 (d, J=8.61 Hz, 1H) 8.73 (d, J=1.57 Hz, 1H) 9.00 (s, 1H).

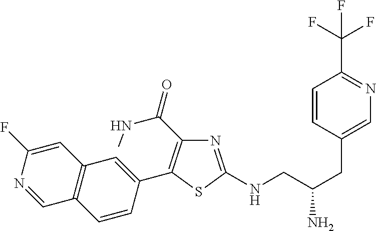

### Example 17

### 2-0S)-2-Amino-3-(6-(trifluoromethyl)pyridin-3-yl)propylamino)-5-(3-fluoroisoquinolin-6-yl)-N-methylthiazole-4-carboxamide

Methylamine in MeOH (1 mL, 40%, Aldrich) was added to methyl 2-((S)-2-amino-3-(6-(trifluoromethyl)pyridin-3-yl)propylamino)-5-(3-fluoroisoquinolin-6-yl)thiazole-4-carboxylate (20 mg, 40 μmol, Example 15) in 1 mL MeOH. The mixture was stirred at room temperature for 1 hour. LCMS indicated a clean conversion. The reaction mixture was purified by preparative HPLC (10-100% CH₃CN/H₂O, 0.1% TFA) to give 2-((S)-2-amino-3-(6-(trifluoromethyl)pyridin-3-yl)propylamino)-5-(3-fluoroisoquinolin-6-yl)-N-methylthiazole-4-carboxamide (14 mg, 70%). MS m/z: 505 (M+1). ¹H NMR (400 MHz, MeOH) δ ppm 3.14 (d, J=1.56 Hz, 2H), 3.48-3.50 (m, 1H), 3.63 (s, 1H), 3.80 (s, 1H), 7.43 (s, 1H), 7.70 (d, J=7.83 Hz, 1H), 7.86 (d, J=8.02 Hz, 1H), 8.00 (s, 1H), 8.09 (s, 1 H), 8.11 (s, 1H), 8.74 (s, 1H), 9.00 (s, 1H).

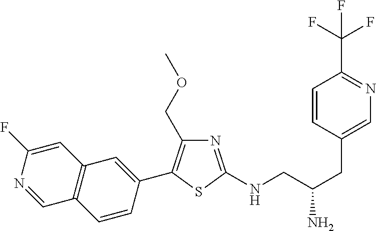

### Example 18

### N—((S)-2-Amino-3-(6-(trifluoromethyl)pyridin-3-yl)propyl)-5-(3-fluoroisoquinolin-6-yl)-4-(methoxymethyl)thiazol-2-amine

The title compound was synthesized in a manner similar to that described for Example 6, but using (4S)-4-((6-(trifluoromethyl)-3-pyridinyl)methyl)-1,2,3-oxathiazolidine-3-carboxylate 2-oxide (prepared as shown in Scheme 2) instead of tert-butyl (4S)-4-((6-(1,1-difluoroethyl)-3-pyridinyl)methyl)-1,2,3-oxathiazolidine-3-carboxylate 2-oxide. MS m/z: 492 (M+1). ¹H NMR (400 MHz, CD₃OD): δ ppm 3.14 (t, J=7.04 Hz, 1H) 3.18-3.23 (m, 1H) 3.45 (s, 3H) 3.57-3.63 (m, 1H) 3.72-3.76 (m, 1H) 3.95 (d, J=3.91 Hz, 1H) 4.44 (s, 2H) 7.44 (s, 1H) 7.66 (dd, J=8.71, 1.27 Hz, 1H) 7.85 (d, J=8.02 Hz, 1H) 7.94 (s, 1H) 8.06 (d, J=8.02 Hz, 1H) 8.16 (d, J=8.61 Hz, 1H) 8.73 (s, 1H) 9.01 (s, 1H).

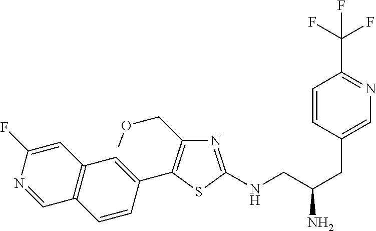

### Example 19

### N—((R)-2-Amino-3-(6-(trifluoromethyl)pyridin-3-yl)propyl)-5-(3-fluoroisoquinolin-6-yl)-4-(methoxymethyl)thiazol-2-amine

The title compound was synthesized in a manner similar to that described for Example 18, but using methyl N-(tert-butoxycarbonyl)-3-iodo-D-alaninate (Fluka) in place of methyl N-(tert-butoxycarbonyl)-3-iodo-L-alaninate. MS m/z: 492 (M+1). ¹H NMR (400 MHz, CD₃OD): δ ppm 3.09-3.15 (m, 1H) 3.18-3.23 (m, 1H) 3.44 (s, 3H) 3.56-3.60 (m, 1H) 3.71-3.75 (m, 1H) 3.93 (dd, J=7.73, 4.01 Hz, 1H) 4.42 (s, 2H) 7.43 (s, 1H) 7.65 (m, 1H), 7.84 (d, J=8.22 Hz, 1H) 7.93 (d, J=0.98 Hz, 1H) 8.05 (dd, J=7.92, 1.66 Hz, 1H) 8.15 (d, J=8.61 Hz, 1H) 8.72 (d, J=1.37 Hz, 1H) 9.00 (s, 1H).

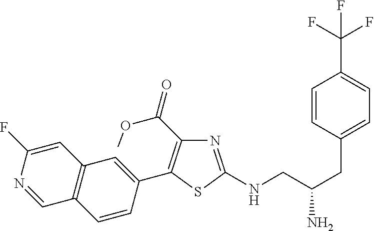

### Example 20

### Methyl 2-((S)-2-amino-3-(4-(trifluoromethyl)phenyl)propylamino)-5-(3-fluoroisoquinolin-6-yl)thiazole-4-carboxylate

The title compound was synthesized in a manner similar to that described for Example 8, but using tert-butyl (4S)-4-(4-(trifluoromethyl)benzyl)-1,2,3-oxathiazolidine-3-carboxylate 2,2-dioxide (prepared as shown for Example 4) instead of tert-butyl (4S)-4-((6-(1,1-difluoroethyl)-3-pyridinyl)methyl)-1,2,3-oxathiazolidine-3-carboxylate 2-oxide. MS m/z: 505 (M+1). ¹H NMR (400 MHz, CD₃OD): δ ppm 3.13 (t, J=7.24 Hz, 2H) 3.57-3.63 (m, 1H) 3.70-3.75 (m, 4H) 3.87 (d, J=3.72 Hz, 1H) 7.47 (s, 1H) 7.57 (d, J=8.22 Hz, 2H) 7.64-7.73 (m, 3H) 8.02 (s, 1H) 8.15 (d, J=8.61 Hz, 1H) 9.04 (s, 1H).

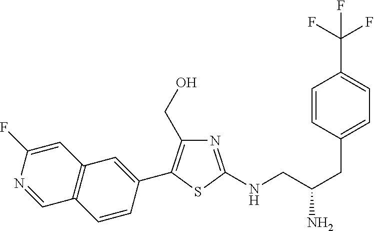

### Example 21

### (2-((S)-2-Amino-3-(4-(trifluoromethyl)phenyl)propylamino)-5-(3-fluoroisoquinolin-6-yl)thiazol-4-yl)methanol

The title compound was synthesized in a manner similar to that described for Example 9 using NaBH₄ to reduce methyl 2-((S)-2-amino-3-(4-(trifluoromethyl)phenyl)propylamino)-5-(3-fluoroisoquinolin-6-yl)thiazole-4-carboxylate (prepared as shown for Example 20) to the corresponding alcohol. MS m/z: 477 (M+1). ¹H NMR (400 MHz, CD₃OD): δ ppm 3.14 (m, 2H) 3.56-3.59 (m, 2H) 3.71-3.74 (m, 1H) 4.61 (s, 2H) 7.44 (s, 1H) 7.58 (s, 2H) 7.67-7.73 (m, 3H) 7.95 (s, 1H) 8.15-8.17 (m, 1H) 9.00 (s, 1H).

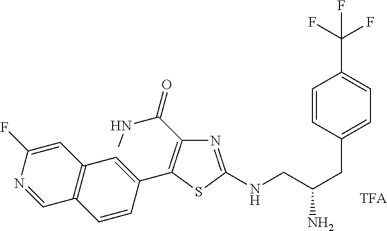

### Example 22

### 2-((S)-2-Amino-3-(4-(trifluoromethyl)phenyl)propylamino)-5-(3-fluoroisoquinolin-6-yl)-N-methylthiazole-4-carboxamide trifluoroacetate

The title compound was synthesized in a manner similar to that described for Example 17 using methylamine in MeOH (1 mL, 40%, Aldrich) reacted with methyl 2-((S)-2-amino-3-(4-(trifluoromethyl)phenyl)propylamino)-5-(3-fluoroisoquinolin-6-yl)thiazole-4-carboxylate (prepared as shown for Example 20). MS m/z: 504 (M+1). ¹H NMR (400 MHz, CD₃OD): δ ppm 2.86 (s, 3H) 3.14 (dd, J=6.94, 3.03 Hz, 2H) 3.59 (dd, J=14.87, 6.46 Hz, 1H) 3.75-3.82 (m, 1H) 3.89 (dd, J=6.75, 4.21 Hz, 1H) 7.42 (s, 1H) 7.57 (d, J=8.02 Hz, 2H) 7.68-7.73 (m, 3H) 8.00 (s, 1H) 8.08 (d, J=8.80 Hz, 1H) 8.99 (s, 1H).

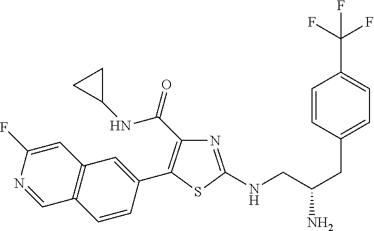

### Example 23

### 2-((S)-2-Amino-3-(4-(trifluoromethyl)phenyl)propylamino)-N-cyclopropyl-5-(3-fluoroisoquinolin-6-yl)thiazole-4-carboxamide

The title compound was synthesized in a manner similar to that described for Example 22, but using cyclopropanamine (Aldrich) instead of methylamine. MS m/z: 530 (M+1). ¹H NMR (400 MHz, CD₃OD): δ ppm 0.53-0.58 (m, 2H) 0.77-0.82 (m, 2H) 2.71-2.77 (m, J=7.31, 7.31, 3.81, 3.67 Hz, 1H) 3.08-3.18 (m, 2H) 3.55-3.61 (m, 1H) 3.74-3.80 (m, 1H) 3.87 (ddd, J=11.00, 7.14, 6.90 Hz, 1H) 7.43 (s, 1H) 7.57 (d, J=8.22 Hz, 2H) 7.67-7.74 (m, 3H) 7.98 (s, 1H) 8.10 (d, J=8.61 Hz, 1H) 9.00 (s, 1H).

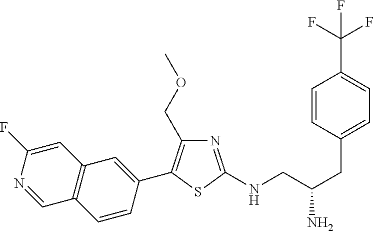

### Example 24

### N—((S)-2-Amino-3-(4-(trifluoromethyl)phenyl)propyl)-5-(3-fluoroisoquinolin-6-yl)-4-(methoxymethyl)thiazol-2-amine

The title compound was synthesized in a manner similar to that described for Example 6, but using tert-butyl (4S)-4-(4-(trifluoromethyl)benzyl)-1,2,3-oxathiazolidine-3-carboxylate 2,2-dioxide (prepared as shown for Example 4) instead of tert-butyl (4S)-4-(6-(1,1-difluoroethyl)-3-pyridinyl)methyl)-1,2,3-oxathiazolidine-3-carboxylate 2-oxide. MS m/z: 491 (M+1). ¹H NMR (400 MHz, CD₃OD): δ ppm 3.06-3.16 (m, 2H) 3.45 (s, 3H) 3.55-3.59 (m, 1H) 3.69-3.73 (m, 1H) 3.86 (dt, J=6.70, 1.83 Hz, 1H) 4.44 (s, 2H) 7.44 (s, 1H) 7.56 (d, J=8.41 Hz, 2H) 7.66-7.71 (m, 3H) 7.94 (m, 1H) 8.16 (d, J=8.80 Hz, 1H) 9.01 (s, 1H).

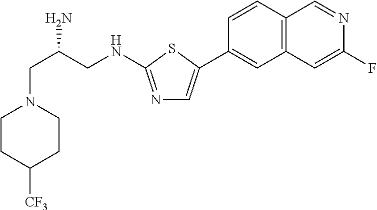

### Example 25

### N—((R)-2-Amino-3-(4-(trifluoromethyl)piperidin-1-yl)propyl)-5-(3-fluoroisoquinolin-6-yl)thiazol-2-amine

This compound was prepared as shown in Scheme 8.

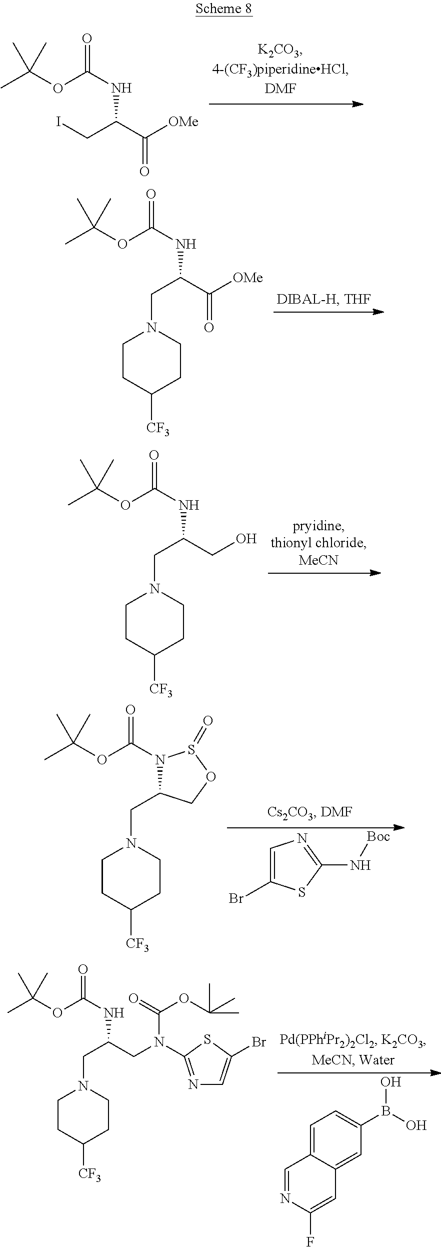

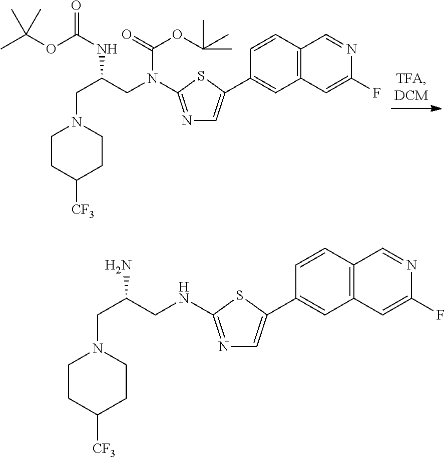

(S)-Methyl 2-(tert-butoxycarbonylamino)-3-(4-(trifluoromethyl)piperidin-1-yl)propanoate. A 250 mL round bottom flask was charged with Boc-3-iodo-1-alanine methyl ester (5.09 g, 15.5 mmol, Aldrich catalog number 426024), 4-(trifluoromethyl)piperidine hydrochloride (2.55 g, 13.4 mmol, Aldrich catalog number 665509), potassium carbonate (4.65 g, 33.6 mmol), and 20 mL of DMF. The resulting mixture was heated at 50° C. for 3 hours and then concentrated. The residue was diluted with water and EtOAc. The layers were separated and the organic layer was concentrated in vacuo to give a colorless oil. The oil was purified by chromatography on silica gel (0-30% EtOAc/hexane) to give (S)-methyl 2-(tert-butoxycarbonylamino)-3-(4-(trifluoromethyl)piperidin-1-yl)propanoate (2.65 g, 55.6%) as a colorless oil. ¹H NMR (400 MHz, CDCl₃) δ 1.46 (s, 9H), 1.55-1.70 (broad m, 2H), 1.78-1.90 (broad m, 2H), 1.95-2.22 (broad m, 3H), 2.63-3.10 (broad m, 4H), 3.76 (s, 3H), 4.28-4.43 (broad m, 1H).

(S)-tert-Butyl 1-hydroxy-3-(4-(trifluoromethyl)piperidin-1-yl)propan-2-ylcarbamate. A 250 mL round bottom flask was charged with (S)-methyl 2-(tert-butoxycarbonylamino)-3-(4-(trifluoromethyl)piperidin-1-yl)propanoate (2.69 g, 7.6 mmol) and 30 mL of THF. After cooling to −78° C., DIBAL-H (30 mL, 30 mmol, Aldrich catalog number 256838) in hexanes was added dropwise. After stirring for 15 minutes at −78° C., the mixture was warmed to room temperature. A saturated aqueous solution of Rochelle's salt (25 mL) and 50 mL of ether was added, and the mixture was stirred for 20 minutes. After that time, two well defined layers were seen. The organic layer was separated and the aqueous layer was further extracted with ether. The combined organic layers were dried and concentrated to give (S)-tert-butyl 1-hydroxy-3-(4-(trifluoromethyl)piperidin-1-yl)propan-2-ylcarbamate (2.37 g, 96%) as a slightly yellow oil, which was used in the next reaction, without further purification. ¹H NMR (400 MHz, CDCl₃) δ 1.46 (s, 9H), 1.55-1.70 (broad m, 2H), 1.95-2.38 (broad m, 5H), 2.88-3.09 (broad m, 2H), 3.45-3.60 (broad m, 1H), 3.70-3.74 (m, 1H), 3.92-4.07 (broad m, 2H), 5.34-5.47 (broad m, 1H).

tert-Butyl (4S)-4-(4-(trifluoromethyl)-1-piperidinyl)methyl)-1,2,3-oxathiazolidine-3-carboxylate 2-oxide. A 100 mL round bottom flask was charged with SOCl₂ (1.3 mL, 18 mmol, Aldrich catalog number 447285) and 35 mL of ACN. After cooling to −55° C., (S)-tert-butyl 1-hydroxy-3-(4-(trifluoromethyl)piperidin-1-yl)propan-2-ylcarbamate (2.37 g, 7.3 mmol) as a solution in ACN was added over 20 minutes. After 15 minutes, pyridine (2.9 mL, 36 mmol) was added and the reaction was warmed to room temperature. The mixture was stirred at room temperature for 1 hour and then the solvent was removed and the residue was partitioned between EtOAc/water. The organic layers were collected, dried, and concentrated to give an oil which was purified by chromatography on silica gel (0-40% EtOAc/hexane) to give tert-butyl (4S)-4-((4-(trifluoromethyl)-1-piperidinyl)methyl)-1,2,3-oxathiazolidine-3-carboxylate 2-oxide (0.96 g, 35%).

tert-Butyl (5-bromo-1,3-thiazol-2-yl)((2S)-2-((tert-butoxycarbonyl)amino)-3-(4-(trifluoromethyl)-1-piperidinyl)propyl)carbamate. A 100 mL round bottom flask was charged with cesium carbonate (1.36 g, 4.19 mmol, Aldrich catalog number 554855), tert-butyl 5-bromothiazol-2-ylcarbamate (0.614 g, 2.20 mmol, prepared as shown in Scheme 2), and 10 mL of DMF. The mixture was warmed to 50° C. and tert-butyl (4S)-4-(4-(trifluoromethyl)-1-piperidinyl)methyl)-1,2,3-oxathiazolidine-3-carboxylate 2-oxide (0.780 g, 2.09 mmol) was added dropwise as a solution in DMF. The mixture was stirred at 50° C. for 6 hours. After cooling to room temperature, water and ether were added. The organics were collected, concentrated in vacuo and purified by chromatography on silica gel (0-30% EtOAc/hexane) to give tert-butyl (5-bromo-1,3-thiazol-2-yl)((2S)-2-((tert-butoxycarbonyl)amino)-3-(4-(trifluoromethyl)-1-piperidinyl)propyl)carbamate (0.625 g, 50.8%) as a white brittle foam: ¹H NMR (400 MHz, CDCl₃) δ 1.40 (s, 9H), 1.63 (s, 9H), 2.05-2.15 (broad m, 3H), 2.21-2.59 (broad m, 3H), 2.74-2.92 (broad m, 1H), 3.03-3.15 (broad m, 1H), 3.25-3.38 (broad m, 1H), 3.65-3.73 (broad m, 1H), 3.88-3.98 (broad m, 1H), 4.15-4.27 (broad m, 1H), 4.35-4.55 (broad m, 2H), 5.91-5.99 (broad m, 1H).

tert-Butyl ((2S)-2-((tert-butoxycarbonyl)amino)-3-(4-(trifluoromethyl)-1-piperidinyl)propyl)(5-(3-fluoro-6-isoquinolinyl)-1,3-thiazol-2-yl)carbamate. A mixture of tert-butyl (5-bromo-1,3-thiazol-2-yl)((2S)-2-((tert-butoxycarbonyl)amino)-3-(4-(trifluoromethyl)-1-piperidinyl)propyl)carbamate (100 mg, 170 μmol), 3-fluoroisoquinolin-6-ylboronic acid (42 mg, 221 μmol, prepared as shown in Scheme 1), and potassium acetate (117 mg, 1.19 mmol) was added to degassed ACN (1.7 mL) and water (0.5 mL) in a microwave reaction vessel. Dichlorobis(diisopropylphenyl-phosphine)palladium(II) (10 mg, 15 μmol, Org. Lett., 2006, 8(9), 1787) was added, and the reaction vessel sealed and the mixture heated for 1 hour at 90° C. in the microwave. The resulting mixture was evaporated and the residue was dissolved in EtOAc and water. The organic layer was separated, dried, filtered, and concentrated. The product was purified by chromatography on silica gel eluting with 0-60% of 10% (2M NH₃ in MeOH) in DCM/DCM to yield tert-butyl ((2S)-2-((tert-butoxycarbonyl)amino)-3-(4-(trifluoromethyl)-1-piperidinyl)propyl)(5-(3-fluoro-6-isoquinolinyl)-1,3-thiazol-2-yl)carbamate (70 mg, 63%): LCMS (API-ES) m/z: (M+H) 654.4.

N—((R)-2-Amino-3-(4-(trifluoromethyl)piperidin-1-yl)propyl)-5-(3-fluoroisoquinolin-6-yl)thiazol-2-amine A 10 mL round bottom flask was charged with tert-butyl ((2S)-2-((tert-butoxycarbonyl)amino)-3-(4-(trifluoromethyl)-1-piperidinyl)propyl)(5-(3-fluoro-6-isoquinolinyl)-1,3-thiazol-2-yl)carbamate (70 mg, 107 μmol), DCM (2 mL) and TFA (0.5 mL) at room temperature. After 2 hours LCMS indicated removal of both Boc groups. The reaction was quenched with NaHCO₃ and diluted with EtOAc. The product was purified by chromatography on silica gel eluting with 0-100% of [10% (2M NH₃ in MeOH)/DCM] to yield N—((R)-2-amino-3-(4-(trifluoromethyl)piperidin-1-yl)propyl)-5-(3-fluoroisoquinolin-6-yl)thiazol-2-amine (30 mg, 62%) as a white solid. LCMS (API-ES) m/z: (M+H) 454.1; ¹H NMR (400 MHz, CD₃OD) δ 1.64 (m, 2H), 1.86 (m, 2H), 2.00 (m, 1H), 2.15 (m, 2H), 2.35 (dd, J=8.0, 12.0 Hz, 1H), 2.44 (dd, J=8.0, 12.0 Hz, 1H), 3.03 (m, 2H), 3.27 (m, 1H), 3.32 (m, 1H), 3.44 (dd, J=4.0, 12.0 Hz, 1H), 7.33 (s, 1H), 7.66 (s, 1H), 7.77 (m, 2H), 8.03 (d, J=8.0 Hz, 1H), 8.87 (s, 1H).

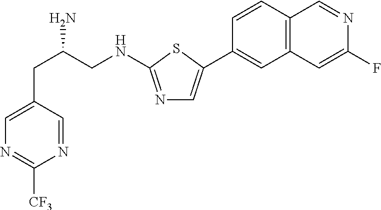

### Example 26

### N—((S)-2-Amino-3-(2-(trifluoromethyl)pyrimidin-5-yl)propyl)-5-(3-fluoroisoquinolin-6-yl)thiazol-2-amine

N—((S)-2-Amino-3-(2-(trifluoromethyl)pyrimidin-5-yl)propyl)-5-(3-fluoroisoquinolin-6-yl)thiazol-2-amine was synthesized by a method similar to that for Example 25, but using (S)-methyl 2-(tert-butoxycarbonylamino)-3-(2-(trifluoromethyl)pyrimidin-5-yl)propanoate instead of (S)-methyl 2-(tert-butoxycarbonylamino)-3-(4-(trifluoromethyl)piperidin-1-yl)propanoate. LCMS (API-ES) m/z: (M+H) 449.1; ¹H NMR (400 MHz, CD₃OD) δ 2.77 (dd, J=8.0, 12.0 Hz, 1H), 3.04 (dd, J=8.0, 16.0 Hz, 1H), 3.38 (m, 2H), 3.50 (m, 1H), 7.32 (s, 1H), 7.64 (s, 1H), 7.75 (m, 2H), 8.02 (d, J=12.0 Hz, 1H), 8.86 (s, 1H), 8.90 (s, 2H). (S)-Methyl 2-(tert-butoxycarbonylamino)-3-(2-(trifluoromethyl)pyrimidin-5-yl)propanoate was prepared as shown in Scheme 9.

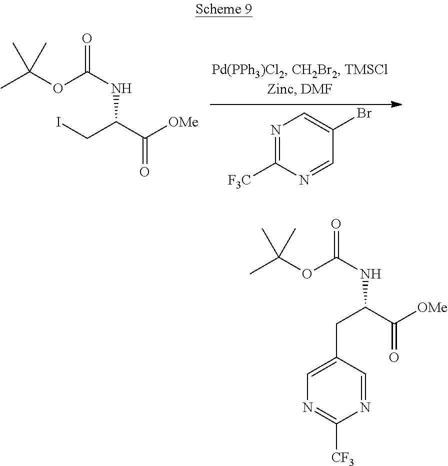

(S)-Methyl 2-(tert-butoxycarbonylamino)-3-(2-(trifluoromethyl)pyrimidin-5-yl)propanoate. A 250 mL round bottom flask was charged with zinc (3.37 g, 51.5 mmol, Aldrich catalog number 36553) and 30 mL of DMF. To this mixture was added methylene bromide (0.18 mL, 2.59 mmol, Aldrich catalog number 66730), and the mixture was heated at 90° C. for 30 minutes. After cooling to room temperature, trimethylsilyl chloride (0.065 mL, 0.52 mmol, Aldrich catalog number 386529) was added and the reaction was stirred for 30 minutes at room temperature. Boc-3-iodo-1-alanine methyl ester (4.81 g, 14.6 mmol, Aldrich catalog number 426024) was added and the resulting mixture was stirred for an additional 4 hours before trans-dichlorobis(triphenyl-phosphine)palladium(II) (0.36 g, 0.52 mmol, Org. Lett., 2006, 8(9), 1787) and 5-bromo-2-(trifluoromethyl)pyrimidine (1.95 g, 8.6 mmol, Anichem catalog number H11419) were added. The mixture was stirred at room temperature 16 hours before concentration. The residue was purified by chromatography on silica gel (0-50% EtOAc/hexane) to give (S)-methyl 2-(tert-butoxycarbonylamino)-3-(2-(trifluoromethyl)pyrimidin-5-yl)propanoate (1.51 g, 50.3%) as a brown viscous oil. ¹H NMR (400 MHz, CDCl₃) δ 1.46 (s, 9H), 3.07-3.17 (m, 1H), 3.32-3.41 (m, 1H), 3.76 (s, 3H), 4.59-4.72 (broad m, 1H), 8.72 (s, 2H).

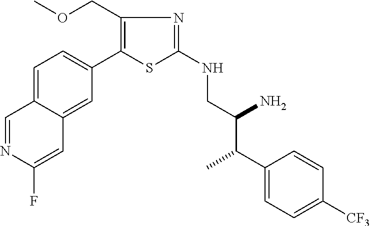

### Example 27

### N-((2R,3S)-2-Amino-3-(4-(trifluoromethyl)phenyl)butyl)-5-(3-fluoroisoquinolin-6-yl)-4-(methoxymethyl)thiazol-2-amine

N-((2R,3S)-2-Amino-3-(4-(trifluoromethyl)phenyl)butyl)-5-(3-fluoroisoquinolin-6-yl)-4-(methoxymethyl)thiazol-2-amine was synthesized as shown in Scheme 10 starting from commercially available 1-methoxypropan-2-one and thiourea.

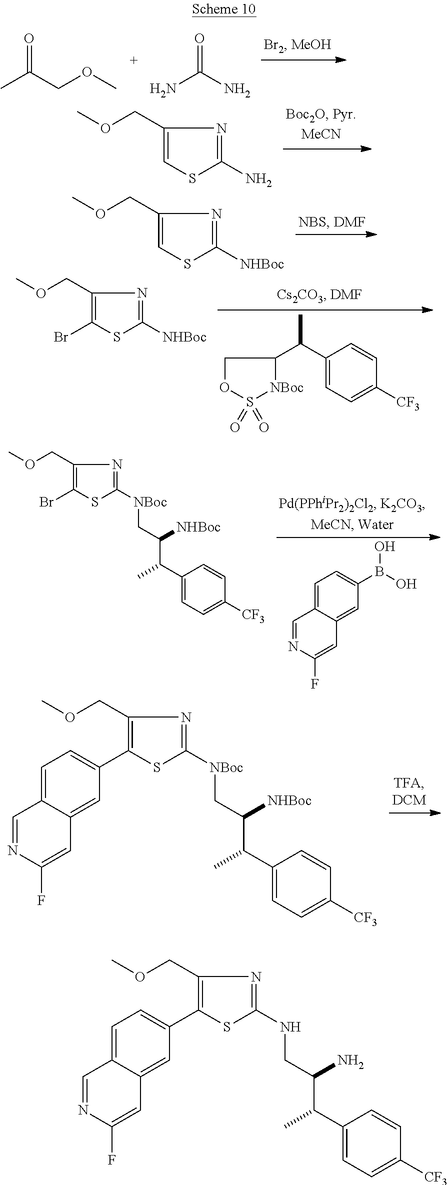

4-(Methoxymethyl)thiazol-2-amine A 250 mL round bottom flask was charged with 1-methoxypropan-2-one (9.0 g, 102 mmol, Aldrich catalog number 177180) and MeOH (100 mL), and bromine (5.3 mL, 102 mmol, Aldrich catalog number 470864) was added dropwise at 0° C. The addition funnel was rinsed with MeOH (25 mL), and the reaction stirred at 0° C. for 20 minutes before warming to room temperature over 2 hours. After this time, TLC indicated that the reaction was complete, and thiourea (7.8 g, 102 mmol, Aldrich 33717) was added. The resulting mixture was then heated at reflux for 3 hours. The reaction solvent was removed and sodium bicarbonate was used to adjust the pH to 8 before extraction with EtOAc. The organic layer was dried, filtered and concentrated to yield 4-(methoxymethyl)thiazol-2-amine (3.3 g, 22%). The product was recrystallized from hexane/EtOAc to give a white solid. LCMS API-ES m/z: 145.1 (M+H)⁺.

tert-Butyl 4-(methoxymethyl)thiazol-2-ylcarbamate: A 500 mL round bottom flask was charged with 4-(methoxymethyl)thiazol-2-amine (3.3 g, 23 mmol) in ACN (200 mL) and pyridine (5.6 mL, 69 mmol). Di-tert-butyl dicarbonate (5.3 mL, 23 mmol, Aldrich catalog number 361941) was then added at room temperature. The reaction was then stirred at room temperature 16 hours. The reaction was quenched by addition of water and extracted with EtOAc. The organic layer was separated, dried, filtered, and concentrated. The residue was purified by chromatography on silica gel eluting with 0-30% hexane/EtOAc to yield tert-butyl 4-(methoxymethyl)thiazol-2-ylcarbamate (1.8 g, 32%) as an oil. LCMS (API-ES) m/z: 245.1 (M+H).

tert-Butyl 5-bromo-4-(methoxymethyl)thiazol-2-ylcarbamate: A 100 mL round bottom flask was charged with tert-butyl 4-(methoxymethyl)thiazol-2-ylcarbamate (1.8 g, 7.4 mmol) in DMF (25 mL), and NB S (1.3 g, 7.4 mmol, Aldrich catalog number B81255) was added at room temperature. The reaction was stirred at room temperature overnight. The reaction was concentrated and the residue was redissolved in brine/EtOAc. The organic layer was separated, dried, filtered, and concentrated. The residue wad purified by chromatography on silica gel eluting with 0-40% hexane/EtOAc to yield tert-butyl 5-bromo-4-(methoxymethyl)thiazol-2-ylcarbamate (2.1 g, 88%) as an oil. LCMS (API-ES) m/z: 323.1/325.1 (M+H) (1:1 bromine pattern).

tert-Butyl (2S,3S)-(5-bromo-4-(methoxymethyl)-1,3-thiazol-2-yl)(2-((tert-butoxycarbonyl)amino)-3-(4-(trifluoromethyl)phenyl)butyl)carbamate: A 50 mL round bottom flask was charged with tert-butyl 5-bromo-4-(methoxymethyl)thiazol-2-ylcarbamate (500 mg, 1.55 mmol) in DMF (10 mL) and cesium carbonate (1.01 g, 3.10 mmol). The resulting mixture was heated at 50° C. for 10 minutes and then tert-butyl (2S,3S)-4-(1-(4-(trifluoromethyl)phenyl)ethyl)-1,2,3-oxathiazolidine-3-carboxylate 2,2-dioxide (734 mg, 1.86 mmol, prepared as shown in Scheme 6 for Example 10) was added as a solution in DMF (10 mL) via cannula. The reaction was heated at 50° C. for a further hour whereupon LCMS indicated that the reaction was complete. The mixture was concentrated and the residue was purified by chromatography on silica gel eluting with 0-30% hex/EtOAc to yield tert-butyl (2S,3S)-(5-bromo-4-(methoxymethyl)-1,3-thiazol-2-yl)(2-((tert-butoxycarbonyl)amino)-3-(4-(trifluoromethyl)phenyl)butyl)carbamate (430 mg, 44%) as an oil. LCMS (API-ES) m/z: 638.1/640.1 (M+H) (1:1 bromine pattern).

tert-Butyl (2S,3S)-(2-((tert-butoxycarbonyl)amino)-3-(4-(trifluoromethyl)phenyl)butyl)(5-(3-fluoro-6-isoquinolinyl)-4-(methoxymethyl)-1,3-thiazol-2-yl)carbamate. A mixture of tert-butyl (2S,3S)-(5-bromo-4-(methoxymethyl)-1,3-thiazol-2-yl)(2-((tert-butoxycarbonyl)amino)-3-(4-(trifluoromethyl)phenyl)butyl)carbamate (210 mg, 329 μmol), 3-fluoroisoquinolin-6-ylboronic acid (94 mg, 493 μmol, prepared as shown in Scheme 1) and potassium acetate (226 mg, 2.30 mmol) was added to degassed ACN (3.3 mL) and water (1.1 mL) in a microwave reaction vessel. Dichlorobis(diisopropylphenylphosphine)palladium(II) (18 mg, 30 μmol, Org. Lett., 2006, 8(9), 1787) was added, the reaction vessel sealed, and the mixture heated for 1 hour at 90° C. in a microwave. The resulting mixture was concentrated, and the residue was dissolved in EtOAc and water. The organic layer was separated and then dried, filtered and concentrated. The product was purified by chromatography on silica gel eluting with 0-70% hex/EtOAc to yield tert-butyl (2S,3S)-(2-((tert-butoxycarbonyl)amino)-3-(4-(trifluoromethyl)phenyl)butyl)(5-(3-fluoro-6-isoquinolinyl)-4-(methoxymethyl)-1,3-thiazol-2-yl)carbamate (40 mg, 17%) as an oil. LCMS (API-ES) m/z: 705.2 (M+H).

N-((2S,3S)-2-Amino-3-(4-(trifluoromethyl)phenyl)butyl)-5-(3-fluoroisoquinolin-6-yl)-4-(methoxymethyl)thiazol-2-amine. A 10 mL round bottom flask was charged with tert-butyl (2S,3S)-(2-((tert-butoxycarbonyl)amino)-3-(4-(trifluoromethyl)phenyl)butyl)(5-(3-fluoro-6-isoquinolinyl)-4-(methoxymethyl)-1,3-thiazol-2-yl)carbamate (40 mg, 57 μmol), DCM (2 mL) and TFA (0.5 mL) at room temperature. After 2 hours LCMS indicated removal of both Boc groups. The reaction was quenched with NaHCO₃ and diluted with EtOAc. The product was purified by chromatography on silica gel eluting with 0-100% of [10% (2M NH₃ in MeOH)/DCM] to yield N-((2S,3S)-2-amino-3-(4-(trifluoromethyl)phenyl)butyl)-5-(3-fluoroisoquinolin-6-yl)-4-(methoxymethyl)thiazol-2-amine (10 mg, 33%) as a white solid. LCMS (API-ES) m/z: 505.2 (M+H); ¹H NMR (400 MHz, CD₃OD) δ 1.42 (d, J=4.0 Hz, 3H), 3.03 (m, 1H), 3.36-3.42 (m, 2H), 3.45 (s, 3H), 3.65 (m, 1H), 4.39 (s, 2H), 7.42 (s, 1H), 7.53 (m, 2H), 7.66-7.69 (m, 3H), 7.93 (s, 1H), 8.13 (d, J=8.0 Hz, 1H), 8.98 (s, 1H).

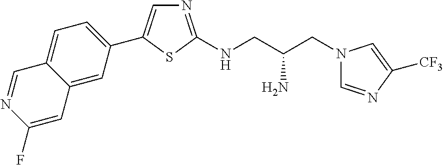

### Example 28

### N—((R)-2-Amino-3-(4-(trifluoromethyl)-1H-imidazol-1-yl)propyl)-5-(3-fluoroisoquinolin-6-yl)thiazol-2-amine

N4R)-2-Amino-3-(4-(trifluoromethyl)-1H-imidazol-1-yl)propyl)-5-(3-fluoroisoquinolin-6-yl)thiazol-2-amine was prepared according to Scheme 11.

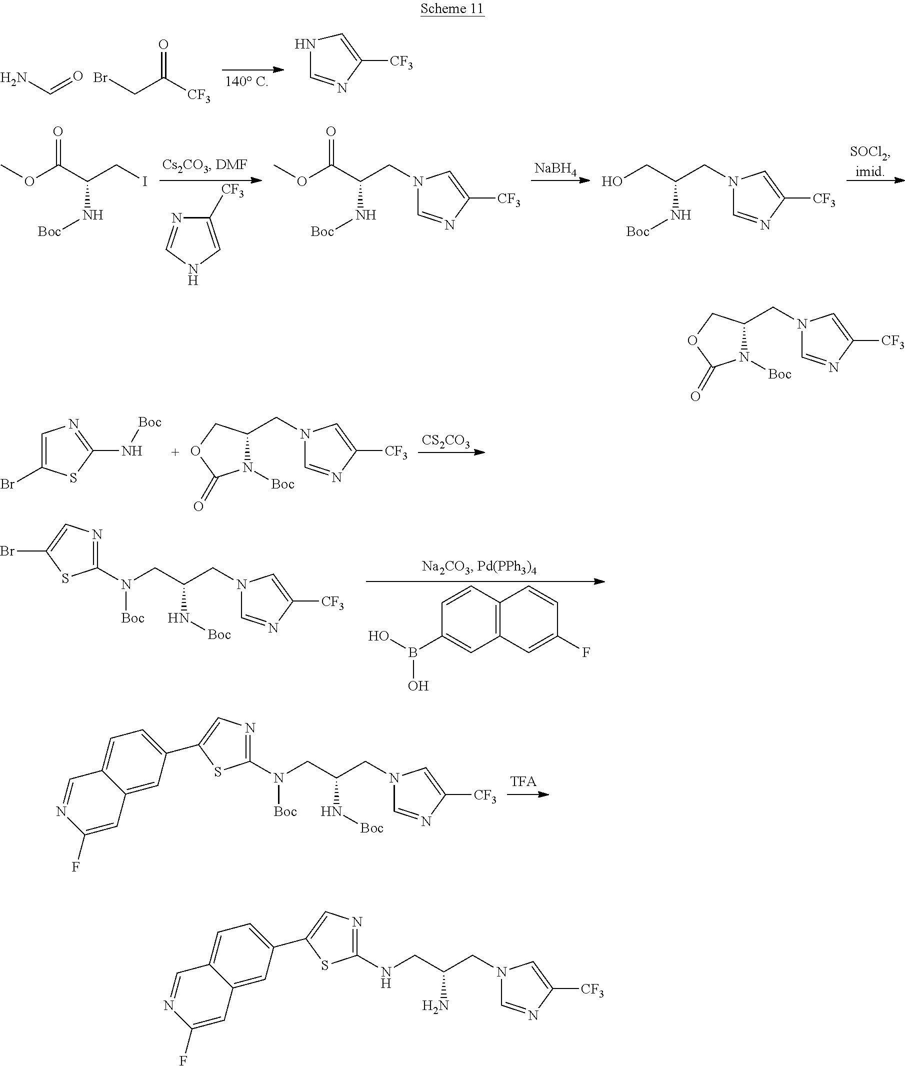

4-(Trifluoromethyl)-1H-imidazole: A 500 mL round-bottomed flask was charged with 3-bromo-1,1,1-trifluoropropan-2-one (25.0 g, 131 mmol, Aldrich catalog number 374059) and formamide (104 mL, 2.62 mol). The mixture was warmed to reflux (˜140° C.) and heated for 2.5 hours. The mixture was then cooled to room temperature and diluted with 200 mL of 10% aqueous K₂CO₃ solution and extracted with ether (5×200 mL). The combined organic extracts were washed with 10% K₂CO₃ and water (2×100 mL), dried with MgSO₄, filtered, and concentrated to give a brown solid. The solid thus obtained was washed with DCM to give 4-(trifluoromethyl)-1H-imidazole (4.0 g, 22%) as a tan solid. ¹H NMR (CD₃OD) δ 7.82 (s, 1H), 7.60 (s, 1H).

(S)-Methyl 2-(tert-butoxycarbonylamino)-3-(4-(trifluoromethyl)-1H-imidazol-1-yl)propanoate: A 100 mL round-bottomed flask was charged with 4-(trifluoromethyl)-1H-imidazole (1.75 g, 12.9 mmol), (R)-methyl 2-(tert-butoxycarbonylamino)-3-iodopropanoate (4.23 g, 12.9 mmol, Fluka catalog number 15126), cesium carbonate (4.82 g, 14.8 mmol, Aldrich), and 20 mL of DMF. The resulting mixture was stirred at room temperature for 12 hours and then was diluted with water and extracted with ether. The organic layers were washed with water (2×100 mL), dried (MgSO₄), filtered, and concentrated to provide an oil. Purification via chromatography on silica gel (10-70% EtOAc/hexane) gave (S)-methyl 2-(tert-butoxycarbonylamino)-3-(4-(trifluoromethyl)-1H-imidazol-1-yl)propanoate (3.00 g, 69%) as a colorless oil. ¹H NMR (CDCl₃) δ 7.46 (s, 1H), 7.19 (s, 1H), 5.24 (br, 1H), 4.64-4.38 (m, 3H), 3.83 (s, 3H), 1.49 (s, 9H).

(S)-tert-Butyl 1-hydroxy-3-(4-(trifluoromethyl)-1H-imidazol-1-yl)propan-2-ylcarbamate: A 250 mL round-bottomed flask was charged with (S)-methyl 2-(tert-butoxycarbonylamino)-3-(4-(trifluoromethyl)-1H-imidazol-1-yl)propanoate (3.0 g, 8.9 mmol) and 50 mL of MeOH. To this was added NaBH₄ (1.3 g, 36 mmol), and the mixture was heated at 50° C. for 2 hours. The reaction was then concentrated and partitioned between DCM and saturated aqueous NaHCO₃. The layers were separated, and the organic layers were dried and concentrated to give (S)-tert-butyl 1-hydroxy-3-(4-(trifluoromethyl)-1H-imidazol-1-yl)propan-2-ylcarbamate as a white foam (2.43 g, 88%). ¹H NMR (CDCl₃) δ 7.60 (s, 1H), 7.35 (s, 1H), 5.11 (br, 1H), 4.29-4.11 (br, 2H), 3.91 (br, 1H), 3.70-3.55 (m, 2H), 3.22-3.05 (br, 1H), 1.44 (s, 9H).

tert-Butyl (4S)-4-(4-(trifluoromethyl)-1H-imidazol-1-yl)methyl)-1,2,3-oxathiazolidine-3-carboxylate 2-oxide: A 500 mL round-bottomed flask was charged with imidazole (4.81 g, 70.7 mmol, Aldrich) and 100 mL of DCM. After cooling to 0° C., SOCl₂ (4.67 g, 39.3 mmol, Aldrich) was added at such a rate that the internal temperature did not rise above 5° C. After 30 minutes, the solution was cooled to −78° C. and a solution of (S)-tert-butyl 1-hydroxy-3-(4-(trifluoromethyl)-1H-imidazol-1-yl)propan-2-ylcarbamate (2.43 g, 7.86 mmol) in 30 mL of DCM was then added over 1 hour. After the addition was complete, the mixture was warmed to room temperature and stirred for 15 minutes. Water (200 mL) was added and the layers were separated. The organic extracts were dried (MgSO₄), filtered, and concentrated to give tert-butyl (4S)-4-(4-(trifluoromethyl)-1H-imidazol-1-yl)methyl)-1,2,3-oxathiazolidine-3-carboxylate 2-oxide (2.11 g, 76%) as a colorless oil.

(R)-tert-Butyl 1-(5-bromothiazol-2-ylamino)-3-(4-(trifluoromethyl)-1H-imidazol-1-yl)propan-2-ylcarbamate: A 250 mL round-bottomed flask was charged with tert-butyl 5-bromothiazol-2-ylcarbamate (1.72 g, 6.15 mmol, prepared as shown in Scheme 2), Cs₂CO₃ (2.38 g, 7.32 mmol), and 100 mL of DMF. After heating to 50° C., tert-butyl (4S)-4-((4-(trifluoromethyl)-1H-imidazol-1-yl)methyl)-1,2,3-oxathiazolidine-3-carboxylate 2-oxide (11.73 g, 31.92 mmol) was added over 30 minutes as a solution in DMF (35 mL). After stirring at 50° C. for 45 minutes, the mixture was carefully quenched with 1M HCl and extracted with ether (400 mL). The ethereal extracts were washed with water (4×150 mL) and brine, dried with MgSO₄, filtered, and concentrated to give an oil. This residue was purified by chromatography on silica gel to give (R)-tert-butyl 1-(5-bromothiazol-2-ylamino)-3-(4-(trifluoromethyl)-1H-imidazol-1-yl)propan-2-ylcarbamate as viscous oil (1.85 g, 67%). ¹H NMR (CDCl₃) δ 7.63 (s, 1H), 7.36 (s, 1H), 7.31 (s, 1H), 5.46 (br, 1H), 4.30-4.02 (m, 5H), 1.53 (s, 9H), 1.39 (s, 9H).

(R)-1-(5-(3-Fluoroisoquinolin-6-yl)thiazol-2-ylamino)-3-(4-(trifluoromethyl)-1H-imidazol-1-yl)propan-2-ylcarbamate. A 20 mL pressure tube was filled with of (R)-tert-butyl 1-(5-bromothiazol-2-ylamino)-3-(4-(trifluoromethyl)-1H-imidazol-1-yl)propan-2-ylcarbamate (0.400 g, 0.701 mmol), Na₂CO₃ (0.223 g, 2.10 mmol), 3-fluoroisoquinolin-6-ylboronic acid (0.147 g, 0.771 mmol, prepared as shown in Scheme 1), Pd(PPh₃)₄ (0.081 g, 0.07 mmol, Aldrich), 6 mL of dioxane, and 1.5 mL of water. The tube was sealed and the mixture was heated at 100° C. for 12 hours. The mixture was then diluted with water and DCM. The precipitate was collected by filtration to give tert-butyl (R)-1-(5-(3-fluoroisoquinolin-6-yl)thiazol-2-ylamino)-3-(4-(trifluoromethyl)-1H-imidazol-1-yl)propan-2-ylcarbamate (0.215 g, 57%). This material was carried on to the next step without further purification.

N—((R)-2-Amino-3-(4-(trifluoromethyl)-1H-imidazol-1-yl)propyl)-5-(3-fluoroisoquinolin-6-yl)thiazol-2-amine: A 20 mL vial was charged with tert-butyl (R)-1-(5-(3-fluoroisoquinolin-6-yl)thiazol-2-ylamino)-3-(4-(trifluoromethyl)-1H-imidazol-1-yl)propan-2-ylcarbamate (0.215 g, 0.401 mmol), 4 mL of DCM, and TFA (0.0309 mL, 0.401 mmol). After stirring at room temperature for 2 hours, the mixture was concentrated and purified via chromatography on silica gel (2-10% 2 M NH₃ in MeOH/DCM) to give N—((R)-2-amino-3-(4-(trifluoromethyl)-1H-imidazol-1-yl)propyl)-5-(3-fluoroisoquinolin-6-yl)thiazol-2-amine (0.125 g, 72%) as a slightly yellow solid. LCMS (API-ES) nm/z: 437.1 (M+H⁺); ¹H NMR (CD₃OD) δ 8.91 (s, 1H), 8.61 (d, J=8.6 Hz, 1H), 7.87 (br, 1H), 7.84-7.80 (m, 2H), 7.75 (br, 1H), 7.71 (s, 1H), 7.38 (s, 1H), 4.26 (dd, J=14.1, 4.5 Hz, 1H), 4.12-4.03 (m, 1H), 3.51-3.36 (m, 3H).

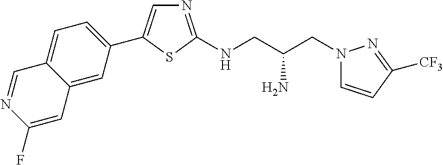

### Example 29

### N—((S)-2-Amino-3-(3-(trifluoromethyl)-1H-pyrazol-1-yl)propyl)-5-(3-fluoroisoquinolin-6-yl)thiazol-2-amine

N—((S)-2-amino-3-(3-(trifluoromethyl)-1H-pyrazol-1-yl)propyl)-5-(3-fluoroisoquinolin-6-yl)thiazol-2-amine was prepared in a similar manner to N—((R)-2-amino-3-(4-(trifluoromethyl)-1H-imidazol-1-yl)propyl)-5-(3-fluoroisoquinolin-6-yl)thiazol-2-amine (Example 28) using 3-trifluoromethylpyrazole (Aldrich catalog number 406228) in place of 4-(trifluoromethyl)-1H-imidazole. LCMS (API-ES) m/z: 437.1 (M+H⁺); ¹H NMR (CD₃OD) δ 8.90 (s, 1H), 8.05 (d, J=8.6 Hz, 1H), 7.84-7.77 (m, 3H), 7.68 (s, 1H), 7.35 (s, 1H), 6.64 (d, J=2.4 Hz, 1H), 4.38 (dd, J=13.9, 4.9 Hz, 1H), 4.22 (dd, J=13.9, 7.0 Hz, 1H), 3.59-3.35 (m, 3H).

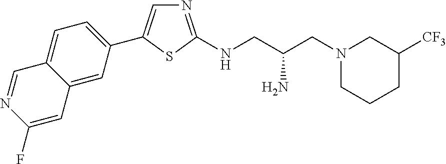

### Example 30

### N—((R)-2-Amino-3-((3S)-(trifluoromethyl)piperidin-1-yl)propyl)-5-(3-fluoroisoquinolin-6-yl)thiazol-2-amine

N—((R)-2-Amino-3-(3-(trifluoromethyl)piperidin-1-yl)propyl)-5-(3-fluoroisoquinolin-6-yl)thiazol-2-amine was prepared in a similar manner to N4R)-2-amino-3-(4-(trifluoromethyl)-1H-imidazol-1-yl)propyl)-5-(3-fluoroisoquinolin-6-yl)thiazol-2-amine (Example 28) using (±)-3-trifluoromethylpiperidine (Aldrich, catalog number 665495) in place of 4-(trifluoromethyl)-1H-imidazole. LCMS (API-ES) m/z: 454.1 (M+H⁺); ¹H NMR (CD₃OD) δ 8.90 (s, 1H), 8.07 (d, J=8.6 Hz, 1H), 7.88-7.79 (m, 2H), 7.70 (s, 1H), 7.38 (s, 1H), 3.35-3.33 (m, 2H), 3.17-2.87 (m, 2H), 2.56-2.35 (m, 3H), 2.19-1.91 (m, 3H), 1.86-1.57 (m, 2H), 1.46-1.26 (m, 2H).

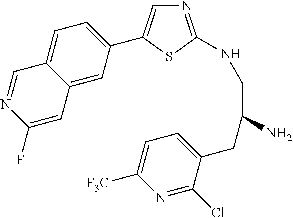

### Example 31

### N—((S)-2-Amino-3-(2-chloro-6-(trifluoromethyl)pyridin-3-yl)propyl)-5-(3-fluoroisoquinolin-6-yl)thiazol-2-amine

N—((S)-2-Amino-3-(2-chloro-6-(trifluoromethyl)pyridin-3-yl)propyl)-5-(3-fluoroisoquinolin-6-yl)thiazol-2-amine was prepared in a similar manner to N—((R)-2-amino-3-(4-(trifluoromethyl)-1H-imidazol-1-yl)propyl)-5-(3-fluoroisoquinolin-6-yl)thiazol-2-amine (Example 28) using (S)-methyl 2-(tert-butoxycarbonylamino)-3-(2-chloro-6-(trifluoromethyl)pyridin-3-yl)propanoate instead of (S)-methyl 2-(tert-butoxycarbonylamino)-3-(4-(trifluoromethyl)-1H-imidazol-1-yl)propanoate. LCMS (API-ES) m/z=482.0, (M+H⁺); ¹H NMR (CD₃OD) δ 8.94 (s, 1H), 8.15-8.08 (m, 2H), 7.88-7.81 (m, 3H), 7.75 (s, 1H), 7.40 (s, 1H), 4.04-3.94 (m, 1H), 3.76-3.61 (m, 2H), 3.35-3.30 (m, 1H), 3.23-3.16 (m, 1H). (S)-Methyl 2-(tert-butoxycarbonylamino)-3-(2-chloro-6-(trifluoromethyl)pyridin-3-yl)propanoate was prepared as shown in Scheme 12.

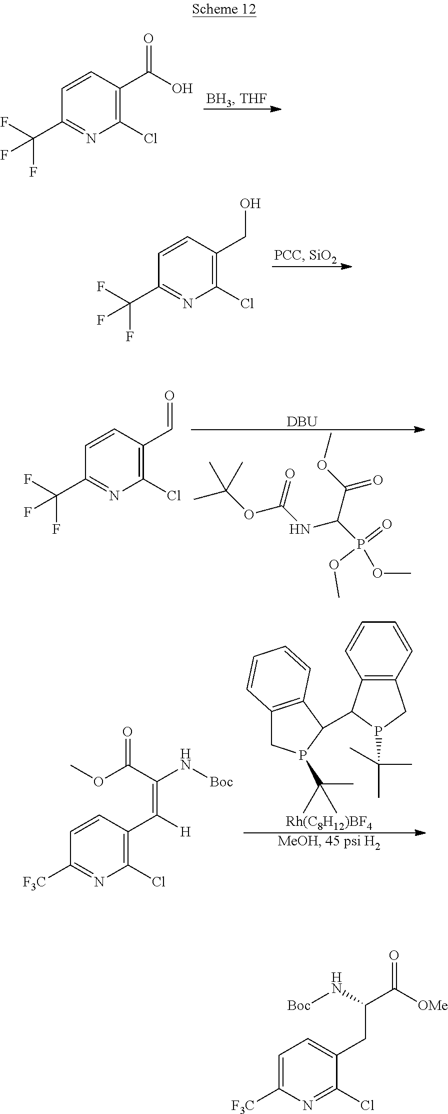

(2-Chloro-6-(trifluoromethyl)pyridin-3-yl)methanol. A 1 L round-bottomed flask was charged with 2-chloro-6-(trifluoromethyl)nicotinic acid (10.10 g, 44.8 mmol, see Eur. J. Org. Chem., 2004, 3793) and 100 mL of THF. To this was added BH₃-THF (67.2 mL, 67.2 mmol, Aldrich), and the resulting mixture was stirred overnight at room temperature. The reaction was concentrated, quenched with saturated aqueous NaHCO₃ and extracted with DCM. The combined extracts were dried and concentrated to give an oil that was purified by chromatography on silica gel (0-30% EtOAc/hexane) to give (2-chloro-6-(trifluoromethyl)pyridin-3-yl)methanol (8.00 g, 84%) as a colorless oil. ¹H NMR (CDCl₃) δ 8.12 (d, J=7.8 Hz, 1H), 7.68 (d, J=7.6 Hz, 1H), 4.86 (s, 2H).

2-Chloro-6-(trifluoromethyl)nicotinaldehyde. A 100 mL round-bottom flask was charged with (2-chloro-6-(trifluoromethyl)pyridin-3-yl)methanol (1.00 g, 4.73 mmol), 10 mL of DCM, PCC (2.04 g, 9.45 mmol, Aldrich), and 6.00 g of silica gel. After stirring at room temperature for 3 hours, the mixture was filtered through a pad of silica rinsing with 4:1 EtOAc/hexane. After concentrating in vacuo 2-chloro-6-(trifluoromethyl)nicotinaldehyde (0.805 g, 81%) was obtained as a colorless oil. ¹H NMR (CDCl₃) δ 10.50 (s, 1H), 8.42 (d, J=7.8 Hz, 1H), 7.80 (d, J=7.8 Hz, 1H).

(E)-Methyl 2-(tert-butoxycarbonylamino)-3-(2-chloro-6-(trifluoromethyl)pyridin-3-yl)acrylate. A 250 mL round-bottom flask was charged with (+/−)-Boc-alpha-phosphonoglycine trimethyl ester (12.50 g, 42.1 mmol, Fluka catalog number 09659) and then 150 mL of DCM was added and the mixture was cooled to 0° C. To this mixture was added DBU (6.41 g, 42.1 mmol, Aldrich). After stirring for 30 minutes at 0° C., 2-chloro-6-(trifluoromethyl)nicotinaldehyde (7.35 g, 35.1 mmol, Aldrich) was added as a solution in DCM (50 mL). After 1 hour at 0° C., no starting material remained. The mixture was quenched with 1N HCl and extracted with DCM. The combined extracts were dried (MgSO₄), filtered, and concentrated to give a solid that was purified by chromatography on silica gel (0-40% EtOAc/hexane) to give (E)-methyl 2-(tert-butoxycarbonylamino)-3-(2-chloro-6-(trifluoromethyl)pyridin-3-yl)acrylate (10.0 g, 75%) as a bright white solid. ¹H NMR (CDCl₃) δ 7.97 (d, J=8.0 Hz, 1H), 7.56 (d, J=8.0 Hz, 1H), 7.30 (d, J=15.3 Hz, 1H), 6.68 (br, 1H), 3.94 (s, 3H), 1.34 (s, 9H).

(S)-Methyl 2-(tert-butoxycarbonylamino)-3-(2-chloro-6-(trifluoromethyl)pyridin-3-yl)propanoate: A 250 mL tube was charged with (Z)-methyl 2-(tert-butoxycarbonylamino)-3-(2-chloro-6-(trifluoromethyl)pyridin-3-yl)acrylate (5.00 g, 13.1 mmol), 50 mL of MeOH, and, [(1S,1′S,2S,2′S)-2,2′-bis(1,1-dimethylethyl)-2,2′,3,3′-tetrahydro-1,1′-bi-1H-isophosphindole-κP2,κP2′][(1,2,5,6-η)-1,5-cyclooctadiene]-rhodium(I) tetrafluoroborate (1.34 g, 1.97 mmol, see Brit. UK Pat. Appl., 2437078, 17 Oct. 2007). The mixture was degassed for 3 minutes, and then placed under 45 psi of hydrogen. After 3 hours, LCMS showed that the reaction was complete. The mixture was concentrated and passed through a small pad of silica, rinsing with 1:2 EtOAc/hexane. The filtrate was concentrated to give (S)-methyl 2-(tert-butoxycarbonylamino)-3-(2-chloro-6-(trifluoromethyl)pyridin-3-yl)propanoate (5.01 g, 100%, >99% ee determined by comparison to a racemic standard) as a white solid. ¹H NMR (CDCl₃) δ 7.78 (d, J=7.8 Hz, 1H) 7.59 (d, J=7.8 Hz, 1H), 5.21-5.11 (m, 1H), 4.74-4.65 (m, 1H), 3.80 (s, 3H), 3.48-3.38 (m, 1H), 3.22-3.12 (m, 1H), 1.38 (s, 9H).

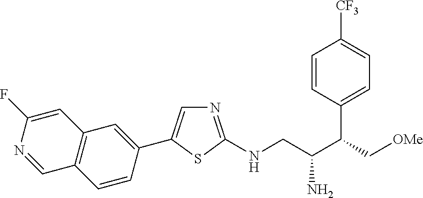

### Example 32

### N-((2S,3S)-2-Amino-4-methoxy-3-(4-(trifluoromethyl)phenyl)butyl)-5-(3-fluoroisoquinolin-6-yl)thiazol-2-amine

The title compound was synthesized in a manner similar to that described in Example 14 using tert-butyl (2S,3S)-1-hydroxy-4-methoxy-3-(4-(trifluoromethyl)phenyl)-butan-2-ylcarbamate instead of (S)-tert-butyl 3-(3,4-dichlorophenyl)-1-hydroxypropan-2-ylcarbamate. MS m/z: 491(M+1). ¹H NMR (400 MHz, DMSO-d₆) δ ppm 8.97 (s, 1H), 8.11 (broad s, 1H), 8.09 (d, J=8.6 Hz, 1H), 7.88 (d, J=8.8 Hz, 1H), 7.81 (s, 1H), 7.76 (s, 1H), 7.67 (d, J=8.2 Hz, 2H), 7.54 (d, J=8.0 Hz, 2H), 7.50 (s, 1H), 3.76 (dd, J=6.5 Hz, 9.6 Hz, 1H), 3.67 (dd, J=7.4 Hz, 9.4 Hz, 1H), 3.38-3.26 (m, 2H), 3.23 (s, 3H), 3.12 (dd, J=6.6 Hz, 11.3 Hz, 1H), 3.00 (dd, J=9.0 Hz, 13.7 Hz, 1H). 10% of the enantiomer, N-((2R,3R)-2-amino-4-methoxy-3-(4-(trifluoromethyl)phenyl)butyl)-5-(3-fluoroisoquinolin-6-yl)thiazol-2-amine, was present as an impurity. tert-Butyl (2S,3S)-1-hydroxy-4-methoxy-3-(4-(trifluoromethyl)phenyl)-butan-2-ylcarbamate was prepared as shown in Scheme 13.

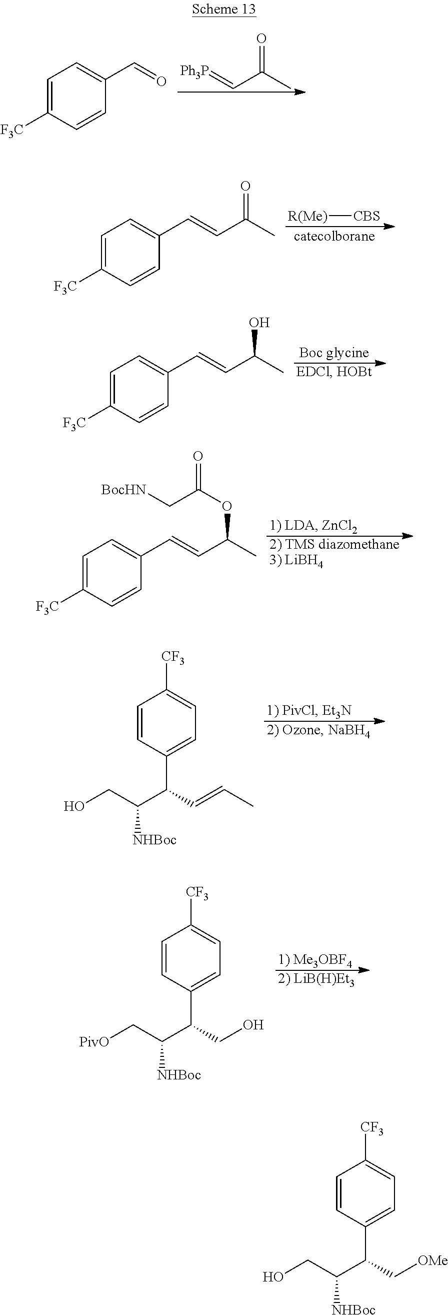

(E)-4-(4-(Trifluoromethyl)phenyl)but-3-en-2-one: 4-(Trifluoromethyl)benzaldehyde (25.0 g, 144 mmol, Aldrich, catalog number 224944) was taken up in 500 mL of DCM. 1-Triphenylphosphoranylidene-2-propanone (48.0 g, 151 mmol, Aldrich, catalog number 158755) was added. After 5 hours, an additional 3 g of 1-triphenylphosphoranylidene-2-propanone was added. The mixture was stirred for 10 hours. The solvent was removed under reduced pressure, and the residue was triturated with 500 mL of 5% EtOAc/hexanes. The mixture was filtered, removing a large amount of P(O)Ph₃. The residue was taken up in 300 mL of 2.5% EtOAc/hexanes and filtered through a pad of silica. The mixture was concentrated under reduced pressure and the residue was found to be (E)-4-(4-(trifluoromethyl)phenyl)but-3-en-2-one (28.3 g, 92.0%). ¹H NMR (400 MHz, CDCl₃) δ ppm 7.68-7.60 (m, 4H), 7.52 (d, J=16.43 Hz, 1H), 6.78 (d, J=16.4 Hz, 1H), 2.41 (s, 3H).

(S,E)-4-(4-(Trifluoromethyl)phenyl)but-3-en-2-ol: (E)-4-(4-(Trifluoromethyl)phenyl)but-3-en-2-one (15 g, 70 mmol) was taken up in 500 mL of toluene. (R)-2-Methyl-CBS-oxazaborolidine (1.09 M in toluene (6.4 mL, 7.0 mmol, Aldrich, catalog number 457698)) was added, and the mixture was chilled to −78° C. Catecholborane (13 mL, 119 mmol, Aldrich) was added dropwise via addition funnel in 125 mL of toluene. The mixture was stirred for 25 minutes and then gradually warmed to −45° C. and stirred for 2 hours. The yellow color taken on during the catecholborane addition faded during this time and the solution cleared. The mixture was quenched with 300 mL of water and warmed to room temperature. The mixture was partitioned in a separatory funnel. The organic portion was washed 3 times with 200 mL of 5% aqueous KOH (to remove the catechol), twice with 200 mL of 10% aqueous HCl (to remove the (R)-2-methyl-CBS-oxazaborolidine catalyst), and once with 200 mL of brine. The organic layer was then dried over MgSO₄. Filtration and concentration under reduced pressure afforded (S,E)-4-(4-(trifluoromethyl)phenyl)but-3-en-2-ol (15 g, 99%) as a yellow oil that slowly crystallized on standing. ¹H NMR (400 MHz, CDCl₃) δ ppm 7.57 (d, J=8.22 Hz, 2H) 7.50-7.45 (m, 2H) 6.62 (d, J=16.04 Hz, 1H) 6.36 (dd, J=16.04, 6.06 Hz, 1H) 4.49-4.57 (m, 1H) 1.61 (d, J=4.30 Hz, 1H) 1.39 (d, J=6.46 Hz, 3H). 10% of the enantiomer, (R,E)-4-(4-(trifluoromethyl)phenyl)but-3-en-2-ol was formed in this step, but was not removed.

(S,E)-4-(4-Trifluoromethyl)phenyl)but-3-en-2-yl 2-(tert-butoxycarbonylamino)acetate: (S,E)-4-(4-(Trifluoromethyl)phenyl)but-3-en-2-ol (11.2 g, 51.8 mmol) was taken up in 240 mL of DMF. N-Boc glycine (22.7 g, 130 mmol, Aldrich), N1-((ethylimino)methylene)-N3,N3-dimethylpropane-1,3-diamine hydrochloride (29.8 g, 155 mmol, Aldrich), 1H-benzo[d][1,2,3]triazol-1-ol (21.0 g, 155 mmol, Aldrich), and Hunig's base (27.1 mL, 155 mmol, Aldrich) were added. After 12 hours, the solvent was removed under reduced pressure. The residue was taken up in 500 mL of EtOAc and transferred to a separatory funnel. The mixture was washed with 200 mL of 10% aqueous HCl, 200 mL of aqueous NaHCO₃, and 200 mL of brine, and then dried over MgSO₄. Filtration and concentration under reduced pressure, followed by flash chromatography on silica gel (2.5% to 15% EtOAc/hexanes) afforded (S,E)-4-(4-(trifluoromethyl)phenyl)but-3-en-2-yl 2-(tert-butoxycarbonylamino)acetate (16.5 g, 85.3%) as a thick oil. ¹H NMR (400 MHz, CDCl₃) δ ppm 7.59-7.52 (m, 2H) 7.47 (d, J=8.22 Hz, 2H) 6.64 (d, J=15.85 Hz, 1H) 6.26 (dd, J=15.94, 6.55 Hz, 1H) 5.60 (dq, J=6.55, 6.29 Hz, 1H) 5.00 (s, 1H) 3.85-4.00 (m, 2H), 1.46-1.43 (m, 12H). 10% of the enantiomer, (R,E)-4-(4-trifluoromethyl)phenyl)but-3-en-2-yl 2-(tert-butoxycarbonylamino)acetate, was present as an impurity.

tert-Butyl (2S,3S,E)-1-hydroxy-3-(4-(trifluoromethyl)phenyl)hex-4-en-2-ylcarbamate: Diisopropylamine (8.3 mL, 59 mmol) was taken up in 45 mL of THF and the mixture was chilled to −20° C. Butyllithium (2.5 M in hexane (19 mL, 48 mmol, Aldrich)) was added, and the mixture was stirred for 20 minutes. The mixture was then chilled to −78° C. (S,E)-4-(4-(Trifluoromethyl)phenyl)but-3-en-2-yl 2-(tert-butoxycarbonylamino)acetate (8.1 g, 22 mmol) in 22 mL of THF was then added at −78° C. by cannula. The mixture immediately turned purple. After 5 minutes, Zinc(II) chloride, 0.5 M in THF (50 mL, 25 mmol, Aldrich), was added slowly to the mixture. The mixture was then gradually warmed to room temperature over 1.5 hours. The mixture was quenched with 30 mL of 10% aqueous HCl. The solvent was removed under reduced pressure. The residue was taken up in 400 mL of ether. The mixture was washed with 100 mL of 10% aqueous HCl. The mixture was then extracted twice with 125 mL of 1 M aqueous NaOH. The combined basic extracts were acidified with concentrated HCl. The mixture was then extracted three times with 200 mL of ether. The combined ether extracts were dried over MgSO₄. Filtration and concentration under reduced pressure afforded (2S,3S,E)-2-(tert-butoxycarbonylamino)-3-(4-(trifluoromethyl)phenyl)hex-4-enoic acid (5.3 g, 65%) which was carried on directly without any further purification. 10% of the enantiomer, (2R,3R,E)-2-(tert-butoxycarbonylamino)-3-(4-(trifluoromethyl)phenyl)hex-4-enoic acid, was present as an impurity.

(2S,3S,E)-Methyl 2-(tert-butoxycarbonylamino)-3-(4-(trifluoromethyl)phenyl)hex-4-enoate. (2S,3S,E)-2-(tert-butoxycarbonylamino)-3-(4-(trifluoromethyl)phenyl)hex-4-enoic acid (5.3 g, 14 mmol) was taken up in 70 mL of 3.5:1 benzene:MeOH. TMS diazomethane (2M in hexane (7.8 mL, 16 mmol, Aldrich)) was slowly added to the mixture and bubbling ensued. Approximately 2 mL excess TMS diazomethane reagent was added. The bubbling was monitored, and the addition was stopped when the bubbling ceased. The solvent was then removed under reduced pressure. The mixture was triturated twice with 50 mL of 10% ether:hexanes, affording (2S,3S,E)-methyl 2-(tert-butoxycarbonylamino)-3-(4-(trifluoromethyl)phenyl)hex-4-enoate (5.4 g, 98%) as a white solid. ¹H NMR (400 MHz, CDCl₃) δ ppm 7.59 (d, J=8.22 Hz, 2H) 7.34 (d, J=8.22 Hz, 2H) 5.75-5.59 (m, 2H) 4.92-4.84 (m, 1H) 4.70-4.63 (m, 1H) 3.78-3.72 (m, 1H) 3.68 (s, 3H) 1.72 (d, J=5.28 Hz, 3H) 1.37 (s, 9H). 10% of the enantiomer, (2R,3R,E)-methyl 2-(tert-butoxycarbonylamino)-3-(4-(trifluoromethyl)phenyl)hex-4-enoate, was present as an impurity.

tert-Butyl (2S,3S,E)-1-hydroxy-3-(4-(trifluoromethyl)phenyl)hex-4-en-2-ylcarbamate. (2S,3S,E)-Methyl 2-(tert-butoxycarbonylamino)-3-(4-(trifluoromethyl)phenyl)hex-4-enoate (5.4 g, 14 mmol) was taken up in 140 mL of diethyl ether and chilled to 0° C. Lithium borohydride (1.2 g, 56 mmol, Aldrich) was added to the mixture. After 1.5 hours, approximately 10 mL of MeOH was added to the reaction. The mixture was stirred an additional 20 minutes and was then quenched by dropwise addition of aqueous NH₄Cl (20 mL). The mixture was then diluted with 50 mL of aqueous NH₄Cl and 50 mL of water. The mixture was partitioned and the aqueous portion was extracted with 120 mL of ether. The combined organic extracts were washed with 100 mL of brine and dried over MgSO₄. Filtration and concentration under reduced pressure, followed by flash chromatography on silica gel (5% to 30% EtOAc/hexanes) afforded tert-butyl (2S,3S,E)-1-hydroxy-3-(4-(trifluoromethyl)phenyl)hex-4-en-2-ylcarbamate (3.9 g, 78%) as a sticky solid. ¹H NMR (400 MHz, CDCl₃) δ ppm 7.56 (d, J=8.03 Hz, 2H) 7.34 (d, J=8.03 Hz, 2H) 5.59-5.69 (m, 2H) 4.60-4.50 (m, 1H) 3.94 (s, 1H) 3.80-3.70 (m, 2H) 3.63-3.55 (m, 1H) 2.15-2.07 (m, 1H) 1.69 (d, J=4.52 Hz, 3H) 1.29 (s, 9H). 10% of the enantiomer, tert-butyl (2R,3R,E)-1-hydroxy-3-(4-(trifluoromethyl)phenyl)hex-4-en-2-ylcarbamate, was present as an impurity.

(2S,3S,E)-2-(tert-Butoxycarbonyl)-3-(4-(trifluoromethyl)-phenyl)hex-4-enyl pivalate. tert-Butyl (2S,3S,E)-1-hydroxy-3-(4-(trifluoromethyl)phenyl)hex-4-en-2-ylcarbamate (2.35 g, 6.5 mmol) was taken up in 60 mL of DCM, and the mixture was chilled to 0° C. TEA (2.7 mL, 20 mmol), pivaloyl chloride (0.97 mL, 7.8 mmol, Aldrich), and N,N-dimethylpyridin-4-amine (0.040 g, 0.33 mmol, Aldrich) were then added. The resulting mixture was stirred for 12 hours. The reaction was quenched with 50 mL of aqueous NaHCO₃ and stirred for 10 minutes. The mixture was partitioned, and the aqueous portion was extracted with 50 mL of DCM. The combined organic extracts were dried over MgSO₄. Filtration and concentration under reduced pressure afforded a yellow solid that was taken up in 10% EtOAc/hexanes and filtered through a plug of silica gel. The solvent was removed under reduced pressure, affording (2S,3S,E)-2-(tert-butoxycarbonylamino)-3-(4-(trifluoromethyl)-phenyl)hex-4-enyl pivalate (1.8 g, 62%) as a light yellow solid that was carried on without any further purification. 10% of the enantiomer, (2R,3R,E)-2-(tert-butoxycarbonylamino)-3-(4-(trifluoromethyl)-phenyl)hex-4-enyl pivalate, was present as an impurity.

(2S,3S)-2-(tert-Butoxycarbonyl)-4-hydroxy-3-(4-(trifluoromethyl)phenyl)butyl pivalate. (2S,3S,E)-2-(tert-Butoxycarbonyl)-3-(4-(trifluoromethyl)phenyl)hex-4-enyl pivalate (1.8 g, 4.1 mmol) was taken up in 40 mL of 1:1 MeOH:DCM, and the mixture was chilled to −78° C. Ozone was bubbled through the mixture until a blue color persisted. Nitrogen was then bubbled through the mixture for 15 minutes. NaBH₄ (0.77 g, 20 mmol, Aldrich) was added, and the mixture was warmed to room temperature. The mixture was stirred for 2 hours. The mixture was quenched with 40 mL of aqueous NH₄Cl. After 30 minutes, the mixture was diluted with 20 mL of water and extracted three times with 40 mL of DCM. The combined organic extracts were dried over MgSO₄. Filtration and concentration under reduced pressure afforded (2S,3S)-2-(tert-butoxycarbonylamino)-4-hydroxy-3-(4-(trifluoromethyl)phenyl)butyl pivalate (1.8 g, 100%) as a white crystalline solid. 10% of the enantiomer, (2R,3R)-2-(tert-butoxycarbonylamino)-4-hydroxy-3-(4-(trifluoromethyl)phenyl)butyl pivalate, was present as an impurity.

(2S,3S)-2-(tert-Butoxycarbonyl)-4-methoxy-3-(4-(trifluoromethyl)phenyl)butyl pivalate. Trimethyloxonium tetrafluoroborate (2.3 g, 16 mmol, Aldrich) was taken up in 10 mL of DCM. Proton sponge (3.3 g, 16 mmol, Aldrich) and (2S,3S)-2-(tert-butoxycarbonylamino)-4-hydroxy-3-(4-(trifluoromethyl)phenyl)butyl pivalate (2.25 g, 5.2 mmol) were added to the mixture in 15 mL of DCM. The mixture was shielded from light and stirred for 3 hours. The mixture was carefully quenched with 70 mL of 10% aqueous HCl and extracted three times with 100 mL of DCM. The combined organic extracts were dried over MgSO₄. Filtration and concentration under reduced pressure, followed by flash chromatography on silica gel (5% to 25% EtOAc/hexanes) afforded (2S,3S)-2-(tert-butoxycarbonylamino)-4-methoxy-3-(4-(trifluoromethyl)phenyl)butyl pivalate (1.4 g, 60%) as a white solid. ¹H NMR (400 MHz, CDCl₃) δ ppm 7.58 (d, J=8.02 Hz, 2H) 7.34 (d, J=8.02 Hz, 2H) 4.42-4.32 (m, 2H) 4.15-4.00 (m, 2H) 3.73-3.63 (m, 2H) 3.34 (s, 3H) 1.35 (s, 9H) 1.19 (s, 9H). 10% of the enantiomer, 2R,3R)-2-(tert-butoxycarbonylamino)-4-methoxy-3-(4-(trifluoromethyl)phenyl)butyl pivalate was present as an impurity.

tert-Butyl (2S,3S)-1-hydroxy-4-methoxy-3-(4-(trifluoromethyl)phenyl)butan-2-ylcarbamate. (2S,3S)-2-(tert-Butoxycarbonyl)-4-methoxy-3-(4-(trifluoromethyl)phenyl)butyl pivalate (1.5 g, 3.4 mmol) was taken up in 30 mL of THF and chilled to −78° C. Super hydride (1.0 M in THF (8.4 mL, 8.4 mmol, Aldrich)) was added slowly to the mixture. After 5 minutes, the mixture was warmed to 0° C. After 30 minutes, the mixture was quenched with 20 mL of aqueous NH₄Cl. The mixture was extracted twice with 30 mL of EtOAc, and the combined organic extracts were washed with 20 mL of brine and dried over MgSO₄. Filtration and concentration under reduced pressure, followed by flash chromatography on silica gel (20% to 50% EtOAc/hexanes) afforded tert-butyl (2S,3S)-1-hydroxy-4-methoxy-3-(4-(trifluoromethyl)phenyl)butan-2-ylcarbamate (0.85 g, 70%) as a white solid. ¹H NMR (400 MHz, CDCl₃) δ ppm 7.57 (d, J=8.22 Hz, 2H) 7.39 (d, J=8.02 Hz, 2H) 4.84 (s, 1H) 4.02-3.91 (m, 1H) 3.78-3.59 (m, 4H) 3.39 (s, 3H) 3.30-3.22 (m, 1H) 2.83-2.76 (m, 1H) 1.33 (s, 9H). 10% of the enantiomer, tert-butyl (2R,3R)-1-hydroxy-4-methoxy-3-(4-(trifluoromethyl)phenyl)butan-2-ylcarbamate, was present as an impurity.

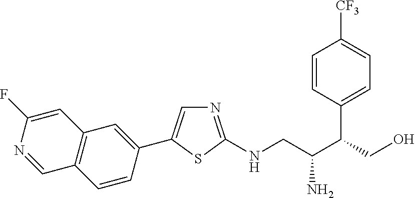

### Example 33

### (2S,3S)-3-Amino-4-(5-(3-fluoroisoquinolin-6-yl)thiazol-2-ylamino)-2-(4-(trifluoromethyl)phenyl)butan-1-ol

The title compound was synthesized in a manner similar to that described in Example 14 using tert-butyl (5-bromo-1,3-thiazol-2-yl)((2S,3S)-2-((tert-butoxycarbonyl)amino)-4-hydroxy-3-(4-(trifluoromethyl)phenyl)butyl)carbamate instead of tert-butyl (5-bromo-1,3-thiazol-2-yl)((2S)-2-((tert-butoxycarbonyl)amino)-3-(3,4-dichlorophenyl)propyl)carbamate. MS m/z: 477 (M+1). ¹H NMR (400 MHz, DMSO-d₆) δ ppm 8.97 (s, 1H), 8.10 (braod s, 1H), 8.09 (d, J=9.0 Hz, 1H), 7.87 (d, J=8.4 Hz, 1H), 7.80 (s, 1H), 7.76 (s, 1H), 7.66 (d, J=8.1 Hz, 2H), 7.54 (d, J=8.0 Hz, 2H), 7.50 (s, 1H), 4.76 (broad s, 1H), 3.84 (dd, J=10.5 Hz, 11.2 Hz, 1H), 3.70 (dd, 6.4 Hz, 10.5 Hz, 1H), 3.44-3.26 (m, 2H), 3.00 (dd, J=7.2 Hz, 12.5 Hz, 1H), 2.94 (dd, J=6.7 Hz, 12.0 Hz, 1H). 10% of the enantiomer, (2R,3R)-3-amino-4-(5-(3-fluoroisoquinolin-6-yl)thiazol-2-ylamino)-2-(4-(trifluoromethyl)phenyl)butan-1-ol, was present as an impurity. tert-Butyl (5-bromo-1,3-thiazol-2-yl)((2S,3S)-2-((tert-butoxycarbonyl)amino)-4-hydroxy-3-(4-(trifluoromethyl)phenyl)butyl)carbamate was prepared as shown in Scheme 14.

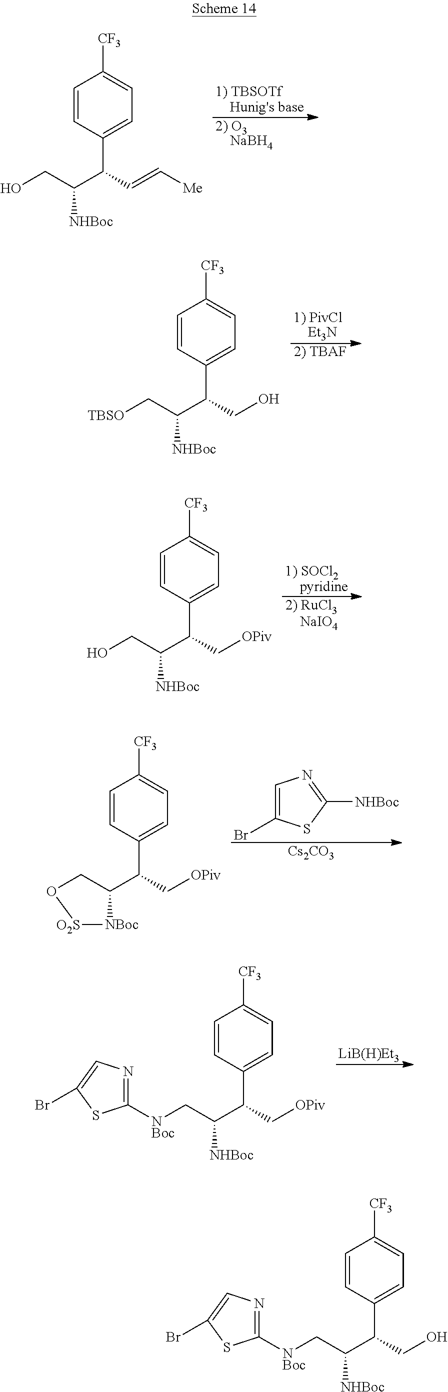

tert-Butyl (2S,3S)-1-(tert-butyldimethylsilyloxy)-4-hydroxy-3-(4-(trifluoromethyl)phenyl)butan-2-ylcarbamate: tert-Butyl (2S,3S,E)-1-hydroxy-3-(4-(trifluoromethyl)phenyl)hex-4-en-2-ylcarbamate (1.94 g, 5.4 mmol, prepared as shown in Scheme 13) was taken up in 50 mL of DCM and chilled to 0° C. N,N-Diisopropylethylamine (2.4 mL, 13 mmol, Aldrich) was added, followed by slow addition of tert-butyldimethylsilyl trifluoromethanesulfonate (TBSOTf) (1.5 mL, 6.5 mmol, Aldrich). After 45 minutes, an additional 0.20 mL of TBSOTf was added. After an additional 20 minutes, the reaction was quenched with 50 mL of aqueous NaHCO₃. The mixture was partitioned, and the aqueous portion was extracted twice with 50 mL of DCM. The combined organic extracts were dried over MgSO₄. Filtration and concentration under reduced pressure, followed by flash chromatography on silica gel (1% to 7.5% EtOAc/hexanes) afforded tert-butyl (2S,3S,E)-1-(tert-butyldimethylsilyloxy)-3-(4-(trifluoromethyl)phenyl)hex-4-en-2-ylcarbamate (2.0 g, 78%) as a clear oil. The oil crystallized on standing over 12 hours. ¹H NMR (400 MHz, CDCl₃) δ ppm 7.54 (d, J=8.02 Hz, 2H) 7.35 (d, J=8.02 Hz, 2H) 5.61 (s, 1H) 5.59 (d, J=5.87 Hz, 1H) 4.62-4.60 (m, 1H) 3.97-3.92 (m, 1H) 3.79-3.76 (m, 1H) 3.66-3.59 (m, 2H) 1.69 (d, J=5.28 Hz, 3H) 1.26 (s, 9H) 0.92-0.97 (m, 9H) 0.06 (s, 6H). 10% of the enantiomer, tert-butyl (2R,3R,E)-1-(tert-butyldimethylsilyloxy)-3-(4-(trifluoromethyl)phenyl)hex-4-en-2-ylcarbamate (2.0 g, 78%), was present as an impurity.

tert-Butyl (2S,3S)-1-(tert-butyldimethylsilyloxy)-4-hydroxy-3-(4-(trifluoromethyl)phenyl)butan-2-ylcarbamate. tert-Butyl (2S,3S,E)-1-(tert-butyldimethylsilyloxy)-3-(4-(trifluoromethyl)phenyl)hex-4-en-2-ylcarbamate (2.0 g, 4.2 mmol) was dissolved in 40 mL of 1:1 MeOH/DCM, and the mixture was chilled to −78° C. Ozone was bubbled through the mixture until a blue color persisted. Nitrogen was then bubbled through the mixture for 15 minutes. NaBH₄ (0.80 g, 21 mmol) was added, and the mixture was warmed to room temperature. After 3 hours, the mixture was quenched with aqueous NH₄Cl. The mixture was extracted three times with 50 mL of DCM. The combined organic extracts were dried over MgSO₄. Filtration and concentration under reduced pressure afforded tert-butyl (2S,3S)-1-(tert-butyldimethylsilyloxy)-4-hydroxy-3-(4-(trifluoromethyl)phenyl)butan-2-ylcarbamate (1.9 g, 97%) as a white solid. ¹H NMR (400 MHz, CDCl₃) δ ppm 7.56 (d, J=8.03 Hz, 2H) 7.32 (d, J=8.03 Hz, 2H) 4.51 (s, 1H) 4.22-4.16 (m, 1H) 3.90-3.66 (m, 3H) 3.47 (d, J=5.52 Hz, 2H) 3.15-3.20 (m, 1H) 1.45 (s, 9H) 0.84 (s, 9H) 0.01 (s, 3H)-0.01 (s, 3H). 10% of the enantiomer, tert-butyl (2R,3R)-1-(tert-butyldimethylsilyloxy)-4-hydroxy-3-(4-(trifluoromethyl)phenyl)butan-2-ylcarbamate, was present as an impurity.

(2S,3S)-3-(tert-Butoxycarbonyl)-4-hydroxy-2-(4-(trifluoromethyl)phenyl)butyl pivalate. tert-Butyl (2S,3S)-1-(tert-butyldimethylsilyloxy)-4-hydroxy-3-(4-(trifluoromethyl)phenyl)butan-2-ylcarbamate (1.9 g, 4.1 mmol) was taken up in 40 mL of DCM, and the mixture was chilled to 0° C. TEA (1.1 mL, 8.2 mmol, Aldrich), N,N-dimethylpyridin-4-amine (0.025 g, 0.20 mmol, Aldrich), and pivaloyl chloride (0.76 mL, 6.1 mmol, Aldrich) were then added. The resulting mixture was warmed to room temperature. After 12 hours, the reaction was quenched with 50 mL of aqueous NaHCO₃ and stirred for 10 minutes. The mixture was partitioned, and the aqueous portion was extracted twice with 50 mL of DCM. The combined organic extracts were washed with 50 mL of aqueous NaCHO₃ and 50 mL of aqueous NH₄Cl, and then dried over MgSO₄. Filtration and concentration under reduced pressure afforded (2S,3S)-3-(tert-butoxycarbonylamino)-4-(tert-butyldimethylsilyloxy)-2-(4-(trifluoromethyl)phenyl)butyl pivalate (2.2 g, 99%) that was carried on without any further purification. 10% of the enantiomer, (2R,3R)-3-(tert-butoxycarbonylamino)-4-(tert-butyldimethylsilyloxy)-2-(4-(trifluoromethyl)phenyl)butyl pivalate, was present as an impurity.

(2S,3S)-3-(tert-Butoxycarbonyl)-4-hydroxy-2-(4-(trifluoromethyl)phenyl)butyl pivalate. (2S,3S)-3-(tert-Butoxycarbonyl)-4-(tert-butyldimethylsilyloxy)-2-(4-(trifluoromethyl)phenyl)butyl pivalate (2.2 g, 4.0 mmol) was taken up in 40 mL of THF, and the mixture was chilled to 0° C. TBAF (1 M in THF (6.0 mL, 6.0 mmol, Aldrich)) was then slowly added to the mixture. After 20 minutes, the mixture was warmed to room temperature and stirred for 1 hour. 0.5 mL of additional TBAF was added, and the mixture was stirred for another 20 minutes. The mixture was quenched with 20 mL of aqueous NH₄Cl. The mixture was then diluted with 40 mL of water and extracted twice with 50 mL of EtOAc. The combined organic extracts were washed with 50 mL of brine and dried over MgSO₄. Filtration and concentration under reduced pressure, followed by flash chromatography on silica gel (5% to 40% EtOAc/hexanes) afforded (2S,3S)-3-(tert-butoxycarbonylamino)-4-hydroxy-2-(4-(trifluoromethyl)phenyl)butyl pivalate (1.5 g, 86%) as a yellow solid. ¹H NMR (400 MHz, CDCl₃) δ ppm 7.59 (d, J=8.22 Hz, 2H) 7.37 (d, J=8.02 Hz, 2H) 4.57 (d, J=6.4 Hz, 1H) 4.45-4.35 (m, 2H) 4.06 (s, 1H) 3.70 (s, 2H) 3.51-3,41 (m, 1H), 1.75 (broad s, 1H) 1.33 (s, 9H) 1.08 (s, 9H). 10% of the enantiomer, (2R,3R)-3-(tert-butoxycarbonylamino)-4-hydroxy-2-(4-(trifluoromethyl)phenyl)butyl pivalate, was present as an impurity.

tert-Butyl (4S)-4-((1S)-2-(2,2-dimethylpropanoyl)oxy)-1-(4-(trifluoromethyl)phenyl)ethyl)-1,2,3-oxathiazolidine-3-carboxylate 2-oxide: SOCl₂ (0.63 mL, 8.7 mmol) was taken up in 30 mL of CAN, and the mixture was chilled to −55° C. (2S,3S)-3-(tert-Butoxycarbonyl)-4-hydroxy-2-(4-(trifluoromethyl)phenyl)butyl pivalate (1.5 g, 3.5 mmol) was then added slowly in 10 mL of ACN. After 15 minutes, pyridine (1.4 mL, 17 mmol, Aldrich) was added, and the mixture was warmed to room temperature. The mixture was concentrated under reduced pressure. The residue was taken up in 100 mL of EtOAc and 100 mL of water. The mixture was partitioned, and the aqueous portion was extracted with 100 mL of EtOAc. The combined organic extracts were washed with 100 mL of brine and dried over MgSO₄. Filtration and concentration under reduced pressure, followed by flash chromatography on silica gel (2.5% to 20% EtOAc/hexanes) afforded tert-butyl (4S)-4-((1S)-2-(2,2-dimethylpropanoyl)oxy)-1-(4-(trifluoromethyl)phenyl)ethyl)-1,2,3-oxathiazolidine-3-carboxylate 2-oxide (1.1 g, 66%) as a white solid. 10% of the enantiomer, tert-butyl (4R)-4-((1R)-2-(2,2-dimethylpropanoyl)oxy)-1-(4-(trifluoromethyl)phenyl)ethyl)-1,2,3-oxathiazolidine-3-carboxylate 2-oxide, was present as an impurity.

tert-Butyl (4S)-4-((1S)-2-(2,2-dimethylpropanoyl)oxy)-1-(4-(trifluoromethyl)phenyl)ethyl)-1,2,3-oxathiazolidine-3-carboxylate 2,2-dioxide. tert-Butyl (4S)-4-((1S)-2-((2,2-dimethylpropanoyl)oxy)-1-(4-(trifluoromethyl)phenyl)ethyl)-1,2,3-oxathiazolidine-3-carboxylate 2-oxide (1.1 g, 2.3 mmol) was taken up in 18 mL of ACN and 3 mL of EtOAc, and the mixture was chilled to 0° C. Sodium periodate (0.74 g, 3.4 mmol, Aldrich) was added in 6 mL of water, followed by ruthenium(III) chloride hydrate (0.0048 g, 0.023 mmol, Aldrich). The mixture was warmed to room temperature. After 1.5 hours, the solvent was removed under reduced pressure. The residue was taken in 20 mL of EtOAc and 20 mL of water. The aqueous portion was extracted twice with 20 mL of EtOAc, and the combined organic extracts were washed with 30 mL of brine. Filtration and concentration under reduced pressure afforded tert-butyl (4S)-4-((1S)-2-((2,2-dimethylpropanoyl)oxy)-1-(4-(trifluoromethyl)phenyl)ethyl)-1,2,3-oxathiazolidine-3-carboxylate 2,2-dioxide (1.1 g, 97%) as a white solid. ¹H NMR (400 MHz, CDCl₃) δ ppm 7.61 (d, J=8.03 Hz, 2H) 7.44 (d, J=8.03 Hz, 2H) 4.59-4.68 (m, 4H) 4.45 (dd, J=11.80, 5.27 Hz, 1H) 3.65 (d, J=5.52 Hz, 1H) 1.39 (s, 9H) 1.11-1.14 (m, 9H). 10% of the enantiomer, tert-butyl (4R)-4-((1R)-2-((2,2-dimethylpropanoyl)oxy)-1-(4-(trifluoromethyl)phenyl)ethyl)-1,2,3-oxathiazolidine-3-carboxylate 2,2-dioxide, was present as an impurity.

(2S,3S)-4-((5-Bromo-1,3-thiazol-2-yl)(tert-butoxycarbonyl)amino)-3-((tert-butoxycarbonyl)amino)-2-(4-(trifluoromethyl)phenyl)butyl 2,2-dimethylpropanoate. tert-Butyl 5-bromothiazol-2-ylcarbamate (0.59 g, 2.1 mmol, prepared as shown in Scheme 2) was taken up in 15 mL of DMF, and the mixture was heated to 50° C. Cs₂CO₃ (1.4 g, 4.2 mmol, Aldrich) was added, followed by slow addition of tert-butyl (4S)-4-((1S)-2-((2,2-dimethylpropanoyl)oxy)-1-(4-(trifluoromethyl)phenyl)ethyl)-1,2,3-oxathiazolidine-3-carboxylate 2,2-dioxide (1.1 g, 2.2 mmol) in 10 mL of DMF. After 1.5 hours, the solvent was removed under reduced pressure. The residue was taken up in 20 mL of EtOAc and 20 mL of 10% aqueous HCl was then slowly added. The mixture was stirred for 20 minutes. The mixture was partitioned, and the aqueous portion was extracted twice with 20 mL of EtOAc. The combined organic extracts were washed with 20 mL of brine and dried over MgSO₄. Filtration and concentration under reduced pressure, followed by flash chromatography on silica gel (2.5% to 25% EtOAc/hexanes) afforded (2S,3S)-4-((5-bromo-1,3-thiazol-2-yl)(tert-butoxycarbonyl)amino)-3-((tert-butoxycarbonyl)amino)-2-(4-(trifluoromethyl)phenyl)butyl 2,2-dimethylpropanoate (1.2 g, 82%) as a white solid. 10% of the enantiomer, (2R,3R)-4-((5-bromo-1,3-thiazol-2-yl)(tert-butoxycarbonyl)amino)-3-((tert-butoxycarbonyl)amino)-2-(4-(trifluoromethyl)phenyl)butyl 2,2-dimethylpropanoate, was present as an impurity.

tert-Butyl (5-bromo-1,3-thiazol-2-yl)((2S,3S)-2-((tert-butoxycarbonyl)amino)-4-hydroxy-3-(4-(trifluoromethyl)phenyl)butyl)carbamate. (2S,3S)-3-(Boc)Amino-4-(5-bromothiazol-2-yl(Boc)amino)-2-(4-(trifluoromethyl)phenyl)butyl pivalate (0.66 g, 0.95 mmol) was taken up in 10 mL of THF, and the mixture was chilled to −78° C. Super hydride (1M in THF (2.4 mL, 2.4 mmol, Aldrich)) was added. The mixture was stirred for 5 minutes and then warmed to 0° C. The mixture was stirred for 15 minutes and then quenched with 5 mL of EtOAc. The mixture was diluted with 10 mL of aqueous NH₄Cl and partitioned in a separatory funnel. The aqueous portion was extracted twice with 20 mL of EtOAc, and the combined organic layers were washed with 10 mL of brine and dried over MgSO₄. Filtration and concentration under reduced pressure, followed by flash chromatography on silica gel (5% to 25% EtOAc/hexanes) afforded tert-butyl (5-bromo-1,3-thiazol-2-yl)₄₋₂S,3S)-2-((tert-butoxycarbonyl)amino)-4-hydroxy-3-(4-(trifluoromethyl)phenyl)butyl)carbamate (0.51 g, 88%) as a white solid. ¹H NMR (400 MHz, CDCl₃) δ ppm 7.61 (d, J=8.02 Hz, 2H) 7.35 (d, J=8.02 Hz, 2H) 5.16-5.02 (m, 1H) 4.60-4.51 (m, 1H), 4.33-4.20 (m 1H) 4.09-3.96 (m, 1H), 3.91-3.65 (m, 2H) 3.15-3.05 (m, 1H) 1.52 (s, 9H) 1.37 (s, 9H). 10% of the enantomer, tert-butyl (5-bromo-1,3-thiazol-2-yl)₄₋₂R,3R)-2-((tert-butoxycarbonyl)amino)-4-hydroxy-3-(4-(trifluoromethyl)phenyl)butyl)carbamate, was present as an impurity.

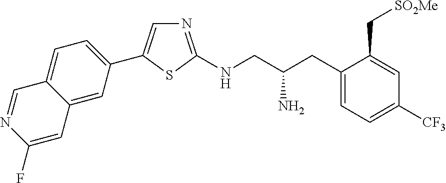

### Example 34

### N-((2S,3S)-2-Amino-4-(methylsulfonyl)-3-(4-(trifluoromethyl)phenyl)butyl)-5-(3-fluoroisoquinolin-6-yl)thiazol-2-amine

The title compound was synthesized in a manner similar to that described in Example 14 using tert-butyl (4S)-4-((1S)-2-(methylsulfonyl)-1-(4-(trifluoromethyl)phenyl)ethyl)-1,2,3-oxathiazolidine-3-carboxylate 2,2-dioxide instead of tert-butyl (4S)-4-(3,4-dichlorobenzyl)-1,2,3-oxathiazolidine-3-carboxylate 2,2-dioxide. MS m/z: 539 (M+1). ¹H NMR (400 MHz, CDCl₃) δ ppm 8.85 (s, 1H), 7.91 (d, J=8.6 Hz, 1H), 7.67 (d, J=8.2 Hz, 2H) 7.66-7.62 (m, 2H) 7.56 (s, 1H), 7.48 (d, J=8.2 Hz, 2H), 7.16 (s, 1H), 5.99 (broad s, 1H), 3.81 (dd, J=5.7 Hz, 13.9 Hz, 1H), 3.64-3.56 (m 2H), 3.51-3.45 (m, 2H), 3.09 (dd, J=7.6 Hz, 13.1 Hz, 1H), 2.72 (s, 3H). 10% of the enantiomer, ((2R,3R)-2-amino-4-(methylsulfonyl)-3-(4-(trifluoromethyl)phenyl)butyl)-5-(3-fluoroisoquinolin-6-yl)thiazol-2-amine, was present as an impurity. tert-Butyl (4S)-4-((1S)-2-(methylsulfonyl)-1-(4-(trifluoromethyl)phenyl)ethyl)-1,2,3-oxathiazolidine-3-carboxylate 2,2-dioxide was prepared as shown in Scheme 15.

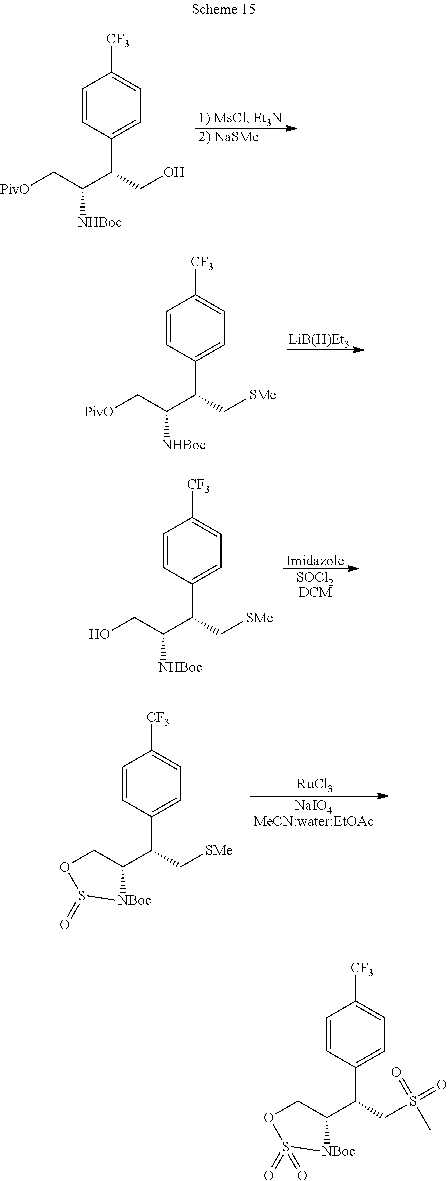

(2 S,3S)-2-(tert-Butoxycarbonyl)-4-(methylthio)-3-(4-(trifluoromethyl)phenyl)butyl pivalate: (2S,3S)-2-(tert-Butoxycarbonyl)-4-hydroxy-3-(4-(trifluoromethyl)phenyl)butyl pivalate (5.0 g, 12 mmol, prepared as shown in Scheme 13) was taken up in 100 mL of DCM, and the mixture was chilled to 0° C. TEA (2.4 mL, 17 mmol) was added, followed by methanesulfonyl chloride (0.99 mL, 13 mmol, Aldrich). After 1 hour, the reaction was quenched with 75 mL of aqueous NaHCO₃ and diluted with 70 mL of water. The mixture was partitioned and the aqueous portion was extracted with 70 mL of DCM. The combined organic extracts were dried over MgSO₄. Filtration and concentration under reduced pressure afforded an initial product that was taken up in 50 mL of DMSO. Sodium methanethiolate (4.9 g, 69 mmol, Aldrich) was added to the mixture. A significant exotherm was observed. The mixture was stirred for 12 hours. The mixture was diluted with 100 mL of EtOAc and 100 mL of water and transferred to a separatory funnel. The mixture was further diluted with 300 mL of EtOAc and partitioned. The organic portion was washed twice with 150 mL of water and once with 150 mL of brine, then dried over MgSO₄. Filtration and concentration under reduced pressure, followed by flash chromatography on silica gel (3% to 10% EtOAc/hexanes) afforded (2S,3S)-2-((tert-butoxycarbonyl)amino)-4-(methylsulfanyl)-3-(4-(trifluoromethyl)phenyl)butyl 2,2-dimethylpropanoate (4.2 g, 79%) as a sticky oil. ¹H NMR (400 MHz, CDCl₃) δ ppm 7.61 (d, J=8.2 Hz, 2H), 7.31 (d, J=8.2 Hz, 2H), 4.43-4.33 (m, 1H), 4.28 (d, J=10.0 Hz, 1H), 4.04-3.94 (m, 2H), 3.17-3.10 (m, 1H), 2.96 (dd, J=6.2 Hz, 13.1 Hz, 1H), 2.90 (dd, J=12.9 Hz, 21.9 Hz, 1H), 2.05 (s, 3H), 1.38 (s, 9H), 1.20 (s, 9H). 10% of the enantiomer, (2R,3R)-2-((tert-butoxycarbonyl)amino)-4-(methylsulfanyl)-3-(4-(trifluoromethyl)phenyl)butyl 2,2-dimethylpropanoate, was present as an impurity.

tert-Butyl ((1S,2S)-1-(hydroxymethyl)-3-(methyl sulfanyl)-2-(4-(trifluoromethyl)phenyl)propyl)carbamate. (2 S,3S)-2-((tert-Butoxycarbonyl)amino)-4-(methylsulfanyl)-3-(4-(trifluoromethyl)phenyl)butyl 2,2-dimethylpropanoate (4.2 g, 9.1 mmol) was taken up in 75 mL of THF, and the mixture was chilled to −78° C. Superhydride (1.0 M in THF (23 mL, 23 mmol, Aldrich)) was added slowly to the mixture. After 5 minutes, the mixture was warmed to 0° C. The mixture was stirred for 30 minutes and was then quenched with 50 mL of aqueous NH₄Cl. The mixture was then diluted with 50 mL of water and extracted twice with 200 mL of EtOAc. The combined organic extracts were dried over MgSO₄. Filtration and concentration under reduced pressure, followed by flash chromatography on silica gel (10% to 50% EtOAc/hexanes) afforded tert-butyl ((1S,2S)-1-(hydroxymethyl)-3-(methylsulfanyl)-2-(4-(trifluoromethyl)phenyl)propyl)carbamate (3.0 g, 87%) as a clear oil. ¹H NMR (400 MHz, CDCl₃) δ ppm 7.60 (d, J=8.2 Hz, 2H), 7.35 (d, J=8.0 Hz, 2H), 4.46 (d, J=7.1 Hz, 1H), 4.17 (s, 1H), 4.08-4.01 (m, 1H), 3.73-3.57 (m, 2H), 3.35-3.28 (m, 1H), 2.98 (dd, J=6.3 Hz, 13.1 Hz, 1H), 2.88 (dd, J=8.4 Hz, 13.1 Hz, 1H), 2.06 (s, 3H), 1.35 (s, 9H). 10% of the enantiomer, tert-butyl ((1R,2R)-1-(hydroxymethyl)-3-(methylsulfanyl)-2-(4-(trifluoromethyl)phenyl)propyl)carbamate.

tert-Butyl (4S)-4-((1S)-2-(methylsulfanyl)-1-(4-(trifluoromethyl)phenyl)ethyl)-1,2,3-oxathiazolidine-3-carboxylate 2-oxide. Imidazole (5.0 g, 74 mmol, Aldrich) was taken up in 50 mL of DCM, and the mixture was chilled to 0° C. SOCl₂ (1.6 mL, 22 mmol, Aldrich) was added. The mixture was warmed to room temperature and stirred for 1 hour. The mixture was then chilled to −78° C. tert-Butyl ((1S,2S)-1-(hydroxymethyl)-3-(methylsulfanyl)-2-(4-(trifluoromethyl)phenyl)propyl)carbamate (3.0 g, 7.9 mmol) was added slowly in 50 mL of DCM via addition funnel. The mixture was gradually warmed to room temperature. After 12 hours, the reaction was quenched with 100 mL of water and extracted twice with 100 mL of DCM. The combined organic extracts were dried over MgSO₄. Filtration and concentration under reduced pressure afforded tert-butyl (4S)-4-((1S)-2-(methylsulfanyl)-1-(4-(trifluoromethyl)phenyl)ethyl)-1,2,3-oxathiazolidine-3-carboxylate 2-oxide (3.2 g, 95%) as a yellow solid. The product was used without any further purification. 10% of the enantiomer, tert-butyl (4R)-4-((1R)-2-(methylsulfanyl)-1-(4-(trifluoromethyl)phenyl)ethyl)-1,2,3-oxathiazolidine-3-carboxylate 2-oxide, was present as an impurity.

tert-Butyl (4S)-4-((1S)-2-(methylsulfonyl)-1-(4-(trifluoromethyl)phenyl)ethyl)-1,2,3-oxathiazolidine-3-carboxylate 2,2-dioxide. tert-Butyl (4S)-4-((1S)-2-(methylsulfanyl)-1-(4-(trifluoromethyl)phenyl)ethyl)-1,2,3-oxathiazolidine-3-carboxylate 2-oxide (0.15 g, 0.35 mmol) was taken up in 3 mL of ACN and 0.5 mL of EtOAc, and the mixture was then chilled to 0° C. Sodium periodate (0.34 g, 1.6 mmol, Aldrich) was added in 1 mL of water, followed by ruthenium(III) chloride hydrate (0.79 mg, 3.5 umol, Aldrich). The mixture was warmed to room temperature and stirred for 30 minutes. The solvent was removed under reduced pressure, and the residue was taken up in 20 mL of EtOAc. The mixture was washed with 10 mL of water and 10 mL of brine, and then dried over MgSO₄. Filtration and concentration under reduced pressure afforded tert-butyl (4S)-4-((1S)-2-(methylsulfonyl)-1-(4-(trifluoromethyl)phenyl)ethyl)-1,2,3-oxathiazolidine-3-carboxylate 2,2-dioxide (0.15 g, 90%) as a white solid. ¹H NMR (400 MHz, CDCl₃) δ ppm 7.68 (d, J=8.2 Hz, 2H), 7.48 (d, J=8.2 Hz, 2H), 4.64-4.51 (m, 3H), 4.06-4.01 (m, 1H), 3.68 (dd, J=8.8 Hz, 14.3 Hz, 1H), 3.62 (dd, J=4.1 Hz, 14.3 Hz, 1H), 2.76 (s, 3H), 1.50 (s, 9H). 10% of the enantiomer, tert-butyl (4R)-4-((1R)-2-(methylsulfonyl)-1-(4-(trifluoromethyl)phenyl)ethyl)-1,2,3-oxathiazolidine-3-carboxylate 2,2-dioxide, was present as an impurity.

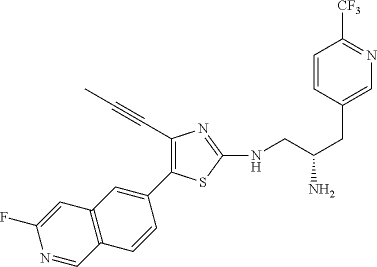

### Example 35

### N—((S)-2-Amino-3-(6-(trifluoromethyl)pyridin-3-yl)propyl)-5-(3-fluoroisoquinolin-6-yl)-4-(prop-1-ynyl)thiazol-2-amine

The title compound was synthesized in a manner similar to that described in Example 2 using tert-butyl (5-bromo-4-(1-propyn-1-yl)-1,3-thiazol-2-yl)((2S)-2-((tert-butoxycarbonyl)amino)-3-(6-(trifluoromethyl)-3-pyridinyl)propyl)carbamate instead of tert-butyl (5-bromo-1,3-thiazol-2-yl)((2S)-2-((tert-butoxycarbonyl)amino)-3-(6-(trifluoromethyl)-3-pyridinyl)propyl)carbamate. MS m/z: 486 (M+1). ¹H NMR (400 MHz, CDCl₃) δ ppm 8.90 (s, 1H), 8.63 (s, 1H), 8.07 (s, 1H), 8.02 (d, A of ABq, 8.8 Hz, 1H), 7.95 (d, B of Abq, 8.8 Hz, 1H), 7.76 (d, J=9.0 Hz, 1H), 7.67 (d, J=8.0 Hz, 1H), 7.20 (s, 1H), 5.89 (broad s, 1H), 3.61-3.55 (m, 1H), 3.42-3.35 (m, 1H), 3.31-3.25 (m, 1H), 3.00 (dd, J=4.9 Hz, 13.7 Hz, 1H), 2.71 (dd, J=8.4 Hz, 13.6 Hz, 1H), 2.15 (s, 3H). tert-Butyl (5-bromo-4-(1-propyn-1-yl)-1,3-thiazol-2-yl)((2S)-2-((tert-butoxycarbonyl)amino)-3-(6-(trifluoromethyl)-3-pyridinyl)propyl)carbamate was prepared as shown in Scheme 16.

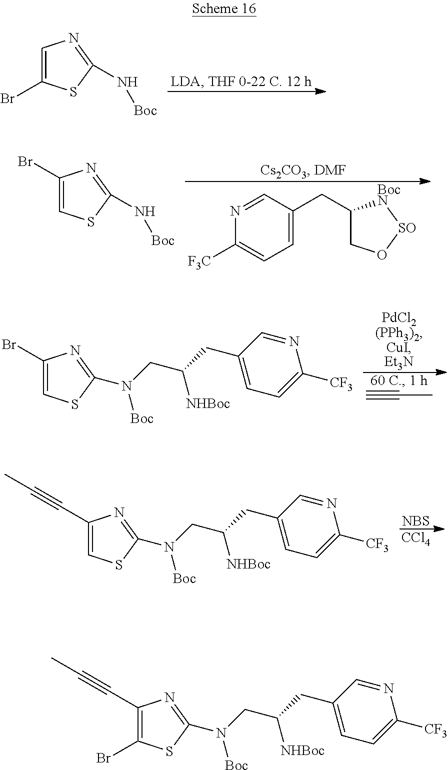

t-Butyl 4-bromothiazol-2-ylcarbamate: Diisopropylamine (2.3 mL, 16 mmol, Aldrich) was taken up in 30 mL of THF, and the mixture was chilled to 0° C. Butyllithium (2.5 M in hexane (6.4 mL, 16 mmol, Aldrich)) was added to the reaction mixture, and the mixture was stirred for 20 minutes. tert-Butyl 5-bromothiazol-2-ylcarbamate (1.5 g, 5.4 mmol, prepared as shown in Scheme 2) was then added slowly in 8 mL of THF. After 15 minutes, approximately 2 mL of water was added, and the mixture was warmed to room temperature and stirred for 12 hours. The mixture was diluted with 30 mL of ½ saturated aqueous NH₄Cl and transferred to a separatory funnel. The mixture was extracted twice with 5 mL of EtOAc, and the combined organic extracts were washed with brine and dried over MgSO₄. Filtration and concentration under reduced pressure afforded tert-butyl 4-bromothiazol-2-ylcarbamate (1.5 g, 100%) as a brown solid.

tert-Butyl 4-bromothiazol-2-ylcarbamate (1.8 g, 6.4 mmol) was taken up in 30 mL of DMF. Cesium carbonate (4.2 g, 13 mmol, Aldrich) was added, and the mixture was heated to 50° C. tert-Butyl (4S)-4-(6-(trifluoromethyl)-3-pyridinyl)methyl)-1,2,3-oxathiazolidine-3-carboxylate 2-oxide (2.5 g, 6.8 mmol) was added slowly in 5 mL of DMF. The mixture was stirred for 12 hours and was then concentrated under reduced pressure. The residue was taken up in 250 mL of EtOAc and washed 2× with 150 mL of water and once with 150 mL of brine, and then dried over MgSO₄. Filtration and concentration under reduced pressure, followed by flash chromatography on silica gel (5% to 25% EtOAc/hexanes) afforded tert-butyl (4-bromo-1,3-thiazol-2-yl)((2S)-2-((tert-butoxycarbonyl)amino)-3-(6-(trifluoromethyl)-3-pyridinyl)propyl)carbamate (2.5 g, 67%) as a white solid. 10% of the enantiomer, tert-butyl (4-bromo-1,3-thiazol-2-yl)((2R)-2-((tert-butoxycarbonyl)amino)-3-(6-(trifluoromethyl)-3-pyridinyl)propyl)carbamate was present as an impurity.

tert-Butyl ((2S)-2-((tert-butoxycarbonyl)amino)-3-(6-(trifluoromethyl)-3-pyridinyl)propyl)(4-(1-propyn-1-yl)-1,3-thiazol-2-yl)carbamate. tert-Butyl (4-bromo-1,3-thiazol-2-yl)((2S)-2-((tert-butoxycarbonyl)amino)-3-(6-(trifluoromethyl)-3-pyridinyl)propyl)carbamate (0.75 g, 1.3 mmol), copper iodide (0.074 g, 0.39 mmol, Aldrich), and bis(triphenylphosphino)palladium(II) dichloride (0.091 g, 0.13 mmol, Aldrich) were taken up in 12 mL of TEA (12 mL, 1.3 mmol) in a sealable tube. Propyne (Aldrich) was bubbled through the mixture for 2 minutes. The tube was sealed, heated to 60° C., and the mixture was stirred overnight. The solvent was removed under reduced pressure and the residue was purified by flash chromatography on silica gel (5% to 20% EtOAc/hexanes) affording tert-butyl ((2S)-2-((tert-butoxycarbonyl)amino)-3-(6-(trifluoromethyl)-3-pyridinyl)propyl)(4-(1-propyn-1-yl)-1,3-thiazol-2-yl)carbamate (0.66 g, 95%) as a yellow solid. ¹H NMR (400 MHz, CDCl₃) δ ppm 8.59 (s, 1H), 7.85 (d, J=8.0 Hz, 1H), 7.64 (d, J=8.1 Hz, 1H), 6.97 (s, 1H), 5.33 (broad s, 1H), 4.37-4.23 (m, 2H), 4.06 (d, J=10.1 Hz, 1H), 3.06-3.01 (m, 1H), 2.95-2.84 (m, 1H), 2.07 (m, 3H), 1.49 (s, 9H), 1.31 (s, 9H). 10% of the enantiomer, affording tert-butyl ((2R)-2-((tert-butoxycarbonyl)amino)-3-(6-(trifluoromethyl)-3-pyridinyl)propyl)(4-(1-propyn-1-yl)-1,3-thiazol-2-yl)carbamate, was present as an impurity.

tert-Butyl (5-bromo-4-(1-propyn-1-yl)-1,3-thiazol-2-yl)((2S)-2-((tert-butoxycarbonyl)amino)-3-(6-(trifluoromethyl)-3-pyridinyl)propyl)carbamate. tert-Butyl ((2S)-2-((tert-butoxycarbonyl)amino)-3-(6-(trifluoromethyl)-3-pyridinyl)propyl)(4-(1-propyn-1-yl)-1,3-thiazol-2-yl)carbamate (0.64 g, 1.2 mmol) was taken up in 12 mL of CCL₄. 1-Bromopyrrolidine-2,5-dione (0.53 g, 3.0 mmol, Aldrich) was added to the mixture. After 2.5 hours, the mixture was quenched with 15 mL of aqueous Na₂S₂O₃ and diluted with 15 mL of water. The mixture was partitioned, and the aqueous portion was extracted with 20 mL of DCM. The combined organic extracts were dried over MgSO₄. Filtration and concentration under reduced pressure, followed by flash chromatography on silica gel (5% to 25% EtOAc/hexanes) afforded tert-butyl (5-bromo-4-(1-propyn-1-yl)-1,3-thiazol-2-yl)((2S)-2-((tert-butoxycarbonyl)amino)-3-(6-(trifluoromethyl)-3-pyridinyl)propyl)carbamate (0.60 g, 82%) as a white solid. ¹H NMR (400 MHz, CDCl₃) δ ppm 8.58 (s, 1H), 7.82 (d, J=8.0 Hz, 1H), 7.63 (d, J=8.0 Hz, 1H), 5.11 (d, J=7.2 Hz, 1H), 4.34-4.15 (m, 2H), 3.98 (d, J=13.1 Hz, 1H), 3.04-2.96 (m, 1H), 2.90-2.85 (m 1H), 2.12 (s, 3H), 1.48 (s, 9H), 1.31 (s, 9H). 10% of the enantiomer, tert-butyl (5-bromo-4-(1-propyn-1-yl)-1,3-thiazol-2-yl)((2R)-2-((tert-butoxycarbonyl)amino)-3-(6-(trifluoromethyl)-3-pyridinyl)propyl)carbamate, was present as an impurity.

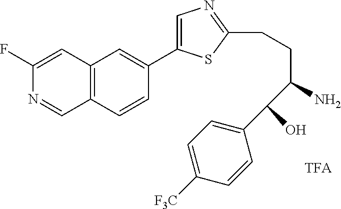

### Example 36

### (1S,2R)-2-Amino-4-(5-(3-fluoroisoquinolin-6-yl)thiazol-2-yl)-1-(4-(trifluoromethyl)phenyl)butan-1-ol trifluoroacetate

This compound was synthesized according to Scheme 17.

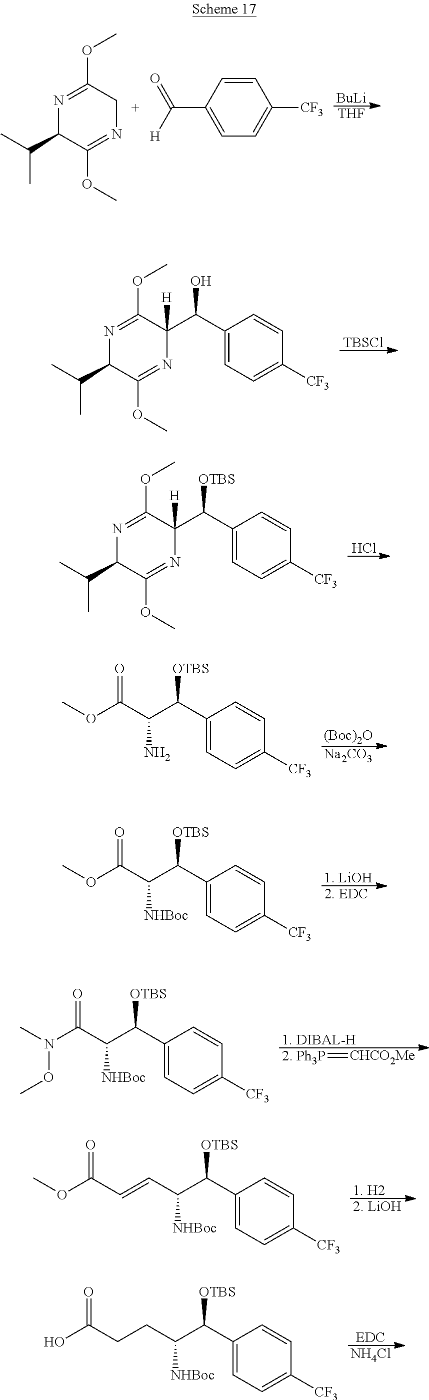

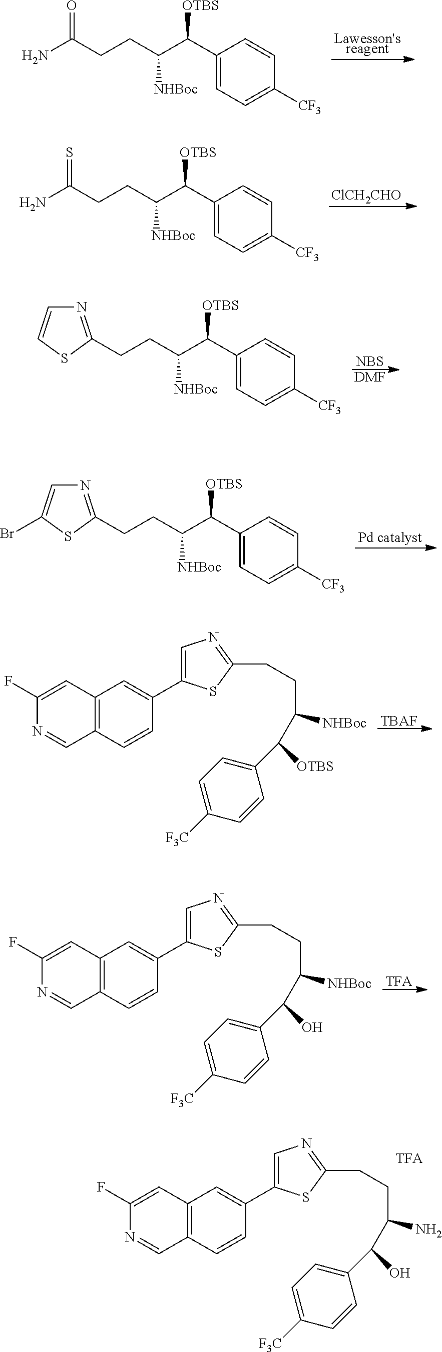

(S)-((2 S,5R)-5-Isopropyl-3,6-dimethoxy-2,5-dihydropyrazin-2-yl)(4-(trifluoromethyl)phenyl)methanol: To a mixture of (R)-2,5-dihydro-3,6-dimethoxy-2-isopropylpyrazine (8.0 mL, 45 mmol, Fluka catalog number 37286-5 mL) and THF (60 mL) at −78° C. was added n-butyllithium (2.5 M solution in hexane, 19 mL, 47 mmol). The mixture was stirred for 15 minutes. The colorless solution turned light brown. Then a solution of 4-(trifluoromethyl)benzaldehyde (Aldrich, 7.2 mL, 54 mmol) in THF (60 mL) was added dropwise through a dropping funnel at −78° C. The mixture was stirred for 1 hour after addition was complete. The reaction mixture was diluted with EtOAc and washed with a mixed solution of aqueous Na₂HPO₄ and KH₂PO₄ solution (pH˜8). The aqueous layer was extracted with EtOAc three times. The combined organic layers were washed with water and brine, dried over Na₂SO₄, filtered, and concentrated in vacuo. The initial product was separated into two isomers by silica gel chromatography (0-2%-10% ACN-DCM). The desired product was obtained as an off-white solid (6.40 g, 40%). LCMS (API-ES) m/z (%): 359 (M⁺+H).

(2S,5R)-2-(S)-(tert-Butyldimethylsilyloxy)(4-(trifluoromethyl)phenyl)methyl)-5-isopropyl-3,6-dimethoxy-2,5-dihydropyrazine: To a solution of (S)-((2S,5R)-5-isopropyl-3,6-dimethoxy-2,5-dihydropyrazin-2-yl)(4-(trifluoromethyl)phenyl)methanol (5.00 g, 14.0 mmol) in DCM (70 mL) was added imidazole (2.85 g, 41.9 mmol, Aldrich) and tert-butylchlorodimethylsilane (3.15 g, 20.9 mmol, Aldrich) at room temperature. The mixture became a suspension. TEA (2.33 mL, 16.7 mmol) was added and the suspension became a clear solution. The mixture was stirred 3 days and was then diluted with DCM and washed with water, saturated NaHCO₃ and brine. The organic layer was dried over Na₂SO₄, filtered, and concentrated in vacuo. The initially obtained product was purified by silica gel chromatography (12%-80% DCM-hexane). The product was obtained as sticky oil (4.97 g, 75%). LCMS (API-ES) m/z (%): 473 (M⁺+H).

(2S,3S)-Methyl 2-amino-3-(tert-butyldimethylsilyloxy)-3-(4-(trifluoromethyl)phenyl)propanoate: To a solution of (2S,5R)-2-(S)-(tert-butyldimethylsilyloxy)(4-(trifluoromethyl)phenyl)methyl)-5-isopropyl-3,6-dimethoxy-2,5-dihydropyrazine (4.97 g, 11 mmol) in THF (24 mL) and CAN (48 mL) was added hydrochloric acid (28 mL, 28 mmol) at 0° C. The mixture was gradually warmed to room temperature. After stirring 16 hours, the mixture was concentrated in vacuo. The residue was neutralized with saturated NaHCO₃ and extracted with EtOAc three times. The organic layer was dried over Na₂SO₄, filtered, and concentrated in vacuo. The initially obtained product was purified by silica gel chromatography (0-25%-50% EtOAc-hexane). The product was obtained as colorless oil (3.29 g, 83%). LCMS (API-ES) m/z (%): 378 (M⁺+H).

(2S,3S)-Methyl 2-(tert-butoxycarbonyl)-3-(tert-butyldimethylsilyloxy)-3-(4-(trifluoromethyl)phenyl)propanoate: To a solution of (2S,3S)-methyl 2-amino-3-(tert-butyldimethylsilyloxy)-3-(4-(trifluoromethyl)phenyl)propanoate (3.29 g, 8.72 mmol) in THF (20 mL) was added di-tert-butyl dicarbonate (2.28 g, 10.5 mmol, Aldrich) and sodium carbonate monohydrate (2.16 g, 17.4 mmol). The mixture was stirred at room temperature overnight, and then it was filtered through a funnel. The filtrate was concentrated in vacuo and the residue was purified by silica gel chromatography: 0-10% EtOAc-hexane. The product was obtained as colorless oil (4.04 g, 102%). LCMS (API-ES) m/z (%): 378 (M⁺+H-100).

(2S,3S)-2-(tert-Butoxycarbonyl)-3-(tert-butyldimethylsilyloxy)-3-(4-(trifluoromethyl)phenyl)propanoic acid: To a solution of (2S,3S)-methyl 2-(tert-butoxycarbonylamino)-3-(tert-butyldimethylsilyloxy)-3-(4-(trifluoromethyl)phenyl)propanoate (3.50 g, 7.33 mmol) in THF (24 mL), and MeOH (8 mL) was added lithium hydroxide monohydrate (0.63 g, 14.84 mmol) and water (8 mL) at room temperature. The mixture was stirred 16 hours. The mixture was then concentrated in vacuo. Ether was added and evaporated to remove water. The white solid was used in next step without further purification (3.40 g, 100%). LCMS (API-ES) m/z (%): 364 (M⁺+H-100).

tert-Butyl (2S,3S)-3-(tert-butyldimethylsilyloxy)-1-(methoxy(methyl)amino)-1-oxo-3-(4-(trifluoromethyl)phenyl)propan-2-ylcarbamate: To a mixture of (2S,3S)-2-(tert-butoxycarbonylamino)-3-(tert-butyldimethylsilyloxy)-3-(4-(trifluoromethyl)phenyl)propanoic acid (3.40 g, 7.33 mmol), DMF (20 mL), 1-(3-dimethylaminopropyl)-3-ethylcarbodiimide hydrochloride (2.11 g, 11.0 mmol, Aldrich), N,O-dimethylhydroxylamine hydrochloride (2.15 g, 22.0 mmol, Aldrich), and N-hydroxybenzotriazole (0.22 g, 1.47 mmol, AnaSpec Inc.) was added N,N-diisopropylethylamine (7.03 mL, 40.3 mmol) at room temperature. The mixture was stirred 16 hours. HPLC-MS showed 73% conversion. 1-(3-Dimethylaminopropyl)-3-ethylcarbodiimide hydrochloride (0.53 g), N, 0-dimethylhydroxylamine hydrochloride (0.54 g) and 1.7 mL of N,N-diisopropylethylamine were then added, and the reaction was continued. The mixture was diluted with EtOAc, washed with water, saturated NaHCO₃, and brine. The organic layer was concentrated in vacuo. The residue was purified by silica gel chromatography: 6-50% EtOAc-hexane. The product was obtained as a white solid (2.29 g, 62%). LCMS (API-ES) m/z (%): 407 (M⁺+H-100).

tert-Butyl (1S,2S)-1-(tert-butyldimethylsilyloxy)-3-oxo-1-(4-(trifluoromethyl)phenyl)propan-2-ylcarbamate: To a solution of tert-butyl (2S,3S)-3-(tert-butyldimethylsilyloxy)-1-(methoxy(methyl)amino)-1-oxo-3-(4-(trifluoromethyl)phenyl)propan-2-ylcarbamate (2.29 g, 4.5 mmol) in THF (40 mL) was added diisobutylaluminum hydride solution in hexane (23 mL, 23 mmol, Aldrich) at −78° C. The mixture was stirred for 1 hour. TLC in 25% EtOAc-hexane showed 100% conversion. The mixture was quenched with saturated NH₄Cl and extracted with EtOAc (extra water was added to dissolve the solid). The organic layer was dried over Na₂SO₄ and filtered through a short column of silica gel. The filtrate was concentrated in vacuo. The product, a sticky solid, was used directly in the next step (1.86 g, 92%).

(4R,5S,E)-Methyl 4-(tert-butoxycarbonylamino)-5-(tert-butyldimethylsilyloxy)-5-(4-(trifluoromethyl)phenyl)pent-2-enoate: To a solution of tert-butyl (1S,2S)-1-(tert-butyldimethylsilyloxy)-3-oxo-1-(4-(trifluoromethyl)phenyl)propan-2-ylcarbamate (1.86 g, 4.16 mmol) in THF (40 mL) was added methyl (triphenylphosphoranylidene)acetate (1.67 g, 4.99 mmol, Aldrich). The mixture was heated at reflux for 1 hour, and then concentrated in vacuo. The residue was dissolved in DCM and purified by gel filtration (12% EtOAc-hexane). The product was obtained as a white solid (1.70 g, 81%). LCMS (API-ES) m/z (%): 404 (M⁺+H-100).

(4R,5S)-Methyl 4-(tert-butoxycarbonylamino)-5-(tert-butyldimethylsilyloxy)-5-(4-(trifluoromethyl)phenyl)pentanoate: To a solution of (4R,5S,E)-methyl 4-(tert-butoxycarbonylamino)-5-(tert-butyldimethylsilyloxy)-5-(4-(trifluoromethyl)phenyl)pent-2-enoate (1.70 g, 3.38 mmol) in MeOH (40 mL) was added palladium, 10 wt. % on activated carbon (0.18 mg, 0.17 mmol, Aldrich). The mixture was purged with hydrogen and stirred for 1 hour. HPLC-MS showed only the product. The mixture was filtered through Celite® brand filter aid and the cake was washed with EtOAc. The filtrate was concentrated in vacuo and the product was obtained as colorless oil (1.71 g, 100%). LCMS (API-ES) m/z (%): 406 (M⁺+H-100).

(4R,5S)-4-(tert-Butoxycarbonyl)-5-(tert-butyldimethylsilyloxy)-5-(4-(trifluoromethyl)phenyl)pentanoic acid: To a solution of (4R,5S)-methyl 4-(tert-butoxycarbonylamino)-5-(tert-butyldimethylsilyloxy)-5-(4-(trifluoromethyl)phenyl)pentanoate (1.70 g, 3.36 mmol) in THF (6 mL), and MeOH (2 mL) was added lithium hydroxide, monohydrate (0.28 g, 6.72 mmol) and water (2 mL) at room temperature. The mixture was stirred 16 hours, and then concentrated in vacuo. Ether was added and evaporated to remove water. The white solid (1.66 g, 100%) was used in the next step without further purification. LCMS (API-ES) m/z (%): 392 (M⁺+H-100).

tert-Butyl (1S,2R)-5-amino-1-(tert-butyldimethylsilyloxy)-5-oxo-1-(4-(trifluoromethyl)phenyl)pentan-2-ylcarbamate: To a mixture of (4R,5S)-4-(tert-butoxycarbonylamino)-5-(tert-butyldimethylsilyloxy)-5-(4-(trifluoromethyl)phenyl)pentanoic acid (1.65 g, 3.4 mmol), DCM (17 mL), 1-(3-dimethylaminopropyl)-3-ethylcarbodiimide hydrochloride (1.3 g, 6.7 mmol, Aldrich), and N-hydroxybenzotriazole (0.26 g, 1.7 mmol, AnaSpec Inc.) was added ammonium chloride (0.27 g, 5.0 mmol), 4-methylmorpholine (1.8 mL, 17 mmol) and DMF (10 mL) at room temperature. The mixture was stirred overnight, diluted with water and extracted with EtOAc. The organic layer was washed with water and brine, dried over Na₂SO₄, filtered, and concentrated in vacuo. The residue was purified by silica gel chromatography (12-100% EtOAc-hexane). The product was obtained as a white solid (1.40 g, 85%). LCMS (API-ES) m/z (%): 491 (M⁺+H).

tert-Butyl (1S,2R)-5-amino-1-(tert-butyldimethylsilyloxy)-5-thioxo-1-(4-(trifluoromethyl)phenyl)pentan-2-ylcarbamate: A mixture of tert-butyl (1S,2R)-5-amino-1-(tert-butyldimethylsilyloxy)-5-oxo-1-(4-(trifluoromethyl)phenyl)pentan-2-ylcarbamate (1.28 g, 2.61 mmol), DCM (45 mL), and Lawesson's reagent (0.63 g, 1.57 mmol, Aldrich) was stirred at room temperature for 2 hours under N₂. The mixture was concentrated in vacuo. The residue was purified by silica gel chromatography (6%-50% EtOAc-hexane). The product was obtained as a white solid (1.23 g, 93%). LCMS (API-ES) m/z (%): 407 (M⁺+H-100).

tert-Butyl (1S,2R)-1-(tert-butyldimethylsilyloxy)-4-(thiazol-2-yl)-1-(4-(trifluoromethyl)phenyl)butan-2-ylcarbamate: A mixture of tert-butyl (1S,2R)-5-amino-1-(tert-butyldimethylsilyloxy)-5-thioxo-1-(4-(trifluoromethyl)phenyl)pentan-2-ylcarbamate (1.23 g, 2.4 mmol), chloroacetaldehyde (−50 wt. % solution in water (2.4 mL, Aldrich)) and toluene (80 mL) was heated at reflux for 1.5 hours. HPLC showed that the starting material was consumed. The mixture was diluted with saturated NH₄Cl and extracted with EtOAc. The organic phase was dried over Na₂SO₄, filtered, and concentrated in vacuo. The residue was purified by silica gel chromatography: 6%-50% EtOAc-hexane. The product was obtained as an off-white solid (0.89 g, 69%). (API-ES) m/z (%): 531 (M⁺+H).

tert-Butyl (1S,2R)-4-(5-bromothiazol-2-yl)-1-(tert-butyldimethylsilyloxy)-1-(4-(trifluoromethyl)phenyl)butan-2-ylcarbamate: To a mixture of tert-butyl (1S,2R)-1-(tert-butyldimethylsilyloxy)-4-(thiazol-2-yl)-1-(4-(trifluoromethyl)phenyl)butan-2-ylcarbamate (0.89 g, 1.7 mmol) and DMF (8 mL) was added NBS (0.72 g, 4.0 mmol) at room temperature. The mixture was stirred for 2 hours. HPLC-MS showed that the reaction was incomplete. NBS (0.26 g) was added. After two more hours, the reaction was quenched with 1M Na₂SO₃ (4 mL) and the mixture was extracted with EtOAc three times. The organic phase was washed with water and brine, dried over Na₂SO₄, filtered, and concentrated in vacuo. The residue was purified by silica gel chromatography: 6%-50% EtOAc-hexane. The product was obtained as a white solid (0.72 g, 71%). LCMS (API-ES) m/z (%):609, 611 (M⁺+H).

tert-Butyl (1S,2R)-1-(tert-butyldimethylsilyloxy)-4-(5-(3-fluoroisoquinolin-6-yl)thiazol-2-yl)-1-(4-(trifluoromethyl)phenyl)butan-2-ylcarbamate: A mixture of potassium acetate (113 mg, 1.15 mmol), 3-fluoroisoquinolin-6-ylboronic acid (47 mg, 0.25 mmol), tert-butyl (1S,2R)-4-(5-bromothiazol-2-yl)-1-(tert-butyldimethylsilyloxy)-1-(4-(trifluoromethyl)phenyl)butan-2-ylcarbamate (100 mg, 0.16 mmol) in ACN (2.1 mL) and water (0.7 mL) was purged with nitrogen and then bis(di-t-butylphenylphosphine)dichloropalladium catalyst (9.2 mg, 0.015 mmol, for preparation see Guram, S., Organic Letters, 2006, 8(9), 1787-1789) was added. The mixture was then heated at 90° C. for 1 hour in a microwave reactor. The bright yellow solution turned dark yellow. The mixture was diluted with EtOAc (50 mL) and washed with saturated NaHCO₃ (twice) and brine. The organic phase was dried over Na₂SO₄, filtered, and concentrated in vacuo. The residue was purified by silica gel chromatography: 10%-25%-50% EtOAc-hexane. The product was obtained as an off-white solid (70 mg, 63%). LCMS (API-ES) m/z (%): 676 (M⁺+H).

tert-Butyl (1S,2R)-4-(5-(3-fluoroisoquinolin-6-yl)thiazol-2-yl)-1-hydroxy-1-(4-(trifluoromethyl)phenyl)butan-2-ylcarbamate: To a mixture of tert-butyl (1S,2R)-1-(tert-butyldimethylsilyloxy)-4-(5-(3-fluoroisoquinolin-6-yl)thiazol-2-yl)-1-(4-(trifluoromethyl)phenyl)butan-2-ylcarbamate (70 mg, 0.10 mmol) in THF (2 mL) was added tetrabutylammonium fluoride (0.21 mL of 1.0M solution in THF, 0.21 mmol, Fluka) at 0° C. After 1 hour, the mixture was diluted with water and extracted with DCM. The organic phase was dried over Na₂SO₄, filtered, and concentrated in vacuo. The residue was purified by silica gel chromatography: 50% EtOAc-hexane. The product was obtained as a white solid, which showed an extra peak in HPLC at 215 nM (82 mg, 142%). LCMS (API-ES) m/z (%): 562 (M⁺+H).

(1S,2R)-2-Amino-4-(5-(3-fluoro-6-isoquinolinyl)-1,3-thiazol-2-yl)-1-(4-(trifluoromethyl)phenyl)-1-butanol trifluoroacetate: To a mixture of tert-butyl (1S,2R)-4-(5-(3-fluoroisoquinolin-6-yl)thiazol-2-yl)-1-hydroxy-1-(4-(trifluoromethyl)phenyl)butan-2-ylcarbamate (58 mg, 0.0527 mmol) in CH₂Cl₂ (1 mL) was added TFA (1 mL) at room temperature. After 30 minutes, the mixture was concentrated in vacuo. The residue was purified by reverse phase preparatory HPLC (Phenomenex Luna C18.5 μM 150×30 mm, flow rate 30 mL/minute, 10%-100% ACN (with 0.1% TFA)-water (with 0.1% TFA) in 15 minutes). The TFA salt of the product was obtained as a white solid (41 mg, 68%). LCMS (API-ES) m/z (%): 462 (M⁺+H). ¹H NMR (400 MHz, CD₃OD) δ ppm 1.97-2.09 (m, 2H) 2.93-3.20 (m, 2H) 3.69 (td, J=6.16, 3.33 Hz, 1H) 5.14 (d, J=2.93 Hz, 1H) 7.45 (s, 1H) 7.64 (d, 2H) 7.71 (d, 2H) 7.86 (dd, J=8.71, 1.66 Hz, 1H) 8.09 (s, 1H) 8.17 (s, 1H) 8.16 (d, 1H) 8.99 (s, 1H).

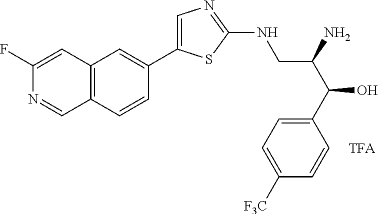

### Example 37

### (1S,2R)-2-Amino-3-((5-(3-fluoro-6-isoquinolinyl)-1,3-thiazol-2-yl)amino)-1-(4-(trifluoromethyl)phenyl)-1-propanol trifluoroacetate: This compound was synthesized as shown in the following scheme

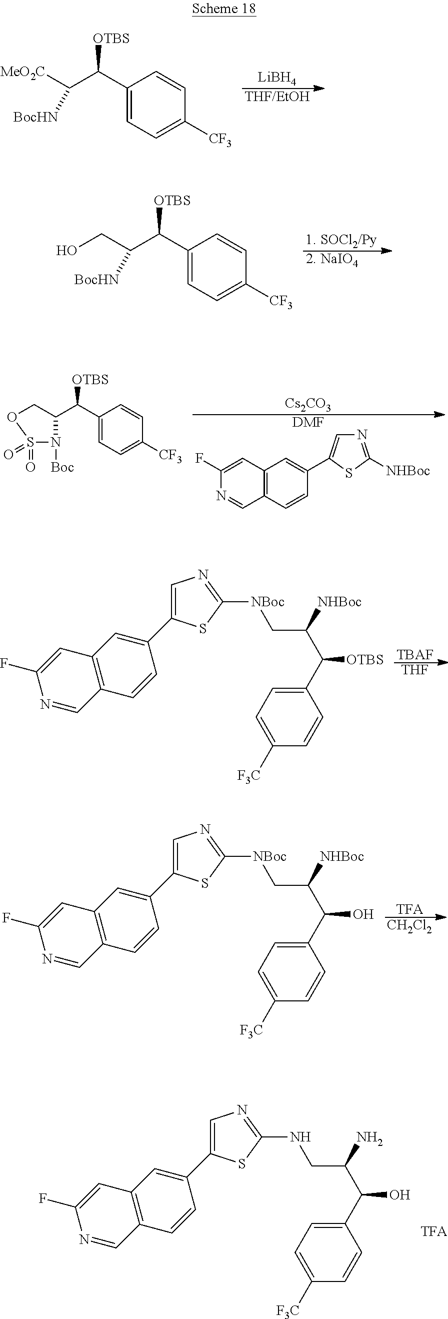

tert-Butyl (1S,2R)-1-(tert-butyldimethylsilyloxy)-3-hydroxy-1-(4-(trifluoromethyl)phenyl)propan-2-ylcarbamate: To a solution of (2S,3S)-methyl 2-(tert-butoxycarbonylamino)-3-(tert-butyldimethylsilyloxy)-3-(4-(trifluoromethyl)phenyl)propanoate (2.6 g, 5.5 mmol, prepared as shown in Scheme 17) in THF (32.4 mL, 400 mmol) and EtOH (9.7 mL, 166 mmol) was added lithium borohydride (2.0 M solution in THF, 5.6 mL, 11.1 mmol, Aldrich) at 0° C. The reaction mixture was stirred at 0° C. for 1 hour and then the cooling bath was removed. After 24 hours at room temperature, HPLC-MS showed over 90% conversion. The reaction was quenched with 5% citric acid in water. The mixture was then concentrated in vacuo and the residue was extracted with EtOAc twice. The organic phase was washed with saturated NaHCO₃, water and brine, dried over Na₂SO₄, filtered, and concentrated in vacuo. The residue was purified by chromatography on silica gel (0-10% to remove the starting material, then 10%-20% EtOAc-hexane) to provide the product as a white solid (1.80 g, 73%). LCMS (API-ES) m/z: 350 (MH⁺-100).

tert-Butyl (4R)-4-(S)-((tert-butyl(dimethyl)silyl)oxy)(4-(trifluoromethyl)phenyl)methyl)-1,2,3-oxathiazolidine-3-carboxylate 2-oxide: To a solution of SOCl₂ (0.74 mL, 10.1 mmol, Aldrich) in ACN (12 mL) and DCM (12 mL) at −78° C. was added a solution of tert-butyl (1S,2R)-1-(tert-butyldimethylsilyloxy)-3-hydroxy-1-(4-(trifluoromethyl)phenyl)propan-2-ylcarbamate (1.82 g, 4.0 mmol) in ACN (20 mL), DCM (20 mL) and THF (4 mL) dropwise via a dropping funnel. After 10 minutes, pyridine (1.82 mL, 22.3 mmol) was added dropwise at −78° C. The mixture was allowed to warm to room temperature and stirred overnight. The solvent was removed in vacuo. The residue was taken up in EtOAc (60 mL), washed with water and brine, dried over Na₂SO₄, filtered, and concentrated in vacuo. The residue was purified by chromatography on silica gel (0%-10% EtOAc-hexane). The product was obtained as a white solid (1.80 g, 90%). LCMS (API-ES) m/z: 440 (M+H⁺-56).

tert-Butyl (4R)-4-(S)-((tert-butyl(dimethyl)silyl)oxy)(4-(trifluoromethyl)phenyl)methyl)-1,2,3-oxathiazolidine-3-carboxylate 2,2-dioxide: A mixture of tert-butyl (4R)-4-(S)-((tert-butyl(dimethyl)silyl)oxy)(4-(trifluoromethyl)phenyl)methyl)-1,2,3-oxathiazolidine-3-carboxylate 2-oxide (1.80 g, 3.63 mmol), sodium periodate (3.11 g, 14.5 mmol, Aldrich), ruthenium(III) chloride hydrate (16 mg, 0.07 mmol) in ACN:water:EtOAc (51 mL: 17 mL: 9 mL) was reacted under sonication for 17 minutes. The dark mixture became a yellow suspension. HPLC-MS showed the starting material was consumed. The reaction mixture was filtered through Celite® brand filter aid, and the solid was washed with EtOAc. The organic phase was washed with water and brine, dried over Na₂SO₄, filtered, and concentrated in vacuo. The residue was purified by chromatography on silica gel (0-4%-10% EtOAc-hexane), and the product was obtained as a white solid (1.75 g, 94%). LCMS (API-ES) m/z: 456 (M+H⁺-56).

tert-Butyl ((2R,3S)-2-((tert-butoxycarbonyl)amino)-3-((tert-butyl(dimethyl)silyl)oxy)-3-(4-(trifluoromethyl)phenyl)propyl)(5-(3-fluoro-6-isoquinolinyl)-1,3-thiazol-2-yl)carbamate: To a flame-dried flask was added tert-butyl 5-(3-fluoroisoquinolin-6-yl)thiazol-2-ylcarbamate (0.08 g, 0.23 mmol, prepared as shown in Scheme 4), cesium carbonate (0.15 g, 0.46 mmol) and DMF (4 mL). The mixture was stirred for 10 minutes and then tert-butyl (4R)-4-((S)-((tert-butyl(dimethyl)silyl)oxy)(4-(trifluoromethyl)phenyl)methyl)-1,2,3-oxathiazolidine-3-carboxylate 2,2-dioxide (0.18 g, 0.35 mmol) was added. The mixture was then stirred at 50° C. for 1 hour. The mixture was diluted with EtOAc, cooled to 0° C. and 1N HCl was added slowly (5 mL). The resulting mixture was stirred for 1 hour. The organic phase was separated and the aqueous phase was extracted twice with EtOAc. The organic layers were combined and saturated NaHCO₃ and Na₂CO₃ were added until the mixture was basic (pH=9). The organic layer was then washed with water and brine, dried over Na₂SO₄, filtered, and concentrated in vacuo. The residue was purified by silica gel chromatography (2-20% EtOAc-hexane) to provide the product as a white solid (0.14 g, 78%). LCMS (API-ES) 677 (MH⁺-100).

tert-Butyl ((2R,3S)-2-((tert-butoxycarbonyl)amino)-3-hydroxy-3-(4-(trifluoromethyl)phenyl)propyl)(5-(3-fluoro-6-isoquinolinyl)-1,3-thiazol-2-yl)carbamate: To a mixture of tert-butyl ((2R,3S)-2-((tert-butoxycarbonyl)amino)-3-((tert-butyl(dimethyl)silyl)oxy)-3-(4-(trifluoromethyl)phenyl)propyl)(5-(3-fluoro-6-isoquinolinyl)-1,3-thiazol-2-yl)carbamate (0.14 g, 0.18 mmol) and THF (2 mL) was added tetrabutylammonium fluoride (0.36 mL of 1.0 M solution in THF, 0.36 mmol, Fluka) at 0° C. After 20 minutes, the mixture was diluted with water and extracted with DCM. The organic phase was dried over Na₂SO₄, and concentrated in vacuo. The residue was purified by silica gel chromatography (50% EtOAc-hexane) to give the product as a light yellow solid (46 mg, 38%). LCMS (API-ES) m/z: 663 (M+H⁺).

(1S,2R)-2-Amino-3-((5-(3-fluoro-6-isoquinolinyl)-1,3-thiazol-2-yl)amino)-1-(4-(trifluoromethyl)phenyl)-1-propanol trifluoroacetate: To a mixture of tert-butyl ((2R,3S)-2-((tert-butoxycarbonyl)amino)-3-hydroxy-3-(4-(trifluoromethyl)phenyl)propyl)(5-(3-fluoro-6-isoquinolinyl)-1,3-thiazol-2-yl)carbamate (34 mg, 0.053 mmol) in DCM (1 mL) was added TFA (1 mL) at room temperature. After 30 minutes, the mixture was concentrated in vacuo. The residue was triturated twice with ether and dried in vacuum oven at 40° C. overnight. The product was obtained as a yellow solid (33 mg, 82%). LCMS (API-ES) m/z: 463 (M+H⁺). ¹H NMR (400 MHz, CD₃OD) δ ppm 3.51-3.69 (m, 2H) 3.88-3.97 (m, 1H) 5.19 (d, J=3.13 Hz, 1H) 7.36 (s, 1H) 7.69-7.82 (m, 7H) 8.07 (d, J=8.61 Hz, 1H) 8.91 (s, 1H).

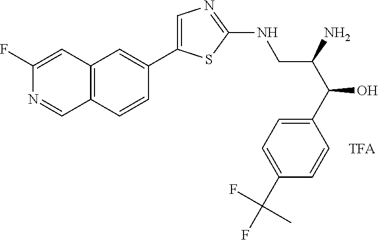

### Example 38

### (1S,2R)-2-Amino-1-(4-(1,1-difluoroethyl)phenyl)-3-((5-(3-fluoro-6-isoquinolinyl)-1,3-thiazol-2-yl)amino)-1-propanol trifluoroacetate

This compound was synthesized as shown in Scheme 19 from 1,1-dimethylethyl (4R)-4-((S)-(4-(1,1-difluoroethyl)phenyl)(((1,1-dimethylethyl)(dimethyl)silyl)oxy)methyl)-1,2,3-oxathiazolidine-3-carboxylate 2,2-dioxide. 1,1-Dimethylethyl (4R)-4-((S)-(4-(1,1-difluoroethyl)phenyl)(((1,1-dimethylethyl)(dimethyl)silyl)oxy)methyl)-1,2,3-oxathiazolidine-3-carboxylate 2,2-dioxide was prepared according to Scheme 18 but using 4-(1,1-difluoroethyl)benzaldehyde instead of 4-(trifluoromethyl)benzaldehyde. 4-(1,1-Difluoroethyl)benzaldehyde was prepared as shown in Scheme 3.

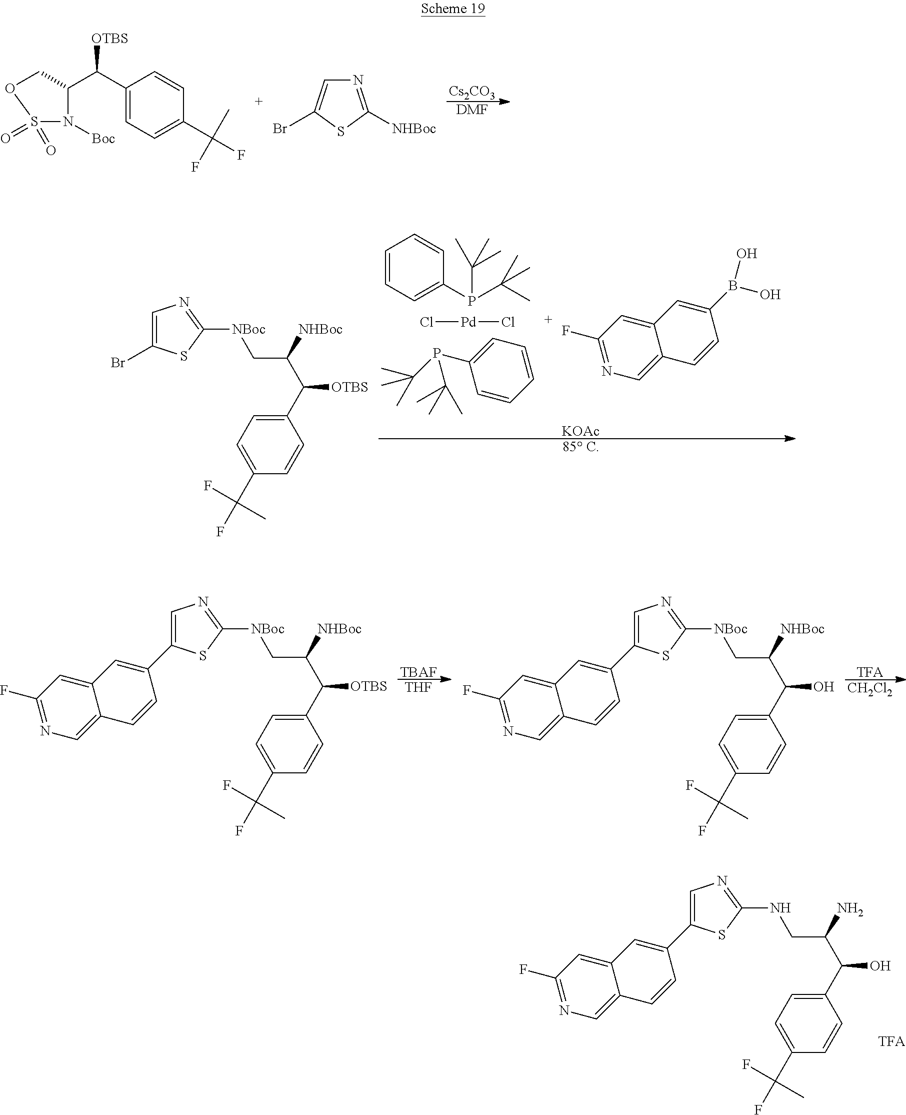

tert-Butyl (5-bromo-1,3-thiazol-2-yl)((2R,3S)-2-((tert-butoxycarbonyl)amino)-3-((tert-butyl(dimethyl)silyl)oxy)-3-(4-(1,1-difluoroethyl)phenyl)propyl)carbamate: To a mixture of tert-butyl 5-bromothiazol-2-ylcarbamate (0.40 g, 1.42 mmol, prepared as shown in Scheme 2) in THF (3 mL) was added cesium carbonate (0.81 g, 2.48 mmol). The mixture was heated at 55° C. for 10 minutes and then a solution of 1,1-dimethylethyl (4R)-4-((S)-(4-(1,1-difluoro ethyl)phenyl)(((1,1-dimethylethyl)(dimethyl)silyl)oxy)methyl)-1,2,3-oxathiazolidine-3-carboxylate 2,2-dioxide (0.60 g, 1.18 mmol) in THF (3 mL) was added. The mixture was heated for 2 hours. The reaction was then evaporated and EtOAc (50 mL) was added. The mixture was cooled to 0° C. and 1N HCl was added slowly (25 mL). The mixture was stirred for 1 hour. The organic phase was separated and the aqueous phase was extracted twice with EtOAc. The organic layers were combined and saturated NaHCO₃ and Na₂CO₃ were added until the mixture was basic (pH=9). The organic layers were then washed with water and brine, dried over Na₂SO₄, filtered, and concentrated in vacuo. The residue was purified by silica gel chromatography (2-20% EtOAc-hexane). The product was obtained as a white solid (0.50 g, 60%). ¹H NMR (300 MHz, CDCl₃) δ ppm-0.08 (s, 3H) 0.12 (s, 3H) 1.00 (s, 9H) 1.30 (s, 9H) 1.37 (s, 9 H) 1.54 (s, 3H) 1.92 (t, J=18.12 Hz, 3H) 3.58-3.80 (m, 1H) 3.95-4.10 (m, 1H) 4.38-4.57 (m, 1H) 5.13 (s, 1H) 5.73 (br. s., 1H) 7.21 (s, 1H) 7.39-7.55 (m, 4H).

tert-Butyl ((2R,3S)-2-((tert-butoxycarbonyl)amino)-3-((tert-butyl(dimethyl)silyl)oxy)-3-(4-(1,1-difluoroethyl)phenyl)propyl)(5-(3-fluoro-6-isoquinolinyl)-1,3-thiazol-2-yl)carbamate: A mixture of potassium acetate (107 mg, 1.09 mmol), 3-fluoroisoquinolin-6-ylboronic acid (45 mg, 0.23 mmol), tert-Butyl (5-bromo-1,3-thiazol-2-yl)((2R,3S)-2-((tert-butoxycarbonyl)amino)-3-((tert-butyl(dimethyl)silyl)oxy)-3-(4-(1,1-difluoroethyl)phenyl)propyl)carbamate (110 mg, 0.16 mmol) in ACN (2.1 mL) and water (0.7 mL) was purged with nitrogen and then bis(di-t-butylphenylphosphine)dichloropalladium (8.7 mg, 0.014 mmol, Guram, S., Organic Letters, 2006, 8(9), 1787-1789) was added. The mixture was heated at 85° C. for 4 hours in a microwave reactor. The bright yellow solution turned dark yellow. The mixture was diluted with EtOAc (50 mL), and washed with saturated NaHCO₃ (twice) and brine. The organic phase was dried over Na₂SO₄, filtered, and concentrated in vacuo. The residue was purified by silica gel chromatography (2%-15%-25% EtOAc-hexane). The product was obtained as a white solid (61 mg, 51%). LCMS (API-ES) m/z: 673 (MH⁺-100).

tert-Butyl ((2R,3S)-2-((tert-butoxycarbonyl)amino)-3-(4-(1,1-difluoroethyl)phenyl)-3-hydroxypropyl)(5-(3-fluoro-6-isoquinolinyl)-1,3-thiazol-2-yl)carbamate: To a mixture of tert-butyl ((2R,3S)-2-((tert-butoxycarbonyl)amino)-3-((tert-butyl(dimethyl)silyl)oxy)-3-(4-(1,1-difluoroethyl)phenyl)propyl)(5-(3-fluoro-6-isoquinolinyl)-1,3-thiazol-2-yl)carbamate (61 mg, 0.079 mmol) and THF (2 mL) was added tetrabutylammonium fluoride (1.0 M solution in THF, 0.16 mL, Fluka) at 0° C. After 1 hour, the mixture was diluted with water and extracted with DCM. The organic phase was dried over Na₂SO₄, filtered, and concentrated in vacuo. The residue was purified by silica gel chromatography (50% EtOAc-hexane). The product was obtained as a white solid (42 mg, 81%). LCMS (API-ES) m/z: 659 (M+H⁺).

(1S,2R)-2-Amino-1-(4-(1,1-difluoro ethyl)phenyl)-3-(5-(3-fluoro-6-isoquinolinyl)-1,3-thiazol-2-yl)amino)-1-propanol trifluoroacetate: To a mixture of tert-butyl ((2R,3S)-2-((tert-butoxycarbonyl)amino)-3-(4-(1,1-difluoroethyl)phenyl)-3-hydroxypropyl)(5-(3-fluoro-6-isoquinolinyl)-1,3-thiazol-2-yl)carbamate (42 mg, 0.065 mmol) in DCM (1 mL) was added TFA (1 mL) at room temperature. After 15 minutes, the mixture was concentrated in vacuo. The residue was washed twice with hexane and then dissolved in MeOH and concentrated in vacuo. The product was obtained as a white solid (44 mg, 100%). LCMS (API-ES) m/z: 459 (M+H⁺). ¹H NMR (400 MHz, CD₃OD) δ ppm 1.92 (t, 3H) 3.51-3.68 (m, 2H) 3.86 (ddd, J=7.48, 4.11, 3.86 Hz, 1H) 5.13 (d, J=3.52 Hz, 1H) 7.36 (s, 1H) 7.56-7.64 (m, 4H) 7.69 (s, 1H) 7.75-7.81 (m, 2H) 8.06 (d, J=9.19 Hz, 1H) 8.90 (s, 1H).

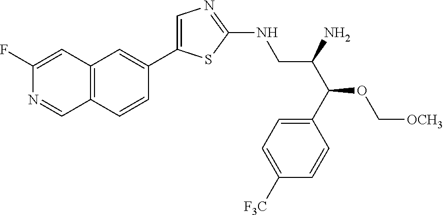

### Example 39

### N-(2R,3S)-2-Amino-3-(methoxymethoxy)-3-(4-(trifluoromethyl)phenyl)propyl)-5-(3-fluoroisoquinolin-6-yl)thiazol-2-amine

This compound was synthesized in a similar manner to that shown in Scheme 19 using 1,1-dimethylethyl 5-bromo-1,3-thiazol-2-yl((2R,3S)-2-((((1,1-dimethylethyl)oxy)carbonyl)amino)-3-(((methyloxy)methyl)oxy)-3-(4-(trifluoromethyl)phenyl)propyl)carbamate instead of 1,1-dimethylethyl 5-bromo-1,3-thiazol-2-yl((2R,3S)-3-(4-(1,1-difluoroethyl)phenyl)-3-(((1,1-dimethylethyl)(dimethyl)silyl)oxy)-2-((((1,1-dimethylethyl)oxy)carbonyl)amino)propyl)carbamate. LCMS (API-ES) m/z: 507 (M+H⁺). ¹H NMR (400 MHz, CDCl₃) δ ppm 3.27-3.36 (m, 2H) 3.39 (s, 3H) 3.58-3.69 (m, 1H) 4.54 (d, J=6.53 Hz, 1H) 4.59-4.70 (m, 2H) 7.15 (s, 1H) 7.50 (d, J=8.03 Hz, 2H) 7.55-7.72 (m, 5H) 7.90 (d, J=8.53 Hz, 1H) 8.85 (s, 1H). 1,1-Dimethylethyl 5-bromo-1,3-thiazol-2-yl((2R,3S)-2-((((1,1-dimethylethyl)oxy)carbonyl)amino)-3-(((methyloxy)methyl)oxy)-3-(4-(trifluoromethyl)phenyl)propyl)carbamate was prepared as shown in Scheme 20.

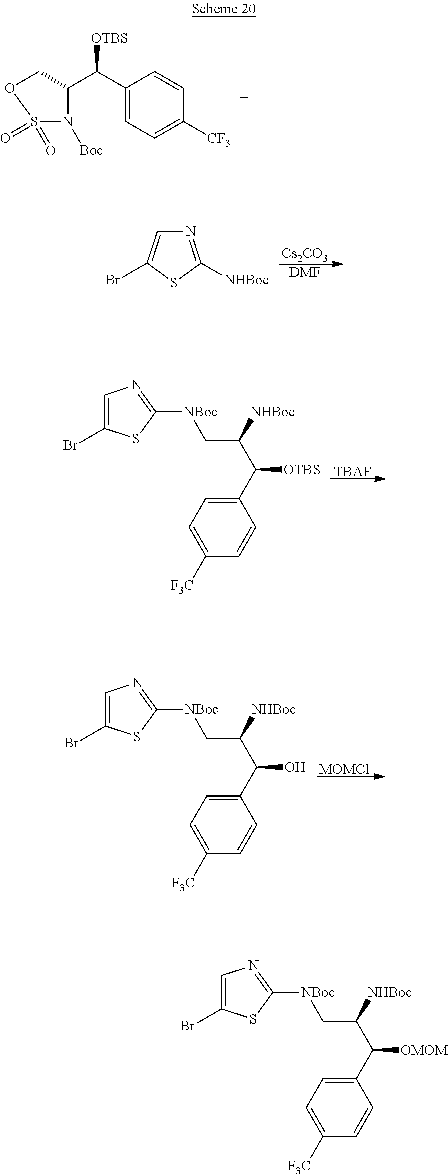

tert-Butyl (5-bromo-1,3-thiazol-2-yl)((2R,3S)-2-((tert-butoxycarbonyl)amino)-3-((tert-butyl(dimethyl)silyl)oxy)-3-(4-(trifluoromethyl)phenyl)propyl)carbamate: To a mixture of tert-butyl 5-bromothiazol-2-ylcarbamate (0.56 g, 2.0 mmol) in THF (3 mL) was added cesium carbonate (1.10 g, 3.5 mmol). The mixture was heated to 55° C. for 10 minutes and then a solution of 1,1-dimethylethyl (4R)-4-((S)-(((1,1-dimethylethyl)(dimethyl)silyl)oxy)(4-(trifluoromethyl)phenyl)methyl)-1,2,3-oxathiazolidine-3-carboxylate 2,2-dioxide (0.85 g, 1.7 mmol, prepared as shown in Scheme 18) in THF (3 mL) was added. The mixture was heated for 80 minutes and then concentrated in vacuo. The residue was dissolved in EtOAc (50 mL). The mixture was cooled to 0° C. and 1N HCl was added slowly (50 mL). The mixture was stirred for 1 hour. The organic phase was separated, and the aqueous phase was extracted twice with EtOAc. The organic layers were combined and saturated NaHCO₃ and Na₂CO₃ were added until it was basic (pH=9). The mixture was washed with water and brine, dried over Na₂SO₄, filtered, and concentrated in vacuo. The residue was purified by silica gel chromatography: 0-6% EtOAc-hexane. The product was obtained as a white solid (0.78 g, 66%). LCMS (API-ES) m/z: 710, 712 (M+H⁺).

tert-Butyl (5-bromo-1,3-thiazol-2-yl)((2R,3S)-2-((tert-butoxycarbonyl)amino)-3-hydroxy-3-(4-(trifluoromethyl)phenyl)propyl)carbamate: To a mixture of tert-butyl (5-bromo-1,3-thiazol-2-yl)((2R,3S)-2-((tert-butoxycarbonyl)amino)-3-((tert-butyl(dimethyl)silyl)oxy)-3-(4-(trifluoromethyl)phenyl)propyl)carbamate (0.20 g, 0.28 mmol) and THF (4 mL) was added tetrabutylammonium fluoride (1.0 M solution in THF (0.48 mL, 0.48 mmol, Fluka)) at 0° C. After 1 hour, the mixture was diluted with water and extracted with DCM. The organic phase was dried over Na₂SO₄, filtered, and concentrated in vacuo. The residue was purified by silica gel chromatography (0-10% EtOAc-hexane) to remove the impurities. The product was obtained as a white solid (0.12 g, 71%). LCMS (API-ES) m/z: 596, 598 (M+H⁺).

tert-Butyl (5-bromo-1,3-thiazol-2-yl)((2R,3S)-2-((tert-butoxycarbonyl)amino)-3-(methoxymethoxy)-3-(4-(trifluoromethyl)phenyl)propyl)carbamate: To a solution of tert-butyl (5-bromo-1,3-thiazol-2-yl)((2R,3S)-2-((tert-butoxycarbonyl)amino)-3-hydroxy-3-(4-(trifluoromethyl)phenyl)propyl)carbamate (100 mg, 0.17 mmol) in DMF (0.026 mL, 0.34 mmol) and dichloroethane (1 mL) was added tetrabutylammonium iodide (68 mg, 0.18 mmol, Alfa Aesar), chloromethyl methyl ether (0.10 mL, 1.34 mmol, Aldrich) and N-ethyl-N-isopropylpropan-2-amine (0.23 mL, 1.34 mmol). In a sealed vial, the mixture was gradually heated to 40° C. and stirred overnight. The reaction was then concentrated in vacuo, diluted with DCM and washed with water three times. The organic phase was dried over Na₂SO₄, filtered, and concentrated in vacuo. The dark residue was purified by silica gel chromatography (6-20% EtOAc-hexane). The product was obtained as an off-white solid (70 mg, 65%). LCMS (API-ES) m/z: 640, 642 (M+H⁺).

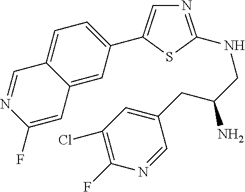

### Example 40

### N—((S)-2-Amino-3-(5-chloro-6-fluoropyridin-3-yl)propyl)-5-(3-fluoroisoquinolin-6-yl)thiazol-2-amine

N—((S)-2-Amino-3-(5-chloro-6-fluoropyridin-3-yl)propyl)-5-(3-fluoroisoquinolin-6-yl)thiazol-2-amine was prepared as shown in Scheme 21.

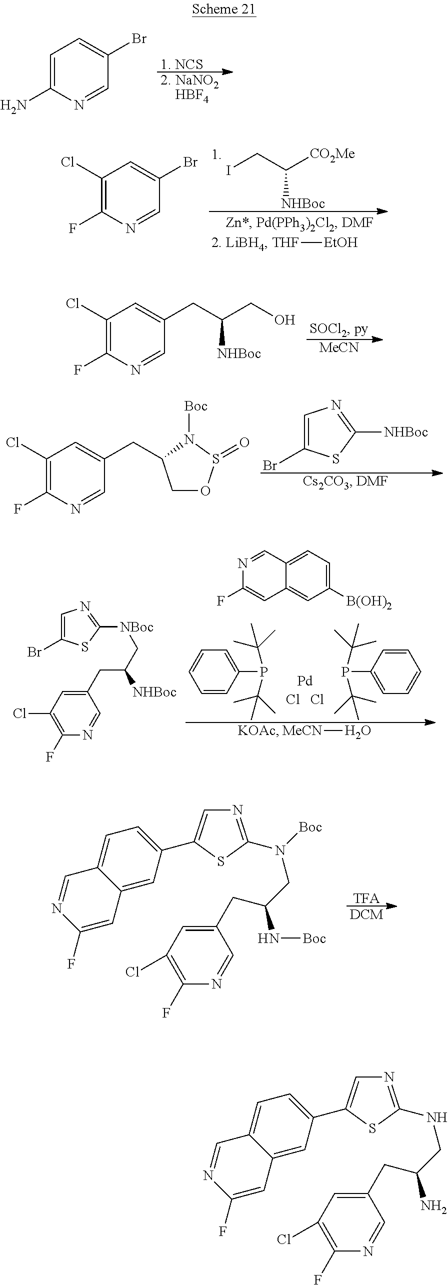

5-Bromo-3-chloropyridin-2-amine: To a stirred and cooled mixture of 2-amino-5-bromopyridine (16.35 g, 94 mmol, Aldrich) in DMF (40 mL) was added N-chlorosuccinimide (14.0 g, 104 mmol, Aldrich) portion-wise at 0° C. in 10 minutes. The resulting mixture was then stirred at 0° C. for 1 hour and at room temperature for 2 hours. The mixture was diluted with ether (100 mL) and brought to pH˜7-8 with 5N NaOH. The layer were separated and the aqueous layer was extracted with ether (100 mL×2). The combined organic layers were washed with brine, dried over Na₂SO₄, filtered, and concentrated to give a residue which was purified by flash column chromatography (ISCO Combiflash system, pure hexanes—20% EtOAc in hexanes) to obtain the desired product 5-bromo-3-chloropyridin-2-amine (17.7 g, 90%) as a white solid. LCMS (API-ES) m/z (%): 208.5 (100%, M⁺+H).

5-Bromo-3-chloro-2-fluoropyridine: To a round-bottom flask containing 5-bromo-3-chloropyridin-2-amine (9.47 g, 45.6 mmol) was added hydrogen tertrafluoroborate (30 mL, Aldrich) and the mixture was cooled to −78° C. Sodium nitrite (8.19 g, 119 mmol, Aldrich) was then added to the suspension and the mixture was stirred at 0° C. for 15 minutes, warmed to room temperature, and then heated at 60° C. for 1 hour. After the reaction was shown to be complete by LC-MS, the reaction mixture was diluted with water (50 mL) and diluted with EtOAc (50 mL). The separated aqueous layer was extracted with EtOAc (50 mL×2) and the combined organic layers were washed with brine, dried over Na₂SO₄, filtered, and concentrated to give a residue. The residue was purified by flash column chromatography (ISCO Combiflash system, pure hexanes—20% EtOAc in hexanes) to provide the desired product (3.66 g, 38%) as a white solid. LCMS (API-ES) m/z (%): 211.4 (100%, M⁺+H).

(S)-Methyl 2-(tert-butoxycarbonylamino)-3-(5-chloro-6-fluoropyridin-3-yl)propanoate: To a stirred suspension of zinc (0.89 mL, 96 mmol, Aldrich) in DMF (15 mL, 195 mmol) was added dibromomethane (0.33 mL, 4.8 mmol, Aldrich). The resulting mixture was then heated at 90° C. for 30 minutes. The mixture was then cooled, trimethylsilyl chloride (0.12 mL, 0.96 mmol, Aldrich) was added, and stirring was continued at room temperature for 30 minutes. Boc-3-iodo-1-alanine methyl ester (7.9 g, 24 mmol, Fluka) in DMF (15 mL) was added, and the mixture was stirred at room temperature for 4 hours prior to the introduction of Pd(PPh₃)₂Cl₂ (0.56 g, 0.80 mmol, Aldrich) and a solution of 5-bromo-3-chloro-2-fluoropyridine (3.38 g, 16 mmol) in DMF (15 mL). The resulting mixture was stirred at room temperature 16 hours and then was passed through a short path of Celite® brand filter aid. The filter cake was washed with EtOAc (30 mL×3) and the combined organic layers were washed with NH₄Cl_{(aq)}, water, and brine successively. The solvent was evaporated and the residue was purified by flash column chromatography (ISCO Combiflash system, pure hexanes—50% EtOAc in hexanes) to provide the desired product (S)-methyl 2-(tert-butoxycarbonylamino)-3-(5-chloro-6-fluoropyridin-3-yl)propanoate (0.80 g, 15%). LCMS (API-ES) m/z (%): 333.8 (100%, M⁺+H).

(S)-tert-Butyl 3-(5-chloro-6-fluoropyridin-3-yl)-1-hydroxypropan-2-ylcarbamate: To a stirred solution of (S)-methyl 2-(tert-butoxycarbonylamino)-3-(5-chloro-6-fluoropyridin-3-yl)propanoate (800 mg, 2.4 mmol) in THF (8 mL) was added lithium borohydride (0.10 g, 4.8 mmol, Aldrich) and EtOH (6 mL) at 0° C. The resulting mixture was stirred at room temperature overnight, quenched with 5% citric acid and water, and concentrated. The resulting mixture was diluted with EtOAc (50 mL). The separated aqueous layer was extracted with EtOAc (50 mL×3) and the combined organic layers were washed with brine, dried over Na₂SO₄, filtered, and concentrated. The residue was purified by flash column chromatography (ISCO Combiflash system, pure hexanes—80% EtOAc in hexanes) to obtain the desired product (S)-tert-butyl 3-(5-chloro-6-fluoropyridin-3-yl)-1-hydroxypropan-2-ylcarbamate as a white solid (458 mg, 62%). LCMS (API-ES) m/z (%): 305.7 (100%, M⁺+H).

tert-Butyl (4S)-4-((5-chloro-6-fluoro-3-pyridinyl)methyl)-1,2,3-oxathiazolidine-3-carboxylate 2-oxide: To a solution of SOCl₂ (0.27 mL, 3.8 mmol, Aldrich) in 30 mL of ACN at −60° C. was added a solution of (S)-tert-butyl 3-(5-chloro-6-fluoropyridin-3-yl)-1-hydroxypropan-2-ylcarbamate (0.46 g, 1.5 mmol) in 125 mL of ACN portion wise. After 10 minutes stirring, pyridine (0.59 g, 7.5 mmol) was added dropwise while maintaining the cold bath temperature at −60° C. The mixture was allowed to warm to room temperature gradually without removing the cold bath, and the mixture was stirred overnight. The solvent was then removed under reduced pressure. The residue was taken up in 200 mL of EtOAc. The mixture was transferred to a separatory funnel and washed twice with 100 mL of water, once with 50 mL of brine, and was then dried over MgSO₄. Filtration and concentration under reduced pressure afforded tert-butyl (4S)-4-((5-chloro-6-fluoro-3-pyridinyl)methyl)-1,2,3-oxathiazolidine-3-carboxylate 2-oxide as a pale brown foam (0.51 g, 97%), which was used in the next reaction without further purification. LCMS (API-ES) m/z (%): 351.1 (100%, M⁺+H).

tert-Butyl (5-bromo-1,3-thiazol-2-yl)((2S)-2-((tert-butoxycarbonyl)amino)-3-(5-chloro-6-fluoro-3-pyridinyl)propyl)carbamate: To a stirred mixture of tert-butyl 5-bromothiazol-2-ylcarbamate (280 mg, 1.01 mmol, prepared as shown in Scheme 2) and Cs₂CO₃ (654 mg, 2.01 mmol) in DMF (2 mL) was added a solution of tert-butyl (4S)-4-((5-chloro-6-fluoro-3-pyridinyl)methyl)-1,2,3-oxathiazolidine-3-carboxylate 2-oxide (528 mg, 1.51 mmol) in DMF (2 mL) at 60° C. The resulting mixture was then stirred at 60° C. for 1 hour. The reaction mixture was then diluted with NH₄Cl_{(aq)}, water, and EtOAc (10 mL). The separated aqueous layer was extracted with EtOAc (20 mL×2) and the combined organic layers were washed with brine, dried over Na₂SO₄, filtered, and concentrated. The residue was purified by flash column chromatography (ISCO Combiflash system, pure hexanes—20% EtOAc in hexanes) to obtain the desired product (412 mg, 73%) as a pale yellow foam. LCMS (API-ES) m/z (%): 566.8 (100%, M⁺+H).

tert-Butyl-((2S)-2-((tert-butoxycarbonyl)amino)-3-(5-chloro-6-fluoro-3-pyridinyl)propyl)(5-(3-fluoro-6-isoquinolinyl)-1,3-thiazol-2-yl)carbamate: To a mixture of 3-fluoroisoquinolin-6-ylboronic acid (37 mg, 196 μmol, prepared as shown in Scheme 1), (S)—N-(2-amino-3-(5-chloro-6-fluoropyridin-3-yl)propyl)-5-bromothiazol-2-N-Boc-amine (74 mg, 131 μmol), and PdCl₂(t-butylPPh₃)₂ (12 mg, 0.13 umol, Johnson Matthey catalog number Pd-122) was added potassium acetate (96 mg, 0.98 mmol), and then acetonitrile (2.5 mL, 0.13 mmol) and water (1 mL) under N₂. The resulting mixture was then heated by microwave irradiation at 100° C. for 1 hour. The mixture was then cooled, and passed through a short path of Na₂SO₄. The filter cake was washed with EtOAc (10 mL×3). The combined organic phases were concentrated to give the product, which was used in the next reaction without purification.

N—((S)-2-Amino-3-(5-chloro-6-fluoropyridin-3-yl)propyl)-5-(3-fluoroisoquinolin-6-yl)thiazol-2-amine: To a stirred mixture of tert-butyl ((2S)-2-((tert-butoxycarbonyl)amino)-3-(5-chloro-6-fluoro-3-pyridinyl)propyl)(5-(3-fluoro-6-isoquinolinyl)-1,3-thiazol-2-yl)carbamate in DCM (2 mL) was added TFA (2 mL), and the mixture was stirred at room temperature for 1 hour. The reaction mixture was concentrated and diluted with DCM, NaHCO_{3(aq)}, and water (10 mL each). The separated aqueous layer was extracted with DCM (10 mL×2) and the combined organic layers were washed with brine, dried over Na₂SO₄, filtered, and concentrated to give a residue which was purified by flash column chromatography (pure DCM—10% MeOH in DCM) and concentrated to obtain the product which was washed with ether to afford N—((S)-2-amino-3-(5-chloro-6-fluoropyridin-3-yl)propyl)-5-(3-fluoroisoquinolin-6-yl)thiazol-2-amine (17 mg, 27%) as a yellow solid. LCMS (API-ES) m/z (%): 432.1 (100%, M⁺+H); ¹H NMR (400 MHz, DMSO-d₆) δ ppm 8.98 (s, 1H) 8.02-8.16 (m, 3H) 7.89 (d, J=8.41 Hz, 1H) 7.82 (s, 1H) 7.77 (s, 1H) 7.50 (s, 1H) 3.07-3.47 (m, 6H) 2.84 (dd, J=13.20, 4.01 Hz, 1H) 2.58 (dd, J=13.50, 8.22 Hz, 1H).

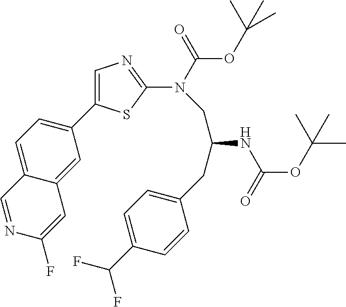

### Example 41

### tert-Butyl ((2S)-2-((tert-butoxycarbonyl)amino)-3-(4-(difluoromethyl)phenyl)propyl)(5-(3-fluoro-6-isoquinolinyl)-1,3-thiazol-2-yl)carbamate

This compound was synthesized in a similar manner to Example 1 but using 1,1-dimethyl (4S)-4-(4-(difluoromethyl)phenyl)methyl-1,2,3-oxathiazolidine-3-carboxylate-2,2-dioxide instead of (S)-3-(tert-Butyloxycarbonyl)-4-((6-(trifluoromethyl)pyridin)[1,2,3]-oxathiazolidine-2-oxide. LCMS (API-ES) m/z (%): 562.4 (100%, M⁺+H); ¹H NMR (400 MHz, CDCl₃) δ ppm 1.29 (s, 9H) 1.41 (br. s., 9H) 2.81 (d, J=7.24 Hz, 1H) 3.01 (d, J=0.98 Hz, 1H) 3.96 (br. s., 1H) 4.05 (d, J=7.04 Hz, 1 H) 4.24 (br. s., 2H) 7.12 (s, 1H) 7.30 (d, J=8.02 Hz, 2H) 7.40 (d, J=8.22 Hz, 2H) 7.66 (dt, J=8.61, 0.88 Hz, 1H) 7.72 (s, 1H) 7.80 (d, J=0.78 Hz, 1H) 7.90 (d, J=8.61 Hz, 1H) 8.83 (s, 1H).

1,1-Dimethyl (4S)-4-(4-(difluoromethyl)phenyl)methyl-1,2,3-oxathiazolidine-3-carboxylate-2,2-dioxide was prepared as shown in Scheme 2 using 1-bromo-4-difluoromethylbenzene (Oakwood, catalog no. 23880) instead of 5-bromo-2-(trifluoromethyl)pyridine and in Scheme 7 using (S)-tert-butyl-3-(4-(difluoromethyl)phenyl-2-yl)-1,2,3-oxathiazolidine-3-carboxylate-2 oxide instead of tert-butyl (4S)-4-(3,4-dichlorobenzyl)-1,2,3-oxathiazolidine-3-carboxylate 2-oxide.

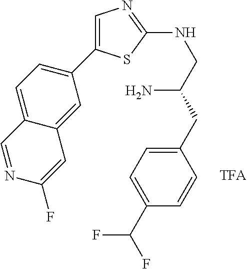

### Example 42

### (2S)-3-(4-(difluoromethyl)phenyl)-N-1-(5-(3-fluoro-6-isoquinolinyl)-1,3-thiazol-2-yl)-1-2-propanediamine trifluoroacetate

This compound was synthesized in a similar manner to Example 2 but using tert-butyl 42S)-2-((tert-butoxycarbonyl)amino)-3-(4-(difluoromethyl)phenyl)propyl)(5-(3-fluoro-6-isoquinolinyl)-1,3-thiazol-2-yl)carbamate prepared as for Example 41 instead of tert-butyl (5-bromo-1,3-thiazol-2-yl)((2S)-2-((tert-butoxycarbonyl)amino)-3-(6-(trifluoromethyl)-3-pyridinyl)propyl)carbamate. LCMS (API-ES) m/z (%): 429.7 (100%, M⁺+H); ¹H NMR (400 MHz, DMSO-d₆) δ ppm 2.65 (dd, J=6.75, 6.36 Hz, 1H) 2.83 (dd, J=4.99, 0.88 Hz, 1 H) 3.13-3.27 (m, 2H) 3.37 (br. s., 1H) 5.76 (s, 1H) 6.87 (s, 1H) 7.01 (s, 1H) 7.15 (s, 1 H) 7.40 (d, J=8.02 Hz, 2H) 7.46-7.55 (m, 3H) 7.76 (s, 1H) 7.81 (s, 1H) 7.88 (dt, J=8.90, 0.93 Hz, 1H) 8.09 (d, J=8.80 Hz, 1H) 8.98 (s, 1H).

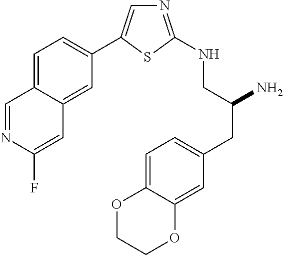

### Example 43

### N—((S)-2-Amino-3-(2,3-dihydrobenzo[b][1,4]dioxin-6-yl)propyl)-5-(3-fluoroisoquinolin-6-yl)thiazol-2-amine

This compound was prepared as shown in Scheme 22.

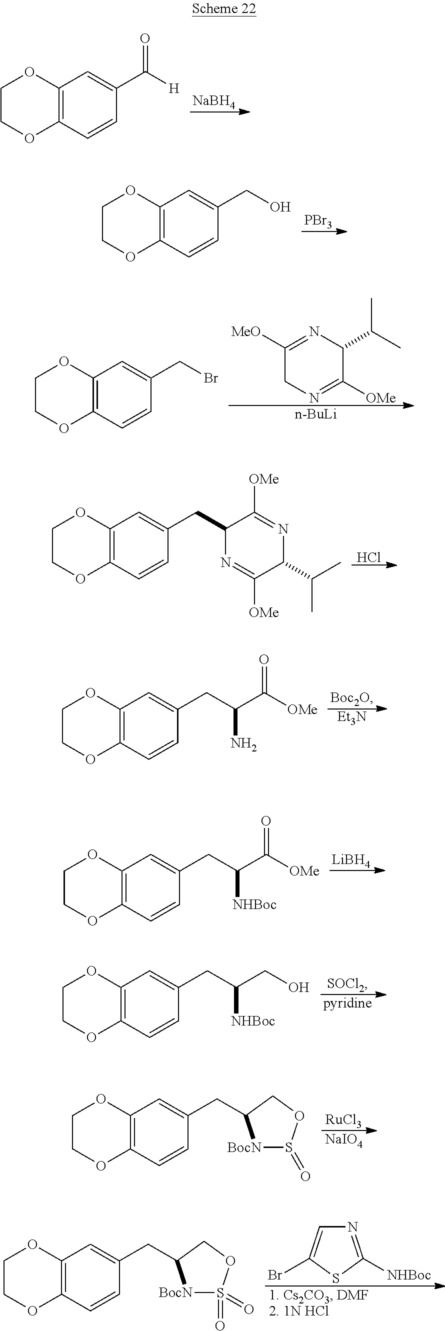

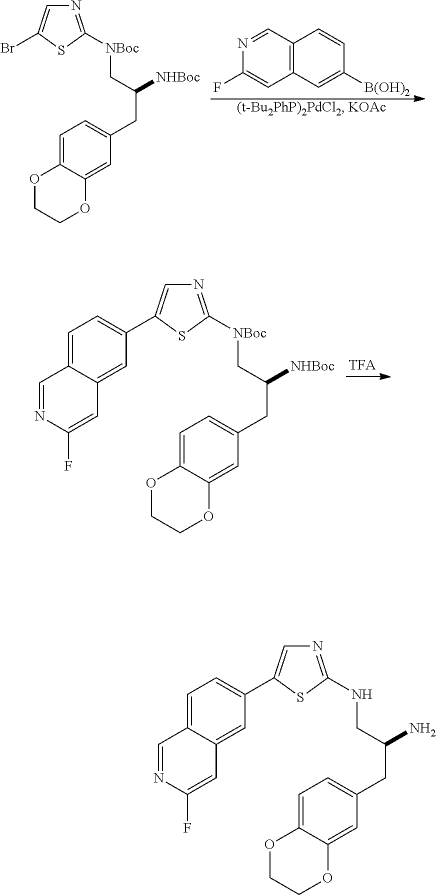

(2,3-Dihydrobenzo[b][1,4]dioxin-6-yl)methanol. To a solution of 2,3-dihydrobenzo[b][1,4]dioxine-6-carbaldehyde (TCI America Laboratory Chemicals B2019) (20 g, 0.12 mol) in MeOH (400 mL) at 0° C. was added NaBH₄ (14 g, 0.36 mol) in portions. After stirring at 0° C. for 30 minutes, the mixture was neutralized to pH=7 by addition of 2 M aqueous HCl. The MeOH was removed under reduced pressure, and the residue was extracted with DCM (2×200 mL). The combined organic layers were concentrated under reduced pressure to afford (2,3-dihydrobenzo[b][1,4]dioxin-6-yl)methanol (19 g, 95%).

6-(Bromomethyl)-2,3-dihydrobenzo[b][1,4]dioxine. To a solution of (2,3-dihydrobenzo[b][1,4]dioxin-6-yl)methanol (46 g, 0.28 mol) in Et₂O (1 L) at −40° C. was added PBr₃ (84 g, 0.3 mol, Aldrich) dropwise. The resulting mixture was stirred for 16 hoursat room temperature. The reaction was quenched by careful addition of H₂O (1 L). The organic phase was separated, washed with H₂O (2×1 L), dried (Na₂SO₄), filtered, and concentrated in vacuo to give the initial product, which was purified by silica gel column chromatography eluting with petroleum ether/EtOAc (100:1 to 20:1) to afford 6-(bromomethyl)-2,3-dihydrobenzo[b][1,4]dioxine (23 g, 37%).

(2S,5R)-2-((2,3-Dihydrobenzo[b][1,4]dioxin-6-yl)methyl)-5-isopropyl-3,6-dimethoxy-2,5-dihydropyrazine. n-BuLi (57 mL, 142 mmol, Aldrich) was added dropwise to a solution of (R)-2,5-dihydro-3,6-dimethoxy-2-isopropylpyrazine (24 g, 132 mmol) in THF (200 mL) at −78° C. under N₂, and the mixture was stirred at the same temperature for 40 minutes. A solution of 6-(bromomethyl)-2,3-dihydrobenzo[b][1,4]dioxine (27 g, 118 mmol) in THF (160 mL) was added dropwise, and the resulting mixture was stirred at −78° C. for an additional 2 hours. The reaction was quenched by addition of saturated aqueous NH₄Cl (200 mL). The THF was then removed under reduced pressure, and the resulting mixture was partitioned between EtOAc (300 mL) and H₂O (300 mL). The organic layer was separated, washed with brine (300 mL), dried (Na₂SO₄), filtered, and concentrated in vacuo. The residue was purified by silica gel column chromatography eluting with petroleum ether/EtOAc (100:1 to 30:1) to afford (2S,5R)-2-((2,3-dihydrobenzo[b][1,4]dioxin-6-yl)methyl)-5-isopropyl-3,6-dimethoxy-2,5-dihydropyrazine (34 g, 73%). LCMS (API-ES) m/z 333.2 (M+H+).

(S)-Methyl 2-amino-3-(2,3-dihydrobenzo[b][1,4]dioxin-6-yl)propanoate. A mixture of (2S,5R)-2-(2,3-dihydrobenzo[b][1,4]dioxin-6-yl)methyl)-5-isopropyl-3,6-dimethoxy-2,5-dihydropyrazine (32.7 g, 0.1 mol) in THF (260 mL) and 1 M aqueous HCl (200 mL) was stirred at room temperature for 20 hours. The THF was removed under reduced pressure and the resulting residue was partitioned between EtOAc (300 mL) and saturated aqueous NaHCO₃ (300 mL). The organic phase was separated, washed with water (8×300 mL) and brine (8×300 mL), dried (Na₂SO₄), filtered, and concentrated to give (S)-methyl 2-amino-3-(2,3-dihydrobenzo[b][1,4]dioxin-6-yl)propanoate (11.6 g, 50%).

(S)-Methyl 2-(tert-butoxycarbonylamino)-3-(2,3-dihydrobenzo[b][1,4]dioxin-6-yl)propanoate. A mixture of (S)-methyl 2-amino-3-(2,3-dihydrobenzo[b][1,4]dioxin-6-yl)propanoate (11.3 g, 47.6 mmol), Et₃N (10 mL), and Boc₂O (10.4 g, 47.6 mmol, Aldrich) in MeOH (180 mL) was stirred at room temperature under N₂ for 3 hours. The solvent was removed under reduced pressure, and the resulting residue was partitioned between EtOAc (200 mL) and saturated aqueous NaHCO₃ (200 mL). The organic layer was separated, washed with brine (300 mL), dried (Na₂SO₄) and evaporated. The initially obtained product was purified by silica gel column chromatography eluting with petroleum ether/EtOAc (100:1) to afford (S)-methyl 2-(tert-butoxycarbonylamino)-3-(2,3-dihydrobenzo[b][1,4]dioxin-6-yl)propanoate (9.0 g, 57%).

(S)-tert-Butyl 3-(2,3-dihydrobenzo[b][1,4]dioxin-6-yl)-1-hydroxypropan-2-ylcarbamate. To a solution of (S)-methyl 2-(tert-butoxycarbonylamino)-3-(2,3-dihydrobenzo[b][1,4]dioxin-6-yl)propanoate (9.6 g, 28.4 mmol) in THF (160 mL) and EtOH (48 mL) at −20° C. was added LiBH₄ (1.25 g, 57 mmol, Aldrich). The resulting mixture was then stirred at room temperature for 4 hours. The mixture was neutralized to pH=7 by the addition of 1 M aqueous HCl and extracted with EtOAc (2×200 mL). The combined organic layers were washed with brine (300 mL), dried (Na₂SO₄), filtered, and evaporated to afford (S)-tert-butyl 3-(2,3-dihydrobenzo[b][1,4]dioxin-6-yl)-1-hydroxypropan-2-ylcarbamate (7.6 g, 86%).

1,1-Dimethylethyl (4S)-4-(2,3-dihydro-1,4-benzodioxin-6-ylmethyl)-1,2,3-oxathiazolidine-3-carboxylate 2-oxide. To a solution of SOCl₂ (4.4 mL, 61 mmol) in 50 mL of ACN and 50 mL DCM at −60° C. was added (S)-tert-butyl 3-(2,3-dihydrobenzo[b][1,4]dioxin-6-yl)-1-hydroxypropan-2-ylcarbamate (7.5 g, 24 mmol) in 50 mL of ACN and 50 mL DCM dropwise via dropping funnel. After 10 minutes, pyridine (9.9 mL, 121 mmol) was added dropwise while maintaining the cold bath temperature at −60° C. The mixture was allowed to warm to room temperature and stirred overnight. The solvent was then removed under reduced pressure. The resulting residue was taken up in 500 mL of EtOAc. The mixture was transferred to a separatory funnel and washed twice with 300 mL of water and once with 300 mL of brine, dried over Na₂SO₄ and filtered. The residue was purified by chromatography on silica gel, eluting with 5% EtOAc in hexane. After removing the solvent, 1,1-dimethylethyl (4S)-4-(2,3-dihydro-1,4-benzodioxin-6-ylmethyl)-1,2,3-oxathiazolidine-3-carboxylate 2-oxide (6.3 g, 73%) was obtained as a light yellow oil. MS (API-ES) m/z (%): 378.1 (100%, M+Na⁺).

1,1-Dimethylethyl (4S)-4-(2,3-dihydro-1,4-benzodioxin-6-ylmethyl)-1,2,3-oxathiazolidine-3-carboxylate 2,2-dioxide. 1,1-Dimethylethyl (4S)-4-(2,3-dihydro-1,4-benzodioxin-6-ylmethyl)-1,2,3-oxathiazolidine-3-carboxylate 2-oxide (5.9 g, 16.6 mmol) was charged into a 500 mL round bottom flask. To the flask were added 150 mL ACN, 50 mL water, 25 mL EtOAc, sodium periodate (14.2 g, 66.4 mmol, Aldrich), and ruthenium(III) chloride hydrate (18.71 mg, 0.083 mmol, Aldrich). The resulting mixture was sonicated for 10 minutes. The reaction mixture was filtered through filter paper and washed with EtOAc a few times. The filtrate was evaporated. The residue was taken up in EtOAc (200 mL). The organic layer was washed with brine, dried over sodium sulfate and evaporated. The desired product, 1,1-dimethylethyl (4S)-4-(2,3-dihydro-1,4-benzodioxin-6-ylmethyl)-1,2,3-oxathiazolidine-3-carboxylate 2,2-dioxide (5.2 g, 82%), was obtained as a white solid. MS (API-ES) m/z (%): 394.1 (100%, M+Na⁺).

tert-Butyl (5-bromo-1,3-thiazol-2-yl)((2S)-2-((tert-butoxycarbonyl)amino)-3-(2,3-dihydro-1,4-benzodioxin-6-yl)propyl)carbamate. To a solution of tert-butyl 5-bromothiazol-2-ylcarbamate (1.6 g, 5.9 mmol, prepared as shown in Scheme 2) in 20 mL of THF was added cesium carbonate (3.9 g, 11.8 mmol). The mixture was heated at 55° C. for 10 minutes, and 1,1-dimethylethyl (4S)-4-(2,3-dihydro-1,4-benzodioxin-6-ylmethyl)-1,2,3-oxathiazolidine-3-carboxylate 2,2-dioxide (2.2 g, 5.9 mmol) was added as a solid. After 80 minutes at 55° C., the reaction mixture was concentrated. The residue was taken up in 50 mL of EtOAc and the mixture was cooled to 0° C. The mixture was stirred for 5 minutes and 50 mL 10% aqueous hydrochloride solution was added. After stirring at 0° C. for 1 hour, saturated sodium bicarbonate solution was added to the mixture to make it slightly basic and 10% sodium carbonate solution was added to make the resulting mixture more basic. The aqueous portion was extracted twice with 200 mL of EtOAc. The combined organic layers were washed once with brine, dried over sodium sulfate, filtered and evaporated. The residue was purified by chromatography on silica gel eluting with 10% EtOAc in hexane. After removing the solvent, the desired product was obtained as a white solid (2.5 g, 74%). MS (API-ES) m/z (%): 570.1 (100%, M+1).

tert-Butyl ((2S)-2-((tert-butoxycarbonyl)amino)-3-(2,3-dihydro-1,4-benzodioxin-6-yl)propyl)(5-(3-fluoro-6-isoquinolinyl)-1,3-thiazol-2-yl)carbamate. A 20 mL flask was charged with potassium acetate (252 mg, 2.57 mmol), bis(di-t-butylphenylphosphine)dichloropalladium (Johnson Matthey) (21 mg, 33 μmol), 3-fluoroisoquinolin-6-ylboronic acid (70 mg, 367 μmol, prepared as shown in Scheme 1), tert-butyl (5-bromo-1,3-thiazol-2-yl)((2S)-2-((tert-butoxycarbonyl)amino)-3-(2,3-dihydro-1,4-benzodioxin-6-yl)propyl)carbamate (230 mg, 403 μmol), 3.0 mL of ACN and 1.0 mL of water. After heating at 90° C. overnight, the mixture was concentrated. The residue was taken up in EtOAc and saturated aqueous sodium bicarbonate. The aqueous layer was extracted with EtOAc (2×100 mL). The combined organic layers were washed with brine, dried over sodium sulfate, filtered and evaporated. The residue was purified by chromatography on silica gel eluting with 0-20% EtOAc in hexane. After removing the solvent, the desired product was obtained as a white solid (70 mg, 30%). MS (API-ES) m/z (%): 637.2 (100%, M+1).

N—((S)-2-Amino-3-(2,3-dihydrobenzo[b][1,4]dioxin-6-yl)propyl)-5-(3-fluoroisoquinolin-6-yl)thiazol-2-amine. tert-Butyl ((2S)-2-((tert-butoxycarbonyl)amino)-3-(2,3-dihydro-1,4-benzodioxin-6-yl)propyl)(5-(3-fluoro-6-isoquinolinyl)-1,3-thiazol-2-yl)carbamate (80 mg, 126 μmol) was dissolved in 5.0 mL anhydrous DCM and 5.0 mL TFA was added. After 30 minutes stirring at room temperature, the solvent was evaporated and the residue was taken up in 100 mL EtOAc. To the resulting solution was added 50 mL saturated aqueous sodium bicarbonate and 30 mL 5% aqueous sodium carbonate. The organic layer was washed with brine and dried over sodium sulfate to afford N—((S)-2-amino-3-(2,3-dihydrobenzo[b][1,4]dioxin-6-yl)propyl)-5-(3-fluoroisoquinolin-6-yl)thiazol-2-amine as a white solid (51 mg, 92%). MS (API-ES) m/z (%): 437.1 (100%, M⁺+H). ¹H NMR (400 MHz, CD₃OD): δ ppm 2.51-2.64 (m, 1H), 2.78 (dd, J=13.30, 5.27 Hz, 1H), 3.23-3.32 (m, 2H), 3.44 (t, J=8.03 Hz, 1H), 4.23 (s, 4 H), 6.63-6.86 (m, 3H), 7.37 (s, 1H), 7.68 (s, 1H), 7.77-7.85 (m, 2H), 8.06 (d, J=9.03 Hz, 1H), 8.89 (s, 1H).

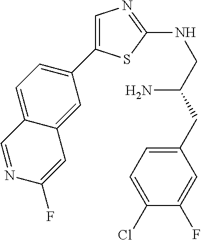

### Example 44

### (2S)-3-(4-Chloro-3-fluorophenyl)-N-1-(5-(3-fluoro-6-isoquinoliny)-1,3-thiazol-2-yl)-1-2-propanediamine

This compound was synthesized in a similar manner as Example 43 but using 4-(bromomethyl)-1-chloro-2-fluorobenzene (Oakwood, catalog number F5731) instead of 6-(bromomethyl)-2,3-dihydrobenzo[b][1,4]dioxine. LCMS (API-ES) m/z (%): 431.3 (100%, M⁺+H); ¹H NMR (400 MHz, DMSO-d₆) δ ppm 2.92 (dd, J=10.96, 6.65 Hz, 2H) 3.60 (br. s., 2H) 3.62 (d, J=0.98 Hz, 1H) 5.76 (s, 1H) 7.19 (ddd, J=8.66, 1.12, 0.78 Hz, 1H) 7.41 (d, J=9.98 Hz, 1 H) 7.52 (s, 1H) 7.57 (t, J=8.12 Hz, 1H) 7.81 (d, J=0.98 Hz, 2H) 7.81 (s, 1H) 7.85 (s, 1 H) 7.91 (dt, J=8.66, 0.95 Hz, 1H) 8.12 (d, J=8.80 Hz, 1H) 9.00 (s, 1H).

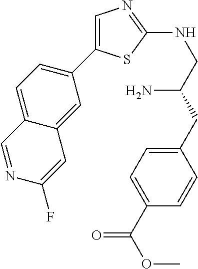

### Example 45

### Methyl 4-((2S)-2-amino-3-β5-(3-fluoro-6-isoquinolinyl)-1,3-thiazol-2-yl)amino)propyl)benzoate

This compound was synthesized in a manner similar to Example 42 using 1-bromo-4-(difluoro(methyloxy)methyl)benzene instead of 1-bromo-4-difluoromethylbenzene, During the Suzuki coupling reaction of 3-fluoroisoquinolin-6-ylboronic acid with tert-butyl (5-bromo-1,3-thiazol-2-yl)((2S)-2-((tert-butoxycarbonyl)amino)-3-(4-(difluoro(methoxy)methyl)phenyl)propyl)carbamate, the difluoro(methyloxy)methyl group hydrolyzed to the methyl ester to provide methyl 4-((2S)-2-((tert-butoxycarbonyl)amino)-3-((tert-butoxycarbonyl)(5-(3-fluoro-6-isoquinolinyl)-1,3-thiazol-2-yl)amino)propyl)benzoate as the cross-coupled product. LCMS (API-ES) m/z (%): 437.5 (100%, M⁺+H). ¹H NMR (400 MHz, DMSO-d₆) δ ppm 2.92 (dd, J=10.9.6, 6.65 Hz, 2H) 3.60 (br. s., 1H) 3.62 (d, J=0.98 Hz, 2H) 3.84 (br. s., 3H) 5.76 (br. s., 3H) 7.45 (br. s., 3H) 7.82 (s, 1H) 7.93 (d, J=8.02 Hz, 3H) 8.10 (d, J=8.80 Hz, 1H) 8.99 (br. s., 1H). 1-Bromo-4-(difluoro(methyloxy)methyl)benzene was prepared as shown in Scheme 23.

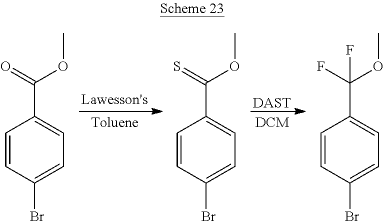

O-Methyl 4-bromobenzenecarbothioate: A glass microwave reaction vessel was charged with methyl 4-bromobenzoate (5.58 g, 26 mmol, Aldrich, catalog number 407593), anhydrous toluene (2.8 mL), and Lawesson's reagent (12 g, 29 mmol, Aldrich catalog number 227439). The reaction mixture was stirred and heated in a Smith Synthesizer® microwave reactor (Personal Chemistry, Inc., Upssala, Sweden) at 200° C. for 40 minutes. The solvent was removed in vacuo, and the residue was adsorbed onto a plug of silica gel and purified by chromatography through a Redi-Sep® pre-packed silica gel column (40 g), eluting with a gradient of 5% to 50% EtOAc in hexane, to provide O-methyl 4-bromobenzenecarbothioate (3.5 g, 58%). LCMS (API-ES) m/z (%): 232.1 (100%, M⁺+H); ¹H NMR (400 MHz, CDCl₃) δ ppm 3.93 (d, J=1.76 Hz, 3H) 7.53-7.66 (m, 2H) 7.92 (dt, J=8.61, 1.08 Hz, 2H).

1-Bromo-4-(difluoro(methyloxy)methyl)benzene: To a 50 mL round-bottomed flask was added O-methyl 4-bromobenzothioate (0.50 g, 2.2 mmol), DCM (10 mL), and (diethylamino)sulfur trifluoride (0.86 mL, 6.5 mmol, Alfa catalog number A1192). To the solution was added NBS (0.44 mL, 5.2 mmol, Fluka catalog number 18350), and the resulting solution was stirred at room temperature for 4 hours. The solvent was removed in vacuo, and the residue was adsorbed onto a plug of silica gel and purified by chromatography through a Redi-Sep® pre-packed silica gel column (40 g), eluting with a gradient of 5% to 50% EtOAc in hexane, to provide 1-bromo-4-(difluoro(methyloxy)methyl)benzene (0.26 g, 51%). ¹H NMR (400 MHz, CDCl₃) δ ppm 3.83 (s, 3H) 7.49 (d, J=8.41 Hz, 2H) 7.81 (d, J=8.61 Hz, 2H).

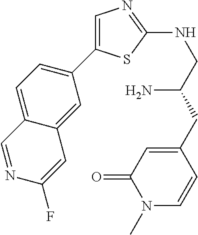

### Example 46, 4-((2S)-2-Amino-3-(5-(3-fluoro-6-isoquinolinyl)-1,3-thiazol-2-yl)amino)propyl)-1-methyl-2(1H)-pyridinone

This compound was synthesized in a manner similar to Example 43 using 4-(bromomethyl)-1-methylpyridine-2(1H)-one, instead of 6-(bromomethyl)-2,3-dihydrobenzo[b][1,4]dioxine. LCMS (API-ES) m/z (%): 410.4 (100%); ¹H NMR (400 MHz, CD₃OD) δ ppm 2.53 (d, J=6.26 Hz, 1 H) 2.81 (d, J=6.26 Hz, 1H) 3.31 (dt, J=3.37, 1.74 Hz, 3H) 3.43 (m, 1H) 3.54 (d, J=14.87 Hz, 1H) 5.49 (s, 1H) 6.36 (dt, J=6.99, 1.00 Hz, 1H) 6.48 (d, J=0.98 Hz, 1H) 7.34 (s, 1H) 7.60 (d, J=6.85 Hz, 1H) 7.67 (s, 1H) 7.74-7.82 (m, 2H) 8.04 (d, J=9.00 Hz, 1H) 8.87 (s, 1H). 4-(Bromomethyl)-1-methylpyridine-2(1H)-one was made as shown in Scheme 24 starting with 1-methyl-2-oxo-1,2-dihydropyridine-4-carboxylic acid.

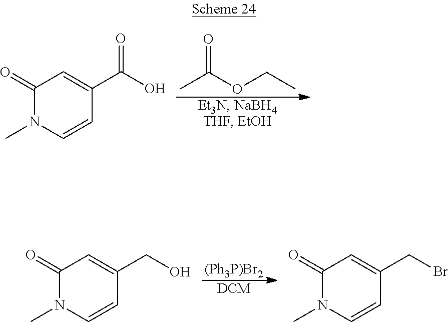

4-(Hydroxymethyl)-1-methylpyridine-2(1H)-one. To a 50 mL round-bottomed flask was added 1-methyl-2-oxo-1,2-dihydropyridine-4-carboxylic acid (700 mg, 4.6 mmol, Matrix catalog number 020304), DCM (5 mL), TEA (0.96 mL), and ethyl chloroformate (0.52 mL, 5.5 mmol, Aldrich catalog number 185892). The resulting solution was stirred at room temperature for 1 hour. The mixture was concentrated, and the residue was diluted with THF (12 mL) and EtOH (3 mL) and treated with NaB H₄ (0.86 g, 23 mmol). The mixture was concentrated to half the volume and diluted with water and EtOAc. The separated aqueous layer was extracted with EtOAc and the combined organic layers were washed with brine, dried over MgSO₄, filtered and concentrated. The initially obtained product was adsorbed onto a plug of silica gel and purified by chromatography through a Redi-Sep® pre-packed silica gel column (40 g), eluting with a gradient of 5% to 50% EtOAc in hexane, to provide 4-(hydroxymethyl)-1-methylpyridine-2(1H)-one as a white solid (0.18 g, 28%). LCMS (API-ES) m/z 140.2 (M+H+).

4-(Bromomethyl)-1-methylpyridine-2(1H)-one: To a 50 mL round-bottomed flask was added 4-(hydroxymethyl)-1-methylpyridine-2(1H)-one (0.20 g, 1.44 mmol) and DCM (3.0 mL). The resulting solution was cooled in an ice bath and treated with triphenylphosphine dibromide (0.728 g, 1.72 mmol, Aldrich, catalog number 270946). The mixture was stirred at room temperature overnight. The mixture was concentrated, and the residue was adsorbed onto a plug of silica gel and purified by chromatography through a Redi-Sep® pre-packed silica gel column (40 g), eluting with a gradient of 5% to 50% EtOAc in hexane, to provide 4-(bromomethyl)-1-methylpyridine-2(1H)-one as a pale yellow solid (0.18 g, 63%). ¹H NMR (400 MHz, CDCl₃) δ 7.28 (d, J=6.53 Hz, 1H) 6.57 (s, 1H) 6.21 (d, J=6.53 Hz, 1H) 4.20 (s, 2H) 3.54 (s, 3H).

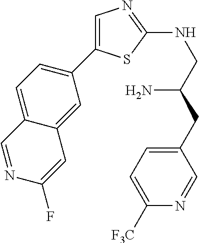

### Example 47

### (2R)—N¹-(5-(3-Fluoro-6-isoquinolinyl)-1,3-thiazol-2-yl)-3-(6-(trifluoromethyl)-3-pyridinyl)-1,2-propanediamine

This compound was synthesized in a manner similar to Example 2 using methyl Boc-3-iodo-D-alanine methyl ester (Fluka, Catalog Number 15124), instead of using Boc-3-iodo-L-alanine methyl ester. LCMS (API-ES) m/z (%): 448 (100%); ¹H NMR (400 MHz, DMSO-d₆) δ ppm 2.90-2.99 (m, 1H) 3.02-3.10 (m, 1H) 3.42-3.56 (m, 2H) 3.62 (d, J=5.28 Hz, 1H) 5.76 (s, 1H) 7.52 (s, 1H) 7.77-7.95 (m, 5H) 8.03 (d, J=7.63 Hz, 1H) 8.12 (d, J=8.80 Hz, 1H) 8.33 (br. s., 1H) 8.71 (br. s., 1H) 9.00 (d, J=0.39 Hz, 1H).

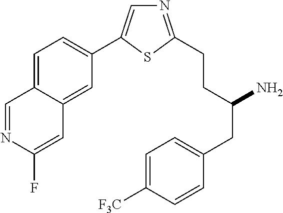

### Example 48

### (2R)-4-(5-(3-Fluoroisoquinolin-6-yl)thiazol-2-yl)-1-(4-(trifluoromethyl)phenyl)butan-2-amine

This compound was prepared as shown in Scheme 25.

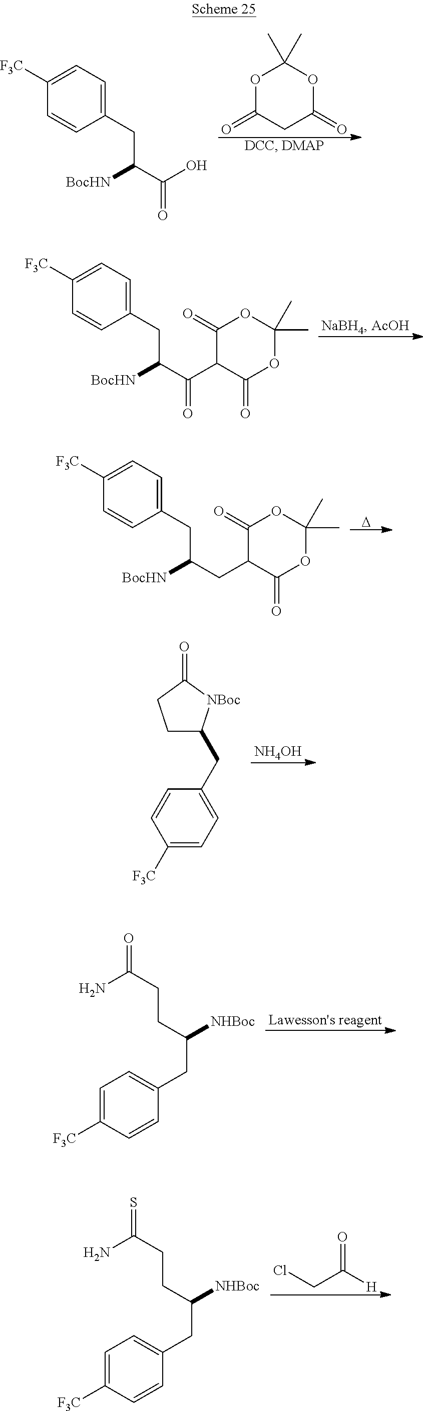

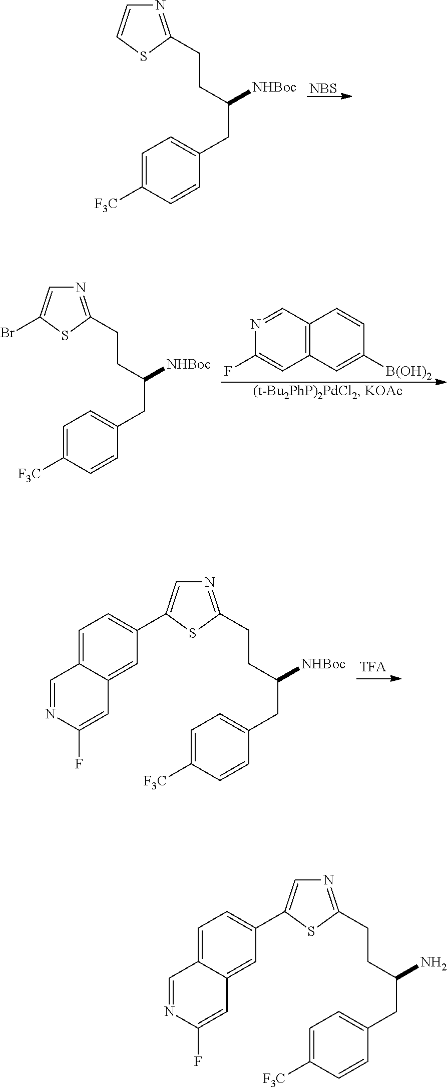

(S)-tert-Butyl 1-(2,2-dimethyl-4,6-dioxo-1,3-dioxan-5-yl)-1-oxo-3-(4-(trifluoromethyl)phenyl)propan-2-ylcarbamate. Boc-L-4-trifluoromethylphenylalanine (Fluka F15017) (16.0 g, 48 mmol), 2,2-dimethyl-1,3-dioxane-4,6-dione (Aldrich 210145) (7.6 g, 53 mmol), and 4-dimethylaminopyridine (Aldrich 522813) (9.1 g, 74 mmol) in 200 mL DCM were cooled to −5° C. 1,3-Dicyclohexylcarbodiimide (Aldrich D80002) (11 g, 53 mmol) in 50 mL DCM was added dropwise over 40 minutes. The resulting mixture was stirred overnight at room temperature. The suspension was filtered washing with DCM. The filtrate was washed with 5% KHSO₄ four times and once with brine. The separated organic layer was dried with sodium sulfate and filtered. Evaporation of the solvent provided the desired product as a white amorphous solid (21.0 g, 95%). No further purification was carried out and the reaction was carried on to the next step. ¹H NMR (400 MHz, CDCl₃) δ ppm 1.22-1.43 (s, 9H), 1.64-1.67 (s, 3H), 1.75-1.77 (s, 3 H), 2.80-2.95 (d, 1H), 3.13-3.31 (d, 1H), 4.91-5.14 (s, 1H), 5.85-5.98 (t, 1H), 7.44 (d, 2H), 7.57 (d, 2H).

(R)-tert-Butyl 3-(2,2-dimethyl-4,6-dioxo-1,3-dioxan-5-yl)-1-(4-(trifluoromethyl)phenyl)propan-2-ylcarbamate. (S)-tert-Butyl 1-(2,2-dimethyl-4,6-dioxo-1,3-dioxan-5-yl)-1-oxo-3-(4-(trifluoromethyl)phenyl)propan-2-ylcarbamate (22.0 g, 48 mmol) in 200 mL DCM was cooled to −5° C. AcOH (32 g, 527 mmol) was added in one portion and sodium tetrahydroborate (Aldrich 213462) (4.5 g, 120 mmol) was added as a solid portionwise over about 40 minutes. The reaction mixture was stirred for another 40 minutes and then stored in the freezer overnight. The resulting mixture was washed with brine (3×150 mL) and water (2×100 mL). The separated organic layer was dried over MgSO₄ and filtered. Evaporation of the solvent provided he desired product as a white amorphous solid (21.0 g, 98%). No further purification was carried out and the reaction was carried on to the next step. ¹H NMR (400 MHz, CDCl₃) δ ppm 1.34 (s, 9H), 1.74 (s, 3H), 1.78 (s, 3H), 2.10-2.32 (m, 2H), 2.79-2.97 (m, 2H), 3.85 (m, 1H), 4.28 (d, J=7.53 Hz, 1H), 7.34 (d, J=7.53 Hz, 2H), 7.57 (d, J=8.03 Hz, 2H).

(R)-tert-Butyl 2-(4-(trifluoromethyl)benzyl)-5-oxopyrrolidine-1-carboxylate. (R)-tert-Butyl 3-(2,2-dimethyl-4,6-dioxo-1,3-dioxan-5-yl)-1-(4-(trifluoromethyl)phenyl)propan-2-ylcarbamate (10.0 g, 22.5 mmol) in 100 mL toluene was heated at 105° C. for 3 hours. Hexane was added, and the resulting mixture was sonicated. The resulting solid was recovered by filtration. The desired product was obtained as a white amorphous solid (21.0 g, 98%). No further purification was carried out and the reaction product was carried on to the next step. ¹H NMR (400 MHz, CDCl₃) δ ppm 1.57 (s, 9H), 1.71-1.82 (m, 1H), 1.88-2.06 (m, 1H), 2.39 (dd, J=10.29, 5.77 Hz, 2H), 2.79 (dd, J=13.05, 9.54 Hz, 1H), 3.22 (dd, J=13.05, 3.01 Hz, 1H), 4.26-4.46 (m, 1H), 7.32 (d, J=7.53 Hz, 2H), 7.58 (d, J=8.28 Hz, 2H).

(R)-tert-Butyl 5-amino-5-oxo-1-(4-(trifluoromethyl)phenyl)pentan-2-ylcarbamate. To a 150 mL round bottom flask containing (R)-tert-Butyl 2-(4-(trifluoromethyl)benzyl)-5-oxopyrrolidine-1-carboxylate (9.5 g, 28 mmol) was added 40 mL THF and 20.0 mL of 28% ammonia hydroxide (from J. T. Baker). After stirring for seven days at room temperature, the organic solvent was evaporated. A solid was recovered by filtration. The solid was transferred into a flask, 50 mL of hexane was added, and the resulting mixture was sonicated for 15 minutes. The resulting solid was recovered by filtration to provide the desired product as a white crystalline solid (8.5 g, 86%). ¹H NMR (400 MHz, CDCl₃) δ ppm 1.37 (s, 9H), 1.59-1.79 (m, 1H), 1.90 (d, J=14.56 Hz, 1H), 2.29 (t, J=6.78 Hz, 2H), 2.84 (d, J=6.02 Hz, 2H), 3.90 (s, 1H), 7.30 (d, J=8.03 Hz, 2H) 7.55 (d, J=8.03 Hz, 2H).

(R)-tert-Butyl 5-amino-5-thioxo-1-(4-(trifluoromethyl)phenyl)pentan-2-ylcarbamate. A mixture of (R)-tert-butyl 5-amino-5-oxo-1-(4-(trifluoromethyl)phenyl)pentan-2-ylcarbamate (7.0 g, 19 mmol), DCM (200 mL), and Lawesson's reagent (Aldrich 227439) (4.7 g, 12 mmol) was stirred at room temperature f under N₂. The mixture was initially a cloudy suspension and became clearer after stirring for 3 hours. After 6 hours, the reaction was complete and the mixture was concentrated. The residue was purified by chromatography on silica eluting with 20-50% EtOAc in hexane to provide the desired product as a white solid (5.5 g, 75%). ¹H NMR (400 MHz, CDCl₃) δ ppm 1.24-1.46 (s, 9H), 1.66-1.85 (m, 1H), 1.99-2.16 (m, 1H), 2.57-2.72 (m, 1H), 2.71-2.85 (m, 2H), 2.87-3.00 (m, 1H), 3.76-3.91 (m, 1H), 7.29 (d, J=8.03 Hz, 2H), 7.56 (d, J=7.53 Hz, 2H).

(R)-tert-Butyl 4-(thiazol-2-yl)-1-(4-(trifluoromethyl)phenyl)butan-2-ylcarbamate. (R)-tert-Butyl 5-amino-5-thioxo-1-(4-(trifluoromethyl)phenyl)pentan-2-ylcarbamate (1.1 g, 3.0 mmol) and 2-chloroacetaldehyde (50% in benzene, Aldrich 317276) (0.5 g, 6 mmol) were mixed in 70 mL THF. The mixture was heated at 60° C. for 4 hours. About 50% conversion was observed via LC-MS. The reaction mixture was taken up in EtOAc and the mixture was washed with saturated ammonium chloride solution. The organic layer was dried over sodium sulfate. After removing the solvent, the remaining residue was treated again with 2-chloroacetaldehyde (50% in benzene Aldrich 317276) (0.5 g, 6 mmol) and heated at reflux for 4 hours using a Dean-Stark trap. A complete and clean conversion was observed by LC-MS. The reaction mixture was taken up in EtOAc, and the mixture was washed with saturated aqueous ammonium chloride solution. The separated organic layer was dried over sodium sulfate and filtered. After removing the solvent, the residue was triturated in MeOH to provide the desired compound (1.1 g, 93%). LCMS (API-ES) m/z (%): 401.2 (100%, M⁺+H).

(R)-tert-Butyl 4-(5-bromothiazol-2-yl)-1-(4-(trifluoromethyl)phenyl)butan-2-ylcarbamate. (R)-tert-Butyl 4-(thiazol-2-yl)-1-(4-(trifluoromethyl)phenyl)butan-2-ylcarbamate (38.7 mg, 97 μmol) was treated with NBS (Fluka 18350) (34 mg, 191 μmol) in DMF at room temperature. After stirring at room temperature for 20 hours, the reaction was not complete. Additional NBS (Fluka 18350) (34 mg, 191 μmol) was added in a DMF solution. After 2 hours, LCMS indicated>95% conversion. The reaction mixture was partitioned between EtOAc and water. The separated organic phase was washed with saturated aqueous ammonium chloride three times, dried over sodium sulfate, filtered and evaporated. The desired product was obtained as a white solid (40 mg, 86%). LCMS (API-ES) m/z (%): 479.2 (100%, M⁺+H).

tert-Butyl 4-(5-(3-fluoroisoquinolin-6-yl)thiazol-2-yl)-1-(4-(trifluoromethyl)phenyl)butan-2-ylcarbamate. A mixture of tert-butyl 4-(5-bromothiazol-2-yl)-1-(4-(trifluoromethyl)phenyl)butan-2-ylcarbamate (126 mg, 263 μmol), 3-fluoroisoquinolin-6-ylboronic acid (75 mg, 394 μmol, prepared as shown in Scheme 1), bis(di-tert-butylphenylphosphine)dichloropalladium (II) (Johnson Matthey) (16 mg, 26 μmol), and potassium acetate (129 mg, 1.31 mmol) in ACN/H₂O (3:1, 2.6 mL) was heated at 80° C. for 3 hours. The mixture was concentrated under reduced pressure, and the residue was dissolved in MeOH/DCM, absorbed onto silica gel, and purified by flash chromatography (silica gel, 40% to 70% EtOAc/hexanes) to provide tert-butyl 4-(5-(3-fluoroisoquinolin-6-yl)thiazol-2-yl)-1-(4-(trifluoromethyl)phenyl)butan-2-ylcarbamate (59 mg, 41%) as a white solid. LCMS (API-ES) m/z (%): 546.0 (100%, M⁺+H).

(2R)-4-(5-(3-Fluoroisoquinolin-6-yl)thiazol-2-yl)-1-(4-(trifluoromethyl)phenyl)butan-2-amine. TFA (1.0 mL) was added to a solution of tert-butyl (R)-4-(5-(3-fluoroisoquinolin-6-yl)thiazol-2-yl)-1-(4-(trifluoromethyl)phenyl)butan-2-ylcarbamate (52 mg, 95 μmol) in DCM (1.5 mL), and the mixture was stirred at room temperature for 1.5 hours. The mixture was concentrated under reduced pressure. The residue was taken up in MeOH, filtered, and purified by reverse phase HPLC (Shimadsu Valiant, Phenomenex Gemini C18.5 μm 100×30 mm, 10% to 70% H₂O/ACN, 0.1% TFA). The fractions containing the product were combined, neutralized by addition of solid Na₂CO₃ and extracted with DCM. The combined extracts were dried over MgSO₄, filtered, and concentrated under reduced pressure to provide (2R)-4-(5-(3-fluoroisoquinolin-6-yl)thiazol-2-yl)-1-(4-(trifluoromethyl)phenyl)butan-2-amine (42 mg, 99%) as a light yellow solid. ¹H NMR (300 MHz, CDCl₃) δ ppm 1.71 (br s, 2H) 1.80-1.97 (m, 1H) 2.01-2.17 (m, 1H) 2.67 (dd, J=13.3, 8.4 Hz, 1H) 2.94 (dd, J=13.3, 4.8 Hz, 1H) 3.10-3.34 (m, 3H) 7.24 (s, 1H) 7.33 (d, J=7.9 Hz, 2H) 7.57 (d, J=7.9 Hz, 2H) 7.72 (d, J=8.6 Hz, 1H) 7.88 (s, 1H) 7.96-8.08 (m, 2H) 8.94 (s, 1H). LCMS (API-ES) m/z (%): 446.0 (100%, M⁺+H).

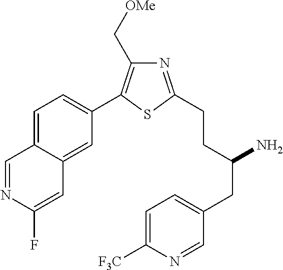

### Example 49

### (2R)-4-(5-(3-Fluoroisoquinolin-6-yl)-4-(methoxymethyl)thiazol-2-yl)-1-(6-(trifluoromethyl)pyridin-3-yl)butan-2-amine

This compound was prepared in a similar manner as Example 48 but using (R)-tert-butyl 4-(4-(methoxymethyl)thiazol-2-yl)-1-(6-(trifluoromethyl)pyridin-3-yl)butan-2-ylcarbamate instead of (R)-tert-butyl 4-(thiazol-2-yl)-1-(4-(trifluoromethyl)phenyl)butan-2-ylcarbamate. ¹H NMR (400 MHz, MeOH) δ ppm 1.89-1.94 (m, 1H), 2.78-2.87 (m, 1H), 2.95-3.03 (m, 1H), 3.13-3.30 (m, 3H), 3.45 (s, 3H), 4.53 (s, 2H), 5.51 (s, 1H), 7.49 (s, 1H), 7.75-7.80 (m, 2H), 7.95 (d, J=6.65 Hz, 1H), 8.10 (s, 1H), 8.21 (d, J=8.61 Hz, 1H), 8.63 (s, 1H), 9.06 (s, 1H). LCMS (API-ES) m/z: 491.0 (M+H+). (R)-tert-Butyl 4-(4-(methoxymethyl)thiazol-2-yl)-1-(6-(trifluoromethyl)pyridin-3-yl)butan-2-ylcarbamate was prepared as shown in Scheme 26.

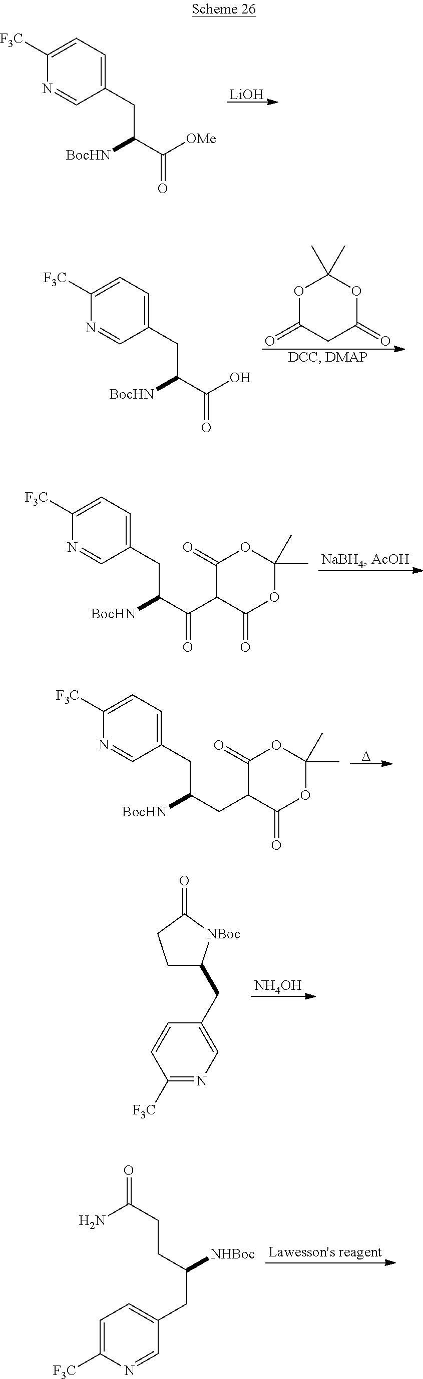

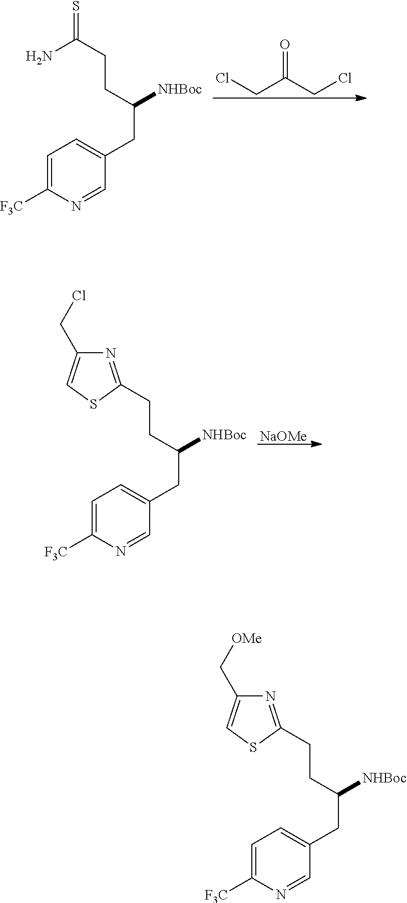

(S)-2-(tert-Butoxycarbonylamino)-3-(6-(trifluoromethyl)pyridin-3-yl)propanoic acid. To LiOH solution (1.0 M in 25 mL distilled water, 25 mL MeOH and 25 mL THF, 75 mL total volume) was added (S)-methyl 2-(tert-butoxycarbonylamino)-3-(6-(trifluoromethyl)pyridin-3-yl)propanoate (3.8 g, 10.9 mmol, prepared as shown in Scheme 2). The reaction was stirred at room temperature for 30 minutes. The organic solvent in the reaction mixture was evaporated. The residue was diluted with EtOAc. The EtOAc layer was washed with 100 mL saturated aqueous ammonium chloride twice, dried over sodium sulfate and filtered. After removing the solvent, the desired product (2.4 g, 66%) was obtained as a white solid. LCMS (API-ES) m/z (%): 335.0 (100%, M⁺+H).

(S)-tert-Butyl 1-(2,2-dimethyl-4,6-dioxo-1,3-dioxan-5-yl)-1-oxo-3-(6-(trifluoromethyl)pyridin-3-yl)propan-2-ylcarbamate. (S)-2-(tert-Butoxycarbonylamino)-3-(6-(trifluoromethyl)pyridin-3-yl)propanoic acid (2.4 g, 7.2 mmol), 2,2-dimethyl-1,3-dioxane-4,6-dione (Aldrich 210145) (1.1 g, 7.9 mmol), and 4-dimethylaminopyridine (Aldrich 522813) (1.4 g, 11 mmol) in 30 mL DCM were cooled in an ice-water-sodium chloride bath (−5° C.). 1,3-Dicyclohexylcarbodiimide (Aldrich D80002) (1.6 g, 7.9 mmol) in 50 mL DCM was added dropwise over about 40 minutes. The reaction mixture was stirred overnight at room temperature. The suspension was filtered, washing with DCM. The filtrate was washed with 5% aqueous KHSO₄ (4×50 mL), once with brine and dried with MgSO₄. After removing the solvent, the desired product (3.0 g, 91%) was obtained as a white solid. LCMS (API-ES) m/z (%): 461.0 (100%, M⁺+H).

(R)-tert-Butyl 3-(2,2-dimethyl-4,6-dioxo-1,3-dioxan-5-yl)-1-(6-(trifluoromethyl)pyridin-3-yl)propan-2-ylcarbamate. (S)-tert-Butyl 1-(2,2-dimethyl-4,6-dioxo-1,3-dioxan-5-yl)-1-oxo-3-(6-(trifluoromethyl)pyridin-3-yl)propan-2-ylcarbamate in 100 mL DCM was cooled to −5° C. AcOH (4.45 g, 74.1 mmol) was added in one portion. The resulting mixture was stirred for 5 minutes and NaBH₄ (0.637 g, 16.8 mmol) was added portion wise over 40 minutes. After stirring for another 40 minutes, the reaction mixture was washed with brine (3×150 mL) and water (2×100 mL). The separated organic layer was dried over MgSO₄ and filtered. After removing the solvent, the desired product (2.5 g, 83%) was obtained as a white solid. LCMS (API-ES) m/z (%): 447.0 (100%, M⁺+H).

(R)-tert-Butyl 2-oxo-5-(6-(trifluoromethyl)pyridin-3-yl)methyl)pyrrolidine-1-carboxylate. (R)-tert-Butyl 3-(2,2-dimethyl-4,6-dioxo-1,3-dioxan-5-yl)-1-(6-(trifluoromethyl)pyridin-3-yl)propan-2-ylcarbamate (2.5 g, 5.6 mmol) in 100 mL tolueTo ane was heated at 105° C. for 8 hours. After removing the solvent, the remaining residue was purified by silica gel column chromatography separation eluting with 20% to 40% EtOAc in hexane. After removing the solvent, the residue was purified by chromatography on silica gel, eluting with 20% to 40% EtOAc in hexane to provide the desired product (1.3 g, 67%) as a white solid. LCMS (API-ES) m/z (%): 345.0 (100%, M⁺+H).

(R)-tert-Butyl 5-amino-5-oxo-1-(6-(trifluoromethyl)pyridin-3-yl)pentan-2-ylcarbamate. To a 150 mL round bottom flask containing (R)-tert-butyl 2-oxo-5-((6-(trifluoromethyl)pyridin-3-yl)methyl)pyrrolidine-1-carboxylate (1.30 g, 3.8 mmol) was added 20 mL THF and 20 mL of 28% ammonium hydroxide. The reaction mixture was a cloudy suspension. After stirring at room temperature for 48 hours, the organic solvent was removed. The resulting precipitate was recovered by filtration. After drying under vacuum, the desired product was obtained as a white solid (1.0 g, 73%). LCMS (API-ES) m/z (%): 306.1 (100%, M⁺-55).

(R)-tert-Butyl 5-amino-5-thioxo-1-(6-(trifluoromethyl)pyridin-3-yl)pentan-2-ylcarbamate. A mixture of (R)-tert-butyl 5-amino-5-oxo-1-(6-(trifluoromethyl)pyridin-3-yl)pentan-2-ylcarbamate (1.0 g, 3.0 mmol), DCM (45 mL), and Lawesson's reagent (Aldrich 227439) (0.7 g, 2.0 mmol) was stirred at room temperature for 30 minutes under N₂. After 2 hours, LCMS indicated that the reaction was complete. The mixture was then concentrated in vacuo. The resulting residue was triturated with EtOAc. The solid was recovered by filtration. The solid was purified by chromatography on silica gel eluting with 20% to 40% EtOAc in hexane. After removing the solvent, the desired product (0.9 g, 86%) was obtained as a white solid. LCMS (API-ES) m/z (%): 322.1 (100%, M⁺-55).

(R)-tert-Butyl 4-(4-(chloromethyl)thiazol-2-yl)-1-(6-(trifluoromethyl)pyridin-3-yl)butan-2-ylcarbamate. (R)-tert-Butyl 5-amino-5-thioxo-1-(6-(trifluoromethyl)pyridin-3-yl)pentan-2-ylcarbamate (900 mg, 2.4 mmol), 1,3-dichloroacetone (363 mg, 2.9 mmol, Aldrich) and 30 mL MeOH were charged into a 1 L round bottom flask. The reaction mixture was stirred at 58° C. overnight, 1,3-Dichloroacetone (Aldrich 168548) (363 mg, 2.9 mmol) was added and the mixture was heated at 65° C. for 4 hours. The reaction mixture was concentrated. The resulting residue was taken up in EtOAc and saturated aqueous sodium bicarbonate was added. The organic layer was separated, washed with brine, dried over sodium sulfate and filtered. After removing the solvent, the desired product (1.0 g, 93%) was obtained as a white solid. LCMS (API-ES) m/z (%): 450.1 (100%, M⁺+H).

(R)-tert-Butyl 4-(4-(methoxymethyl)thiazol-2-yl)-1-(6-(trifluoromethyl)pyridin-3-yl)butan-2-ylcarbamate. (R)-tert-Butyl 4-(4-(chloromethyl)thiazol-2-yl)-1-(6-(trifluoromethyl)pyridin-3-yl)butan-2-ylcarbamate (270 mg, 600 μmol) and sodium methoxide (81.1 mg, 1.5 mmol) were mixed together in 30 mL MeOH. After heating at 60° C. overnight, sodium methoxide (81.1 mg, 1.5 mmol) was added and the mixture was heated at 65° C. for 4 h. The reaction mixture was concentrated. The resulting residue was taken up in EtOAc and saturated sodium bicarbonate was added. The organic layer was separated, washed with brine, dried over sodium sulfate and filtered. After removing the solvent, the desired product (250 mg, 94%) was obtained as a white solid. LCMS (API-ES) m/z (%): 446.1 (100%, M⁺+H).

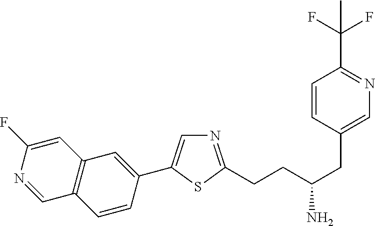

### Example 50

### N—((S)-2-Amino-3-(6-(1,1-difluoroethyl)pyridin-3-yl)propyl)-5-(3-fluoroisoquinolin-6-yl)thiazol-2-amine

The title compound was synthesized in a manner similar to that described for Example 48, but using (S)-methyl 2-(tert-butoxycarbonylamino)-3-(6-(1,1-difluoroethyl)pyridin-3-yl)propanoate instead of (S)-methyl 2-(tert-butoxycarbonylamino)-3-(4-(trifluoromethyl)phenyl)propanoate. MS m/z: 443 (M+1); ¹H NMR (400 MHz, CD₃OD): δ ppm 1.93-2.04 (m, 3H) 2.15-2.27 (m, 2H) 3.06-3.26 (m, 4H) 3.71-3.78 (m, 1H) 7.46 (s, 1H) 7.73 (d, J=8.22 Hz, 1H) 7.88-7.94 (m, 2H) 8.13-8.25 (m, 3H) 8.58 (s, 1H) 9.01 (s, 1H).

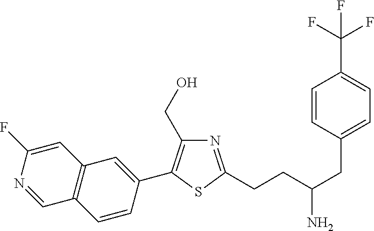

### Example 51

### (±)-(2-(3-Amino-4-(4-(trifluoromethyl)phenyl)butyl)-5-(3-fluoroisoquinolin-6-yl)thiazol-4-yl)methanol

This compound was prepared in a similar manner to Example 48 but using (±)-tert-butyl 4-(5-bromo-4-(hydroxymethyl)thiazol-2-yl)-1-(4-(trifluoromethyl)phenyl)butan-2-ylcarbamate instead of (S)-methyl 2-(tert-butoxycarbonylamino)-3-(4-(trifluoromethyl)phenyl)propanoate: MS m/z: 476 (M+1); ¹H NMR (400 MHz, CD₃OD) δ ppm 2.14-2.24 (m, 2H) 3.07-3.20 (m, J=14.16, 14.16, 14.04, 7.14 Hz, 2H) 3.24 (t, J=7.43 Hz, 2H) 3.71-3.79 (m, 1H) 4.71 (s, 2H) 7.48-7.56 (m, 3H) 7.68-7.77 (m, 3H) 8.11 (s, 1H) 8.22 (d, J=8.61 Hz, 1H) 9.06 (s, 1H). (±)-tert-Butyl 4-(5-bromo-4-(hydroxymethyl)thiazol-2-yl)-1-(4-(trifluoromethyl)phenyl)butan-2-ylcarbamate was prepared as shown in Scheme 27.

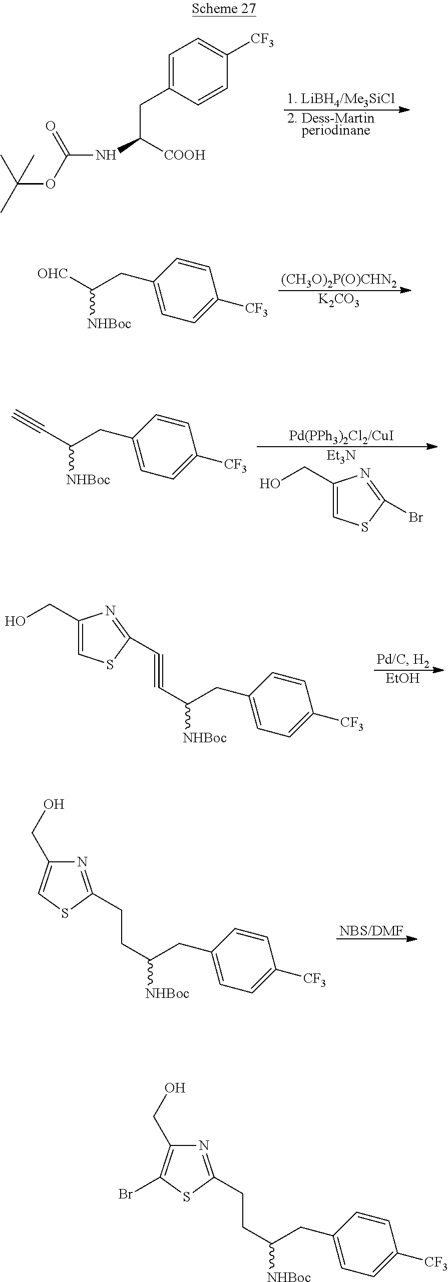

(S)-tert-Butyl 1-hydroxy-3-(3-(trifluoromethyl)phenyl)propan-2-ylcarbamate. In a 500 mL round bottom flask, LiBH₄ (1.0 g, 45 mmol, Fluka) was dissolved in THF (100 mL) at 0° C., and Me₃SiCl (12 mL, 90 mmol, Aldrich) was added dropwise. The reaction mixture was stirred for 20 minutes at 23° C. The mixture was then cooled to 0° C. and (S)-2-(tert-butoxycarbonylamino)-3-(3-(trifluoromethyl)phenyl)propanoic acid (5.0 g, 15 mmol, Chem-Impex catalog number 07390) was added. The reaction mixture was stirred for 2 hours at 23° C. and then cooled to 0° C. and 20 mL of MeOH was added dropwise via an addition funnel., Next, 60 mL of 10 N NaOH was added. The reaction mixture was extracted twice with 70 mL of EtOAc. The organic layers were combined, washed with brine and concentrated. The residue was then purified by silica gel chromatography eluting with 0-40% EtOAc/hexane to give the title compound (3.0 g, 63%). MS m/z: 320 (M+1).

(±)-tert-Butyl 1-oxo-3-(3-(trifluoromethyl)phenyl)propan-2-ylcarbamate. In a 250 mL round bottom flask, (S)-tert-butyl 1-hydroxy-3-(3-(trifluoromethyl)phenyl)propan-2-ylcarbamate (2.5 g, 7.8 mmol) was dissolved in DCM (30 mL). Sodium bicarbonate (6.6 g, 78 mmol) and Dess-Martin periodinane (5.0 g, 12 mmol, Aldrich) were added in one portion. The reaction mixture was stirred at 23° C., and monitored by TLC. After 1 hour, the reaction was quenched with aqueous sodium thiosulphate and sodium bicarbonate, and the resulting mixture was stirred for 1 hour until the biphasic solution was clear. The layers were separated and the aqueous layer was extracted with DCM The combined organic layers were then dried over magnesium sulfate, filtered, and concentrated. The residue was dried under vacuum to give the title compound which was used without further purification. MS m/z: 318 (M+1). ¹H NMR (400 MHz, CDCl₃) δ ppm 1.43 (s, 9H) 3.09-3.30 (m, 2H), 4.40-44.8 (m, 1H), 5.05 (d, J=6.46 Hz, 1H) 7.30 (d, J=8.02 Hz, 2H) 7.57 (d, J=8.02 Hz, 2H) 9.65 (s, 1H).

(±)-tert-Butyl 1-(4-(trifluoromethyl)phenyl)but-3-yn-2-ylcarbamate. In a 150 mL round bottom flask, tert-butyl 1-oxo-3-(4-(trifluoromethyl)phenyl)propan-2-ylcarbamate (1.5 g, 4.8 mmol) was dissolved in dry MeOH (50 mL), and then potassium carbonate (1.3 g, 9.5 mmol) and Ohira reagent (1.1 g, 5.5 mmol, prepared according to Synthetic Communications, 14(2), 155-61; 1984) were added in one portion. The reaction mixture was stirred for 15 hours at 23° C. The reaction mixture was then adsorbed onto silica gel, and purified by silica gel chromatography, eluting with 0-25% EtOAc/hexane to give the title compound (1.4 g, 95%).

(±)-tert-Butyl 4-(4-(hydroxymethyl)thiazol-2-yl)-1-(4-(trifluoromethyl)phenyl)but-3-yn-2-ylcarbamate. In a 20 mL microwave tube, tert-butyl 1-(4-(trifluoromethyl)phenyl)but-3-yn-2-ylcarbamate (0.70 g, 2.23 mmol) was dissolved in TEA (10 mL), and bis(triphenylphosphine)palladium(II) chloride (78.1 mg, 111 mmol, Aldrich) and copper(I) iodide (63.6 mg, 0.33 mmol, Aldrich) were added in one portion. The reaction mixture was heated to 110° C. for 30 minutes in the microwave. The reaction mixture was concentrated. The residue was purified by silica gel chromatography, eluting with 0-50% EtOAc/hexane to give the title compound (0.60 g, 63%). MS m/z: 427 (M+1).

(±)-tert-Butyl 4-(4-(hydroxymethyl)thiazol-2-yl)-1-(4-(trifluoromethyl)phenyl)butan-2-ylcarbamate. To a solution of tert-butyl 4-(4-(hydroxymethyl)thiazol-2-yl)-1-(4-(trifluoromethyl)phenyl)but-3-yn-2-ylcarbamate (0.53 g, 1.24 mmol) in 25 mL of EtOH was added palladium (10 wt. % on activated carbon (0.66 g, 6.21 μmol, Aldrich)). The reaction mixture was stirred under H₂ overnight, and was then filtered through Celite® brand filter aid, eluted with EtOAc, and concentrated under reduced pressure to give the title compound. This product was carried on without any further purification. MS m/z: 431 (M+1).

(±)-tert-Butyl 4-(5-bromo-4-(hydroxymethyl)thiazol-2-yl)-1-(4-(trifluoromethyl)phenyl)butan-2-ylcarbamate. tert-Butyl 4-(4-(hydroxymethyl)thiazol-2-yl)-1-(4-(trifluoromethyl)phenyl)butan-2-ylcarbamate (0.45 g, 1.05 mmol) was treated with NB S (0.19 g, 1.05 mmol, Fluka) in 10 mL of DMF at room temperature for five hours. The reaction mixture was partioned between EtOAc and water. The organic phase was separated and washed with saturated aqueous ammonium chloride twice, dried over sodium sulfate and filtered. The organic layer was concentrated and the residue was purified by silica gel chromatography, eluting with 0-40% EtOAc/hexane to give the title compound (0.46 g, 86%). MS m/z: 509 (M+1).

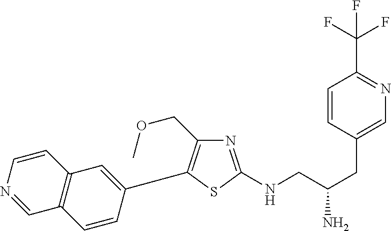

### Comparative Example 18′

### (S)—N-1-(5-(Isoquinolin-6-yl)-4-(methoxymethyl)thiazol-2-yl)-3-(6-(trifluoromethyl)pyridin-3-yl)propane-1,2-diamine

The title compound was synthesized in a manner similar to that described for Example 18, but using isoquinolin-6-ylboronic acid hydrochloride instead of 3-fluoroisoquinolin-6-ylboronic acid. MS m/z: 474 (M+1). ¹H NMR (400 MHz, CD₃OD): δ ppm 3.13-3.25 (m, 2H) 3.48 (s, 3H) 3.60-3.66 (m, 1H) 3.79-3.71 (m, 1H) 3.94-3.40 (m, 1H) 4.49 (s, 2H) 7.85 (d, J=8.02 Hz, 1H) 8.00 (dd, J=7.83, 1.17 Hz, 1H) 8.06 (s, 1H) 8.17 (s, 1H) 8.26 (d, J=6.46 Hz, 1H) 8.40-8.42 (m, 1H) 8.53 (d, J=6.26 Hz, 1H) 8.73 (s, 1H) 9.56 (s, 1H).

Isoquinolin-6-ylboronic acid hydrochloride was prepared as shown in Scheme 28 starting from commercially available 6-bromoisoquinoline.

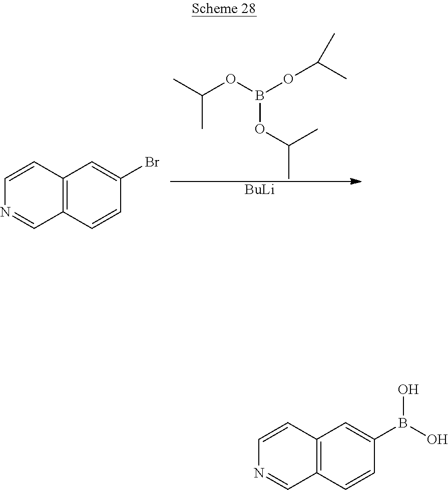

Isoquinolin-6-ylboronic acid hydrochloride. A flame-dried 100 mL round bottom flask was charged with 10 mL THF, triisopropyl borate (1 g, 5.8 mmol, Aldrich) and 6-bromoisoquinoline (1 g, 4.8 mmol, Astatech). The mixture was cooled to −78° C., and n-butyllithium (3.6 mL, 5.8 mmol, Aldrich) was added dropwise to the reaction over about 1 hour. The mixture was stirred for 0.5 hours at −78° C. and then warmed to −20° C. After 5.0 mL 2.0 N HCl was added to the reaction mixture, it was concentrated under reduced pressure in a rotary evaporator until a precipitate formed. The white solid (0.6 g) HCl salt of isoquinolin-6-ylboronic acid was recovered by filtration. The product was used directly without further purification. LCMS (API-ES) m/z (%): 174 (100%, M⁺+H).

### 2.1 PKB Assay Testing

The kinase assay for evaluating PKB activity comprises active PKB enzymes, a PKB specific substrate, and P³³-labeled ATP. Two form of PKBα enzymes were used, the full length PKBα and a kinase domain of PKBα with pleckstrin domain (amino acids 1-117) deleted. Both PKB enzymes were obtained from Upstate cell signaling solutions (Cat.# 14-276 and 14-341). The PKB substrate used is a synthetic peptide (ARKRERTYSFGHHA (SEQ ID NO: 1)) as described in Obata et al., J. Biol. Chem. 275 (46), 36108-36115 (2000). The phosphorylated substrate was captured by a phosphocellulose membrane filter plate (MILLIPORE) and measured by a Wallac Microbeta liquid scintillation counter (Perkin Elmer). Table 1 provides the IC₅₀ values obtained for each of the examples with respect to PKBα.

PKB activity in cells was assayed in a PTEN null human breast tumor cell line MDA-MB-468 and U87-MG. The phosphorylation status of PKB substrate PRAS40, FKHRL1, GSK3a/b, and Tuberin were measured by immunoassays utilizing phospho-specific antibodies (Invitrogen, Cell signaling technology).

The effect of PKB inhibition on cell viability was measured in a range of human tumor cell lines including, but not limiting to, MDA-MB-468, MDA-MB-231, U87-MG, LN-229, PC3, DU145. The cells were treated in regular growth media for 72 hours and cell viability was measured by Alamar Blue (Invitrogen).

The effect of PKB inhibition on tumor growth in vivo was assessed in an established U87MG xenograft model for Examples 2 and 18. Athymic nude mice bearing U87MG tumors (approximately 200 mm³) in the right flank were treated with the compound orally at the dosage of 10, 30, 60, and 100 mg/kg/day (n=10) for 13 days (Example 2) and for 10 days and 17 days (Example 18). Tumor volume and body weight were measured twice per week. Data were expressed as means plus or minus standard errors and plotted as a function of time. Statistical significance of the effect was evaluated by Repeated Measures Analysis of Variance (RMANOVA) followed by Scheffe post hoc testing for multiple comparisons. Dose dependent tumor growth inhibition was observed in the studies with Examples 2 and 18.

TABLE 1

| Example | Structure^{a} | IC₅₀^{b} |
|---|---|---|
| 1 | 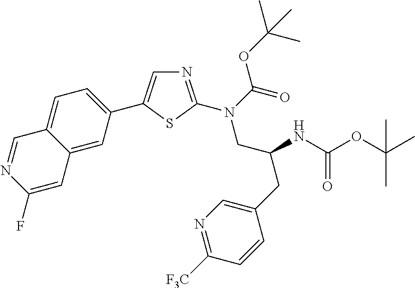 | ++ |
| 2 | 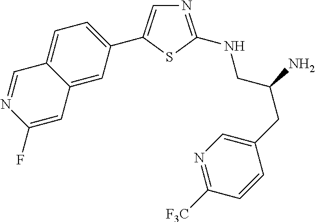 | ++++ |
| 3 | 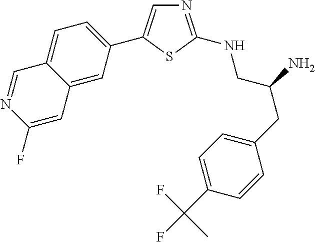 | ++++ |
| 4 | 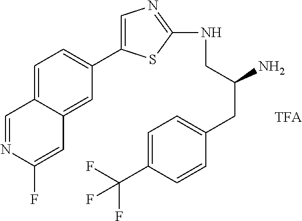 | ++++ |
| 5 | 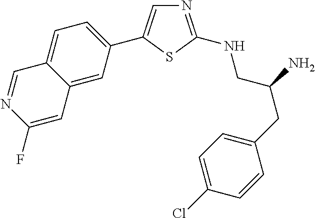 | ++++ |
| 6 | 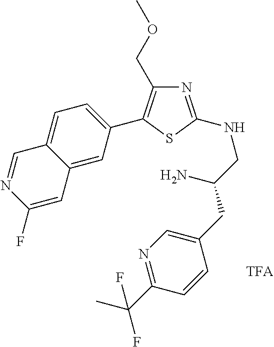 | ++++ |
| 7 | 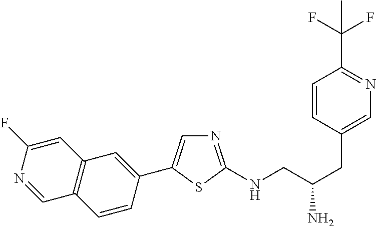 | ++++ |
| 8 | 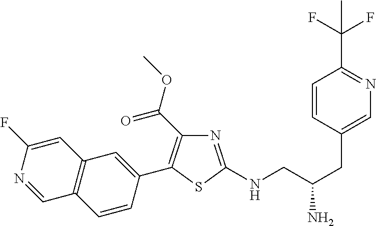 | ++++ |
| 9 | 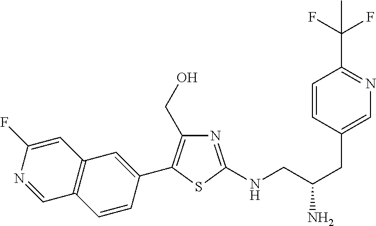 | ++++ |
| 10 | 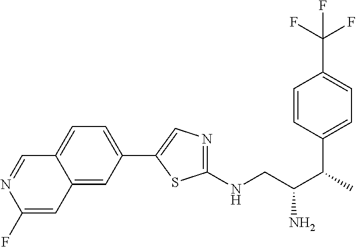 | ++++ |
| 11 | 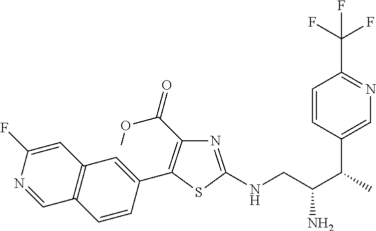 | +++ |
| 12 | 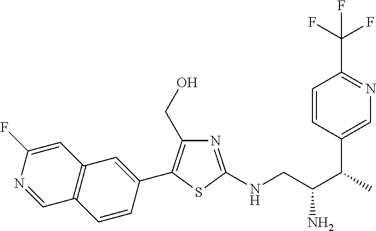 | ++++ |
| 13 | 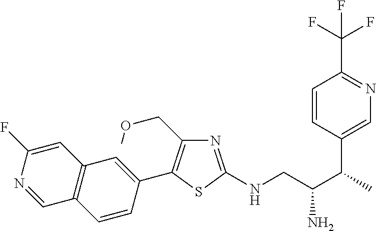 | ++++ |
| 14 | 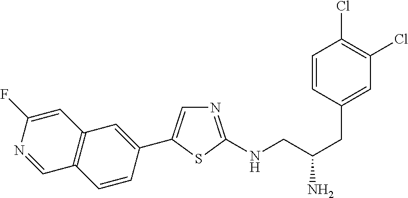 | ++++ |
| 15 | 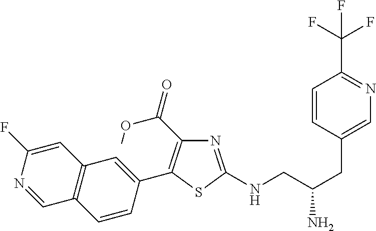 | ++++ |
| 16 | 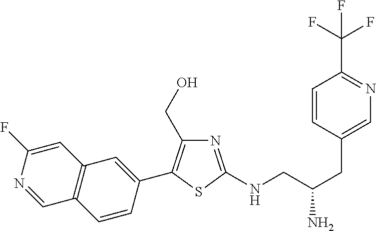 | ++++ |
| 17 | 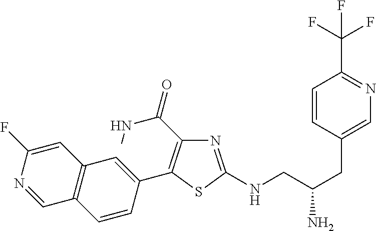 | +++ |
| 18 | 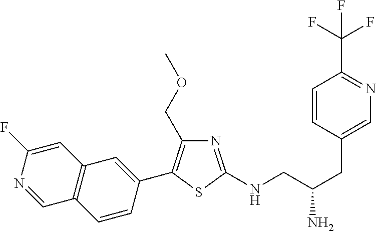 | ++++ |
| 19 | 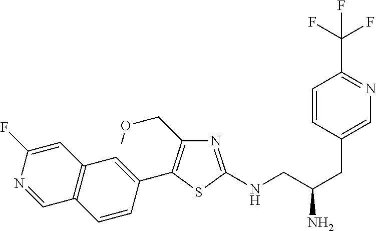 | ++ |
| 20 | 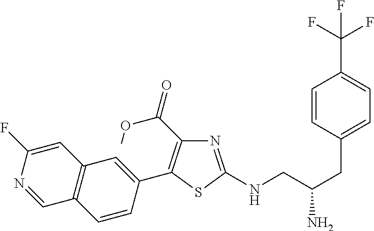 | ++++ |
| 21 | 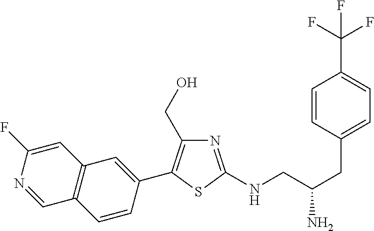 | ++++ |
| 22 | 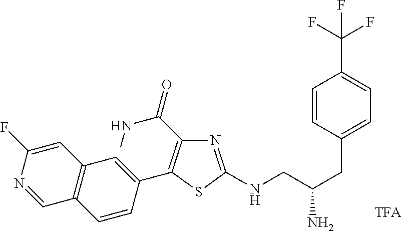 | +++ |
| 23 | 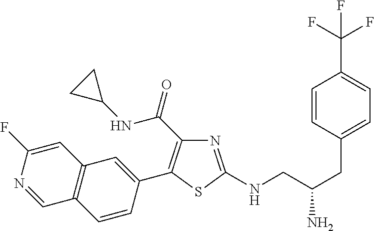 | ++ |
| 24 | 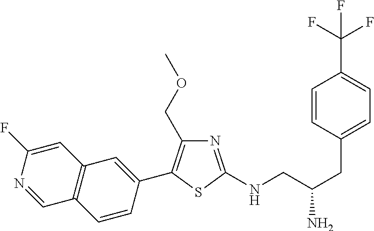 | ++++ |
| 25 | 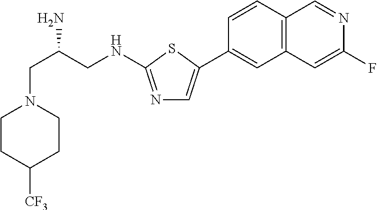 | +++ |
| 26 | 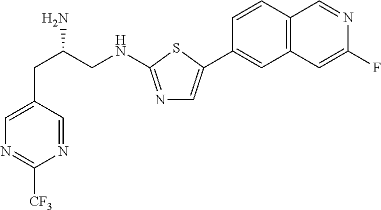 | ++++ |
| 27 | 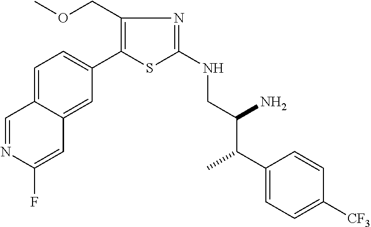 | ++++ |
| 28 | 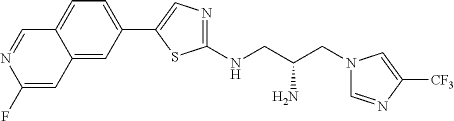 | +++ |
| 29 | 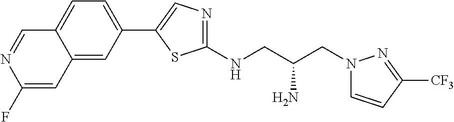 | +++ |
| 30 | 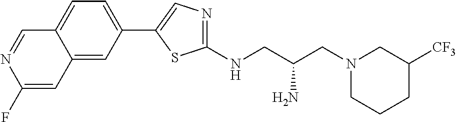 | ++ |
| 31 | 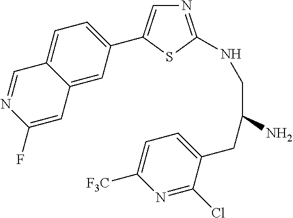 | ++++ |
| 32 | 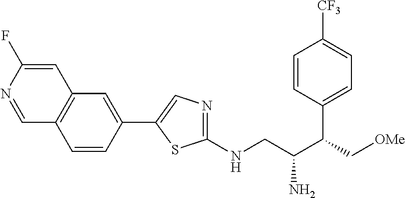 | ++++ |
| 33 | 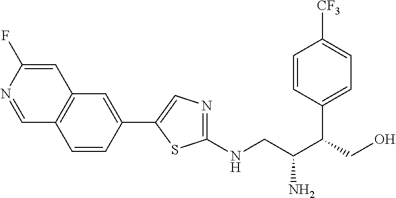 | ++++ |
| 34 | 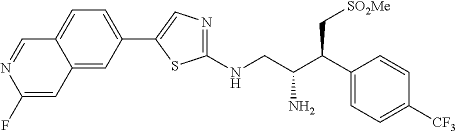 | +++ |
| 35 | 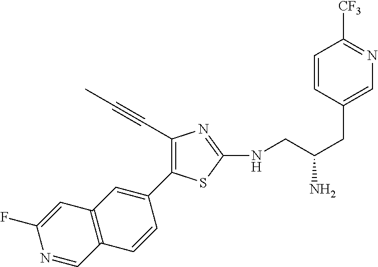 | ++++ |
| 36 | 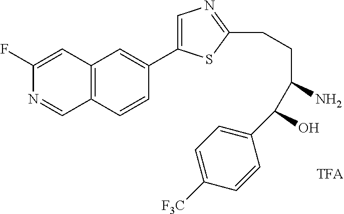 | ++++ |
| 37 | 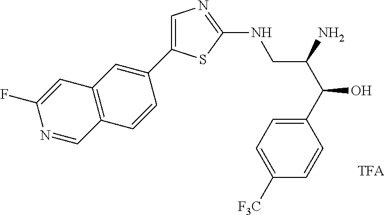 | ++++ |
| 38 | 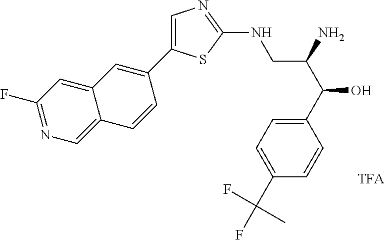 | ++++ |
| 39 | 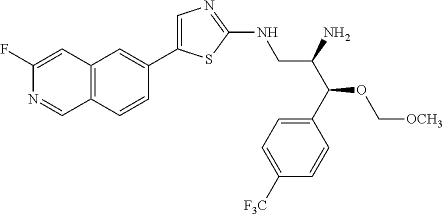 | ++++ |
| 40 | 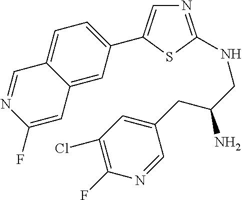 | +++ |
| 41 | 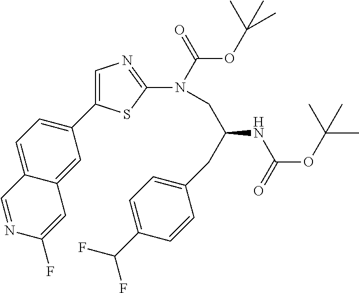 | ++ |
| 42 | 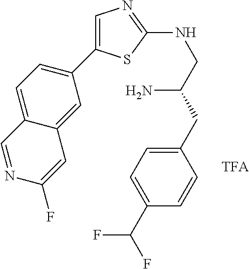 | ++++ |
| 43 | 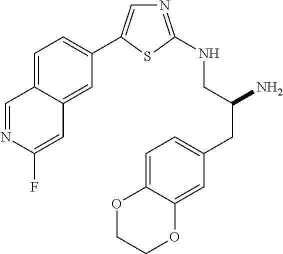 | ++++ |
| 44 | 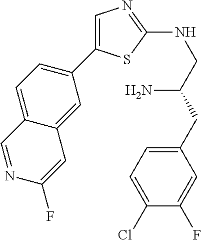 | ++++ |
| 45 | 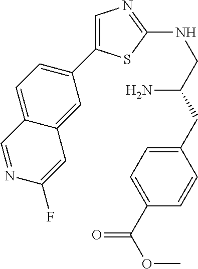 | +++ |
| 46 | 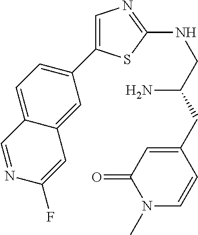 | +++ |
| 47 | 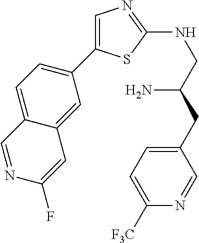 | ++ |
| 48 | 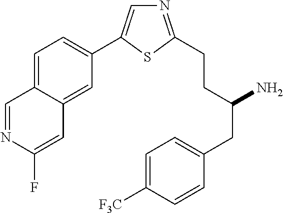 | ++ |
| 49 | 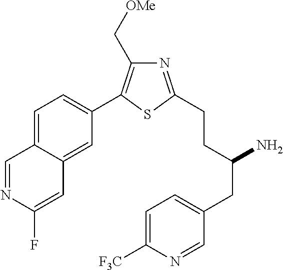 | ++++ |
| 50 | 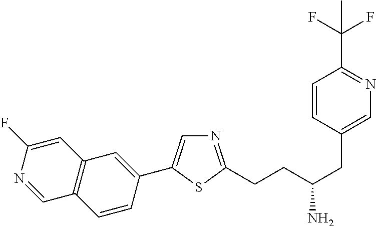 | ++++ |
| 51 | 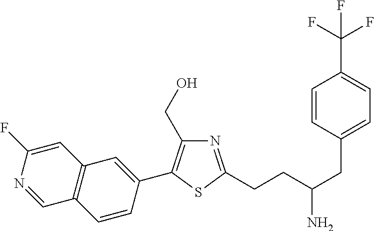 | +++ |

^{a}When the stereochemistry is not specified at a carbon bonded to four different groups, this indicates a mixture of stereoisomers is present.
^{b}IC₅₀ Ranges:
+ IC₅₀ > 10 μM
++ 1 μM ≦ IC₅₀ ≦ 10 μM
+++ 0.05 μM ≦ IC₅₀ < 1 μM
++++ IC₅₀ < 0.05 μM

Each of the compounds in the above table and tautomers, salts, neutral forms, solvates including hydrates, and stereoisomers thereof is preferred both individually and as a member of a group. Each of the groups in these compounds that corresponds to any of the variables in the compounds of Formula I is also preferred.

The following table provides comparative examples identical to the Examples of the present invention except that they do not include the fluorine substituent on the isoquinoline ring.

TABLE 2

| Comparative | | Synthesis |
|---|---|---|
| Example | Structure^{a} | Reference |
| 2′ | 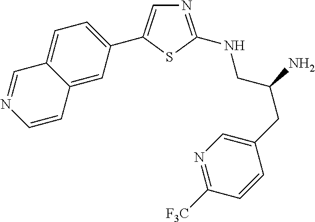 | Example 170^{b} |
| 4′ | 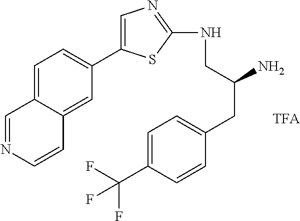 | Example 6^{b} |
| 7′ | 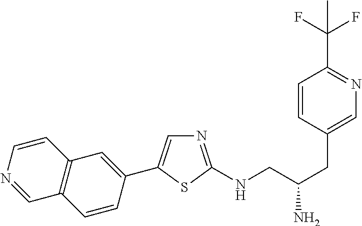 | Synthesized using the same procedure as Example 7, but using isoquinolin-6- ylboronic acid hydrochloride |
| 10′ | 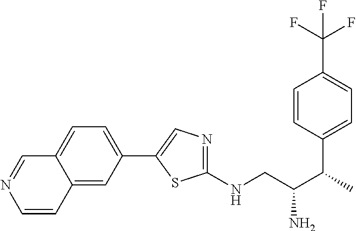 | Example 110^{b} |
| 14′ | 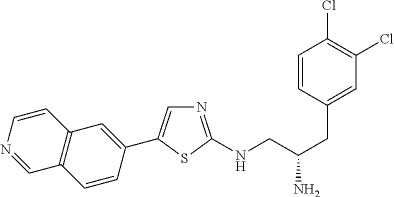 | Example 132^{b} |
| 18′ | 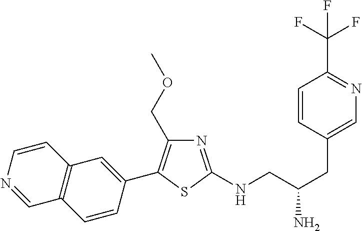 | See preparation provided herein |
| 20′ | 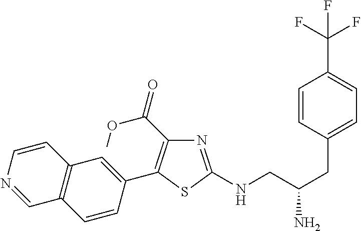 | Example 117^{b} |
| 21′ | 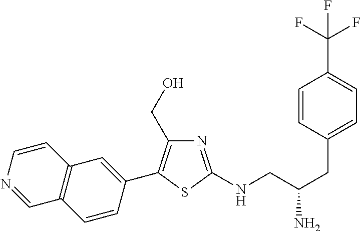 | Example 10^{b} |
| 24′ | 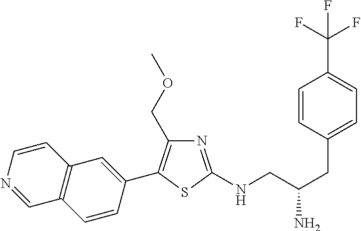 | Example 13^{b} |
| 25′ | 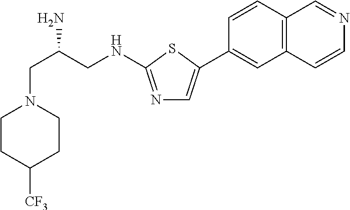 | Synthesized using the same procedure as Example 25, but using isoquinolin-6- ylboronic acid hydrochloride |
| 26′ | 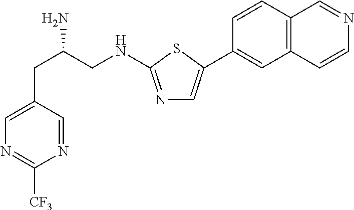 | Synthesized using the same procedure as Example 26, but using isoquinolin-6- ylboronic acid hydrochloride |
| 33′ | 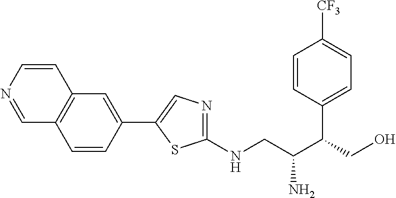 | Example 159^{b} |
| 36′ | 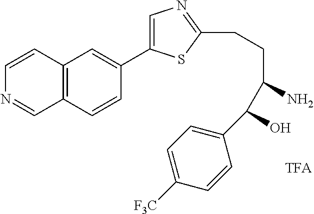 | Synthesized using the same procedure as Example 36, but using isoquinolin-6- ylboronic acid hydrochloride |
| 37′ | 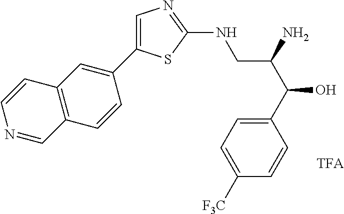 | Synthesized using the same procedure as Example 37, but using the thiazole compound prepared from 6- bromoisoquinoline |
| 38′ | 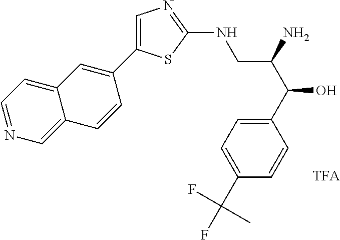 | Synthesized using the same procedure as Example 38, but using isoquinolin-6- ylboronic acid hydrochloride |
| 42′ | 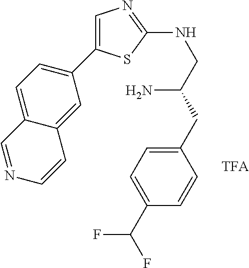 | Synthesized using the same procedure as Example 42, but using isoquinolin-6- ylboronic acid hydrochloride |
| 43′ | 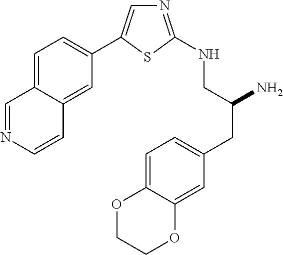 | Synthesized using the same procedure as Example 43, but using isoquinolin-6- ylboronic acid hydrochloride |
| 44′ | 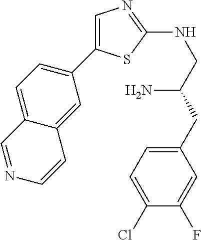 | Example 85^{b} |
| 45′ | 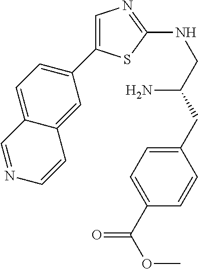 | Synthesized using the same procedure as Example 45, but using isoquinolin-6- ylboronic acid hydrochloride |
| 46′ | 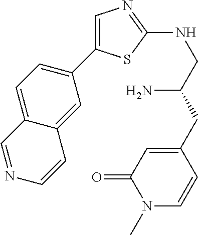 | Synthesized using the same procedure as Example 46, but using isoquinolin-6- ylboronic acid hydrochloride |
| 50′ | 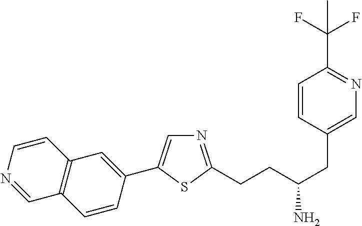 | Synthesized using the same procedure as Example 50, but using isoquinolin-6- ylboronic acid hydrochloride |
| 51′ | 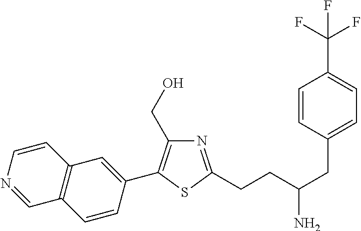 | Synthesized using the same procedure as Example 51, but using isoquinolin-6- ylboronic acid hydrochloride |

^{a}When the stereochemistry is not specified at a carbon bonded to four different groups, this indicates a mixture of stereoisomers is present.
^{b}This is the Example number of the Comparative Example compound in U.S. Patent Publication No. US 2007/0173506, published July 25, 2007, and incorporated herein by reference in its entirety and for all purposes as if specifically set forth in its entirety.

Cytochrome P450 Inhibition Assays

Materials

Midazolam was obtained from BD Gentest (Waltham, Mass.); 1′ hydroxy midazolam and 1-hydroxy bufuralol maleate were obtained from Ultrafine Chemicals (Manchester, UK); bufuralol HCl, ketoconazole, quinidine, potassium phosphate, and NADPH were obtained from Sigma (St. Louis, Mo.). Test compounds were prepared at 10 mM concentration in DMSO (Mallinckrodt Inc., St. Louis, Mo.). NADPH was prepared at 10 mM concentration in a pH 7.4 buffer (8.3 mg/mL 66.7 mM potassium phosphate). Midazolam was prepared at 1 mM concentration in deionized water (Amgen, Thousand Oaks, Calif.), and bufuralol was prepared at 2 mM concentration in deionized water. A stock solution of ketoconazole was prepared at 37 mM concentration in DMSO, and quinidine was prepared at 500 uM concentration in DMSO. Formic acid in acetonitrile (0.05%, ACN) was used as a quench solution (Sigma-Aldrich, St. Louis, Mo.). All solvents used for LC/MS were of chromatographic grade. Pooled human liver microsomes (Lot 0610351) were purchased from XenoTech LLC, (Lenexa, Kans.).

CYP3A4 Assay

Pooled human liver microsomes (0.1 mg/mL) were incubated at 37° C. in a phosphate buffer (66.7 mM potassium phosphate at pH 7.4) with the selective CYP3A substrate midazolam at a concentration of 2.5 μM in the presence and absence of test compound (3 μM). The reaction was started with the addition of NADPH (1 mM final concentration). The reaction was stopped after 10 minutes by addition of formic acid in acetonitrile. 1′-Hydroxymidazolam metabolite formation was measured by an HPLC MS detection method (mobile phases were 0.1% AcOH in 5% MeOH, and 0.1% AcOH in 95% MeOH; the HPLC column was an Onyx Monolithic C18 CHO-7645 HPLC column obtained from Phenomenex; Shimadzu LC-10AD_{VP} equipped Biomek FX Liquid handling system and CTC Analytics PAL well auto-sampler; Mass spectrometer: Applied Biosystems API 3000 using Analyst 1.4.1 software). Inhibition was determined by the ratio of the amount of 1′-hydroxy midazolam metabolite formed in the presence of test compound to the amount of metabolite found in the absence of test compound Inhibition was measured relative to the rate of formation of 1′-hydroxy midazolam incubated at 2.5 μM without test compound.

CYP2D6 Assay

Pooled human liver microsomes (0.25 mg/mL) were incubated at 37° C. in a phosphate buffer (66.7 mM potassium phosphate at pH 7.4) with the selective CYP2D6 substrate bufuralol at a concentration of 5 μM in the presence and absence of test compound (3 μM). The reaction was started with the addition of NADPH (1 mM final concentration). The reaction was stopped after 10 minutes by addition of formic acid in acetonitrile. 1-Hydroxybufuralol metabolite formation was measured by an HPLC MS detection method (mobile phases for HPLC were: 0.1% AcOH in 5% MeOH, and 0.1% AcOH in 95% MeOH; the HPLC column was an Onyx Monolithic C18 CHO-7645 HPLC column from Phenomenex; Shimadzu LC-10AD_{VP} equipped Biomek FX Liquid handling system and CTC Analytics PAL well auto-sampler; Mass spectrometer: Applied Biosystems API 3000 using Analyst 1.4.1 software). Inhibition was determined by the ratio of the amount of 1-hydroxy bufuralol metabolite formed metabolite in the presence of test compound to the amount of metabolite in the absence of test compound.

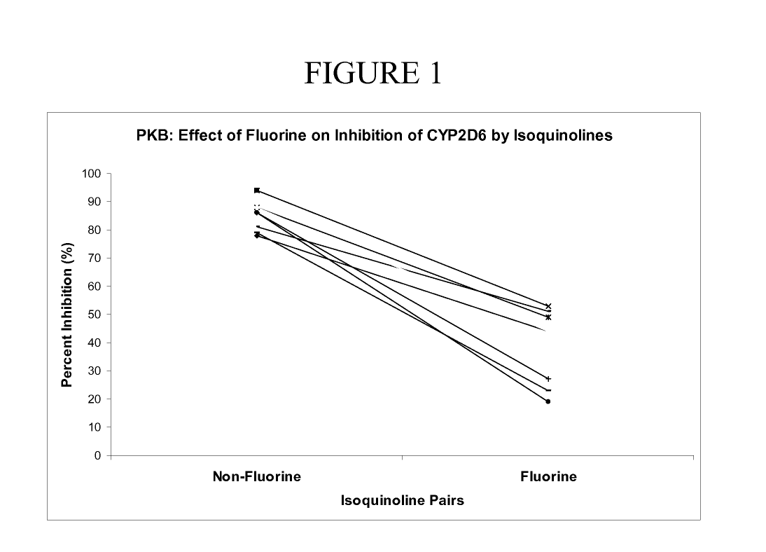

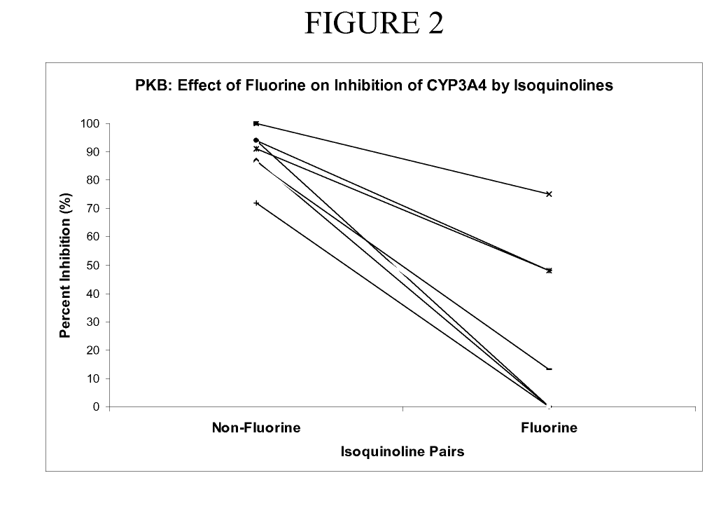

Tables 3 and 4 provide data showing that the addition of the fluorine group on the isoquinoline group resulted in surprisingly dramatic and highly favorable decreases in inhibition of CYP2D6 and CYP3A4. This is graphically illustrated in FIGS. 1 and 2. The decrease in inhibition is particularly true with respect to inhibition of CYP3A4.

TABLE 3

| | |
|---|---|
| Isoquinoline Thiazole | Fluoroisoquinoline Thiazole |
| Compounds | Compounds |

| Comparative | CYP2D6 | Example | CYP2D6 |
|---|---|---|---|
| Example No | Inhibition (%) | No. | Inhibition (%) |
| 4′ | 78 | 4 | 44 |
| 24′ | 94 | 24 | ND^{a} |
| 44′ | 75 | 44 | 42 |
| 2′ | 94 | 2 | 53 |
| 33′ | 88 | 33 | 49 |
| 10′ | 86 | 10 | 19 |
| 36′ | 86 | 36 | 27 |
| 25′ | 81 | 25 | 51 |
| 7′ | 79 | 7 | 23 |
| 38′ | 88 | 38 | 44 |
| 18′ | ND^{a} | 18 | 75 |
| 50′ | ND^{a} | 50 | 71 |

^{a}ND means this measurement was not determined

TABLE 4

| | |
|---|---|
| Isoquinoline Thiazole | Fluoroisoquinoline Thiazole |
| Compounds | Compounds |

| Comparative | CYP3A4 | Example | CYP3A4 |
|---|---|---|---|
| Example No | Inhibition (%) | No. | Inhibition (%) |
| 4′ | 87 | 4 | 0 |
| 24′ | 100 | 24 | ND^{a} |
| 44′ | 94 | 44 | 48 |
| 2′ | 100 | 2 | 75 |
| 33′ | 91 | 33 | 48 |
| 10′ | 94 | 10 | 0 |
| 36′ | 72 | 36 | 0 |
| 25′ | 94 | 25 | 48 |
| 7′ | 86 | 7 | 13 |
| 38′ | 86 | 38 | 9 |
| 18′ | ND^{a} | 18 | 0 |
| 50′ | ND^{a} | 50 | 21 |

^{a}ND means this measurement was not determined

In VivoPharmacokinetics

In vivo pharmacokinetic parameters were determined by measuring systemic plasma exposure in male Sprague-Dawley rats (Charles River Laboratories or Taconic, Oxnard, Calif.) by intravenous administration (N=3 fed rats: 2 mg/kg tail vein injection in DMSO) and oral gavage (N=3 fasted rats: 5 mg/kg in 1% Tween 80-2% HPMC-97% deionized water-pH 2.0 vehicle) (Comparative Example 18′ was only dosed IV and at 0.5 mg/kg). DMSO was obtained from Mallinckrodt Inc.; Tween 80 was obtained from Croda; HPMC is Methocel E50 Premium LV, obtained from Dow Excipients. Following oral administration, blood was drawn (250 μL) at 0.25, 0.5, 1, 2, 4, 6, 8, 12, and 16 hours. Following iv administration, blood was drawn (250 μL) at 0.083, 0.25, 0.5, 1, 2, 4, 6, 8, and 16 hours. Blood samples were drawn using a Culex® In Vivo Sampling System (BASi) and collected in BD Microtainer® tubes containing Lithium Heparin (BD). Plasma was obtained by centrifugation of blood at 14000 g and 2-8° C. for five minutes using an Eppendorf 5417R Centrifuge System. Plasma samples were mixed with 2× volume of acetonitrile (Sigma-Aldrich, St. Louis, Mo.). The mixture was stirred briefly using a vortex mixer and then centrifuged at 2867 g for 5 minutes in a Beckman Coulter Allegra 25R equipped with a Type S5700 rotor. The supernatant was removed and diluted 2× with mobile phase A (5/94.9/0.1 v/v/v MeOH/water/AcOH). Plasma concentrations were determined using liquid chromatography/tandem mass spectrometry, with electrospray ionization (ESI) and multiple reaction monitoring in the positive ion mode (Sciex API 3000 mass spectrometer connected to a Shimadzu LC-20AD pump and a LEAP PAL autosampler. Typical conditions used for HPLC were: mobile phase A=5/94.9/0.1 v/v/v MeOH/water/AcOH and mobile phase B=95/4.9/0.1 v/v/v MeOH/water/AcOH; HPLC column was a Varian MonoChrom C18.3μ for 2×30 mm and 51.1 for 2×50 mm) Instrument response was calibrated by a standard curve over 0.1-2000 ng/mL concentration range, and the lower limit of quantitation was 0.1-2.5 ng/mL. Peak areas were integrated on a Windows 2000 computer using the Sciex program Analyst® (Version 1.4.1). Following peak area integration, the data were exported to the software package (Watson® Non-GLP PROD (Version 7.0.0.01, Thermo Electron Corp., Waltham, Mass.)), where concentrations were determined by a weighted linear regression of peak area versus the nominal concentrations of the calibration standards. Calculations were performed on unrounded numbers. The Watson® Non-GLP PROD software was used to determine precision and accuracy for the calibration standards. Noncompartmental analysis of plasma concentration data was used to generate PK parameters such as clearance (CL, L/h/kg), volume of distribution (Vss, L/kg), area under the curve (AUC, ng·h/mL) and secondary parameters such as half-life (t1/2, h) and oral bioavailability (% F, %). This was done using WinNonlin Enterprise software (Version 5.1.1, from Pharsight Corp., Mountain View, Calif.). References for pharmacokinetic analysis include: Gabrielsson and Weiner (1997) Pharmacokinetic and Pharmacodynamic Data Analysis Concepts and Applications, 2nd ed., Swedish Pharmaceutical Press, Stockholm; Gibaldi and Perrier (1982) Pharmacokinetics, 2nd ed., Marcel Dekker, New York. Rowland and Tozer (1995) Clinical Pharmacokinetics: Concepts and Applications, 3^{rd} ed., Lippincott Williams & Wilkins, Philadelphia.

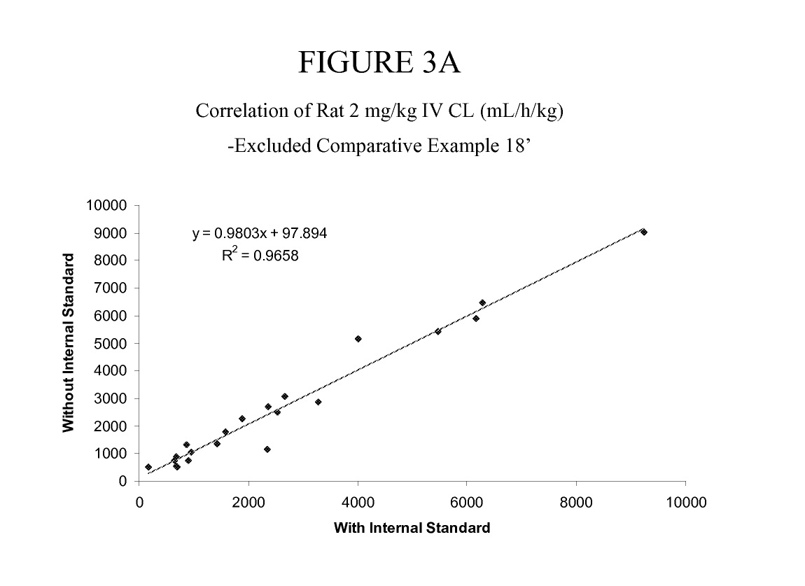

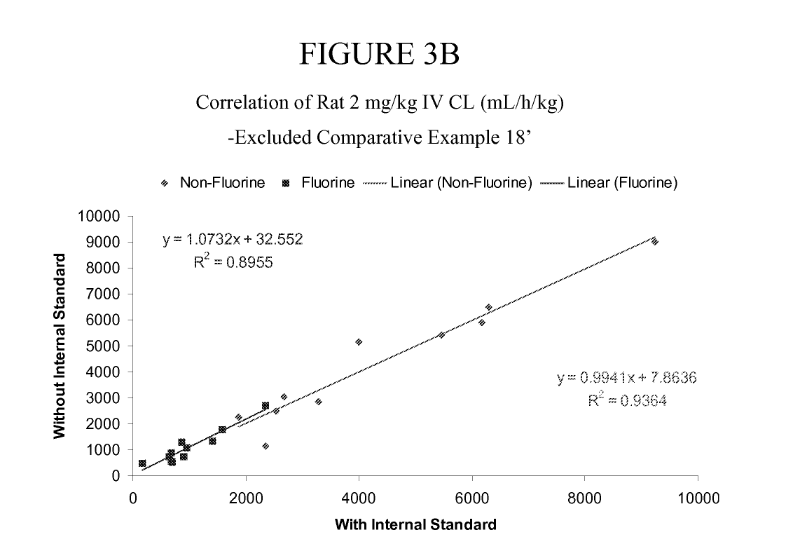

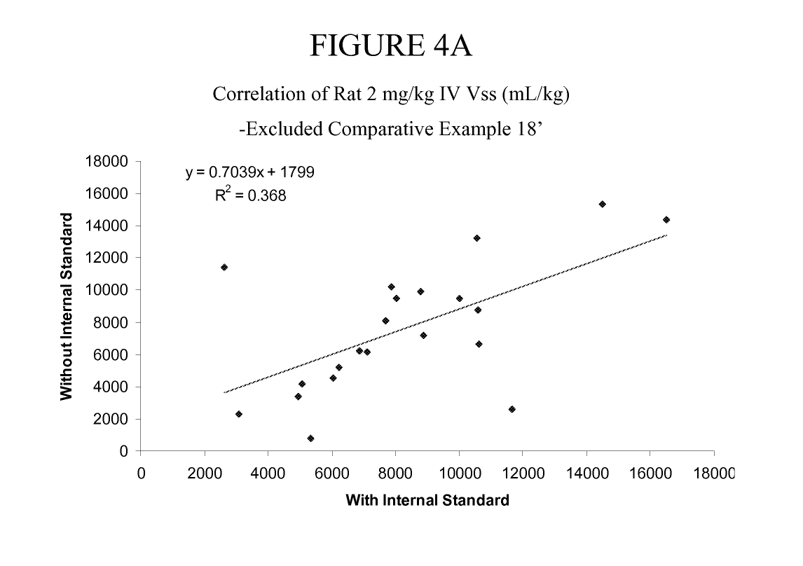

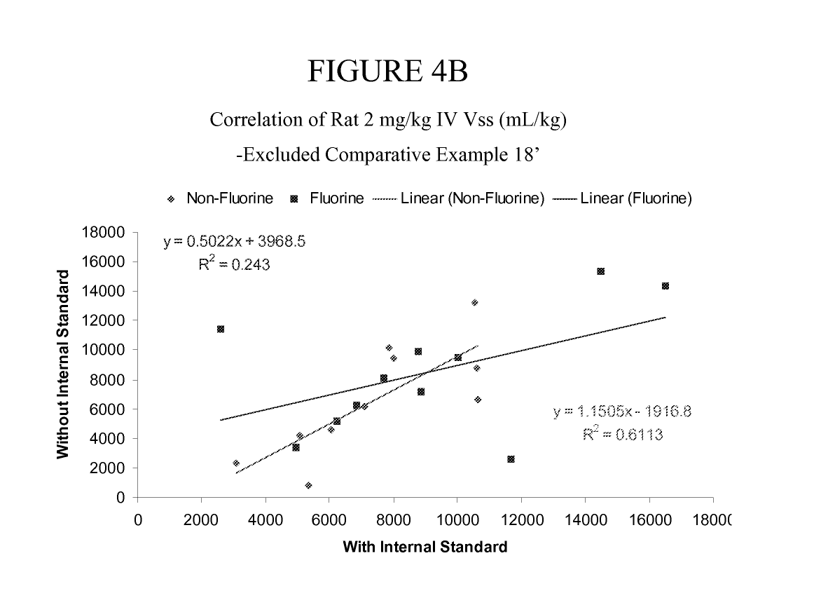

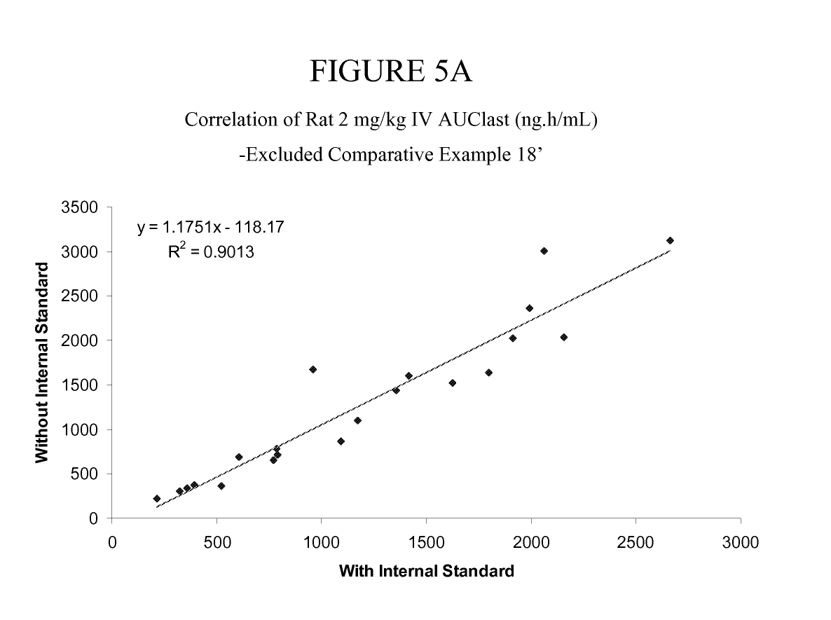

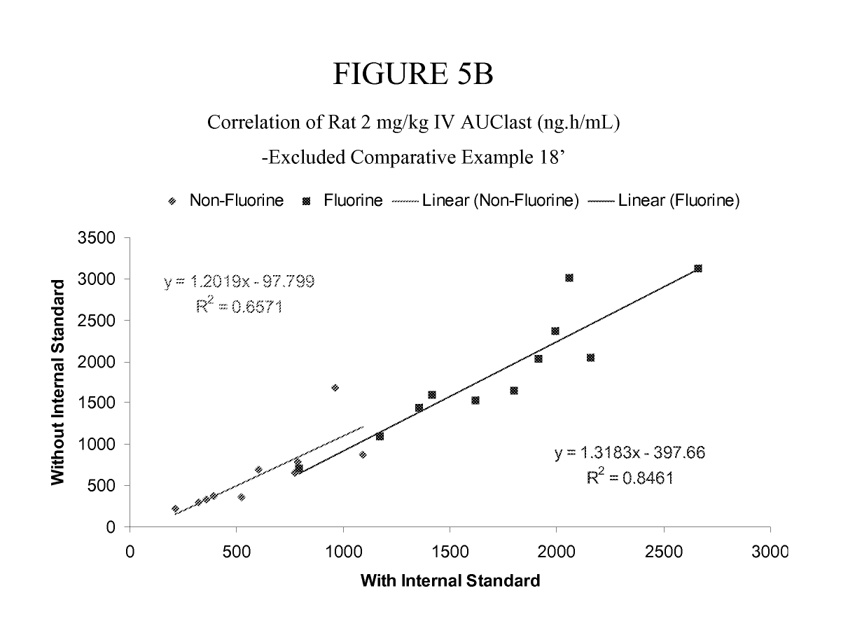

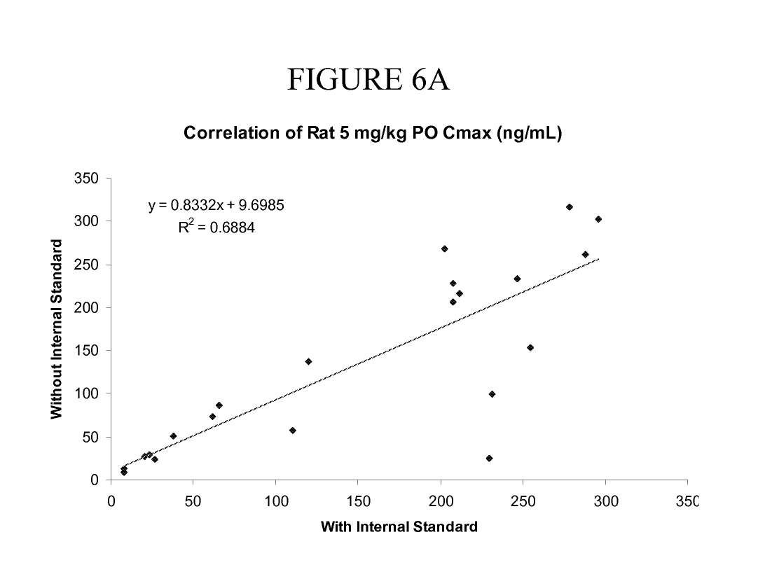

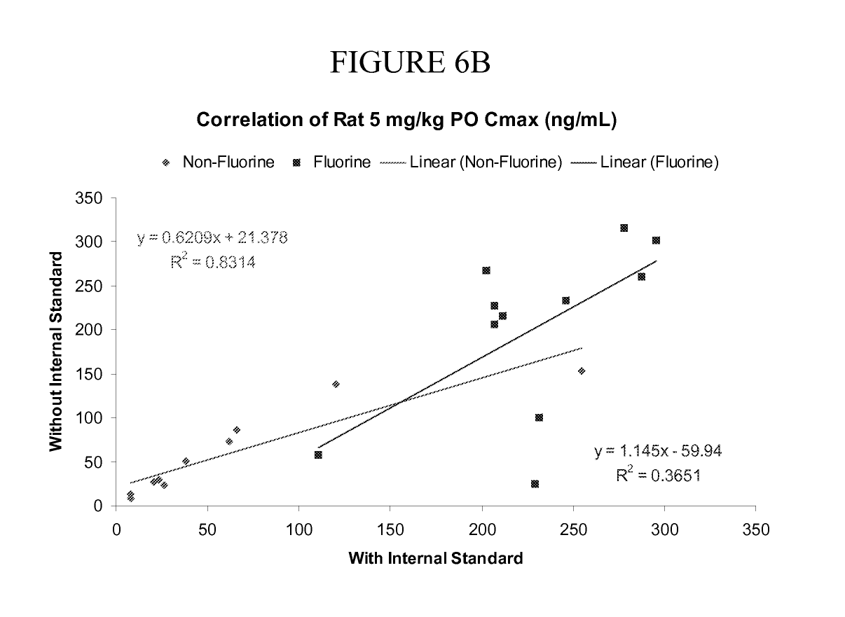

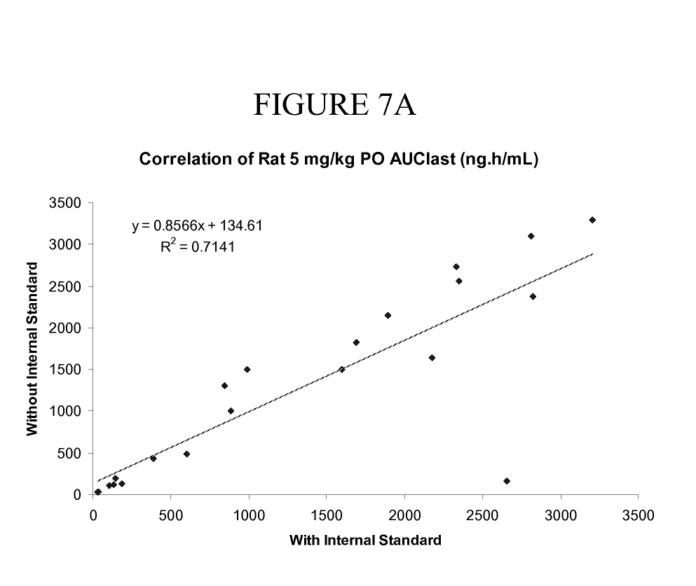

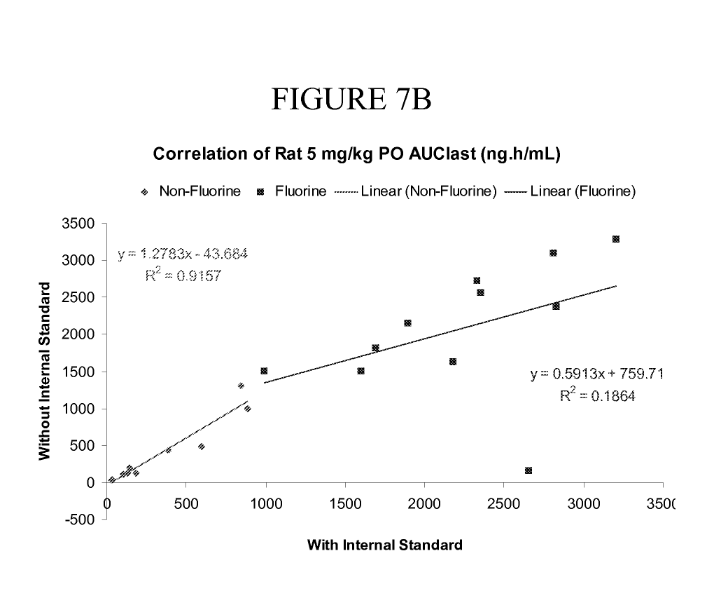

Tables 5 and 6 provide in vivo pharmacokinetic data for Examples of the invention and Comparative Examples. As shown in Tables 5 and 6, where matching pairs of Example and Comparative Example compounds are available, the oral bioavailability was significantly improved in the compounds with the fluorine group (the fluoroisoquinoline substituted thiazole compounds) in all cases except for the Example 25 and Comparative Example 25′ isoquinoline and fluoroisoquinoline pair. As shown in FIGS. 3A, 3B, 4A, and 4B, the clearance (CL) with and without a bioanalytical internal standard correlated well and is lowered by addition of the fluorine group although the volume of distribution (Vss) was not markedly impacted by the fluorine group. As shown in FIGS. 5A and 5B, fluorine substitution increased the IV plasma exposure of the compounds in rats. Finally, as shown in FIGS. 6A, 6B, 7A, and 7B, fluorine substitution increased the oral plasma exposure of the thiazole compounds in rats. The PK plots shown in FIGS. 3A, 3B, 4A, 4B, 5A, 5B, 6A, 6B, 7A, and 7B are with and without an internal standard.

TABLE 5

| | | | | | | | | | |
|---|---|---|---|---|---|---|---|---|---|
| Example or | IV | PO | IV | | | IV | Oral | Oral | |
| Comparative | Dose | Dose | Cmax | CL | Vss | AUClast | Cmax | AUClast | FPO |
| Example No. | (mg/kg) | (mg/kg) | (ng/mL) | (mL/h/kg) | (mL/kg) | (ng · h/mL) | (ng/mL) | (ng · h/mL | (%) |

Isoquinoline Thiazole Compound Data

| | | | | | | | | | |
|---|---|---|---|---|---|---|---|---|---|
| 4′ | 2 | 5 | 422 | 3052 | 6169 | 651 | 86 | 432 | 27 |
| 24′ | 2 | 5 | 465 | 5405 | 10191 | 369 | 51 | 129 | 14 |
| 44′ | 2 | 5 | 816 | 2488 | 766 | 787 | 138 | 1000 | 51 |
| 2′ | 2 | 5 | 1258 | 2242 | 2323 | 870 | 29 | 117 | 5 |
| 33′ | 2 | 5 | 797 | 2853 | 4182 | 691 | 23 | 199 | 12 |
| 10′ | 2 | 5 | 730 | 1145 | 9460 | 1679 | 73 | 487 | 12 |
| 36′ | 2 | 5 | 347 | 9014 | 13249 | 221 | 27 | 113 | 20 |
| 25′ | 2 | 5 | 284 | 5135 | 8779 | 359 | 153 | 1308 | 146 |
| 7′ | 2 | 5 | 490 | 5876 | 4551 | 336 | 8 | 30 | 4 |
| 38′ | 2 | 5 | 412 | 6477 | 6665 | 299 | 13 | 30 | 4 |
| 18′ | 0.5 | ND | 73 | 14234 | 6441 | 33 | 0 | 0 | ND |

Fluoroisoquinoline Thiazole Compound Data

| | | | | | | | | | |
|---|---|---|---|---|---|---|---|---|---|
| 4 | 2 | 5 | 959 | 1297 | 2586 | 1527 | 25 | 165 | 4 |
| 2 | 2 | 5 | 531 | 728 | 5189 | 2366 | 302 | 2378 | 40 |
| 36 | 2 | 5 | 210 | 877 | 14379 | 1600 | 206 | 2565 | 64 |
| 38 | 2 | 5 | 473 | 512 | 9478 | 3011 | 100 | 2732 | 36 |
| 33 | 2 | 5 | 803 | 541 | 3407 | 3126 | 268 | 2153 | 28 |
| 10 | 2 | 5 | 426 | 752 | 7170 | 2041 | 316 | 3105 | 61 |
| 7 | 2 | 5 | 432 | 1334 | 6224 | 1438 | 216 | 1504 | 42 |
| 18 | 2 | 5 | 422 | 1058 | 8079 | 1643 | 233 | 1637 | 40 |
| 25 | 2 | 5 | 396 | 1796 | 9880 | 1096 | 58 | 1505 | 55 |
| 44 | 2 | 5 | 292 | 492 | 11445 | 2029 | 261 | 3292 | 65 |
| 50 | 2 | 5 | 165 | 2707 | 15348 | 709 | 228 | 1822 | 103 |

Comparative Example 18′ appears to be an outlier (lower IV dosage; no oral PK).

TABLE 6

| | |
|---|---|
| Isoquinoline Thiazole | Fluoroisoquinoline Thiazole |
| Compounds | Compounds |

| Comparative | | Example | |
|---|---|---|---|
| Example No | Fpo (%) | No. | Fpo (%) |
| 4′ | 20 | 4 | 65 |
| 24′ | 19 | 24 | ND^{a} |
| 44′ | 45 | 44 | 67 |
| 2′ | 5 | 2 | 57 |
| 33′ | 10 | 33 | 28 |
| 10′ | 25 | 10 | 52 |
| 36′ | 19 | 36 | 66 |
| 25′ | 65 | 25 | 34 |
| 7′ | 3 | 7 | 47 |
| 38′ | 4 | 38 | 45 |
| 18′ | ND^{a} | 18 | 48 |
| 50′ | ND^{a} | 50 | 85 |

^{a}ND means this measurement was not determined

The foregoing has demonstrated the pertinent and important features of the present invention. Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

All references cited herein are incorporated herein by reference in their entireties and for all purposes as if specifically set forth herein and to the same extent as if each individual publication or patent or patent application was specifically and individually indicated to be incorporated by reference in its entirety for all purposes.

1. A compound of Formula I 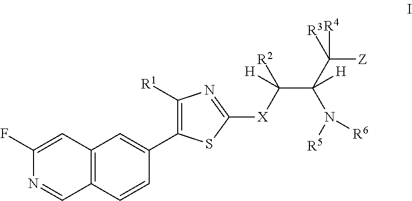
wherein:
X is selected from —N(R^{7a})— or —C(R^{7b}R^{7c})—;
R¹ is —H, halo, —OR⁸, C₁-C₆ alkyl, —(C₁-C₆ alkyl)-O—R⁸, —(C₁-C₆ haloalkyl)-O—R⁸, —(C₂-C₆ alkenyl)-O—R⁸, —(C₁-C₆ alkyl)N(R^{7d})₂, —(C₁-C₆ alkyl)aryl, —C(O)R⁸, —C(O)O—R⁸, —C(O)N(R^{7d})₂, —CHR¹¹—N(H)—R⁸, —CHR¹¹—O—R⁸, C₂-C₆ alkynyl, (C₂-C₆ alkynyl)-O—R⁸, —C≡N, —(C₂-C₆ alkynyl)(C₃-C₈ cycloalkyl), —(C₂-C₆ alkynyl)(C₅-C₈ cycloalkenyl),-(C₂-C₆ alkynyl)-N(R^{7d})S(O)₂—R⁸, aryl, heteroaryl, cycloalkyl, or heterocyclyl;
R² is —H, —OR⁸, —O—(C₁-C₆ alkyl)-O—R⁸, C₁-C₆ alkyl, C₁-C₆ alkenyl, —(C₁-C₆ alkyl)-O—R⁸, or —(C₁-C₆ alkyl)-O—C(O)—R⁸;
R³ is —H, or C₁-C₆ alkyl;
R⁴ is —H, —OR⁸, —O—(C₁-C₆ alkyl)-O—R⁸, C₁-C₆ alkyl, C₁-C₆ alkenyl, —(C₁-C₆ alkyl)-O—R⁸, —(C₁-C₆ alkyl)-O—C(O)—R⁸, —(C₁-C₆ alkyl)-S(O)—R⁸, or —(C₁-C₆ alkyl)-S(O)₂—R⁸;
R⁵ is —H, C₁-C₈ alkyl, —C(O)(CR⁹R¹⁰)ₜ)N(R^{7d})₂, —C(O)(CR⁹R¹⁰)ₜ(CR^{12a}R^{12b}R^{12c}), —C(O)₂(CR⁹R¹⁰)ₜ(CR^{12a}R^{12b}R^{12c}), —(CR⁹R¹⁰)ₜ(aryl), —(CR⁹R¹⁰)ₜ(heteroaryl), —(CR⁹R¹⁰)ₜ(cycloalkyl), or —(CR⁹R¹⁰)ₜ(heterocyclyl);
R⁶ is selected from —H, C₁-C₈ alkyl, —(C₁-C₆ alkyl)aryl, or —C(O)(C₁-C₆ alkyl);
R^{7a} is absent if X is —C(R^{7b}R^{7c})— or is selected from —H, C₁-C₈ alkyl, —(C₁-C₆ alkyl)aryl, —C(O)O—(C₁-C₆ alkyl), or —C(O)(C₁-C₆ alkyl);
R^{7b} and R^{7c} are absent if X is —N(R^{7a})— or are independently selected from H and (C₁-C₄)alkyl;
R^{7d} may be absent or, if present, is in each instance selected from —H, C₁-C₈ alkyl, —(C₁-C₆ alkyl)aryl, C₃-C₇ cycloalkyl, or —C(O)(C₁-C₆ alkyl);
R⁸ may be absent or, if present, is selected from —H, C₁-C₆ alkyl, C₁-C₆ haloalkyl, —(C₁-C₆ alkyl)aryl, aryl, heteroaryl, C₁-C₆ hydroxyalkyl, or —(C₁-C₆ alkyl)-O—(C₁-C₆ alkyl), cycloalkyl, or heterocyclyl;
R⁹, R¹⁰ and R¹¹ may be absent or, if present, are independently selected from —H, C₁-C₆ alkyl, or aryl;
R^{12a}, R^{12b}), and R^{12c}, may be absent or, if present, are in each instance independently selected from —H, or C₁-C₆ alkyl;
each t is independently selected from 0, 1, 2, or 3; and
Z is selected from aryl, heteroaryl, C₃-C₇ heterocyclyl comprising 1 or 2 heteroatoms selected from O, S, or N, or a C₃-C₇ cycloalkyl;
wherein each of the above alkyl, aryl, heteroaryl, cycloalkyl, and heterocyclyl moieties are optionally and independently substituted by 1-3 substituents selected from
amino,
aryl, heteroaryl, cycloalkyl, or heterocyclyl optionally substituted by 1-5 substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—(C₁-C₆ alkyl);
C₁-C₆ alkyl, C₁-C₆ haloalkyl, C₁-C₆ hydroxyalkyl, C₁-C₆ alkoxy, C₁-C₆ haloalkoxy, C₁-C₆ hydroxyalkoxy, C₁-C₆ alkylamino, C₂-C₆ alkenyl, or C₂-C₆ alkynyl, wherein each of which may be interrupted by one or more hetero atoms,
cyano,
halo,
hydroxyl,
nitro,
oxo,
—NH(CO)—O—(C₁-C₆ alkyl)aryl, —NH(CO)—O—(C₁-C₆ alkyl), —N(C₁-C₆ alkyl)(CO)—O—(C₁-C₆ alkyl)aryl, —N(C₁-C₆ alkyl)(CO)—O—(C₁-C₆ alkyl), —C(O)OH, —C(O)O(C₁-C₆ alkyl), —C(O)NH₂, —C(O)N(H)—(C₁-C₆ alkyl), —C(O)N(C₁-C₆ alkyl)₂, —NH(C₁-C₆ alkyl), —N(C₁-C₆ alkyl)₂, —(C₂-C₄ alkenyl)heterocyclyl, or —(C₂-C₄ alkenyl)cycloalkyl, or
—O-aryl;
or a pharmaceutically acceptable salt, stereoisomer, or mixture thereof.

2. The compound of claim 1, wherein X is —N(R^{7a})—.

3. The compound of claim 2, wherein R^{7a} is H.

4. The compound of claim 1, wherein X is —C(R^{7b}R^{7c})—.

5. The compound of claim 5, wherein R^{7b} and R^{7c} are both H.

6. The compound of claim 1, wherein R¹ is selected from —H, C₁-C₆ alkyl, —(C₁-C₆ alkyl)-O—R⁸, —C(O)O—R⁸, —C(O)N(R^{7d})₂, —CHR¹¹—O—R⁸, or C₂-C₆ alkynyl.

7. The compound of claim 6, wherein R¹ is —H.

8. The compound of claim 6, wherein R¹ is selected from —CH₂OCH₃, —CH₂OH, —C(O)₂Me, —C(O)N(H)(C₁-C₄ alkyl), —C(O)N(H)(C₃-C₇ cycloalkyl), or —C≡C—CH₃.

9. The compound of claim 1, wherein R⁵ and R⁶ are each H.

10. The compound of claim 9, wherein R² is H.

11. The compound of claim 9, wherein R³ is H.

12. The compound of claim 9, wherein R⁴ is —H.

13. The compound of claim 1, wherein R⁴ is —OR⁸, —O—(C₁-C₆ alkyl)-O—R⁸, C₁-C₆ alkyl, —(C₁-C₆ alkyl)-O—R⁸, or —(C₁-C₆ alkyl)-S(O)₂—R⁸.

14. The compound of claim 13, wherein R⁴ is selected from —CH₃, —CH₂OCH₃, —CH₂OH, —CH₂S(O)₂CH₃, —OH, or —OCH₂OCH₃.

15. The compound of claim 1, wherein Z is selected from optionally substituted phenyl, optionally substituted indolyl, optionally substituted naphthyl, optionally substituted pyridinyl, optionally substituted imidazolyl, optionally substituted pyrazolyl, optionally substituted pyrimidinyl, optionally substituted pyridinonyl, optionally substituted thiophenyl, or optionally substituted piperidinyl.

16. The compound of claim 15, wherein Z is selected from optionally substituted phenyl, optionally substituted pyridinyl, optionally substituted imidazolyl, optionally substituted pyrazolyl, optionally substituted pyrimidinyl, optionally substituted pyridinonyl, or optionally substituted piperidinyl

17. The compound of claim 15, wherein Z is selected from optionally substituted phenyl and optionally substituted pyridinyl.

18. The compound of claim 15, wherein Z is selected from phenyl, indolyl, naphthyl, pyridinyl, imidazolyl, pyrazolyl, pyrimidinyl, pyridinonyl, thiophenyl, or piperidinyl, each of which is optionally substituted with 1-3 substituents selected from —Cl, —F, —CF₃, —CF₂CH₃, —CH₃, —CHF₂, or —C(O)O(C₁-C₆ alkyl).

19. The compound of claim 1, wherein Z is selected from one of the following groups, wherein the wavy line indicates the point of attachment to the rest of the molecule

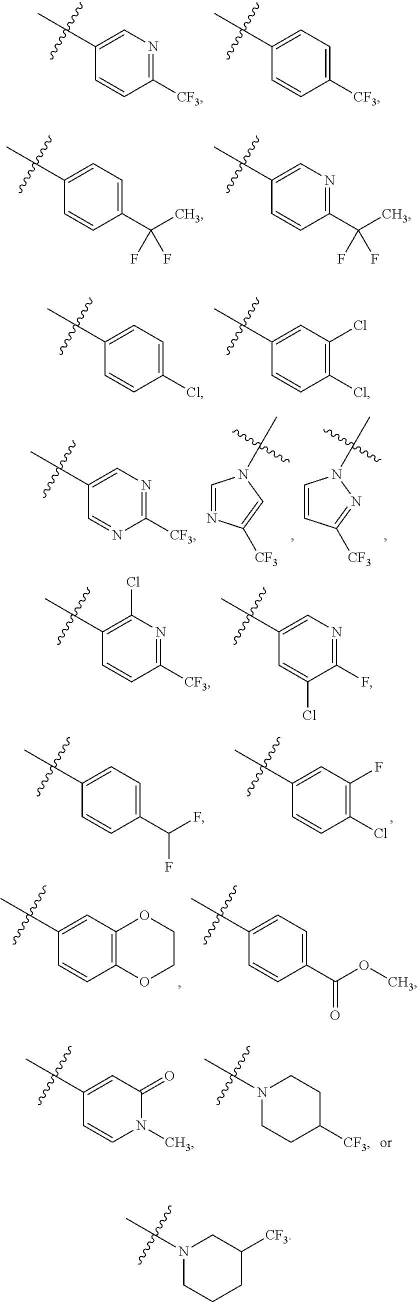

20. The compound of claim 1, wherein the compound of Formula I has the Formula IA

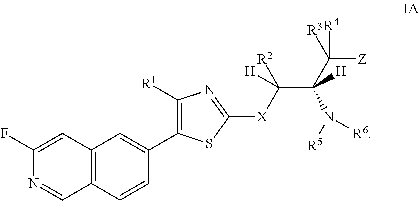

21. The compound of claim 1, wherein the compound of Formula I has the Formula IB

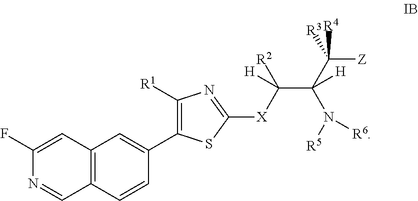

22. The compound of claim 1, wherein the compound of Formula I has the Formula IC

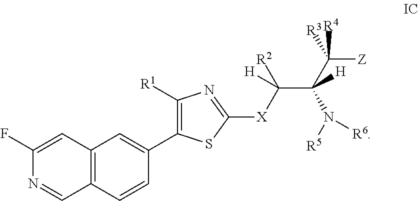

23. The compound of claim 1, wherein the compound of Formula I has the Formula ID

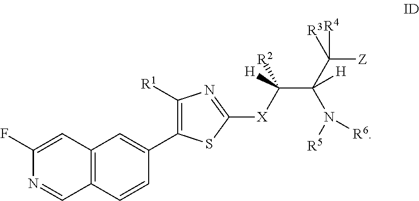

24. The compound of claim 1, wherein the compound of Formula I has the Formula IE

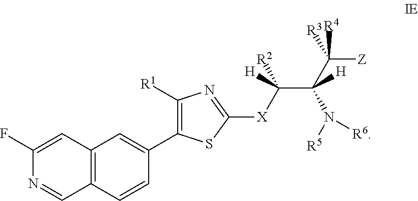

25. A pharmaceutical composition, comprising: a pharmaceutically-acceptable carrier and the compound of claim 1.

26. (canceled)

27. A method for treating cancer in a mammal in need thereof, the method comprising: administering to the mammal a therapeutically effective amount of the compound of claim 1.

28-39. (canceled)

40. The method of claim 27, wherein the mammal is a human cancer patient.
